(19) United States
(12) Patent Application Publication
(10) Pub. No.: US 2026/0108627 A1
(43) Pub. Date: Apr. 23, 2026

(54) COMPOSITIONS AND METHODS FOR TREATMENT OF HYPERCHOLESTEROLEMIA AND/OR CARDIOVASCULAR DISEASE

(71) Applicant: ACCUREDIT THERAPEUTICS (SUZHOU) CO., LTD., Suzhou, Jiangsu (CN)

(72) Inventors: Fuxin Shi, Cambridge, MA (US); Wenhu Cao, Suzhou, Jiangsu (CN); Ye Chen, Suzhou, Jiangsu (CN); Huanle Liu, Suzhou, Jiangsu (CN); Han Qiu, Suzhou, Jiangsu (CN); Leqi Liao, Suzhou, Jiangsu (CN)

(21) Appl. No.: 19/113,964

(22) PCT Filed: Sep. 21, 2023
(86) PCT No.: PCT/CN2023/120234

§ 371 Date: Mar. 21, 2025

(30) Foreign Application Priority Data
Sep. 22, 2022 (WO) PCT/CN2022/120376

(51) Int. Cl. A61K 48/00; A61K 9/1271; A61K 9/51; A61P 3/06; C12N 9/22; C12N 15/11; C12N 15/88

(52) U.S. Cl. CPC A61K 48/005; A61K 9/1271; A61K 9/5123; A61K 48/0083; A61P 3/06; C12N 9/226; C12N 15/111; C12N 15/88; C12N 2310/20

(57) ABSTRACT

Compositions and methods are described herein for treating subjects having hypercholesterolemia and/or cardiovascular disease.

### CROSS REFERENCE TO RELATED APPLICATIONS

This application is a National Phase application of and claims the benefit of priority under 35 U.S.C. § 371 to International Application No. PCT/CN2023/120234, filed Sep. 21, 2023, which claims priority to PCT/CN2022/120376, filed Sep. 22, 2022, entitled COMPOSITIONS AND METHODS FOR TREATMENT OF HYPERCHOLESTEROLEMIA AND/OR CARDIOVASCULAR DISEASE, the contents of which are incorporated by reference in their entirety herein.

### TECHNICAL FIELD

This disclosure relates to compositions and methods for the treatment of hypercholesterolemia and/or cardiovascular disease associated with proprotein convertase subtilisin/kexin type 9 (PCSK9).

### SEQUENCE LISTING

The instant application contains a Sequence Listing which has been submitted herewith and is hereby incorporated by reference in its entirely. Said .xml copy, created on Oct. 8, 2023 is named 53333-0005WO1, and is 1,172,853 bytes in size.

### BACKGROUND

Proprotein convertase subtilisin/kexin type 9 (PCSK9) is a serine protease that plays a regulatory role in cholesterol homeostasis, mainly by reducing both hepatic and extrahepatic low-density lipoprotein (LDL) receptor (LDLR) levels on the plasma membrane, thereby increasing plasma LDL cholesterol. PCSK9 is ubiquitously expressed in many tissues and cell types, but is expressed most abundantly in liver, small intestine, and kidney. PCSK9 is also highly expressed in arterial walls such as endothelium, smooth muscle cells, and macrophages, with a local effect that can regulate vascular homeostasis and atherosclerosis. PCSK9 binds to the receptor for LDL particles, which typically transport 3,000 to 6,000 fat molecules (including cholesterol) per particle, within extracellular fluid. The LDLR, on liver and other cell membranes, binds and initiates ingestion of LDL-particles from extracellular fluid into cells, thus reducing LDL particle concentrations. If PCSK9 activity is inhibited, for example, by mutation or by pharmacological intervention, more LDLRs are recycled and are present on the surface of cells to remove LDL-particles from the extracellular fluid. Therefore, inhibiting PCSK9 or reducing PCSK9 abundance can lower blood LDL-particle concentrations.

In human patients, variants of PCSK9 can reduce or increase circulating cholesterol. For example, hypercholesterolemia-associated gain-of-function PCSK9 mutations (e.g., R218S, F216L, and D374Y) resulted in total or partial loss of processing of mature PCSK9 at the furin cleavage motif RFHR (SEQ ID NO: 974). In contrast, the hypocholesterolemia-associated loss-of-function PCSK9 mutations (e.g., A443T and C679X) resulted in abnormal subcellular localization and enhanced susceptibility to furin cleavage (A443T) or to the inability of PCSK9 to exit the endoplasmic reticulum (C679X).

Accordingly, the potential use of PCSK9 inhibitors for the treatment of hypercholesterolemia has been explored. Antibody-based therapeutics alirocumab and evolocumab have been studied in phase III clinical trials. Additionally, RNAi-based therapeutics for the inhibition of PCSK9 have been studied. While results for these PCSK9-inhibiting therapeutics show encouraging results, a need exists for treatments that can produce long-lasting inhibition of PCSK9 for the treatment of hypercholesterolemia and cardiovascular disease.

### SUMMARY

This disclosure relates to compositions and methods to reduce the expression of the PCSK9 gene using CRISPR/Cas system, thereby substantially reducing or eliminating the production of mutant PCSK9 proteins or wild-type PCSK9 proteins in, for example, the liver, small intestine, kidney, or vascular tissues. This disclosure is based, at least in part, on the findings that novel guide RNA (gRNA) with high editing efficiency can knockout or knock down mutant or wildtype PCSK9 gene expression, thereby offering a long-lasting treatment for hypercholesterolemia and/or cardiovascular disease.

In a first aspect, this disclosure features a guide RNA comprising:
a) a sequence selected from SEQ ID NOs: 915, 933, 934, 1-296, 908-914, 916-932, and 935-940;
b) at least 15, 16, 17, 18, 19, or 20 contiguous nucleotides of a sequence selected from SEQ ID NOs: 915, 933, 934, 1-296, 908-914, 916-932, and 935-940; or
c) a sequence that is at least 90%, 91%, 92%, 93%, 94%, 95%, 96%, 97%, 98%, or 99% identical to a sequence selected from SEQ ID NOs: 915, 933, 934, 1-296, 908-914, 916-932, and 935-940.

In a second aspect, this disclosure features a vector comprising one or more nucleic acids encoding one or more guide RNAs, wherein the one or more guide RNAs comprise:
a) one or more sequences selected from SEQ ID NOs: 915, 933, 934, 1-296, 908-914, 916-932, and 935-940;
b) at least 15, 16, 17, 18, 19, or 20 contiguous nucleotides of one or more sequences selected from SEQ ID NOs: 915, 933, 934, 1-296, 908-914, 916-932, and 935-940;
c) one or more sequences that is at least 90%, 91%, 92%, 93%, 94%, 95%, 96%, 97%, 98%, or 99% identical to a sequence selected from SEQ ID NOs: 915, 933, 934, 1-296, 908-914, 916-932, and 935-940.

In a third aspect, this disclosure features composition comprising:
(i) a nucleic acid, or a vector comprising the nucleic acid encoding a guide RNA, wherein the guide RNA comprises:
   a) a sequence selected from SEQ ID NOs: 915, 933, 934, 1-296, 908-914, 916-932, and 935-940;
   b) at least 15, 16, 17, 18, 19, or 20 contiguous nucleotides of a sequence selected from SEQ ID NOs: 915, 933, 934, 1-296, 908-914, 916-932, and 935-940; or
   c) a sequence that is at least 90%, 91%, 92%, 93%, 94%, 95%, 96%, 97%, 98%, or 99% identical to a sequence selected from SEQ ID NOs: 915, 933, 934, 1-296, 908-914, 916-932, and 935-940; and
(ii) an RNA-guided DNA binding agent, a nucleic acid encoding an RNA-guided DNA binding agent, or a vector comprising the nucleic acid encoding an RNA-guided DNA binding agent.

In a fourth aspect, the disclosure features a method of modifying the human proprotein convertase subtilisin/kexin type 9 (PCSK9) gene and/or inducing a double-stranded break (DSB) within the PCSK9 gene, comprising administering the composition of the disclosure to a cell, wherein the composition recognizes and cleaves a PCSK9 target sequence.

In a fifth aspect, the disclosure features a method of treating hypercholesterolemia and/or cardiovascular disease in a subject, a method of reducing LDL levels in the circulation of a subject, a method of reducing the risk of atherosclerosis in a subject, and/or a method of treating or preventing coronary artery disease in a subject comprising administering the composition of the disclosure to a cell of the subject in need thereof, wherein the composition recognizes and cleaves a PCSK9 target sequence, thereby reducing the expression and/or abundance of PCSK9 in cells of one or more tissues of the subject, reducing LDL levels in the circulation of the subject, reducing the risk of atherosclerosis in the subject, treating or preventing coronary artery disease in the subject in the subject, and/or treating hypercholesterolemia and/or cardiovascular disease in the subject.

In some embodiments, the RNA-guided DNA binding agent comprises a Cas nuclease or a Cas nickase. In some embodiments, the nucleic acid encoding the RNA-guided DNA binding agent is a Cas9 nucleic acid comprising the nucleic acid sequence set forth in SEQ ID NO: 902 or 903. In some embodiments, the nucleic acid encoding the RNA-guided DNA binding agent is a Cas9-encoding nucleic acid comprising the polynucleotide sequence set forth in one or more of SEQ ID NOs: 941-953, 954-960, and 963-972. In some embodiments, the RNA-guided DNA binding agent is a Cas9 comprising the amino acid sequence set forth in SEQ ID NO: 901. In some embodiments, the Cas nuclease is a Class 2 Cas nuclease. In some embodiments, the Cas nuclease is Cas9, Cpf1, C2c1, C2c2, and C2c3, or a modified protein thereof. In some embodiments, the Cas nuclease is an S. pyogenes or an S. aureus Cas9 nuclease or a modified protein thereof. In some embodiments, the Cas nuclease is from a Type-II CRISPR/Cas system.

In some embodiments, the compositions of the disclosure are for use in editing of the proprotein convertase subtilisin/kexin type 9 (PCSK9) gene. In some embodiments, the editing is calculated as a percentage of a population of cells that is edited (percent editing). In some embodiments, between about 30% and 99% of the population of cells are edited. In some embodiments, the percent editing is between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the population of cells.

In some embodiments, the composition of the disclosure increases the abundance low-density lipoprotein receptors (LDLR) on the plasma membrane of cells in at least one tissue or organ. In some embodiments, the tissue or organ is liver, small intestine, kidney, or vascular tissue. In some embodiments, the composition of the disclosure decrease the amount of LDL cholesterol in the circulation of a subject. In some embodiments, the LDL cholesterol in the circulation is determined 8 weeks after administration of the composition. In some embodiments, the LDL cholesterol in the circulation is compared to a negative control or a level determined in the subject before administration of the composition. In some embodiments, the LDL cholesterol in the circulation is reduced by at least 20% relative to that in a corresponding negative control or a level determined in the subject before administration of the composition.

In some embodiments, the composition is administered or delivered at least once. In some embodiments, the administration or delivery occurs at an interval of (a) 1, 2, 3, 4, 5, 6, 7, 8, 9, 10, 11, 12, 13, 14, or 15 days; or (b) 1, 2, 3, 4, 5, 6, 7, 8, 9, 10, 11, 12, 13, 14, or 15 weeks; or (c) 1, 2, 3, 4, 5, 6, 7, 8, 9, 10, 11, 12, 13, 14, or 15 months; or (d) 1, 2, 3, 4, 5, 6, 7, 8, 9, or 10 years.

In some embodiments, the guide RNA is at least partially complementary to a target sequence present in the human PCSK9 gene. In some embodiments, the target sequence is in exon 1, 2, 3, 4, 5, 6, 7, 8, 9, 10, 11, or 12 of the human PCSK9 gene. In some embodiments, the guide RNA sequence is complementary to a target sequence in the positive strand of the PCSK9 gene. In some embodiments, the guide RNA sequence is complementary to a target sequence in the negative strand of PCSK9. In some embodiments, the first guide sequence is complementary to a first target sequence in the positive strand of the PCSK9 gene, and wherein the composition further comprises a second guide sequence that is complementary to a second target sequence in the negative strand of the PCSK9 gene.

In some embodiments, the guide RNA comprises a crRNA and further comprises a tracrRNA or a portion thereof, wherein the tracrRNA (trRNA) comprises the nucleotide sequence set forth in SEQ ID NO: 904 wherein the trRNA is operably linked to the crRNA.

In some embodiments, the guide RNA is a dual guide RNA (dgRNA). In some embodiments, the guide RNA is a single guide (sgRNA). In some embodiments, the guide RNA comprises at least one modification. In some embodiments, the at least one modification comprises a 2′-O-methyl (2′-O-Me) modified nucleotide, a phosphorothioate (PS) bond between nucleotides, a 2′-fluoro (2′-F) modified nucleotide, or a DNA-RNA hybrid. In some embodiments, the at least one modification comprises a modification at one or more of the first five nucleotides at the 5′ end of the guide RNA and/or one or more of the last five nucleotides at the 3′ end of the guide RNA. In some embodiments, the at least one modification comprises a modification of at least 50% of the nucleotides of the guide RNA.

In some embodiments, the sgRNA comprises a guide sequence that is at least 90% identical to a sequence selected from SEQ ID NOs: 915, 933, 934, 1-296, 908-914, 916-932, and 935-940. In some embodiments, the sgRNA comprises a nucleotide sequence set forth in any one of SEQ ID NOs: 593-888. In some embodiments, the sgRNA comprises a nucleotide sequence that is at least 90% identical to the nucleotide sequence set forth in any one of SEQ ID NOs: 593-888.

In some embodiments, the guide RNA is associated with a lipid nanoparticle (LNP). In some embodiments, the composition is a pharmaceutical formulation and further comprises a pharmaceutically acceptable carrier.

In some embodiments, the composition reduces the risk of or prevents cardiovascular disease in a subject. In some embodiments, the composition reduces the risk of or prevents atherosclerosis in a subject. In some embodiments, the composition reduces the risk of or prevents the formation of atherosclerotic plaques in the vascular tissue of a subject.

In some embodiments, administering the composition leads to a deletion or insertion of one or more nucleotide(s) in the PCSK9 gene. In some embodiments, the deletion or insertion of a nucleotide(s) induces a frameshift or nonsense mutation in the PCSK9 gene. In some embodiments, a frameshift or nonsense mutation is induced in the PCSK9 gene of about 20% to about 30% of cells. In some embodiments, the cells are liver cells, kidney cells, intestinal epithelial cells, or vascular epithelial cells. In some embodiments, a deletion or insertion of a nucleotide(s) occurs in the PCSK9 gene at least 50-fold or more than in off-target sites.

In some embodiments, the composition reduces levels of PCSK9 proteins in the cells of the subject. In some embodiments, the levels of PCSK9 proteins are reduced by at least 30%. In some embodiments, the levels of PCSK9 proteins are measured in serum, plasma, blood, or cerebral spinal fluid. In some embodiments, the levels of PCSK9 proteins are measured in liver cells, kidney cells, intestinal epithelial cells, or vascular epithelial cells.

In some embodiments, the composition increases the levels of LDL receptor proteins on the plasma membrane of cells of the subject. In some embodiments, the levels of LDL receptor proteins are increased by at least 10%. In some embodiments, the levels of LDL receptor proteins are measured liver cells, kidney cells, intestinal epithelial cells, or vascular epithelial cells.

In some embodiments, the composition decreases the levels of LDL cholesterol in the circulation of the subject. In some embodiments, the levels of LDL cholesterol are measured in serum, plasma, or blood.

In some embodiments, the subject has hypercholesterolemia, familial hypercholesterolemia, or a family history of hypercholesterolemia. In some embodiments, the subject has cardiovascular disease, familial cardiovascular disease, or a family history of cardiovascular disease. In some embodiments, the subject has atherosclerosis, familial atherosclerosis, or a family history of atherosclerosis. In some embodiments, the subject exhibits cardiovascular symptoms of atherosclerotic plaques. In some embodiments, the subject exhibits cardiovascular symptoms of coronary artery disease.

In some embodiments, the subject expresses a wild-type PCSK9 or a PCSK9 having one or more mutations selected from the group consisting of the following mutations: R46L, S127R, Y142X, R218S, F216L, D374Y, A443T, or C679X. In some embodiments, the subject is homozygous for wild-type PCSK9.

In some embodiments, after administration of the composition of the disclosure, the subject exhibits an improvement, stabilization, or slowing of change in symptoms of hypercholesterolemia. In some embodiments, the improvement, stabilization, or slowing of change in hypercholesterolemia is measured using a lipid panel. In some embodiments, the subject exhibits an improvement, stabilization, or slowing of change in symptoms of hypercholesterolemia, cardiovascular disease, coronary artery disease, or atherosclerosis.

In some embodiments, the composition or pharmaceutical formulation is administered via a viral vector. In some embodiments, the composition or pharmaceutical formulation is administered via lipid nanoparticles.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the methods and materials described herein will be apparent from the following detailed description and figures, and from the claims.

### BRIEF DESCRIPTION OF DRAWINGS

FIG. 1 shows a plot of editing efficiency for various sgRNAs targeting the human PCSK9 gene in HepG2 cells.

FIG. 2 shows a plot of the EC50 and maximum editing of human PCSK9 sgRNAs delivered with Cas9 mRNA in Cos-7 cells.

FIG. 3 shows a plot of the EC50 and maximum editing of human PCSK9 sgRNAs delivered with Cas9 mRNA in primary cynomolgus liver hepatocytes (PCH) cells.

FIG. 4 shows a plot of the EC50 and maximum editing of human PCSK9 sgRNAs delivered with different Cas9 mRNAs comprising various engineered untranslated regions (UTRs) in Huh7 cells.

FIG. 5 shows a plot of the EC50 and maximum editing of human PCSK9 sgRNAs delivered with Cas9 mRNAs comprising various engineered coding sequences in Huh7 cells.

### DETAILED DESCRIPTION

This disclosure features compositions and methods for editing the human proprotein convertase subtilisin/kexin type 9 (PCSK9) gene. The compositions and methods described herein are for treating subjects having hypercholesterolemia and/or cardiovascular disease associated with PCSK9.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

### Definitions

Unless stated otherwise, the following terms and phrases as used herein are intended to have the following meanings:

As used herein, the term “nucleic acid” refers to a multimeric compound that has nucleosides or nucleoside analogs which have nitrogenous heterocyclic bases or base analogs linked together along a backbone, including conventional RNA, DNA, mixed RNA-DNA, and polymers that are analogs thereof. The terms “nucleic acid,” “polynucleotide,” “nucleotide,” “nucleotide sequence,” and “oligonucleotide” are used interchangeably. They refer to a polymeric form of nucleotides of any length, either deoxyribonucleotides or ribonucleotides, or analogs thereof. The following are non-limiting examples of nucleic acids: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A nucleic acid backbone can be made up of a variety of linkages, including one or more of sugar-phosphodiester linkages, peptide-nucleic acid bonds (“peptide nucleic acids” or PNAs such as those described in International Patent Publication No. WO1995032305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 06-methylguanine, 4-thio-pyrimidines, 4-amino-pyrimidines, 4-dimethylhydrazine-pyrimidines, and 04-alkyl-pyrimidines; (See e.g., U.S. Pat. No. 5,378,825 and International Patent Publication No. WO1993013121). For general discussion, see The Biochemistry of the Nucleic Acids 5-36, Adams et al., ed., 11th ed., 1992). Nucleic acids can include one or more “abasic” residues where the backbone includes no nitrogenous base for position(s) of the polymer (See e.g., U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42): 13233-41). RNA and DNA have different sugar moieties and can differ by the presence of uracil or analogs thereof in RNA and thymine or analogs thereof in DNA.

As used herein, the term “guide RNA” refer to the combination of a CRISPR RNA (crRNA) and a tracr RNA (trRNA). “Guide RNA” can be used interchangeably with “gRNA,” or “guide”. The crRNA and trRNA may be associated as a single RNA molecule (single guide RNA, sgRNA) or in two separate RNA molecules (dual guide RNA, dgRNA). “Guide RNA” or “gRNA” can refer to each type, i.e., sgRNA or dgRNA. The trRNA may be a naturally-occurring sequence, or a trRNA sequence can have modifications or variations compared to naturally-occurring sequences. Guide RNAs can include modified RNAs as described herein.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be about 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the guide sequence and the targeting sequence may be 100% complementary or identical in sequence to one another. In other embodiments, the guide sequence and the targeting sequence may contain at least one mismatch. For example, the guide sequence and the targeting sequence may contain 1, 2, 3, or 4 mismatches, where the total length of the targeting sequence is at least 17, 18, 19, 20 or more base pairs. In some embodiments, the guide sequence and the targeting sequence may contain 1-4 mismatches where the guide sequence comprises at least 17, 18, 19, 20 or more nucleotides. In some embodiments, the guide sequence and the targeting sequence may contain 1, 2, 3, or 4 mismatches where the guide sequence comprises at least 20 nucleotides.

In some embodiments, the guide RNA comprises a crRNA that has a guide sequence (e.g., a guide sequence from Table 4) and further includes a nucleotide sequence GUU UUA GAG CUA UGC UGU UUU G (SEQ ID NO: 889), wherein SEQ ID NO: 889 follows the guide sequence at its 3′ end. In some embodiments, the crRNA is any crRNA selected from the nucleotide sequences set forth in SEQ ID NOs: 297-592. In some embodiments, the guide RNA comprises any one of the crRNA nucleotide sequences set forth in SEQ ID NOs: 297-592.

In some embodiments, the guide RNA comprises a crRNA and further includes a tracrRNA (trRNA) sequence comprising the nucleotide sequence set forth in SEQ ID NO: 904 or a portion thereof. AAC AGC AUA GCA AGU UAA AAU AAG GCU AGU CCG UUA UCA ACU UGA AAA AGU GGC ACC GAG UCG GUG CUU UUU UU (SEQ ID NO: 904).

In some embodiments, the guide RNA comprises additional nucleotides to form a sgRNA, e.g., with the following exemplary nucleotide sequence following the 3′ end of the guide sequence: GUU UUA GAG CUA GAA AUA GCA AGU UAA AAU AAG GCU AGU CCG UUA UCA ACU UGA AAA AGU GGC ACC GAG UCG GUG CUU UU (SEQ ID NO: 890) in the 5′ to 3′ orientation. In some embodiments, the sgRNA is any sgRNA selected from the nucleotide sequences set forth in SEQ ID NOs: 593-888. In some embodiments, the gRNA comprises any one of the nucleotide sequences set forth in SEQ ID NOs: 593-888. In some embodiments, the gRNA consists of any one of the nucleotide sequences set forth in SEQ ID NOs: 593-888.

In some embodiments, the guide RNA comprises a portion of SEQ ID NO: 889 covalently linked to a trRNA. For instance, the guide RNA comprises a guide sequence (e.g., a guide sequence from Table 4) linked to GUUUUAGAGCUA (SEQ ID NO: 905) further linked to a trRNA (SEQ ID NO: 904 or a portion thereof). For instance, the guide RNA comprises a guide sequence (e.g., a guide sequence from Table 4) linked to GUU UUA GAG CUA (SEQ ID NO: 905) further linked to the nucleotide sequence AUA GCA AGU UAA AAU AAG GCU AGU CCG UUA UCA ACU UGA AAA AGU GGC ACC GAG UCG GUG CUU UU (SEQ ID NO: 906).

Targeting sequences for Cas proteins include both the positive and negative strands of genomic DNA (i.e., the sequence given and the sequence's reverse complement), since the nucleic acid substrate for a Cas protein is double stranded. Accordingly, where a guide sequence is said to be “complementary to a target sequence”, it is to be understood that the guide sequence may direct a guide RNA to bind to the reverse complement of a target sequence. Thus, in some embodiments where the guide sequence binds the reverse complement of a target sequence, the guide sequence is identical to certain nucleotides of the target sequence (e.g., the target sequence not including the protospacer adjacent motif (PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents (such as those described in International Patent Application Nos. WO2020198697, incorporated herein in its entirety) include Cas nickases and inactivated forms thereof, such as dCas DNA binding agents”).

As used herein, the term “Cas” refers to any Cas protein that is operable for gene editing using a guide molecule. “Cas nuclease” also encompasses Cas nickases, and endonuclease-deficient or dead Cas (dCas) DNA binding agents. Cas nickases and dCas DNA binding agents can include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity, such as a Cas9 nuclease or a Cpf1 nuclease. Class 2 Cas nucleases include Class 2 Cas nickases (e.g., H840A, D10A, or N863A variants), which further have RNA-guided DNA nickase activity, and Class 2 dCas DNA binding agents, in which nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al, Cell, 163:1-13 (2015), is homologous to Cas9, and contains a RuvC-like nuclease domain. Cpf1 sequences of Zetsche are incorporated by reference in their entirety. See, e.g., Zetsche, Tables SI and S3. “Cas9” encompasses Spy Cas9, the variants of Cas9 listed herein, and equivalents thereof. See, e.g., Makarova et al, Nat Rev Microbiol, 13(11):722-36 (2015); Shmakov et al., Molecular Cell, 60:385-397 (2015).

dCas DNA binding agents can be used in CRISPR interference (CRISPRi) as well as CRISPR activation (CRISPRa). In CRISPRi, dCas9 binds to its DNA target but does not cleave it. Without being bound by theory, it is believed that the binding of Cas9 alone will prevent the cell's transcription machinery from accessing the promoter, hence inhibiting the gene expression. On the other hand, dCas9's ability to bind target DNA can be exploited for activation, i.e., CRISPRa. A transcriptional activator is fused to dCas9, which can activate gene expression without changing DNA sequence. In some embodiments, the dCas DNA binding agent is fused to a repressor, such as a Krüppel-associated box (KRAB).

“Modified uridine” is used herein to refer to a nucleoside including but not restricting to a thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton, e.g., Nl-methyl pseudouridine. In some embodiments, a modified uridine is any of a substituted uridine, pseudouridine, or a substituted pseudouridine.

As used herein, a first sequence is considered to “comprise a sequence that is at least X % identical to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) bind the same complement nucleotide(s) (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, the term “mRNA” refers to a polynucleotide that is RNA or modified RNA and includes an open reading frame that can be translated into a polypeptide (i.e., can serve as a substrate for translation by a ribosome and amino-acylated tRNAs). mRNA can include a phosphate-sugar backbone having ribose residues or analogs thereof, e.g., 2′-methoxy ribose residues. In some embodiments, the sugars of a nucleic acid phosphate-sugar backbone consist essentially of ribose residues, 2′-methoxy ribose residues, or a combination thereof.

As used herein, the term “PCSK9” refers to proprotein convertase subtilisin/kexin type 9, which is the expressed product of a PCSK9 gene. The human wild-type PCSK9 sequence is available at NCBI Gene ID: 255738; Ensembl: ENSG00000169174. The PCSK9 comprises four major components in the pre-processed protein: the signal peptide (amino acid residues 1-30); the N-terminal prodomain (residues 31-152); the catalytic domain (residues 153-425); and the C-terminal domain (residues 426-692), which is further divided into three modules (Du F, et al. Novel domain interaction regulates secretion of proprotein convertase subtilisin/kexin type 9 (PCSK9) protein. J Biol Chem. 2011 Dec. 16; 286(50):43054-61.). The PCSK9 gene is located at cytogenetic location 1p32.3 and comprises a total of 14 exons which may be alternatively spliced. The PCSK9 protein is a member of the subtilisin-like proprotein convertase family, which includes proteases that process protein and peptide precursors trafficking through regulated or constitutive branches of the secretory pathway. The encoded protein undergoes an autocatalytic processing event within its prosegment in the ER and is constitutively secreted as an inactive protease into the extracellular matrix and trans-Golgi network. It is expressed in liver, intestine, vascular epithelial and kidney tissues and escorts specific receptors for lysosomal degradation. It plays a role in cholesterol and fatty acid metabolism. Mutations in this gene have been associated with autosomal dominant familial hypercholesterolemia. Alternative splicing results in multiple transcript variants. As used herein, “mutant PCSK9” refers to a gene product of PCSK9 (i.e., the PCSK9 protein) having a change in the amino acid sequence of PCSK9 compared to the wild-type amino acid sequence of PCSK9. Mutant forms of PCSK9 associated with LDLR levels in patients include, e.g., R46L, S127R, Y142X, R218S, F216L, D374Y, A443T, and C679X.

As used herein, “low-density lipoprotein” (LDL) refers to particles comprising multiple proteins (e.g. about 80-100 proteins) that transfer lipids through aqueous fluid, thereby making lipids available to cells for receptor-mediated endocytosis. A single LDL particle can be about 220-275 angstroms in diameter, typically transporting about 3,000 to about 6,000 lipid molecules per particle, and varying in size according to the number and composition of lipid molecules contained within the particle. LDL particles can carry, for example, a mixture of cholesterol, phospholipids, and triglycerides. It is well known in the art that elevated levels of LDL measured in the blood is associated with increased risk of cardiovascular diseases.

As used herein, “low-density lipoprotein receptor” (LDLR) refers to a cell-surface receptor that mediates the endocytosis of LDL particles. LDLR recognizes, for example, apolipoprotein B100, which is embedded in the outer phospholipid layer of LDL particles. The LDLR protein is encoded by the LDLR gene on chromosome 19 of the human genome. It is well known in the art that LDLR function is associated with cholesterol metabolism and that disruption of LDLR can increase risk for disease related to cholesterol metabolism.

As used herein, “hypercholesterolemia” refers to a subject having levels of cholesterol in the blood that are higher than normal levels. Normal blood cholesterol level is a number derived by laboratory analysis. A normal or desirable cholesterol level is defined as less than 200 mg of cholesterol per deciliter of blood (mg/dL). Blood cholesterol is considered to be borderline when it is in the range of 200 to 239 mg/dL. Elevated cholesterol level is 240 mg/dL or above, however, there is no absolute cutoff between normal and abnormal cholesterol levels, and values must be considered in relation to other health conditions and risk factors. Elevated blood cholesterol is considered to be hypercholesterolemia.

As used herein, “familial hypercholesterolemia” refers to a hereditary form of hypercholesterolemia that may be caused by, for example, an elevated polygenic risk for hypercholesterolemia or an inherited single-gene mutation that increases risk for hypercholesterolemia. It is known in the art that familial hypercholesterolemia may be inherited, for example, in an autosomal dominant or autosomal recessive pattern.

As used herein, “atherosclerosis” refers to the accumulation of fats, cholesterol and other substances in and on the arterial walls. This buildup is called plaque. The plaque can cause arteries to narrow, blocking blood flow. The plaque can also burst, leading to a blood clot.

As used herein, the term “pathological mutation” refers to a mutation that renders a gene product, for example the PCSK9 protein, more likely to cause, promote, contribute to, or fail to inhibit the development of a disease, such as hypercholesterolemia or cardiovascular disease.

As used herein, “indels” refer to insertion/deletion mutations consisting of a number of nucleotides that are either inserted or deleted into a polynucleotide sequence. Indels can occur, for example, at the site of double-stranded breaks (DSBs) in a target nucleic acid.

As used herein, “knockdown” refers to a decrease in expression of a particular gene product (e.g., protein, mRNA, or both). Knockdown of a protein can be measured either by detecting protein secreted by tissue or population of cells (e.g., in serum or cell media) or by detecting total cellular amount of the protein from a tissue or cell population of interest before and after knockdown. Methods for measuring knockdown of mRNA are known in the art, and include sequencing of mRNA isolated from a tissue or cell population of interest. In some embodiments, “knockdown” may refer to some loss of expression of a particular gene product, for example, a decrease in the amount of mRNA transcribed or a decrease in the amount of protein expressed or secreted by a population of cells (including in vivo populations such as those found in tissues).

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” or “treating” refers to an improvement, alleviation, or amelioration of at least one symptom of a disclosed condition upon administration or application of a therapeutic for the condition. The term includes inhibiting the condition or disease, arresting its development, relieving one or more symptoms of the condition or disease, curing the condition or disease, or preventing reoccurrence of one or more symptoms of the condition or disease. In the context of this disclosure, treatment of hypercholesterolemia and/or cardiovascular disease may comprise alleviating symptoms of hypercholesterolemia and/or cardiovascular disease. A treatment with the compositions of this disclosure is said to have “treated” the condition if the treatment results in a reduction in the pathology of the condition.

As used herein, the term “lipid nanoparticle” (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. See also, e.g., WO2015006747, WO2016118724, WO2021026358, WO2017173054 and WO2019067992, the contents of which are incorporated herein by reference in their entireties. Any LNP known to those of skill in the art to be capable of delivering nucleotides to subjects may be utilized with the guide RNAs and the nucleic acid encoding an RNA-guided DNA binding agent described herein.

As used herein, the term “pharmaceutically acceptable” means a biologically acceptable formulation, gaseous, liquid or solid, or mixture thereof, which is suitable for one or more routes of administration, in vivo delivery or contact. A “pharmaceutically acceptable” composition is a material that is not biologically or otherwise undesirable, e.g., the material may be administered to a subject without causing substantial undesirable biological effects.

As used herein, “infusion” refers to an active administration of one or more agents with an infusion time of, for example, between approximately 30 minutes and 12 hours. In some embodiments, the one or more agents comprise an LNP, e.g., having an mRNA encoding an RNA-guided DNA binding agent (such as Cas9) described herein and a gRNA described herein.

The term “about” or “approximately” means an acceptable error for a particular value as determined by one of ordinary skill in the art, which depends, in part, on how the value is measured or determined. In some embodiments, about refers to a difference of, for example, plus or minus less than 5% (e.g., plus or minus less than 1%, less than 0.5%, or less than 0.1%).

Numeric ranges are inclusive of the numbers defining the range. Measured and measureable values are understood to be approximate, taking into account significant digits and the error associated with the measurement. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

### Compositions and Methods Targeting the PCSK9 Gene

Disclosed herein are compositions for use in methods targeting the PCSK9 gene. The methods disclosed herein induce a double-stranded break (DSB) within the PCSK9 gene in a subject, modify the PCSK9 gene in a cell or subject, treat hypercholesterolemia and/or cardiovascular disease associated with PCSK9 in a subject, reduce PCSK9 abundance in the cells of a subject, increase the abundance of LDLR on the surface of cells of a subject, and/or reduce LDL levels in the circulation of a subject. In some embodiments, the disclosed compositions and methods inhibit the transcription of the PCSK9 gene and translation of the PCSK9 protein, thereby preventing the accumulation of PCSK9 in tissues. In general, the disclosed compositions comprise a guide RNA targeting PCSK9 (itself or in a vector), and an RNA-guided DNA binding agent, or a nucleic acid encoding an RNA-guided DNA binding agent (e.g., a CRISPR/Cas system). The subjects treated with such methods and compositions may have wild-type or non-wild type PCSK9 gene sequences, such as, for example, subjects with hypercholesterolemia or familial hypercholesterolemia, wherein such patients may harbor inherited mutations of PCSK9. In some embodiments, the composition is administered by infusion for 0.5-6 hours. In some embodiments, the composition is administered by subcutaneous injection. In some embodiments, the composition is administered by intrathecal injection.

A. Guide RNA (gRNAs)

The guide RNA used in the disclosed methods and compositions comprises a guide sequence targeting the PCSK9 gene. Exemplary guide sequences targeting the PCSK9 gene are shown in Table 4 as SEQ ID NOs: 1-296. Guide sequences useful in the guide RNA compositions and methods described herein are shown in Table 4 and throughout the application.

Each of the guide sequences in Table 4 may further comprise additional nucleotides to form a crRNA, e.g., with the following exemplary nucleotide sequence following the guide sequence at its 3′ end: GUU UUA GAG CUA UGC UGU UUU G (SEQ ID NO: 889). In the case of a sgRNA, the guide sequences of Table 4 may further comprise additional nucleotides to form a sgRNA, e.g., with the following exemplary nucleotide sequence following the 3′ end of the guide sequence, wherein the sgRNA has a custom-designed short crRNA component followed by the trRNA component: GUU UUA GAG CUA GAA AUA GCA AGU UAA AAU AAG GCU AGU CCG UUA UCA ACU UGA AAA AGU GGC ACC GAG UCG GUG CUU UU (SEQ ID NO: 890) in the 5′ to 3′ orientation.

SEQ ID NO: 890 is attached to the 3′ end of the guide sequence in the in the 5′ to 3′ orientation. sgRNA sequences useful in the compositions and methods of this disclosure are described in Table 5.

In some embodiments, the sgRNA is modified. In some embodiments, the sgRNA comprises the modification pattern shown below in SEQ ID NO: 907, where N is any natural or non-natural nucleotide, and where the totality of the N's comprise a guide sequence as described herein and the modified sgRNA comprises the following sequence: mN*mN*mN* NNN NNN NNN NNN NNN NNG UUU UAG AmGmCm UmAmGm AmAmAm UmAmGm CAA GUU AAA AUA AGG CUA GUC CGU UAU CAmAm CmUmUm GmAmAm AmAmAm GmUmGm GmCmAm CmCmGm AmGmUm CmGmGm UmGmCm U*mU*mU *mU (SEQ ID NO: 907), where “N” may be any natural or non-natural nucleotide; *=PS linkage; ‘m’=2′-O-Me nucleotide. The modifications remain as shown in SEQ ID NO: 907 despite the substitution of N's for the nucleotides of a guide. That is, although the nucleotides of the guide replace the “N's”, the first three nucleotides are 2′OMe modified and there are phosphorothioate linkages between the first and second nucleotides, the second and third nucleotides and the third and fourth nucleotides.

In some embodiments, the gRNA sequence has the modification pattern described in WO2016164356 and WO2016089433, each of which is incorporated herein in its entirety.

In some embodiments, the gRNA comprises a guide sequence that direct an RNA-guided DNA binding agent, which can be a nuclease (e.g., a Cas nuclease such as Cas9), to a target DNA sequence in PCSK9. The gRNA includes a crRNA having a guide sequence shown in Table 4. The gRNA includes a guide sequence having at least 15, 16, 17, 18, 19, or 20 contiguous nucleotides of any one of the guide sequences of SEQ ID NOs: 1-296 shown in Table 4. In some embodiments, the gRNA comprises a guide sequence having a sequence with about 75%, 80%, 85%, 90%, 91%, 92%, 93%, 94%, 95%, 96%, 97%, 98%, or 99% identity to at least 16, 17, 18, 19, or 20 contiguous nucleotides of any one of the guide sequences of SEQ ID NOs: 1-296 shown in Table 4. The gRNA may further comprise a tracr RNA (trRNA). In each composition and method embodiment described herein, the crRNA and trRNA may be associated as a single RNA (sgRNA), or may be on separate RNAs (dgRNA). In the context of sgRNAs, the crRNA and trRNA components may be covalently linked, e.g., via a phosphodiester bond or other covalent bond.

In each of the composition, use, and method embodiments described herein, the guide RNA may comprise two RNA molecules as a “dual guide RNA” or “dgRNA”. The dgRNA comprises a first RNA molecule comprising a crRNA having, e.g., a guide sequence shown in Table 4, and a second RNA molecule having a trRNA. The first and second RNA molecules may not be covalently linked, but may form a RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) having a guide sequence shown in Table 4 covalently linked to a trRNA. The sgRNA may comprise at least 15, 16, 17, 18, 19, or 20 contiguous nucleotides of any one of the guide sequences of SEQ ID NOs: 1-296 shown in Table 4. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures. In some embodiments, the composition comprises a gRNA that comprises a guide sequence that is at least 90%, 91%, 92%, 93%, 94%, 95%, 96%, 97%, 98%, or 99% identity to at least 16, 17, 18, 19, or 20 contiguous nucleotides of any one of the guide sequences of SEQ ID NOs: 1-296 shown in Table 4.

In some embodiments, the composition includes a guide RNA having a guide sequence selected from SEQ ID NOs: 1-296. The guide RNA having a guide sequence selected from SEQ ID NOs: 1-296 may be a chemically modified sgRNA, such as an end modified RNA. The guide RNA having a guide sequence selected from SEQ ID NOs: 1-296 may be dgRNA, such as a chemically modified dgRNA.

In other embodiments, the composition comprises at least one, e.g., at least two gRNAs having guide sequences selected from any two or more of the guide sequences of SEQ ID NOs: 1-296. In some embodiments, the composition comprises at least two gRNAs that each comprise a guide sequence at least 90%, 90%, 91%, 92%, 93%, 94%, 95%, 96%, 97%, 98%, or 99% identical to any of the nucleic acids of SEQ ID NOs: 1-296.

In some embodiments, the gRNA is a sgRNA having any one of SEQ ID NOs. 593-888. In some embodiments, the gRNA is a sgRNA having any one of SEQ ID NOs. 593-888, but without the modifications described in this disclosure (i.e., unmodified SEQ ID NOs. 593-888). In some embodiments, the gRNA is a sgRNA having any one of SEQ ID NOs. 593-888, but with at least one chemical modification. In some embodiments, the chemically modified SEQ ID NOs. 593-888 have 5′ and/or 3′ end modifications. In some embodiments, the gRNA is a sgRNA having any one of SEQ ID NOs. 593-888, but with the modification pattern shown in SEQ ID NO: 907.

The guide RNAs provided herein can be useful for recognizing (e.g., hybridizing to) a target sequence in the PCSK9 gene. For example, the PCSK9 target sequence may be recognized and cleaved by a provided Cas nuclease having a guide RNA. Thus, an RNA-guided DNA binding agent, such as a Cas nuclease, may be directed by a guide RNA to a target sequence of the PCSK9 gene, where the guide sequence of the guide RNA hybridizes with the target sequence and the RNA-guided DNA binding agent, such as a Cas nuclease, cleaves the target sequence.

In some embodiments, the selection of the one or more guide RNAs is determined based on target sequences within the PCSK9 gene. For example, the one or more guide RNAs is based on target sequences within any one of Exons 1-14 or the 5′ UTR or 3′ UTR of the PCSK9 gene.

Without being bound by any particular theory, mutations (e.g., frameshift mutations resulting from indels occurring as a result of a nuclease-mediated DSB) in certain regions of the gene may be less tolerable than mutations in other regions of the gene, thus, the location of a DSB is an important factor in the amount or type of protein knockdown that may result. In some embodiments, a gRNA complementary or having complementarity to a target sequence within the PCSK9 gene is used to direct the RNA-guided DNA binding agent to a particular location in the PCSK9 gene. In some embodiments, gRNAs are designed to have guide sequences that are complementary or have complementarity to target sequences in exon 1, exon 2, exon 3, exon 4, exon 5, exon 6, exon 7, exon 8, exon 9, exon 10, exon 11, exon 12, exon 13, or exon 14 of PCSK9. In some embodiments, a frameshift or nonsense mutation is induced in the PCSK9 gene of about 10%, about 15%, about 20%, about 25%, about 30% of cells to about 35% of the cells.

B. Modifications of gRNAs

In some embodiments, the gRNA is chemically modified. A gRNA having one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs having nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxy methyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e., at sugar modification. For example, the 2′ hydroxyl group (OH) can be modified, e.g., replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH2CH2O)nCH2CH2OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH2; alkylamino, dialkylamino, heterocyclyl, arylamino, diarylamino, heteroarylamino, or diheteroarylamino, ethylenediamine, or polyamino) and aminoalkoxy, O(CH2)n-amino, (wherein amino can be, e.g., NH2; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxy ethyl group (MOE), (OCH2CH2OCH3, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NEE; alkylamino, dialkylamino, heterocyclyl, arylamino, diarylamino, heteroarylamino, diheteroarylamino, or amino acid); NH(CH2CH2NH)nCH2CH2- amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with, e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g, arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments having an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a guide RNA molecule are deoxynucleotides.

In some embodiments, a gRNA can have one or more modifications. In some embodiments, the modification includes a 2′-O-methyl (2′-O-Me) modified nucleotide. In some embodiments, the modification includes a phosphorothioate (PS) bond between nucleotides.

In some embodiments, a gRNA is a DNA-RNA hybrid. In some embodiments, a guide RNA is a hybrid DNA-RNA guide. In some embodiments, the hybrid DNA-RNA guide includes a sequence selected from SEQ ID NOs: 908-940. In some embodiments, at least a portion of an sgRNA is a hybrid DNA-RNA guide. Exemplary DNA-RNA hybrid guide sequences are provided in Table 1 below. For the sequences provided in Table 1 below, a “d” indicates that the base following the “d” character is a deoxyribonucleotide, while characters that are not preceded by a “d” are ribonucleotides.

TABLE 1
Exemplary DNA-RNA hybrid guide sequences

| | | SEQ |
|---|---|---|
| | | ID |
| Modified Sequence | Sequence Name | NO |
| dCGUGCGCAGGAGGACGAGGA | P9-hc-023-seq2 | 908 |
| dCdGUGCGCAGGAGGACGAGGA | P9-hc-023-seq3 | 909 |
| CGUGCGCAGGAGGACGAGdGA | P9-hc-023-seq4 | 910 |
| CGUGCGCAGGAGGACGAGdGdA | P9-hc-023-seq5 | 911 |
| CdGUGCGCAGGAGGACGAGGdA | P9-hc-023-seq6 | 912 |
| dCdGUGCGCAGGAGGACGAGdGdA | P9-hc-023-seq7 | 913 |
| CGdTGCGCAGGAGGACGAGGA | P9-hc-023-seq8 | 914 |
| CGdTdGCGCAGGAGGACGAGGA | P9-hc-023-seq9 | 915 |
| CGdTdGdCGCAGGAGGACGAGGA | P9-hc-023-seq10 | 916 |
| CGdTdGdCdGCAGGAGGACGAGGA | P9-hc-023-seq11 | 917 |
| CGdTdGdCdGdCAGGAGGACGAGGA | P9-hc-023-seq12 | 918 |
| dGGUGCUAGCCUUGCGUUCCG | P9-hc-028-seq2 | 919 |
| GGUGCUAGCCUUGCGUUCCdG | P9-hc-028-seq3 | 920 |
| dGGUGCUAGCCUUGCGUUCCdG | P9-hc-028-seq4 | 921 |
| GGdTGCUAGCCUUGCGUUCCG | P9-hc-028-seq5 | 922 |
| GGdTGCdTAGCCUUGCGUUCCG | P9-hc-028-seq6 | 923 |
| GGCdTUCCUGGUGAAGAUGAG | P9-hc-082-seq2 | 924 |
| GGCdTdTCCUGGUGAAGAUGAG | P9-hc-082-seq3 | 925 |
| GGCdTdTCCdTGGUGAAGAUGAG | P9-hc-082-seq4 | 926 |
| GGCdTdTCCUGGdTGAAGAUGAG | P9-hc-082-seq5 | 927 |
| GGCdTdTCCUGGUGAAGAdTGAG | P9-hc-082-seq6 | 928 |
| GGCdTdTCCdTGGdTGAAGAUGAG | P9-hc-082-seq7 | 929 |
| dGUGCUCAACUGCCAAGGGAA | P9-hc-162-seq2 | 930 |
| GUGCUCAACUGCCAAGGGdAA | P9-hc-162-seq3 | 931 |
| dGUGCUCAACUGCCAAGGGdAA | P9-hc-162-seq4 | 932 |
| GdTGCUCAACUGCCAAGGGAA | P9-hc-162-seq5 | 933 |
| GdTGCdTCAACUGCCAAGGGAA | P9-hc-162-seq6 | 934 |
| GdTGCdTCAACdTGCCAAGGGAA | P9-hc-162-seq7 | 935 |
| GdTGUGGACCUCUUUGCCCCA | P9-hc-212-seq2 | 936 |
| GdTGdTGGACCUCUUUGCCCCA | P9-hc-212-seq3 | 937 |
| GdTGUGGACCdTCUUUGCCCCA | P9-hc-212-seq4 | 938 |
| GdTGUGGACCUCdTUUGCCCCA | P9-hc-212-seq5 | 939 |
| GdTGdTGGACCdTCUUUGCCCCA | P9-hc-212-seq6 | 940 |

The terms “mA,” “mC,” “mU,” or “mG” may be used to denote a nucleotide that has been modified with 2′-O-Me.

In some embodiments, the guide RNA includes a sgRNA having a guide sequence selected from SEQ ID NOs: 1-296 and the nucleotides of SEQ ID NO: 890, wherein the nucleotides of SEQ ID NO: 890 are on the 3′ end of the guide sequence, and wherein the guide sequence may be modified as shown in SEQ ID NO: 907.

Further examples of gRNA modifications are shown in e.g., WO2020198697, WO2016164356, and WO2016089433, incorporated by reference herein in its entirety.

### C. PAM Sequences

The PAM, also known as the protospacer adjacent motif, is a short specific sequence complementary to a portion of the gRNA, following the target DNA sequence that is essential for cleavage by Cas nuclease. The PAM is about 2-8 nucleotides downstream of the DNA sequence targeted by the guide RNA and the Cas cuts 3-4 nucleotides upstream of it. PAM sequences are exemplified below in Tables 2-3. A PAM in the context of this disclosure can be any one of the sequences in Tables 2-3 or any other sequence known in the art.

TABLE 2
PAM of synthetic spCas9 variants

| SpCas9 variant | Mutations (relative to SpCas9) | PAM sequence |
|---|---|---|
| D1135E variant | D1135E | NGG (SEQ ID NO: 891) |
| VQR variant | D1135V, R1335Q and T1337R | NGAN (SEQ ID NO: 892) or |
| | | NGNG (SEQ ID NO: 893) |
| EQR variant | D1135E, R1335Q and T1337R | NGAG (SEQ ID NO: 894) |
| VRER variant | D1135V, G1218R, R1335E and T1337R | NGCG (SEQ ID NO: 895) |

N is A, G, C or T.

TABLE 3
PAM of different Cas9 species

| Cas9 species | PAM sequence |
|---|---|
| Streptococcus pyogenes (Sp) | NGG (SEQ ID NO: 891) |
| Staphylococcus aureus (Sa) | NGRRN (SEQ ID NO: 896) |
| Neisseria meningitidis (Nm or Nme) | NNNNGATT (SEQ ID NO: 897) |
| Campylobacter jejuni (Cj) | NNNNRYAC (SEQ ID NO: 898) |
| Streptococcus thermophilus (St) | NNAGAAW (SEQ ID NO: 899) |
| Treponema denticola (Td) | NAAAAC (SEQ ID NO: 900) |

N is A, G, C or T.

### D. RNA-Guided DNA Binding Agent

Any nucleic acid having an open reading frame encoding an RNA-guided DNA binding agent, e.g. a Cas9 nuclease such as an S. pyogenes Cas9, may be combined in a composition or method with any of the gRNAs disclosed herein. In some embodiments, the nucleic acid having an open reading frame encoding an RNA-guided DNA binding agent is an mRNA. In some embodiments, the RNA-guided DNA binding agent is administered in its amino acid form, i.e., as a protein. In some embodiments, the nucleic acid encoding the RNA-guided DNA binding agent is part of a vector described herein. The nucleic acid encoding the RNA-guided DNA binding agent may have any of the characteristics described in WO2020198697, incorporated by reference herein in its entirety.

In some embodiments, the RNA-guided DNA binding agent for use in the compositions and methods described herein the RNA-guided DNA-binding agent is a Class 2 Cas nuclease. In some embodiments, the RNA-guided DNA-binding agent has double-strand endonuclease activity. In some embodiments, the RNA-guided DNA-binding agent comprises a Cas nuclease, such as a Class 2 Cas nuclease (which may be, e.g., a Cas nuclease of Type II, V, or VI). Class 2 Cas nucleases include, for example, Cas9, Cpf1, C2c1, C2c2, and C2c3 proteins and modifications thereof.

Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. Other examples of Cas nucleases include a Csm or Cmr complex of a type III CRISPR system or the Cas 10, Csm1, or Cmr2 subunit thereof; and a Cascade complex of a type I CRISPR system, or the Cas3 subunit thereof. In some embodiments, the Cas nuclease may be from a Type-IIA, Type-11B, or Type-IIC system For discussion of various CRISPR systems and Cas nucleases see, e.g., Makarova et al., Nat. Rev. Microbiol. 9:467-477 (2011); Makarova et al., Nat. Rev. Microbiol, 13:722-36 (2015); Shmakov et al., Molecular Cell, 60:385-397 (2015). In some embodiments, the RNA-guided DNA binding agent is a Cas nickase, e.g. a Cas9 nickase. In some embodiments, the RNA-guided DNA binding agent is an S. pyogenes Cas9 nuclease.

Non-limiting exemplary species that the RNA-guided DNA binding agent (e.g., the Cas nuclease) can be derived from include but are not limited to Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter Jejuni, Pasteurella multocida, Fibrobacter succinogene, Rhodospirillum rubrum, Nocardiopsis dassonvillei, Streptomyces pristinaespiralis, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter Zari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In some embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In some embodiments, the Cas nuclease is a Cpf1 nuclease from an Acidaminococcus or Lachnospiraceae.

Wild type Cas9 has two nuclease domains: RuvC and HNH. The RuvC domain cleaves the non-target DNA strand, and the HNH domain cleaves the target strand of DNA. In some embodiments, the Cas9 nuclease comprises more than one RuvC domain and/or more than one HNH domain. In some embodiments, the Cas9 nuclease is a wild type Cas9. In some embodiments, the Cas9 is capable of inducing a double strand break in target DNA. In certain embodiments, the Cas nuclease can cleave one or both strands of dsDNA. In some embodiments, the Cas nuclease can cleave a single strand of DNA. In some embodiments, the Cas nuclease may not have DNA nickase activity. An exemplary Cas9 amino acid sequence is provided as SEQ ID NO: 901.

(SEQ ID NO: 901)
MDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFKEDIQKAQVSG
QGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQS
FLKDDSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIME
RSSFFKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRIDLSQL
GGDGGGSPKKKRKV

An exemplary Cas9 mRNA ORF sequence, which includes start and stop codons, is provided as SEQ ID NO: 902.

(SEQ ID NO: 902)
AUGGACAAGAAGUACAGCAUCGGACUGGACAUCGGAACAAACAGCGUCGGAUGGGCAGUCAUCAC
AGACGAAUACAAGGUCCCGAGCAAGAAGUUCAAGGUCCUGGGAAACACAGACAGACACAGCAUCA
AGAAGAACCUGAUCGGAGCACUGCUGUUCGACAGCGGAGAAACAGCAGAAGCAACAAGACUGAAG
AGAACAGCAAGAAGAAGAUACACAAGAAGAAAGAACAGAAUCUGCUACCUGCAGGAAAUCUUCAG
CAACGAAAUGGCAAAGGUCGACGACAGCUUCUUCCACAGACUGGAAGAAAGCUUCCUGGUCGAAG
AAGACAAGAAGCACGAAAGACACCCGAUCUUCGGAAACAUCGUCGACGAAGUCGCAUACCACGAA
AAGUACCCGACAAUCUACCACCUGAGAAAGAAGCUGGUCGACAGCACAGACAAGGCAGACCUGAG
ACUGAUCUACCUGGCACUGGCACACAUGAUCAAGUUCAGAGGACACUUCCUGAUCGAAGGAGACC
UGAACCCGGACAACAGCGACGUCGACAAGCUGUUCAUCCAGCUGGUCCAGACAUACAACCAGCUG
UUCGAAGAAAACCCGAUCAACGCAAGCGGAGUCGACGCAAAGGCAAUCCUGAGCGCAAGACUGAG
CAAGAGCAGAAGACUGGAAAACCUGAUCGCACAGCUGCCGGGAGAAAAGAAGAACGGACUGUUCG
GAAACCUGAUCGCACUGAGCCUGGGACUGACACCGAACUUCAAGAGCAACUUCGACCUGGCAGAA
GACGCAAAGCUGCAGCUGAGCAAGGACACAUACGACGACGACCUGGACAACCUGCUGGCACAGAU
CGGAGACCAGUACGCAGACCUGUUCCUGGCAGCAAAGAACCUGAGCGACGCAAUCCUGCUGAGCG
ACAUCCUGAGAGUCAACACAGAAAUCACAAAGGCACCGCUGAGCGCAAGCAUGAUCAAGAGAUAC
GACGAACACCACCAGGACCUGACACUGCUGAAGGCACUGGUCAGACAGCAGCUGCCGGAAAAGUA
CAAGGAAAUCUUCUUCGACCAGAGCAAGAACGGAUACGCAGGAUACAUCGACGGAGGAGCAAGCC
AGGAAGAAUUCUACAAGUUCAUCAAGCCGAUCCUGGAAAAGAUGGACGGAACAGAAGAACUGCUG
GUCAAGCUGAACAGAGAAGACCUGCUGAGAAAGCAGAGAACAUUCGACAACGGAAGCAUCCCGCA
CCAGAUCCACCUGGGAGAACUGCACGCAAUCCUGAGAAGACAGGAAGACUUCUACCCGUUCCUGA
AGGACAACAGAGAAAAGAUCGAAAAGAUCCUGACAUUCAGAAUCCCGUACUACGUCGGACCGCUG
GCAAGAGGAAACAGCAGAUUCGCAUGGAUGACAAGAAAGAGCGAAGAAACAAUCACACCGUGGAA
CUUCGAAGAAGUCGUCGACAAGGGAGCAAGCGCACAGAGCUUCAUCGAAAGAAUGACAAACUUCG
ACAAGAACCUGCCGAACGAAAAGGUCCUGCCGAAGCACAGCCUGCUGUACGAAUACUUCACAGUC
UACAACGAACUGACAAAGGUCAAGUACGUCACAGAAGGAAUGAGAAAGCCGGCAUUCCUGAGCGG
AGAACAGAAGAAGGCAAUCGUCGACCUGCUGUUCAAGACAAACAGAAAGGUCACAGUCAAGCAGC
UGAAGGAAGACUACUUCAAGAAGAUCGAAUGCUUCGACAGCGUCGAAAUCAGCGGAGUCGAAGAC
AGAUUCAACGCAAGCCUGGGAACAUACCACGACCUGCUGAAGAUCAUCAAGGACAAGGACUUCCU
GGACAACGAAGAAAACGAAGACAUCCUGGAAGACAUCGUCCUGACACUGACACUGUUCGAAGACA
GAGAAAUGAUCGAAGAAAGACUGAAGACAUACGCACACCUGUUCGACGACAAGGUCAUGAAGCAG
CUGAAGAGAAGAAGAUACACAGGAUGGGGAAGACUGAGCAGAAAGCUGAUCAACGGAAUCAGAGA
CAAGCAGAGCGGAAAGACAAUCCUGGACUUCCUGAAGAGCGACGGAUUCGCAAACAGAAACUUCA
UGCAGCUGAUCCACGACGACAGCCUGACAUUCAAGGAAGACAUCCAGAAGGCACAGGUCAGCGGA
CAGGGAGACAGCCUGCACGAACACAUCGCAAACCUGGCAGGAAGCCCGGCAAUCAAGAAGGGAAU
CCUGCAGACAGUCAAGGUCGUCGACGAACUGGUCAAGGUCAUGGGAAGACACAAGCCGGAAAACA
UCGUCAUCGAAAUGGCAAGAGAAAACCAGACAACACAGAAGGGACAGAAGAACAGCAGAGAAAGA
AUGAAGAGAAUCGAAGAAGGAAUCAAGGAACUGGGAAGCCAGAUCCUGAAGGAACACCCGGUCGA
AAACACACAGCUGCAGAACGAAAAGCUGUACCUGUACUACCUGCAGAACGGAAGAGACAUGUACG
UCGACCAGGAACUGGACAUCAACAGACUGAGCGACUACGACGUCGACCACAUCGUCCCGCAGAGC
UUCCUGAAGGACGACAGCAUCGACAACAAGGUCCUGACAAGAAGCGACAAGAACAGAGGAAAGAG
CGACAACGUCCCGAGCGAAGAAGUCGUCAAGAAGAUGAAGAACUACUGGAGACAGCUGCUGAACG
CAAAGCUGAUCACACAGAGAAAGUUCGACAACCUGACAAAGGCAGAGAGAGGAGGACUGAGCGAA
CUGGACAAGGCAGGAUUCAUCAAGAGACAGCUGGUCGAAACAAGACAGAUCACAAAGCACGUCGC
ACAGAUCCUGGACAGCAGAAUGAACACAAAGUACGACGAAAACGACAAGCUGAUCAGAGAAGUCA
AGGUCAUCACACUGAAGAGCAAGCUGGUCAGCGACUUCAGAAAGGACUUCCAGUUCUACAAGGUC
AGAGAAAUCAACAACUACCACCACGCACACGACGCAUACCUGAACGCAGUCGUCGGAACAGCACU
GAUCAAGAAGUACCCGAAGCUGGAAAGCGAAUUCGUCUACGGAGACUACAAGGUCUACGACGUCA
GAAAGAUGAUCGCAAAGAGCGAACAGGAAAUCGGAAAGGCAACAGCAAAGUACUUCUUCUACAGC
AACAUCAUGAACUUCUUCAAGACAGAAAUCACACUGGCAAACGGAGAAAUCAGAAAGAGACCGCU
GAUCGAAACAAACGGAGAAACAGGAGAAAUCGUCUGGGACAAGGGAAGAGACUUCGCAACAGUCA
GAAAGGUCCUGAGCAUGCCGCAGGUCAACAUCGUCAAGAAGACAGAAGUCCAGACAGGAGGAUUC
AGCAAGGAAAGCAUCCUGCCGAAGAGAAACAGCGACAAGCUGAUCGCAAGAAAGAAGGACUGGGA
CCCGAAGAAGUACGGAGGAUUCGACAGCCCGACAGUCGCAUACAGCGUCCUGGUCGUCGCAAAGG
UCGAAAAGGGAAAGAGCAAGAAGCUGAAGAGCGUCAAGGAACUGCUGGGAAUCACAAUCAUGGAA
AGAAGCAGCUUCGAAAAGAACCCGAUCGACUUCCUGGAAGCAAAGGGAUACAAGGAAGUCAAGAA
GGACCUGAUCAUCAAGCUGCCGAAGUACAGCCUGUUCGAACUGGAAAACGGAAGAAAGAGAAUGC
UGGCAAGCGCAGGAGAACUGCAGAAGGGAAACGAACUGGCACUGCCGAGCAAGUACGUCAACUUC
CUGUACCUGGCAAGCCACUACGAAAAGCUGAAGGGAAGCCCGGAAGACAACGAACAGAAGCAGCU
GUUCGUCGAACAGCACAAGCACUACCUGGACGAAAUCAUCGAACAGAUCAGCGAAUUCAGCAAGA
GAGUCAUCCUGGCAGACGCAAACCUGGACAAGGUCCUGAGCGCAUACAACAAGCACAGAGACAAG
CCGAUCAGAGAACAGGCAGAAAACAUCAUCCACCUGUUCACACUGACAAACCUGGGAGCACCGGC
AGCAUUCAAGUACUUCGACACAACAAUCGACAGAAAGAGAUACACAAGCACAAAGGAAGUCCUGG
ACGCAACACUGAUCCACCAGAGCAUCACAGGACUGUACGAAACAAGAAUCGACCUGAGCCAGCUG
GGAGGAGACGGAGGAGGAAGCCCGAAGAAGAAGAGAAAGGUCUAG

An exemplary Cas9 mRNA coding sequence, suitable for inclusion in a fusion protein, is provided as SEQ ID NO: 903.

(SEQ ID NO: 903)
GACAAGAAGUACAGCAUCGGACUGGACAUCGGAACAAACAGCGUCGGAUGGGCAGUCAUCACAGA
CGAAUACAAGGUCCCGAGCAAGAAGUUCAAGGUCCUGGGAAACACAGACAGACACAGCAUCAAGA
AGAACCUGAUCGGAGCACUGCUGUUCGACAGCGGAGAAACAGCAGAAGCAACAAGACUGAAGAGA
ACAGCAAGAAGAAGAUACACAAGAAGAAAGAACAGAAUCUGCUACCUGCAGGAAAUCUUCAGCAA
CGAAAUGGCAAAGGUCGACGACAGCUUCUUCCACAGACUGGAAGAAAGCUUCCUGGUCGAAGAAG
ACAAGAAGCACGAAAGACACCCGAUCUUCGGAAACAUCGUCGACGAAGUCGCAUACCACGAAAAG
UACCCGACAAUCUACCACCUGAGAAAGAAGCUGGUCGACAGCACAGACAAGGCAGACCUGAGACU
GAUCUACCUGGCACUGGCACACAUGAUCAAGUUCAGAGGACACUUCCUGAUCGAAGGAGACCUGA
ACCCGGACAACAGCGACGUCGACAAGCUGUUCAUCCAGCUGGUCCAGACAUACAACCAGCUGUUC
GAAGAAAACCCGAUCAACGCAAGCGGAGUCGACGCAAAGGCAAUCCUGAGCGCAAGACUGAGCAA
GAGCAGAAGACUGGAAAACCUGAUCGCACAGCUGCCGGGAGAAAAGAAGAACGGACUGUUCGGAA
ACCUGAUCGCACUGAGCCUGGGACUGACACCGAACUUCAAGAGCAACUUCGACCUGGCAGAAGAC
GCAAAGCUGCAGCUGAGCAAGGACACAUACGACGACGACCUGGACAACCUGCUGGCACAGAUCGG
AGACCAGUACGCAGACCUGUUCCUGGCAGCAAAGAACCUGAGCGACGCAAUCCUGCUGAGCGACA
UCCUGAGAGUCAACACAGAAAUCACAAAGGCACCGCUGAGCGCAAGCAUGAUCAAGAGAUACGAC
GAACACCACCAGGACCUGACACUGCUGAAGGCACUGGUCAGACAGCAGCUGCCGGAAAAGUACAA
GGAAAUCUUCUUCGACCAGAGCAAGAACGGAUACGCAGGAUACAUCGACGGAGGAGCAAGCCAGG
AAGAAUUCUACAAGUUCAUCAAGCCGAUCCUGGAAAAGAUGGACGGAACAGAAGAACUGCUGGUC
AAGCUGAACAGAGAAGACCUGCUGAGAAAGCAGAGAACAUUCGACAACGGAAGCAUCCCGCACCA
GAUCCACCUGGGAGAACUGCACGCAAUCCUGAGAAGACAGGAAGACUUCUACCCGUUCCUGAAGG
ACAACAGAGAAAAGAUCGAAAAGAUCCUGACAUUCAGAAUCCCGUACUACGUCGGACCGCUGGCA
AGAGGAAACAGCAGAUUCGCAUGGAUGACAAGAAAGAGCGAAGAAACAAUCACACCGUGGAACUU
CGAAGAAGUCGUCGACAAGGGAGCAAGCGCACAGAGCUUCAUCGAAAGAAUGACAAACUUCGACA
AGAACCUGCCGAACGAAAAGGUCCUGCCGAAGCACAGCCUGCUGUACGAAUACUUCACAGUCUAC
AACGAACUGACAAAGGUCAAGUACGUCACAGAAGGAAUGAGAAAGCCGGCAUUCCUGAGCGGAGA
ACAGAAGAAGGCAAUCGUCGACCUGCUGUUCAAGACAAACAGAAAGGUCACAGUCAAGCAGCUGA
AGGAAGACUACUUCAAGAAGAUCGAAUGCUUCGACAGCGUCGAAAUCAGCGGAGUCGAAGACAGA
UUCAACGCAAGCCUGGGAACAUACCACGACCUGCUGAAGAUCAUCAAGGACAAGGACUUCCUGGA
CAACGAAGAAAACGAAGACAUCCUGGAAGACAUCGUCCUGACACUGACACUGUUCGAAGACAGAG
AAAUGAUCGAAGAAAGACUGAAGACAUACGCACACCUGUUCGACGACAAGGUCAUGAAGCAGCUG
AAGAGAAGAAGAUACACAGGAUGGGGAAGACUGAGCAGAAAGCUGAUCAACGGAAUCAGAGACAA
GCAGAGCGGAAAGACAAUCCUGGACUUCCUGAAGAGCGACGGAUUCGCAAACAGAAACUUCAUGC
AGCUGAUCCACGACGACAGCCUGACAUUCAAGGAAGACAUCCAGAAGGCACAGGUCAGCGGACAG
GGAGACAGCCUGCACGAACACAUCGCAAACCUGGCAGGAAGCCCGGCAAUCAAGAAGGGAAUCCU
GCAGACAGUCAAGGUCGUCGACGAACUGGUCAAGGUCAUGGGAAGACACAAGCCGGAAAACAUCG
UCAUCGAAAUGGCAAGAGAAAACCAGACAACACAGAAGGGACAGAAGAACAGCAGAGAAAGAAUG
AAGAGAAUCGAAGAAGGAAUCAAGGAACUGGGAAGCCAGAUCCUGAAGGAACACCCGGUCGAAAA
CACACAGCUGCAGAACGAAAAGCUGUACCUGUACUACCUGCAGAACGGAAGAGACAUGUACGUCG
ACCAGGAACUGGACAUCAACAGACUGAGCGACUACGACGUCGACCACAUCGUCCCGCAGAGCUUC
CUGAAGGACGACAGCAUCGACAACAAGGUCCUGACAAGAAGCGACAAGAACAGAGGAAAGAGCGA
CAACGUCCCGAGCGAAGAAGUCGUCAAGAAGAUGAAGAACUACUGGAGACAGCUGCUGAACGCAA
AGCUGAUCACACAGAGAAAGUUCGACAACCUGACAAAGGCAGAGAGAGGAGGACUGAGCGAACUG
GACAAGGCAGGAUUCAUCAAGAGACAGCUGGUCGAAACAAGACAGAUCACAAAGCACGUCGCACA
GAUCCUGGACAGCAGAAUGAACACAAAGUACGACGAAAACGACAAGCUGAUCAGAGAAGUCAAGG
UCAUCACACUGAAGAGCAAGCUGGUCAGCGACUUCAGAAAGGACUUCCAGUUCUACAAGGUCAGA
GAAAUCAACAACUACCACCACGCACACGACGCAUACCUGAACGCAGUCGUCGGAACAGCACUGAU
CAAGAAGUACCCGAAGCUGGAAAGCGAAUUCGUCUACGGAGACUACAAGGUCUACGACGUCAGAA
AGAUGAUCGCAAAGAGCGAACAGGAAAUCGGAAAGGCAACAGCAAAGUACUUCUUCUACAGCAAC
AUCAUGAACUUCUUCAAGACAGAAAUCACACUGGCAAACGGAGAAAUCAGAAAGAGACCGCUGAU
CGAAACAAACGGAGAAACAGGAGAAAUCGUCUGGGACAAGGGAAGAGACUUCGCAACAGUCAGAA
AGGUCCUGAGCAUGCCGCAGGUCAACAUCGUCAAGAAGACAGAAGUCCAGACAGGAGGAUUCAGC
AAGGAAAGCAUCCUGCCGAAGAGAAACAGCGACAAGCUGAUCGCAAGAAAGAAGGACUGGGACCC
GAAGAAGUACGGAGGAUUCGACAGCCCGACAGUCGCAUACAGCGUCCUGGUCGUCGCAAAGGUCG
AAAAGGGAAAGAGCAAGAAGCUGAAGAGCGUCAAGGAACUGCUGGGAAUCACAAUCAUGGAAAGA
AGCAGCUUCGAAAAGAACCCGAUCGACUUCCUGGAAGCAAAGGGAUACAAGGAAGUCAAGAAGGA
CCUGAUCAUCAAGCUGCCGAAGUACAGCCUGUUCGAACUGGAAAACGGAAGAAAGAGAAUGCUGG
CAAGCGCAGGAGAACUGCAGAAGGGAAACGAACUGGCACUGCCGAGCAAGUACGUCAACUUCCUG
UACCUGGCAAGCCACUACGAAAAGCUGAAGGGAAGCCCGGAAGACAACGAACAGAAGCAGCUGUU
CGUCGAACAGCACAAGCACUACCUGGACGAAAUCAUCGAACAGAUCAGCGAAUUCAGCAAGAGAG
UCAUCCUGGCAGACGCAAACCUGGACAAGGUCCUGAGCGCAUACAACAAGCACAGAGACAAGCCG
AUCAGAGAACAGGCAGAAAACAUCAUCCACCUGUUCACACUGACAAACCUGGGAGCACCGGCAGC
AUUCAAGUACUUCGACACAACAAUCGACAGAAAGAGAUACACAAGCACAAAGGAAGUCCUGGACG
CAACACUGAUCCACCAGAGCAUCACAGGACUGUACGAAACAAGAAUCGACCUGAGCCAGCUGGGA
GGAGACGGAGGAGGAAGCCCGAAGAAGAAGAGAAAGGUC

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the Cas nuclease is an engineered Cas nuclease. In some embodiments, the nucleic acid encoding the Cas nuclease includes one or more of an engineered 5′ untranslated region, 3′ untranslated region, coding region, or sequence encoding a poly A tail. In some embodiments, the nucleic acid encoding the Cas nuclease comprises a 5′ untranslated region (UTR) comprising any one of SEQ ID NOs: 941-947. In some embodiments, the nucleic acid encoding the Cas nuclease comprises a 3′ untranslated region (UTR) comprising any one of SEQ ID NOs: 948-953. In some embodiments, the nucleic acid encoding the Cas nuclease comprises a coding region (CDS) comprising any one of SEQ ID NOs: 954-960. In some embodiments, the nucleic acid encoding the Cas nuclease comprises a polynucleotide sequence encoding a poly A tail comprising any one of SEQ ID NOs: 963-972. In some embodiments, the engineered Cas nuclease is provided to cells with one or more guide RNAs selected from the group consisting of SEQ ID NOs: 915, 933, 934, 1-296, 908-914, 916-932, and 935-940.

E. Determination of Efficacy of gRNAs

In some embodiments, the efficacy of a gRNA is determined when delivered together with other components, e.g., a nucleic acid encoding an RNA-guided DNA binding agent such as any of those described herein. In some embodiments, the efficacy of a combination of a gRNA and a nucleic acid encoding an RNA-guided DNA binding agent is determined.

As described herein, use of an RNA-guided DNA nuclease and a guide RNA disclosed herein can lead to double-stranded breaks in the DNA, which can produce errors in the form of insertion/deletion (indel) mutations upon repair by cellular machinery. Many mutations due to indels alter the reading frame or introduce premature stop codons and, therefore, produce a non-functional protein.

In some embodiments, the efficacy of particular gRNAs or combinations is determined based on in vitro models. In some embodiments, the in vitro model is HEK293 cells. In some embodiments, the in vitro model is HUH7 human hepatocarcinoma cells. In some embodiments, the in vitro model is HepG2 cells. In some embodiments, the in vitro model is primary human hepatocytes. In some embodiments, the in vitro model is primary rodent hepatocytes. In some embodiments, the in vitro model is primary cynomolgus hepatocytes. With respect to using primary human hepatocytes, commercially available primary human hepatocytes can be used to provide greater consistency between experiments. In some embodiments, the number of off-target sites at which a deletion or insertion occurs in an in vitro model (e.g., in primary human hepatocytes) is determined, e.g., by analyzing genomic DNA from primary human hepatocytes transfected in vitro with Cas9 mRNA and the guide RNA In some embodiments, such a determination comprises analyzing genomic DNA from primary human hepatocytes transfected in vitro with Cas9 mRNA and the guide RNA. Exemplary procedures for such determinations are provided in the working examples below.

In some embodiments, the efficacy of particular gRNAs or combinations is determined across multiple in vitro cell models for a gRNA selection process. In some embodiments, a cell line comparison of data with selected gRNAs is performed. In some embodiments, cross screening in multiple cell models is performed.

In some embodiments, the efficacy of particular gRNAs or combinations is determined based on in vivo models. In some embodiments, the in vivo model is a rodent model. In some embodiments, the rodent model is a mouse, which expresses a human PCSK9 gene, which may be a mutant human PCSK9 gene. In some embodiments, the in vivo model is a non-human primate, for example, a cynomolgus monkey.

In some embodiments, the efficacy of a guide RNA or combination is measured by percent editing of PCSK9. In some embodiments, the percent editing of PCSK9 is compared to the percent editing necessary to achieve knockdown of PCSK9 protein, e.g., in the cells or cell culture media in the case of an in vitro model or in serum, cells, or tissue in the case of an in vivo model. In some embodiments, the percent editing is between 30 and 99% of the population of cells. In some embodiments, the percent editing is between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the population of cells. In some embodiments, the percent editing is between 30%-95%, 40%-90%, or 50%-85%, 30%-60%, 40%-80%, 50%-75%, 60%-90%.

In some embodiments, the efficacy of a guide RNA or combination is measured by the number and/or frequency of indels at off-target sequences within the genome of the target cell type. In some embodiments, efficacious guide RNAs and combinations are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., a hepatocyte), or which produce a frequency of off-target indel formation of <5% in a cell population and/or relative to the frequency of indel creation at the target site. In some embodiments, the disclosure provides guide RNAs and combinations which do not exhibit any off target indel formation in the target cell type (e.g., hepatocyte).

In some embodiments, guide RNAs and combinations are provided which produce indels at less than 20 off-target sites, e.g., as evaluated by one or more methods described herein. In some embodiments, guide RNAs and combinations are provided which produce indels at less than or equal to 4, 3, 2, or 1 off-target site(s), e.g., as evaluated by one or more methods described herein. In some embodiments, the off-target site(s) does not occur in a protein coding region in the target cell (e.g., hepatocyte) genome.

In some embodiments, detecting gene editing events, such as the formation of insertion/deletion (“indel”) mutations and homology directed repair (HDR) events in target DNA utilize linear amplification with a tagged primer and isolating the tagged amplification products (herein after referred to as “LAM-PCR,” or “Linear Amplification (LA)” method), as described in WO2018/067447 or Schmidt et al., Nature Methods 4:1051-1057 (2007).

In some embodiments, detecting gene editing events, such as the formation of insertion/deletion (“indel”) mutations and homology directed repair (HDR) events in target DNA, further comprises sequencing the linear amplified products or the further amplified products. Sequencing may comprise any method known to those of skill in the art, including, next generation sequencing, and cloning the linear amplification products or further amplified products into a plasmid and sequencing the plasmid or a portion of the plasmid. Exemplary next generation sequencing methods are discussed, e.g., in Shendure et al., Nature 26:1135-1145 (2008). In other aspects, detecting gene editing events, such as the formation of insertion/deletion (“indel”) mutations and homology directed repair (HDR) events in target DNA, further comprises performing digital PCR (dPCR) or droplet digital PCR (ddPCR) on the linear amplified products or the further amplified products, or contacting the linear amplified products or the further amplified products with a nucleic acid probe designed to identify DNA having Homology-directed repair (HDR) template sequence and detecting the probes that have bound to the linear amplified product(s) or further amplified product(s). In some embodiments, the method further comprises determining the location of the HDR template in the target DNA.

In certain embodiments, the method further comprises determining the sequence of an insertion site in the target DNA, wherein the insertion site is the location where the HDR template incorporates into the target DNA, and wherein the insertion site may include some target DNA sequence and some HDR template sequence.

In some embodiments, the amount of PCSK9 in cells (including cells from tissue) measures efficacy of a gRNA or combination. In some embodiments, the amount of PCSK9 in cells is measured using western blot. In some embodiments, the cell used is HUH7 cells. In some embodiments, the cell used is a primary human hepatocyte. In some embodiments, the cell used is a primary cell obtained from an animal. In some embodiments, the amount of PCSK9 is compared to the amount of glyceraldehyde 3-phosphate dehydrogenase GAPDH (a housekeeping gene) to control for changes in cell number.

In some embodiments, the amount of PCSK9 is reduced by between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the PCSK9 in cells detected in the subject before administration of the composition. In some embodiments, the amount of PCSK9 is reduced by between 30%-95%, 40%-90%, or 50%-85%, 30%-60%, 40%-80%, 50%-75%, or 60%-90% of the PCSK9 in cells detected in the subject before administration of the composition.

In some embodiments, the levels or amount of LDL in the circulation of a subject measure efficacy of a gRNA or combination. In some embodiments, the levels or amount of LDL in the circulation of a subject is measured by methods known in the art. For example, LDL in a subject can be measured using a lipid panel, which can include measurements of total cholesterol, LDL cholesterol, high-density lipoprotein (HDL) cholesterol, and triglycerides (Cooper G R, et al. Blood lipid measurements. Variations and practical utility. JAMA. 1992 Mar. 25; 267 (12):1652-60.).

In some embodiments, LDL in the circulation of a subject is reduced by between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the LDL in the circulation of a subject before administration of the composition. In some embodiments, the LDL in the circulation of a subject is reduced by between 30%-95%, 40%-90%, or 50%-85%, 30%-60%, 40%-80%, 50%-75%, or 60%-90% of the LDL in the circulation of a subject before administration of the composition.

### F. Methods of Treatment

In some embodiments, the disclosure provides a method of treating hypercholesterolemia and/or cardiovascular disease which includes administering a composition including a guide RNA having any one or more of the guide sequences of SEQ ID NOs: 1-296, or any one or more of the sgRNAs of SEQ ID NOs: 593-888, or any one or more of the crRNAs of SEQ ID NOs: 297-592. In some embodiments, the gRNAs have any one or more of the guide sequences of SEQ ID NOs: 1-296, or any one or more of the sgRNAs of SEQ ID NOs: 593-888 are administered to treat hypercholesterolemia and/or cardiovascular disease. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP having a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, the disclosure provides a method of inducing a double-stranded break (DSB) within the PCSK9 gene including administering a composition having a guide RNA as described herein, e.g. having any one or more guide sequences of SEQ ID NOs: 1-296, or any one or more of the sgRNAs of SEQ ID NOs: 593-888. In some embodiments, gRNAs such as any one or more of the guide sequences of SEQ ID NOs: 1-296 are administered to recognize and bind to the PCSK9 gene. The guide RNA is administered together with a nucleic acid (e.g., mRNA) or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease. In some embodiments, a method of inducing a double-stranded break (DSB) within the PCSK9 gene is provided comprising administering a composition comprising a guide RNA, such as a chemically modified guide RNA, comprising any one or more guide sequences of SEQ ID NOs: 1-296, or any one or more of the sgRNAs of SEQ ID NOs: 593-888. In some embodiments, any one or more of the sgRNAs of SEQ ID NOs: 593-888 or gRNAs comprising any one or more of the guide sequences of SEQ ID NOs: 1-296 are administered to induce a DSB in the PCSK9 gene. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, a method of modifying the PCSK9 gene is provided comprising administering a composition comprising a guide RNA as described herein, e.g. having any one or more of the guide sequences of SEQ ID NOs: 1-296, or any one or more of the sgRNAs of SEQ ID NOs: 593-888. In some embodiments, gRNAs comprising any one or more of the guide sequences of SEQ ID NOs: 1-296, or any one or more of the sgRNAs of SEQ ID NOs: 593-888, are administered to modify the PCSK9 gene. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, a method of modifying the PCSK9 gene is provided comprising administering a composition comprising a guide RNA comprising any one or more of the guide sequences of SEQ ID NOs: 1-296, or any one or more of the sgRNAs of SEQ ID NOs: 593-888. In some embodiments, gRNAs comprising any one or more of the guide sequences of SEQ ID NOs: 1-296, or any one or more of the sgRNAs of SEQ ID NOs: 593-888, are administered to modify the PCSK9 gene. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, a method of treating hypercholesterolemia and/or cardiovascular disease is provided comprising administering a composition comprising a guide RNA as described herein, e.g. having any one or more of the guide sequences of SEQ ID NOs: 1-296, or any one or more of the sgRNAs of SEQ ID NOs: 593-888. In some embodiments, gRNAs comprising any one or more of the guide sequences of SEQ ID NOs: 1-296, or any one or more of the sgRNAs of SEQ ID NOs: 593-888 are administered to treat hypercholesterolemia and/or cardiovascular disease. The guide RNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, the disclosure features a method of reducing LDL levels in the circulation of a subject including administering a guide RNA as described herein, e.g. having any one or more of the guide sequences of SEQ ID NOs: 1-296, or any one or more of the sgRNAs of SEQ ID NOs: 593-888. In some embodiments, gRNAs comprising any one or more of the guide sequences of SEQ ID NOs: 1-296 or any one or more of the sgRNAs of SEQ ID NOs: 593-888 are administered to reduce LDL levels in the circulation of a subject and/or prevent atherosclerosis in the vascular tissue of a subject. The gRNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, the disclosure features a method of reducing the risk of atherosclerosis in a subject including administering a guide RNA as described herein, e.g., comprising any one or more of the guide sequences of SEQ ID NOs: 1-296, or any one or more of the sgRNAs of SEQ ID NOs: 593-888. In some embodiments, gRNAs comprising any one or more of the guide sequences of SEQ ID NOs: 1-296, or any one or more of the sgRNAs of SEQ ID NOs: 593-888 are administered to reduce or prevent the incidence of atherosclerosis in the vascular tissue of a subject. The gRNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, the disclosure features a method of treating or preventing coronary artery disease in a subject including comprising administering a composition comprising a guide RNA as described herein, e.g. having any one or more of the guide sequences of SEQ ID NOs: 1-296, or any one or more of the sgRNAs of SEQ ID NOS: 593-888. In some embodiments, a method of treating or preventing coronary artery disease in a subject is provided comprising administering a composition comprising any one or more of the sgRNAs of SEQ ID NOs: 593-888. In some embodiments, gRNAs comprising any one or more of the guide sequences of SEQ ID NOs: 1-296 or any one or more of the sgRNAs of SEQ ID NOs: 593-888 are administered to treat or prevent coronary artery disease in a subject. The gRNA is administered together with a nucleic acid or vector described herein encoding an RNA-guided DNA nuclease such as a Cas nuclease (e.g., Cas9). The RNA-guided DNA nuclease may be an S. pyogenes Cas9. In particular embodiments, the guide RNA is chemically modified. In some embodiments, the guide RNA and the nucleic acid encoding an RNA-guided DNA nuclease are administered in an LNP described herein, such as an LNP comprising a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

In some embodiments, the gRNA includes a guide sequence of Table 4 together with an RNA-guided DNA nuclease such as a Cas nuclease translated from the nucleic acid induce DSBs, and non-homologous ending joining (NHEJ) during repair leads to a mutation in the PCSK9 gene. In some embodiments, NHEJ leads to a deletion or insertion of a nucleotide(s), which induces a frameshift or nonsense mutation in the PCSK9 gene.

In some embodiments, administering the guide RNA and nucleic acid encoding an RNA-guided DNA binding agent (e.g., in a composition provided herein) reduces the abundance of PCSK9 in the cells of the subject, for example in the liver, intestine, kidney, or vascular epithelial tissues of the subject, and therefore reduces the LDL levels in the circulation of the subject.

In some embodiments, reducing the abundance of PCSK9 in the cells of the subject comprises reducing the abundance of PCSK9 in the cells of one or more tissues of the subject, such as liver, intestine, kidney, or vascular epithelial tissue. In some embodiments, the vascular epithelial tissue comprises blood vessels, for example arteries. In some embodiments, reducing the abundance of PCSK9 in the cells of the subject is inferred based on measuring LDL levels in the circulation of the subject, for example by a lipid panel. In some embodiments, the abundance of PCSK9 in the cells of one or more tissues of the subject can result in reducing the levels of LDL in the circulation of the subject, e.g., as measured 8 weeks after administration of the composition.

In some embodiments, abundance of PCSK9 in the cells of the subject is reduced by between 30% and 35%, 35% and 40%, 40% and 45%, 45% and 50%, 50% and 55%, 55% and 60%, 60% and 65%, 65% and 70%, 70% and 75%, 75% and 80%, 80% and 85%, 85% and 90%, 90% and 95%, or 95% and 99% of the abundance of PCSK9 in the cells of the subject before administration of the composition. In some embodiments, abundance of PCSK9 in the cells of the subject is reduced by between 30%-95%, 40%-90%, or 50%-85%, 30%-60%, 40%-80%, 50%-75%, or 60%-90% of the abundance of PCSK9 in the cells of the subject before administration of the composition.

In some embodiments, the subject is mammalian. In some embodiments, the subject is human. In some embodiments, the subject is cow, pig, monkey, sheep, dog, cat, fish, or poultry. In some embodiments, the subject is a companion animal or a livestock animal.

In some embodiments, the use of one or more guide RNAs as described herein, e.g. including any one or more of the guide sequences in Table 4 (e.g., in a composition provided herein) and of a nucleic acid (e.g. mRNA) described herein encoding an RNA-guided DNA-binding agent is provided for the preparation of a medicament for treating a human subject having hypercholesterolemia and/or cardiovascular disease. The RNA-guided DNA-binding agent may be a Cas9, e.g. an S. pyogenes Cas9. In particular embodiments, the guide RNA is chemically modified.

In some embodiments, the composition that includes the guide RNA and nucleic acid is administered intravenously. In some embodiments, the composition that includes the guide RNA and nucleic acid is administered into the hepatic circulation.

In some embodiments, a single administration of a composition that includes a guide RNA and nucleic acid provided herein is sufficient to knock down expression of the mutant protein, for example mutant PCSK9. In some embodiments, a single administration of a composition that includes a guide RNA and nucleic acid provided herein is sufficient to knock out expression of the mutant protein in a population of cells. In other embodiments, more than one administration of a composition that includes a guide RNA and nucleic acid provided herein may be beneficial to maximize editing via cumulative effects. For example, a composition provided herein can be administered 2, 3, 4, 5, or more times, such as 2 times. Administrations can be separated by a period of time ranging from, e.g., 1 day to 2 years, such as 1 to 7 days, 7 to 14 days, 14 days to 30 days, 30 days to 60 days, 60 days to 120 days, 120 days to 183 days, 183 days to 274 days, 274 days to 366 days, or 366 days, 2 years, 5 years, or 10 years.

In some embodiments, a composition is administered in an effective amount in the range of 0.01 to 20 mg/kg (mpk), e.g., 0.01 to 0.1 mpk, 0.1 to 0.3 mpk, 0.3 to 0.5 mpk, 0.5 to 1 mpk, 1 to 2 mpk, 2 to 3 mpk, 3 to 5 mpk, 5 to 10 mpk, or 0.1, 0.2, 0.3, 0.5, 1, 2, 3, 5, 6, 8, 10, 15 or 20 mpk. In some embodiments, a composition is administered in the amount of 2-4 mg/kg, such as 2.5-3.5 mg/kg. In some embodiments, a composition is administered in the amount of about 3 mg/kg.

In some embodiments, the efficacy of treatment with the compositions described herein is assessed at 1 year, 2 years, 3 years, 4 years, 5 years, or 10 years after delivery. In some embodiments, efficacy of treatment with the compositions described herein is assessed by measuring levels of LDL in the circulation of the subject before and after treatment. In some embodiments, efficacy of treatment with the compositions assessed via a reduction of levels of LDL in the circulation of the subject is seen at 1 week, 2 weeks, 3 weeks, 4 weeks, 2 months, 3 months, 4 months, 5 months, 6 months, 7 months, 8 months, 9 months, 10 months, or at 11 months. In some embodiments, the levels of LDL in the circulation of the subject are reduced by at least 10%, at least 20%, at least 30%, at least 40%, at least 50%, at least 60%, at least 70%, at least 80%, at least 90%, at least 95%, or at least 99%.

In some embodiments, treatment slows, halts, or reverses disease progression.

In some embodiments, treatment slows or halts progression of cardiovascular disease. In some embodiments, treatment slows or halts progression of coronary artery disease. In some embodiments, treatment slows or halts progression of atherosclerosis. In some embodiments, treatment results in improvement, stabilization, or slowing of change in symptoms of cardiovascular disease.

In some embodiments, efficacy of treatment is measured by increased survival time of the subject.

### Additional Treatments

In some embodiments, combination therapies are described that include administering any one of the gRNAs as described herein, e.g., including any one or more of the guide sequences disclosed in Table 4 and a nucleic acid encoding an RNA-guided DNA-binding agent (e.g., in a composition provided herein) as described herein, such as a nucleic acid (e.g. mRNA) or vector described herein encoding an S. pyogenes Cas9, together with an additional therapy suitable for alleviating symptoms of hypercholesterolemia and/or cardiovascular disease.

In some embodiments, the additional therapy is a treatment for hypercholesterolemia and/or cardiovascular disease. In some embodiments, the treatment for hypercholesterolemia and/or cardiovascular disease is a statin, for example, atorvastatin, fluvastatin, lovastatin, pitavastatin, pravastatin, rosuvastatin, or simvastatin.

In some embodiments, the treatment for hypercholesterolemia and/or cardiovascular disease is a cholesterol absorption inhibitor, for example, ezetimibe. In some embodiments, the treatment for hypercholesterolemia and/or cardiovascular disease is bempedoic acid. In some embodiments, the treatment for hypercholesterolemia and/or cardiovascular disease is a bile-acid-binding resin, for example, cholestyramine, colesevelam, or colestipol.

In some embodiments, the combination therapy comprises administering any one of the gRNAs that includes any one or more of the guide sequences disclosed in Table 4 and a nucleic acid encoding an RNA-guided DNA-binding agent (e.g., in a composition provided herein) together with an antibody that targets and/or inhibits PCSK9. In some embodiments, the antibody is any antibody composition capable of further reducing the abundance PCSK9, thereby promoting the removal of LDL cholesterol from circulation. In some embodiments, the antibody is evolocumab, bococizumab, or alirocumab. In some embodiments, the antibody compositions is administered after any one of the gRNAs that includes any one or more of the guide sequences disclosed in Table 4 (e.g., in a composition provided herein). In some embodiments, the antibody composition is administered on a regular basis following treatment with any of the gRNA compositions provided herein.

In some embodiments, the combination therapy comprises administering any one of the gRNAs that includes any one or more of the guide sequences disclosed in Table 4 and a nucleic acid encoding an RNA-guided DNA-binding agent (e.g., in a composition provided herein) together with a siRNA that targets PCSK9 or mutant PCSK9. In some embodiments, the siRNA is any siRNA capable of further reducing or eliminating the expression of wild type or mutant PCSK9. In some embodiments, the siRNA is the drug inclisiran. In some embodiments, the siRNA is administered after any one of the gRNAs that includes any one or more of the guide sequences disclosed in Table 4 (e.g., in a composition provided herein). In some embodiments, the siRNA is administered on a regular basis following treatment with any of the gRNA compositions provided herein.

In some embodiments, the combination therapy comprises administering any one of the gRNAs that includes any one or more of the guide sequences described herein, e.g., disclosed in Table 4 and a nucleic acid encoding an RNA-guided DNA-binding agent described herein (e.g., in a composition provided herein) together with antisense nucleotide that targets PCSK9 or mutant PCSK9. In some embodiments, the antisense nucleotide is any antisense nucleotide capable of further reducing or eliminating the expression of wild type or mutant PCSK9. In some embodiments, the antisense nucleotide is administered after any one of the gRNAs that includes any one or more of the guide sequences disclosed in Table 4 and a nucleic acid encoding an RNA-guided DNA-binding agent (e.g., in a composition provided herein). In some embodiments, the antisense nucleotide is administered on a regular basis following treatment with any of the gRNA compositions provided herein.

In any of the foregoing embodiments, the guide sequences disclosed in Table 4, and/or the guide RNA may be a chemically modified guide RNA.

In some embodiments, a method described herein comprises infusion prophylaxis. In some embodiments, an infusion prophylaxis is administered to a subject before the gene editing composition. In some embodiments, an infusion prophylaxis is administered to a subject 8-24 hours or 1-2 hours prior to the administration of the nucleic acid composition.

In some embodiments, an infusion prophylaxis comprises corticosteroid. In some embodiments, the infusion prophylaxis comprises one or more, or all, of corticosteroid, an antipyretic (e.g. oral acetaminophen (also called paracetamol), which may reduce pain and fever and/or inhibit COX enzymes and/or prostaglandins), H1 blocker, or H2 blocker. In some embodiments, the infusion prophylaxis comprises an intravenous corticosteroid (e.g., dexamethasone 8-12 mg, such as 10 mg or equivalent) and an antipyretic (e.g. oral acetaminophen or paracetamol 500 mg). In some embodiments, the H1 blocker (e.g., diphenhydramine 50 mg or equivalent) and/or H2 blocker (e.g., ranitidine 50 mg or equivalent) are administered orally. In some embodiments, the H1 blocker (e.g., diphenhydramine 50 mg or equivalent) and/or H2 blocker (e.g., ranitidine 50 mg or equivalent) are administered intravenously. In some embodiments, an infusion prophylaxis is administered intravenously 1-2 hour before infusion of the nucleic acid composition.

In some embodiments an intravenous H1 blocker and/or an intravenous H2 blocker is substituted with an oral equivalent. The infusion prophylaxis may function to reduce adverse reactions associated with administering the nucleic acid composition. In some embodiments, the infusion prophylaxis is administered as a required premedication prior to administering the nucleic acid composition. The dosage, frequency and mode of administration of the corticosteroid, infusion prophylaxis, and the guide-RNA containing composition described herein can be controlled independently.

The corticosteroid used in the disclosed methods may be administered according to regimens known in the art, e.g., US FDA-approved regimens. In some embodiments, e.g., administration to or for use in a human subject, the corticosteroid can be administered in an amount that ranges from about 0.75 mg to about 25 mg. In some embodiments, e.g., administration to or for use in a human subject, the corticosteroid can be administered in an amount that ranges from about 0.01-0.5 mg/kg, such as 0.1-0.40 mg/kg or 0.25-0.40 mg/kg.

In some embodiments, the corticosteroid is administered before the guide RNA-containing composition described herein. In some embodiments, the corticosteroid is administered after the guide RNA-containing composition described herein. In some embodiments, the corticosteroid is administered simultaneously with the guide RNA-containing composition described herein. In some embodiments, multiple doses of the corticosteroid are administered before or after the administration of the guide RNA-containing composition. In some embodiments, multiple doses of the guide RNA-containing composition are administered before or after the administration of the corticosteroid. In some embodiments, multiple doses of the corticosteroid and multiple doses of the guide RNA-containing composition are administered.

If appropriate, a dose of corticosteroid may be administered as at least two sub doses administered separately at appropriate intervals. In some embodiments, the corticosteroid is administered at least two times before the administration of the guide RNA-containing composition described herein. In some embodiments, a dose of corticosteroid is administered at least two times after the administration of the guide RNA-containing composition described herein. In some embodiments, the corticosteroid is administered (e.g., before, with, and/or after the administration of the guide RNA-containing composition described herein) at an interval of 1 hour, 2 hours, 3 hours, 4 hours, 6 hours, 12 hours, 18 hours; 1, 2, 3, 4, 5, 6, 7, 8, 9, 10, 11, 12, 13, 14, or 15 days; 3, 4, 5, 6, 7, 8, 9, 10, 11, 12, 13, 14, or 15 weeks; or an amount of time in a range bounded by any two of the preceding values. In some embodiments, the corticosteroid is administered before the administration of the guide RNA-containing composition described herein at an interval of 1 hour, 2 hours, 3 hours, 4 hours, 6 hours, 12 hours, 18 hours; 1, 2, 3, 4, 5, 6, 7, 8, 9, 10, 11, 12, 13, 14, 15 days; 3, 4, 5, 6, 7, 8, 9, 10, 11, 12, 13, 14, or 15 weeks; or an amount of time in a range bounded by any two of the preceding values. In some embodiments, the corticosteroid is administered after the administration of the guide RNA-containing composition described herein at an interval of 1 hour, 2 hours, 3 hours, 4 hours, 6 hours, 12 hours, 18 hours; 1, 2, 3, 4, 5, 6, 7, 8, 9, 10, 11, 12, 13, 14, 15 days; 3, 4, 5, 6, 7, 8, 9, 10, 11, 12, 13, 14, or 15 weeks; or an amount of time in a range bounded by any two of the preceding values.

In some embodiments, the corticosteroid is administered at least two times. In some embodiments, the corticosteroid is administered at least three times. In some embodiments, the corticosteroid is administered at least four times. In some embodiments, the corticosteroid is administered up to five, six, seven, eight, nine, or ten times. A first dose may be oral and a second or subsequent dose may be by parenteral administration, e.g. infusion. Alternatively, a first dose may be parenteral and a second or subsequent dose may be by oral administration.

In some embodiments, the corticosteroid is administered orally before intravenous administration of a guide RNA-containing composition described herein. In some embodiments, the corticosteroid is administered orally at or after intravenous administration of a guide RNA-containing composition described herein.

In some embodiments, corticosteroid is dexamethasone. In some embodiments, dexamethasone is administered intravenously 1-2 hour before infusion of the nucleic acid composition. In some embodiments, dexamethasone is administered intravenously in the amount of 8-12 mg, such as 10 mg, 1-2 hour before infusion of the nucleic acid composition. In some embodiments, dexamethasone is administered orally 8 to 24 hours before infusion of the nucleic acid composition. In some embodiments, dexamethasone is administered orally in the amount of 8-12 mg, such as 8 mg, 8 to 24 hours before infusion of the nucleic acid composition. In some embodiments, dexamethasone is administered orally in the amount of 8-12 mg, such as 8 mg, 8 to 24 hours before infusion of the nucleic acid composition and dexamethasone is administered intravenously in the amount of 8-12 mg, such as 10 mg, 1-2 hour before infusion of the nucleic acid composition.

### Delivery of Nucleic Acid Compositions

In some embodiments, the nucleic acid compositions described herein, that include a gRNA and a nucleic acid encoding an RNA-guided DNA-binding agent as RNA or encoded on one or more vectors, are formulated in or administered via a lipid nanoparticle (LNP); see e.g., WO2017173054A1 and WO2019067992A1, the contents of which are hereby incorporated by reference in their entireties. Any LNP known to those of skill in the art to be capable of delivering nucleotides to subjects may be utilized with the guide RNAs described herein and the nucleic acid encoding an RNA-guided DNA nuclease.

In some embodiments, the guide RNA and the nucleic acid encoding an RNA-guided DNA nuclease are administered in an LNP described herein, such as an LNP that includes a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

Disclosed herein are various embodiments of LNP formulations for RNAs, including CRISPR/Cas cargoes. Such LNP formulations may include (i) a CCD lipid, such as an amine lipid, (ii) a neutral lipid, (iii) a helper lipid, and (iv) a stealth lipid, such as a PEG lipid. Some embodiments of the LNP formulations include an amine lipid, along with a helper lipid, a neutral lipid, and a stealth lipid such as a PEG lipid. In some embodiments, the LNP formulations include less than 1 percent neutral phospholipid. In some embodiments, the LNP formulations include less than 0.5 percent neutral phospholipid. A “lipid nanoparticle” could be a particle that comprises a plurality of (i.e. more than one) lipid molecules physically associated with each other by intermolecular forces. CCD Lipids, Amine Lipids, Neutral Lipids, and other lipids that can be used in the LNP formulations disclosed herein are described in WO2020198697, WO2015006747, WO2016118724, and WO2021026358, each of which is incorporated herein in its entirety.

Further technologies that can be used for delivery of the compositions of this disclosure include those that utilize encapsulation by biodegradable polymers, liposomes, viral like particles, or nanoparticles. In some embodiments, the compositions of this disclosure are administered in any suitable delivery vehicle, including, but not limited to, polymers, engineered viral particles (e.g., adeno-associated virus), exosomes, liposomes, supercharged proteins, implantable devices, or red blood cells. Suitable delivery methods are described in U.S. Pat. Nos. 10,851,357, 10,709,797, and US20170349914, each of which is incorporated herein in its entirety.

### EXAMPLES

The practice of the methods and compositions of the disclosure employs, unless otherwise indicated, conventional techniques of molecular biology (including recombinant techniques), cell culture, immunology, cell biology, and biochemistry, which are well within the purview of the skilled artisan. Such techniques are explained in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the methods and compositions of the disclosure. Particularly useful techniques for particular embodiments will be discussed in the sections that follow. The materials, reagents, and methods, further described below, are used in the following examples. The embodiments described in the following examples do not limit the scope of the claims.

### Example 1. Design of Guide RNA Sequences

The initial guide design was completed using custom computational tools and workflows, a human reference genome (e.g., GRCh38), and user-defined target genomic regions (e.g, PCSK9). The first step in determining guide sequences (i.e., gRNAs) was to scan the region of interest for PAMs. The candidate guides were then ranked using a variety of criteria (cutting efficiency and binding specificity scores, GC content, poly-T and free energy) that are expected to ensure high on-target cutting efficiency and low off-target potential. A total of 296 sgRNAs targeting the coding regions of PCSK9 (ENST00000302118.5) exons 1-12 were created. About 10 percent of these guides are 100% homologous in the reference genome of crab-eating monkeys (Macaca fascularis). Guide sequences and genomic coordinates are provided in Table 4.

The selected guide sequences are shown in Table 4. Table 4 below shows these 296 guide sequences that were designed to be targeted to the PCSK9 gene. The corresponding sgRNAs are shown in Table 5.

TABLE 4
Guide Sequences

| Name | Guide Sequence | SEQ ID NO |
|---|---|---|
| P9-h-100 | CCAGGUUCCACGGGAUGCUC | 1 |
| P9-h-102 | UAAUCCGCUCCAGGUUCCAC | 2 |
| P9-h-115 | UGGGGGUCUUACCGGGGGGC | 3 |
| P9-h-122 | UUGGAAAGACGGAGGCAGCC | 4 |
| P9-hc-127 | CAGCAUACAGAGUGACCACC | 5 |
| P9-hc-128 | CCGGUGGUCACUCUGUAUGC | 6 |
| P9-h-138 | AGAAUGUGCCCGAGGAGGAC | 7 |
| P9-h-150 | AGUCAUGGCACCCACCUGGC | 8 |
| P9-h-151 | GUCAUGGCACCCACCUGGCA | 9 |
| P9-hc-152 | CCGGGAUGCCGGCGUGGCCA | 10 |
| P9-hc-153 | CGGGAUGCCGGCGUGGCCAA | 11 |
| P9-hc-154 | CCUUGGCCACGCCGGCAUCC | 12 |
| P9-h-155 | GCUGGCACCCUUGGCCACGC | 13 |
| P9-hc-168 | UCCCAGGCCUGGAGUUUAUU | 14 |
| P9-hc-170 | UUCCGAAUAAACUCCAGGCC | 15 |
| P9-h-176 | GCGGCUGUACCCACCCGCCA | 16 |
| P9-h-177 | CGCGGCUGUACCCACCCGCC | 17 |
| P9-h-178 | GGCAGGCGGCGUUGAGGACG | 18 |
| P9-hc-179 | CAACGCCGCCUGCCAGCGCC | 19 |
| P9-hc-180 | GGCGCUGGCAGGCGGCGUUG | 20 |
| P9-hc-181 | CCGCCUGCCAGCGCCUGGCG | 21 |
| P9-hc-182 | CGCCUGCCAGCGCCUGGCGA | 22 |
| P9-hc-183 | AGCCCUCGCCAGGCGCUGGC | 23 |
| P9-hc-184 | GCACGACCCCAGCCCUCGCC | 24 |
| P9-hc-193 | CCUUUCCAGGUCAUCACAGU | 25 |
| P9-hc-202 | GCCGGUGACCCUGGGGACUU | 26 |
| P9-hc-203 | AAGUCCCCAGGGUCACCGGC | 27 |
| P9-hc-204 | CCGGUGACCCUGGGGACUUU | 28 |
| P9-hc-205 | GUUGGUCCCCAAAGUCCCCA | 29 |
| P9-hc-206 | AGUUGGUCCCCAAAGUCCCC | 30 |
| P9-hc-207 | GGGACUUUGGGGACCAACUU | 31 |
| P9-hc-211 | UGUGUGGACCUCUUUGCCCC | 32 |
| P9-hc-213 | UGUGGACCUCUUUGCCCCAG | 33 |
| P9-hc-214 | GGACCUCUUUGCCCCAGGGG | 34 |
| P9-hc-216 | GCACCAAUGAUGUCCUCCCC | 35 |
| P9-hc-217 | AAAGCAGGUGCUGCAGUCGC | 36 |
| P9-h-222 | AUCACAGGCUGCUGCCCACG | 37 |
| P9-hc-225 | AGCAUCAUGGCUGCAAUGCC | 38 |
| P9-hc-226 | CAUGAUGCUGUCUGCCGAGC | 39 |
| P9-hc-227 | CGGCUCGGCAGACAGCAUCA | 40 |
| P9-h-231 | AGCUCACCCUGGCCGAGUUG | 41 |
| P9-hc-232 | UCUCUGCCUCAACUCGGCCA | 42 |
| P9-hc-237 | GGCCUCAUUGAUGACAUCUU | 43 |
| P9-hc-240 | GUCAGUACCCGCUGGUCCUC | 44 |
| P9-h-245 | UACCUGCCCCAUGGGUGCUG | 45 |
| P9-h-247 | CUUACCUGCCCCAUGGGUGC | 46 |
| P9-h-248 | AUCCUGCUUACCUGCCCCAU | 47 |
| P9-h-249 | CCCAGGCCCUUUUUGCAGGU | 48 |
| P9-hc-251 | CAGGUUGGCAGCUGUUUUGC | 49 |
| P9-h-258 | CGCCCGCUGCGCCCCAGAUG | 50 |
| P9-h-259 | CUCCUCAUCUGGGGCGCAGC | 51 |
| P9-hc-261 | UCCAGGAGUGGGAAGCGGCG | 52 |
| P9-h-262 | GAAGCGGCGGGGCGAGCGCA | 53 |
| P9-h-263 | GCGGCGGGGCGAGCGCAUGG | 54 |
| P9-h-264 | GUCUUUGACUCUAAGGCCCA | 55 |
| P9-h-265 | UCUUUGACUCUAAGGCCCAA | 56 |
| P9-h-266 | UUUGACUCUAAGGCCCAAGG | 57 |
| P9-h-267 | UAAGGCCCAAGGGGGCAAGC | 58 |
| P9-h-269 | AAGGGGGCAAGCUGGUCUGC | 59 |
| P9-h-270 | GGCAGACCAGCUUGCCCCCU | 60 |
| P9-h-271 | AGGGGGCAAGCUGGUCUGCC | 61 |
| P9-hc-276 | CCCCAAAAGCGUUGUGGGCC | 62 |
| P9-hc-278 | CACAACGCUUUUGGGGGUGA | 63 |
| P9-hc-280 | CCUCACCCCCAAAAGCGUUG | 64 |
| P9-h-001 | AACCUCUCCCCUGGCCCUCA | 65 |
| P9-h-002 | ACCUCUCCCCUGGCCCUCAU | 66 |
| P9-h-003 | ACGGUGCCCAUGAGGGCCAG | 67 |
| P9-h-004 | GACGGUGCCCAUGAGGGCCA | 68 |
| P9-h-005 | UGACGGUGCCCAUGAGGGCC | 69 |
| P9-h-006 | UCAUGGGCACCGUCAGCUCC | 70 |
| P9-h-007 | GGAGCUGACGGUGCCCAUGA | 71 |
| P9-h-008 | UGGAGCUGACGGUGCCCAUG | 72 |
| P9-h-009 | UGGGCACCGUCAGCUCCAGG | 73 |
| P9-hc-010 | CCGUCAGCUCCAGGCGGUCC | 74 |
| P9-hc-011 | CCAGGACCGCCUGGAGCUGA | 75 |
| P9-hc-012 | UCAGCUCCAGGCGGUCCUGG | 76 |
| P9-h-013 | CAGCGGCCACCAGGACCGCC | 77 |
| P9-h-014 | CAGCAGUGGCAGCGGCCACC | 78 |
| P9-h-015 | GCUGCUGCUCCUGGGUCCCG | 79 |
| P9-h-016 | CUGCUGCUCCUGGGUCCCGC | 80 |
| P9-h-017 | CACGGGCGCCCGCGGGACCC | 81 |
| P9-h-018 | UCCCGCGGGCGCCCGUGCGC | 82 |
| P9-h-019 | CGCGGGCGCCCGUGCGCAGG | 83 |
| P9-h-020 | UCCUGCGCACGGGCGCCCGC | 84 |
| P9-h-021 | CUCCUGCGCACGGGCGCCCG | 85 |
| P9-hc-022 | CGCCCGUGCGCAGGAGGACG | 86 |
| P9-hc-023 | CGUGCGCAGGAGGACGAGGA | 87 |
| P9-hc-024 | GUCCUCGUCCUCCUGCGCAC | 88 |
| P9-hc-025 | CGUCCUCGUCCUCCUGCGCA | 89 |
| P9-hc-026 | GGACGAGGACGGCGACUACG | 90 |
| P9-hc-027 | GGACGGCGACUACGAGGAGC | 91 |
| P9-hc-028 | GGUGCUAGCCUUGCGUUCCG | 92 |
| P9-hc-029 | GCUAGCCUUGCGUUCCGAGG | 93 |
| P9-hc-030 | GCCUUGCGUUCCGAGGAGGA | 94 |
| P9-hc-031 | GCCGUCCUCCUCGGAACGCA | 95 |
| P9-hc-032 | GCGUUCCGAGGAGGACGGCC | 96 |
| P9-h-033 | UUCGGCCAGGCCGUCCUCCU | 97 |
| P9-h-034 | CUGGCCGAAGCACCCGAGCA | 98 |
| P9-h-035 | CGUGCUCGGGUGCUUCGGCC | 99 |
| P9-h-036 | GGUUCCGUGCUCGGGUGCUU | 100 |
| P9-h-037 | GUGGCUGUGGUUCCGUGCUC | 101 |
| P9-h-038 | GGUGGCUGUGGUUCCGUGCU | 102 |
| P9-hc-039 | CACCUUCCACCGCUGCGCCA | 103 |
| P9-hc-040 | CUUGGCGCAGCGGUGGAAGG | 104 |
| P9-hc-041 | UCCACCGCUGCGCCAAGGUG | 105 |
| P9-hc-042 | CACCUUGGCGCAGCGGUGGA | 106 |
| P9-hc-043 | CCACCGCUGCGCCAAGGUGC | 107 |
| P9-hc-044 | CCCGCACCUUGGCGCAGCGG | 108 |
| P9-h-045 | ACACCCGCACCUUGGCGCAG | 109 |
| P9-h-046 | CUGCGCCAAGGUGCGGGUGU | 110 |
| P9-h-047 | UGCGCCAAGGUGCGGGUGUA | 111 |
| P9-h-048 | CCAAGGUGCGGGUGUAGGGA | 112 |
| P9-h-049 | CAAGGUGCGGGUGUAGGGAU | 113 |
| P9-h-050 | CCAUCCCUACACCCGCACCU | 114 |
| P9-h-051 | GAUCCUGGCCCCAUGCAAGG | 115 |
| P9-h-052 | UUGCAUGGGGCCAGGAUCCG | 116 |
| P9-h-053 | ACGGAUCCUGGCCCCAUGCA | 117 |
| P9-h-054 | CAUGGGGCCAGGAUCCGUGG | 118 |
| P9-h-055 | CAGGAUCCGUGGAGGUUGCC | 119 |
| P9-h-056 | CAGGCAACCUCCACGGAUCC | 120 |
| P9-h-057 | UAGGUGCCAGGCAACCUCCA | 121 |
| P9-h-058 | GAGGUUGCCUGGCACCUACG | 122 |
| P9-h-059 | GUUGCCUGGCACCUACGUGG | 123 |
| P9-hc-060 | AGCACCACCACGUAGGUGCC | 124 |
| P9-hc-061 | CACCUACGUGGUGGUGCUGA | 125 |
| P9-hc-062 | CUACGUGGUGGUGCUGAAGG | 126 |
| P9-hc-063 | CUCCUUCAGCACCACCACGU | 127 |
| P9-h-064 | GCGCUCUGACUGCGAGAGGU | 128 |
| P9-h-065 | UGCGCUCUGACUGCGAGAGG | 129 |
| P9-hc-066 | CAGUGCGCUCUGACUGCGAG | 130 |
| P9-hc-067 | GCGCACUGCCCGCCGCCUGC | 131 |
| P9-hc-068 | UGCCCGCCGCCUGCAGGCCC | 132 |
| P9-h-069 | UGCAGGCCCAGGCUGCCCGC | 133 |
| P9-h-070 | CAGGCCCAGGCUGCCCGCCG | 134 |
| P9-h-071 | GUAUCCCCGGCGGGCAGCCU | 135 |
| P9-h-072 | GGUAUCCCCGGCGGGCAGCC | 136 |
| P9-hc-073 | CUUGGUGAGGUAUCCCCGGC | 137 |
| P9-hc-074 | UCUUGGUGAGGUAUCCCCGG | 138 |
| P9-hc-075 | GGAUCUUGGUGAGGUAUCCC | 139 |
| P9-hc-076 | AGACAUGCAGGAUCUUGGUG | 140 |
| P9-h-077 | AAGAUCCUGCAUGUCUUCCA | 141 |
| P9-hc-078 | AUGGAAGACAUGCAGGAUCU | 142 |
| P9-h-079 | GAAGGCCAUGGAAGACAUGC | 143 |
| P9-h-080 | GUCUUCCAUGGCCUUCUUCC | 144 |
| P9-hc-081 | CUCAUCUUCACCAGGAAGCC | 145 |
| P9-hc-082 | GGCUUCCUGGUGAAGAUGAG | 146 |
| P9-hc-083 | GGUCGCCACUCAUCUUCACC | 147 |
| P9-hc-084 | GAAGAUGAGUGGCGACCUGC | 148 |
| P9-hc-085 | GAGUGGCGACCUGCUGGAGC | 149 |
| P9-hc-086 | GGUGGCUCACCAGCUCCAGC | 150 |
| P9-h-087 | AGCUGGUGAGCCACCCUUUU | 151 |
| P9-h-088 | GCUGGUGAGCCACCCUUUUU | 152 |
| P9-h-089 | AAGGCCUGCAGAAGCCAGAG | 153 |
| P9-hc-090 | GUCGACAUGGGGCAACUUCA | 154 |
| P9-hc-091 | GCCCCAUGUCGACUACAUCG | 155 |
| P9-hc-092 | CCAUGUCGACUACAUCGAGG | 156 |
| P9-hc-093 | UCCUCGAUGUAGUCGACAUG | 157 |
| P9-hc-094 | CUCCUCGAUGUAGUCGACAU | 158 |
| P9-hc-095 | CCUCCUCGAUGUAGUCGACA | 159 |
| P9-h-096 | GAUGCUCUGGGCAAAGACAG | 160 |
| P9-h-097 | UCUUUGCCCAGAGCAUCCCG | 161 |
| P9-h-098 | CCAGAGCAUCCCGUGGAACC | 162 |
| P9-h-099 | CAGGUUCCACGGGAUGCUCU | 163 |
| P9-h-101 | GCAUCCCGUGGAACCUGGAG | 164 |
| P9-h-103 | GUAAUCCGCUCCAGGUUCCA | 165 |
| P9-h-104 | UGGAGCGGAUUACCCCUCCA | 166 |
| P9-h-105 | GUGGAGGGGUAAUCCGCUCC | 167 |
| P9-h-106 | GGAUUACCCCUCCACGGUAC | 168 |
| P9-h-107 | GAUUACCCCUCCACGGUACC | 169 |
| P9-h-108 | UACCCCUCCACGGUACCGGG | 170 |
| P9-h-109 | AUCCGCCCGGUACCGUGGAG | 171 |
| P9-h-110 | CAUCCGCCCGGUACCGUGGA | 172 |
| P9-h-111 | UCAUCCGCCCGGUACCGUGG | 173 |
| P9-h-112 | UAUUCAUCCGCCCGGUACCG | 174 |
| P9-hc-113 | GGGGCUGGUAUUCAUCCGCC | 175 |
| P9-hc-114 | GCGGAUGAAUACCAGCCCCC | 176 |
| P9-h-116 | CAGAUGGGGGUCUUACCGGG | 177 |
| P9-h-117 | ACAGAUGGGGGUCUUACCGG | 178 |
| P9-h-118 | CUUUCCAAGGCGACAUUUGU | 179 |
| P9-h-119 | UCUUUCCAAGGCGACAUUUG | 180 |
| P9-h-120 | CAAAUGUCGCCUUGGAAAGA | 181 |
| P9-h-121 | AUGUCGCCUUGGAAAGACGG | 182 |
| P9-h-123 | GAAAGACGGAGGCAGCCUGG | 183 |
| P9-hc-124 | CUAGGAGAUACACCUCCACC | 184 |
| P9-hc-125 | CACUCUGUAUGCUGGUGUCU | 185 |
| P9-hc-126 | CCAGCAUACAGAGUGACCAC | 186 |
| P9-hc-129 | GAGUGACCACCGGGAAAUCG | 187 |
| P9-hc-130 | AGUGACCACCGGGAAAUCGA | 188 |
| P9-hc-131 | ACCACCGGGAAAUCGAGGGC | 189 |
| P9-hc-132 | CCACCGGGAAAUCGAGGGCA | 190 |
| P9-hc-133 | CCCUGCCCUCGAUUUCCCGG | 191 |
| P9-hc-134 | UGACCCUGCCCUCGAUUUCC | 192 |
| P9-h-135 | CGACUUCGAGAAUGUGCCCG | 193 |
| P9-h-136 | CUCGGGCACAUUCUCGAAGU | 194 |
| P9-h-137 | CUUCGAGAAUGUGCCCGAGG | 195 |
| P9-hc-139 | AAGCGGGUCCCGUCCUCCUC | 196 |
| P9-hc-140 | GAAGCGGGUCCCGUCCUCCU | 197 |
| P9-hc-141 | CGGGACCCGCUUCCACAGAC | 198 |
| P9-hc-142 | GCUUACCUGUCUGUGGAAGC | 199 |
| P9-h-143 | CGGCCGUGCUUACCUGUCUG | 200 |
| P9-h-144 | AGGUAAGCACGGCCGUCUGA | 201 |
| P9-h-145 | GGUAAGCACGGCCGUCUGAU | 202 |
| P9-h-146 | GGUCCUUGUGUUCGUCGAGC | 203 |
| P9-h-147 | UGGCCUGCUCGACGAACACA | 204 |
| P9-h-148 | GCCAGCAAGUGUGACAGUCA | 205 |
| P9-h-149 | GCCAUGACUGUCACACUUGC | 206 |
| P9-h-156 | GCUGCGCAUGCUGGCACCCU | 207 |
| P9-h-157 | CACGCGCAGGCUGCGCAUGC | 208 |
| P9-hc-158 | CUGCGCGUGCUCAACUGCCA | 209 |
| P9-hc-159 | UGCGCGUGCUCAACUGCCAA | 210 |
| P9-hc-160 | CUUGGCAGUUGAGCACGCGC | 211 |
| P9-hc-161 | CGUGCUCAACUGCCAAGGGA | 212 |
| P9-hc-162 | GUGCUCAACUGCCAAGGGAA | 213 |
| P9-h-163 | CAAGGGAAGGGCACGGUUAG | 214 |
| P9-h-164 | CGCUAACCGUGCCCUUCCCU | 215 |
| P9-h-165 | ACGGUUAGCGGCACCCUCAU | 216 |
| P9-h-166 | GGGCCAUCACUUACCUAUGA | 217 |
| P9-h-167 | GGGGCCAUCACUUACCUAUG | 218 |
| P9-hc-169 | UCCGAAUAAACUCCAGGCCU | 219 |
| P9-hc-171 | GGCUUUUCCGAAUAAACUCC | 220 |
| P9-hc-172 | GUUUAUUCGGAAAAGCCAGC | 221 |
| P9-h-173 | GCCAGCUGGUCCAGCCUGUG | 222 |
| P9-h-174 | AGCACCACCAGUGGCCCCAC | 223 |
| P9-hc-175 | CGGCUGUACCCACCCGCCAG | 224 |
| P9-hc-185 | GUCGUGCUGGUCACCGCUGC | 225 |
| P9-hc-186 | UCACCGCUGCCGGCAACUUC | 226 |
| P9-hc-187 | CACCGCUGCCGGCAACUUCC | 227 |
| P9-hc-188 | GUCCCGGAAGUUGCCGGCAG | 228 |
| P9-hc-189 | GGCAUCGUCCCGGAAGUUGC | 229 |
| P9-hc-190 | AGUAGAGGCAGGCAUCGUCC | 230 |
| P9-h-191 | CCAGCCUCAGCUCCCGAGGU | 231 |
| P9-h-192 | AGCACCUACCUCGGGAGCUG | 232 |
| P9-hc-194 | CUUUCCAGGUCAUCACAGUU | 233 |
| P9-hc-195 | UUUCCAGGUCAUCACAGUUG | 234 |
| P9-hc-196 | UGGCCCCAACUGUGAUGACC | 235 |
| P9-h-197 | CGGCUGGUCUUGGGCAUUGG | 236 |
| P9-h-198 | CACCGGCUGGUCUUGGGCAU | 237 |
| P9-h-199 | CAAGACCAGCCGGUGACCCU | 238 |
| P9-h-200 | AAGACCAGCCGGUGACCCUG | 239 |
| P9-h-201 | CAGGGUCACCGGCUGGUCUU | 240 |
| P9-hc-208 | GACCAACUUUGGCCGCUGUG | 241 |
| P9-hc-209 | GUCCACACAGCGGCCAAAGU | 242 |
| P9-hc-210 | GGGCAAAGAGGUCCACACAG | 243 |
| P9-hc-212 | GUGUGGACCUCUUUGCCCCA | 244 |
| P9-hc-215 | UGUCCUCCCCUGGGGCAAAG | 245 |
| P9-h-218 | ACCUGCUUUGUGUCACAGAG | 246 |
| P9-h-219 | CCUGCUUUGUGUCACAGAGU | 247 |
| P9-h-220 | CCCACUCUGUGACACAAAGC | 248 |
| P9-h-221 | GUCACAGAGUGGGACAUCAC | 249 |
| P9-hc-223 | UGGUGACUUACCAGCCACGU | 250 |
| P9-hc-224 | GUGGUGACUUACCAGCCACG | 251 |
| P9-h-228 | UGCCGAGCCGGAGCUCACCC | 252 |
| P9-h-229 | GGCCAGGGUGAGCUCCGGCU | 253 |
| P9-h-230 | AACUCGGCCAGGGUGAGCUC | 254 |
| P9-h-233 | GUCUCUGCCUCAACUCGGCC | 255 |
| P9-hc-234 | GAUCAGUCUCUGCCUCAACU | 256 |
| P9-hc-235 | UGACAUCUUUGGCAGAGAAG | 257 |
| P9-hc-236 | AAGAUGUCAUCAAUGAGGCC | 258 |
| P9-hc-238 | CAAUGAGGCCUGGUUCCCUG | 259 |
| P9-hc-239 | UCAGUACCCGCUGGUCCUCA | 260 |
| P9-hc-241 | GCGGGUACUGACCCCCAACC | 261 |
| P9-hc-242 | GGUUGGGGGUCAGUACCCGC | 262 |
| P9-hc-243 | GGUACUGACCCCCAACCUGG | 263 |
| P9-h-244 | CCUGCCCCCCAGCACCCAUG | 264 |
| P9-h-246 | UUACCUGCCCCAUGGGUGCU | 265 |
| P9-h-250 | GCCAACCUGCAAAAAGGGCC | 266 |
| P9-h-252 | GACUGUAUGGUCAGCACACU | 267 |
| P9-h-253 | ACUGUAUGGUCAGCACACUC | 268 |
| P9-h-254 | CUGUAUGGUCAGCACACUCG | 269 |
| P9-h-255 | CAGCACACUCGGGGCCUACA | 270 |
| P9-h-256 | ACACUCGGGGCCUACACGGA | 271 |
| P9-hc-257 | ACGGCUGUGGCCAUCCGUGU | 272 |
| P9-h-260 | GCUCCUCAUCUGGGGCGCAG | 273 |
| P9-h-268 | GCAGACCAGCUUGCCCCCUU | 274 |
| P9-hc-272 | UGCCGGGCCCACAACGCUUU | 275 |
| P9-hc-273 | GCCGGGCCCACAACGCUUUU | 276 |
| P9-hc-274 | CCGGGCCCACAACGCUUUUG | 277 |
| P9-hc-275 | CGGGCCCACAACGCUUUUGG | 278 |
| P9-hc-277 | CCACAACGCUUUUGGGGGUG | 279 |
| P9-hc-279 | CUCACCCCCAAAAGCGUUGU | 280 |
| P9-hc-281 | AGGGUGUCUACGCCAUUGCC | 281 |
| P9-hc-282 | GUGGACACGGGUCCCCAUGC | 282 |
| P9-hc-283 | AAGGUCCUCCACCUCCCAGU | 283 |
| P9-hc-284 | CCUCAGCACAGGCGGCUUGU | 284 |
| P9-hc-285 | CACUGGUUGGGCUGACCUCG | 285 |
| P9-h-286 | AGGUCAGCCCAACCAGUGCG | 286 |
| P9-h-287 | GGUCAGCCCAACCAGUGCGU | 287 |
| P9-h-288 | CAACCAGUGCGUGGGCCACA | 288 |
| P9-h-289 | UAGACAACACGUGUGUAGUC | 289 |
| P9-h-290 | GACUACACACGUGUUGUCUA | 290 |
| P9-h-291 | CACGUGUGUAGUCAGGAGCC | 291 |
| P9-h-292 | AGCCGGGACGUCAGCACUAC | 292 |
| P9-h-293 | UGCCUGUAGUGCUGACGUCC | 293 |
| P9-h-294 | ACUACAGGCAGCACCAGCGA | 294 |
| P9-h-295 | UACAGGCAGCACCAGCGAAG | 295 |
| P9-h-296 | GCAGAUGGCAACGGCUGUCA | 296 |

TABLE 5
crRNA and sgRNA sequences corresponding to the guide sequences in
Table 4

| | Guide | | crRNA | | sgRNA |
|---|---|---|---|---|---|
| | SEQ ID | | SEQ | | SEQ |
| Name | NO: | crRNA sequence | ID NO | sgRNA sequence | ID NO |
| P9-h- | 1 | CCAGGUUCCACG | 297 | CCAGGUUCCACGGGAUGCUC | 593 |
| 100 | | GGAUGCUCGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 2 | UAAUCCGCUCCA | 298 | UAAUCCGCUCCAGGUUCCAC | 594 |
| 102 | | GGUUCCACGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 3 | UGGGGGUCUUA | 299 | UGGGGGUCUUACCGGGGGG | 595 |
| 115 | | CCGGGGGGCGU | | CGUUUUAGAGCUAGAAAUAG | |
| | | UUUAGAGCUAU | | CAAGUUAAAAUAAGGCUAGU | |
| | | GCUGUUUUG | | CCGUUAUCAACUUGAAAAAG | |
| | | | | UGGCACCGAGUCGGUGCUUU | |
| | | | | U | |
| P9-h- | 4 | UUGGAAAGACG | 300 | UUGGAAAGACGGAGGCAGCC | 596 |
| 122 | | GAGGCAGCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 5 | CAGCAUACAGA | 301 | CAGCAUACAGAGUGACCACC | 597 |
| hc- | | GUGACCACCGU | | GUUUUAGAGCUAGAAAUAGC | |
| 127 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 6 | CCGGUGGUCAC | 302 | CCGGUGGUCACUCUGUAUGC | 598 |
| hc- | | UCUGUAUGCGU | | GUUUUAGAGCUAGAAAUAGC | |
| 128 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 7 | AGAAUGUGCCC | 303 | AGAAUGUGCCCGAGGAGGAC | 599 |
| 138 | | GAGGAGGACGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 8 | AGUCAUGGCAC | 304 | AGUCAUGGCACCCACCUGGC | 600 |
| 150 | | CCACCUGGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 9 | GUCAUGGCACCC | 305 | GUCAUGGCACCCACCUGGCA | 601 |
| 151 | | ACCUGGCAGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 10 | CCGGGAUGCCG | 306 | CCGGGAUGCCGGCGUGGCCA | 602 |
| hc- | | GCGUGGCCAGU | | GUUUUAGAGCUAGAAAUAGC | |
| 152 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 11 | CGGGAUGCCGG | 307 | CGGGAUGCCGGCGUGGCCAA | 603 |
| hc- | | CGUGGCCAAGU | | GUUUUAGAGCUAGAAAUAGC | |
| 153 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 12 | CCUUGGCCACGC | 308 | CCUUGGCCACGCCGGCAUCC | 604 |
| hc- | | CGGCAUCCGUU | | GUUUUAGAGCUAGAAAUAGC | |
| 154 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 13 | GCUGGCACCCU | 309 | GCUGGCACCCUUGGCCACGC | 605 |
| 155 | | UGGCCACGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 14 | UCCCAGGCCUG | 310 | UCCCAGGCCUGGAGUUUAUU | 606 |
| hc- | | GAGUUUAUUGU | | GUUUUAGAGCUAGAAAUAGC | |
| 168 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 15 | UUCCGAAUAAA | 311 | UUCCGAAUAAACUCCAGGCC | 607 |
| hc- | | CUCCAGGCCGU | | GUUUUAGAGCUAGAAAUAGC | |
| 170 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 16 | GCGGCUGUACC | 312 | GCGGCUGUACCCACCCGCCAG | 608 |
| 176 | | CACCCGCCAGUU | | UUUUAGAGCUAGAAAUAGCA | |
| | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 17 | CGCGGCUGUAC | 313 | CGCGGCUGUACCCACCCGCCG | 609 |
| 177 | | CCACCCGCCGUU | | UUUUAGAGCUAGAAAUAGCA | |
| | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 18 | GGCAGGCGGCG | 314 | GGCAGGCGGCGUUGAGGACG | 610 |
| 178 | | UUGAGGACGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 19 | CAACGCCGCCUG | 315 | CAACGCCGCCUGCCAGCGCCG | 611 |
| hc- | | CCAGCGCCGUU | | UUUUAGAGCUAGAAAUAGCA | |
| 179 | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9- | 20 | GGCGCUGGCAG | 316 | GGCGCUGGCAGGCGGCGUUG | 612 |
| hc- | | GCGGCGUUGGU | | GUUUUAGAGCUAGAAAUAGC | |
| 180 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 21 | CCGCCUGCCAGC | 317 | CCGCCUGCCAGCGCCUGGCG | 613 |
| hc- | | GCCUGGCGGUU | | GUUUUAGAGCUAGAAAUAGC | |
| 181 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 22 | CGCCUGCCAGCG | 318 | CGCCUGCCAGCGCCUGGCGA | 614 |
| hc- | | CCUGGCGAGUU | | GUUUUAGAGCUAGAAAUAGC | |
| 182 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 23 | AGCCCUCGCCAG | 319 | AGCCCUCGCCAGGCGCUGGC | 615 |
| hc- | | GCGCUGGCGUU | | GUUUUAGAGCUAGAAAUAGC | |
| 183 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 24 | GCACGACCCCAG | 320 | GCACGACCCCAGCCCUCGCCG | 616 |
| hc- | | CCCUCGCCGUU | | UUUUAGAGCUAGAAAUAGCA | |
| 184 | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9- | 25 | CCUUUCCAGGU | 321 | CCUUUCCAGGUCAUCACAGU | 617 |
| hc- | | CAUCACAGUGU | | GUUUUAGAGCUAGAAAUAGC | |
| 193 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 26 | GCCGGUGACCC | 322 | GCCGGUGACCCUGGGGACUU | 618 |
| hc- | | UGGGGACUUGU | | GUUUUAGAGCUAGAAAUAGC | |
| 202 | | | | AAGUUAAAAUAAGGCUAGUC | |
| | | UUUAGAGCUAU | | CGUUAUCAACUUGAAAAAGU | |
| | | GCUGUUUUG | | GGCACCGAGUCGGUGCUUUU | |
| P9- | 27 | AAGUCCCCAGG | 323 | AAGUCCCCAGGGUCACCGGC | 619 |
| hc- | | GUCACCGGCGU | | GUUUUAGAGCUAGAAAUAGC | |
| 203 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 28 | CCGGUGACCCU | 324 | CCGGUGACCCUGGGGACUUU | 620 |
| hc- | | GGGGACUUUGU | | GUUUUAGAGCUAGAAAUAGC | |
| 204 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 29 | GUUGGUCCCCA | 325 | GUUGGUCCCCAAAGUCCCCA | 621 |
| hc- | | AAGUCCCCAGU | | GUUUUAGAGCUAGAAAUAGC | |
| 205 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 30 | AGUUGGUCCCC | 326 | AGUUGGUCCCCAAAGUCCCC | 622 |
| hc- | | AAAGUCCCCGU | | GUUUUAGAGCUAGAAAUAGC | |
| 206 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 31 | GGGACUUUGGG | 327 | GGGACUUUGGGGACCAACUU | 623 |
| hc- | | GACCAACUUGU | | GUUUUAGAGCUAGAAAUAGC | |
| 207 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 32 | UGUGUGGACCU | 328 | UGUGUGGACCUCUUUGCCCC | 624 |
| hc- | | CUUUGCCCCGU | | GUUUUAGAGCUAGAAAUAGC | |
| 211 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 33 | UGUGGACCUCU | 329 | UGUGGACCUCUUUGCCCCAG | 625 |
| hc- | | UUGCCCCAGGU | | GUUUUAGAGCUAGAAAUAGC | |
| 213 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 34 | GGACCUCUUUG | 330 | GGACCUCUUUGCCCCAGGGG | 626 |
| hc- | | CCCCAGGGGGU | | GUUUUAGAGCUAGAAAUAGC | |
| 214 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 35 | GCACCAAUGAU | 331 | GCACCAAUGAUGUCCUCCCC | 627 |
| hc- | | GUCCUCCCCGU | | GUUUUAGAGCUAGAAAUAGC | |
| 216 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 36 | AAAGCAGGUGC | 332 | AAAGCAGGUGCUGCAGUCGC | 628 |
| hc- | | UGCAGUCGCGU | | GUUUUAGAGCUAGAAAUAGC | |
| 217 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 37 | AUCACAGGCUG | 333 | AUCACAGGCUGCUGCCCACG | 629 |
| 222 | | CUGCCCACGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 38 | AGCAUCAUGGC | 334 | AGCAUCAUGGCUGCAAUGCC | 630 |
| hc- | | UGCAAUGCCGU | | GUUUUAGAGCUAGAAAUAGC | |
| 225 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 39 | CAUGAUGCUGU | 335 | CAUGAUGCUGUCUGCCGAGC | 631 |
| hc- | | CUGCCGAGCGU | | GUUUUAGAGCUAGAAAUAGC | |
| 226 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 40 | CGGCUCGGCAG | 336 | CGGCUCGGCAGACAGCAUCA | 632 |
| hc- | | ACAGCAUCAGU | | GUUUUAGAGCUAGAAAUAGC | |
| 227 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 41 | AGCUCACCCUGG | 337 | AGCUCACCCUGGCCGAGUUG | 633 |
| 231 | | CCGAGUUGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 42 | UCUCUGCCUCAA | 338 | UCUCUGCCUCAACUCGGCCA | 634 |
| hc- | | CUCGGCCAGUU | | GUUUUAGAGCUAGAAAUAGC | |
| 232 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 43 | GGCCUCAUUGA | 339 | GGCCUCAUUGAUGACAUCUU | 635 |
| hc- | | UGACAUCUUGU | | GUUUUAGAGCUAGAAAUAGC | |
| 237 | | | | AAGUUAAAAUAAGGCUAGUC | |
| | | UUUAGAGCUAU | | CGUUAUCAACUUGAAAAAGU | |
| | | GCUGUUUUG | | GGCACCGAGUCGGUGCUUUU | |
| P9- | 44 | GUCAGUACCCGC | 340 | GUCAGUACCCGCUGGUCCUC | 636 |
| hc- | | UGGUCCUCGUU | | GUUUUAGAGCUAGAAAUAGC | |
| 240 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 45 | UACCUGCCCCAU | 341 | UACCUGCCCCAUGGGUGCUG | 637 |
| 245 | | GGGUGCUGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 46 | CUUACCUGCCCC | 342 | CUUACCUGCCCCAUGGGUGC | 638 |
| 247 | | AUGGGUGCGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 47 | AUCCUGCUUACC | 343 | AUCCUGCUUACCUGCCCCAU | 639 |
| 248 | | UGCCCCAUGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 48 | CCCAGGCCCUUU | 344 | CCCAGGCCCUUUUUGCAGGU | 640 |
| 249 | | UUGCAGGUGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 49 | CAGGUUGGCAG | 345 | CAGGUUGGCAGCUGUUUUG | 641 |
| hc- | | CUGUUUUGCGU | | CGUUUUAGAGCUAGAAAUAG | |
| 251 | | UUUAGAGCUAU | | CAAGUUAAAAUAAGGCUAGU | |
| | | GCUGUUUUG | | CCGUUAUCAACUUGAAAAAG | |
| | | | | UGGCACCGAGUCGGUGCUUU | |
| | | | | U | |
| P9-h- | 50 | CGCCCGCUGCGC | 346 | CGCCCGCUGCGCCCCAGAUG | 642 |
| 258 | | CCCAGAUGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 51 | CUCCUCAUCUG | 347 | CUCCUCAUCUGGGGCGCAGC | 643 |
| 259 | | GGGCGCAGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 52 | UCCAGGAGUGG | 348 | UCCAGGAGUGGGAAGCGGCG | 644 |
| hc- | | GAAGCGGCGGU | | GUUUUAGAGCUAGAAAUAGC | |
| 261 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 53 | GAAGCGGCGGG | 349 | GAAGCGGCGGGGCGAGCGCA | 645 |
| 262 | | GCGAGCGCAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 54 | GCGGCGGGGCG | 350 | GCGGCGGGGCGAGCGCAUGG | 646 |
| 263 | | AGCGCAUGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 55 | GUCUUUGACUC | 351 | GUCUUUGACUCUAAGGCCCA | 647 |
| 264 | | UAAGGCCCAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 56 | UCUUUGACUCU | 352 | UCUUUGACUCUAAGGCCCAA | 648 |
| 265 | | AAGGCCCAAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 57 | UUUGACUCUAA | 353 | UUUGACUCUAAGGCCCAAGG | 649 |
| 266 | | GGCCCAAGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 58 | UAAGGCCCAAG | 354 | UAAGGCCCAAGGGGGCAAGC | 650 |
| 267 | | GGGGCAAGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 59 | AAGGGGGCAAG | 355 | AAGGGGGCAAGCUGGUCUGC | 651 |
| 269 | | CUGGUCUGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 60 | GGCAGACCAGC | 356 | GGCAGACCAGCUUGCCCCCU | 652 |
| 270 | | UUGCCCCCUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 61 | AGGGGGCAAGC | 357 | AGGGGGCAAGCUGGUCUGCC | 653 |
| 271 | | UGGUCUGCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 62 | CCCCAAAAGCGU | 358 | CCCCAAAAGCGUUGUGGGCC | 654 |
| hc- | | UGUGGGCCGUU | | GUUUUAGAGCUAGAAAUAGC | |
| 276 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 63 | CACAACGCUUU | 359 | CACAACGCUUUUGGGGGUGA | 655 |
| hc- | | UGGGGGUGAGU | | GUUUUAGAGCUAGAAAUAGC | |
| 278 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 64 | CCUCACCCCCAA | 360 | CCUCACCCCCAAAAGCGUUGG | 656 |
| hc- | | AAGCGUUGGUU | | UUUUAGAGCUAGAAAUAGCA | |
| 280 | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 65 | AACCUCUCCCCU | 361 | AACCUCUCCCCUGGCCCUCAG | 657 |
| 001 | | GGCCCUCAGUU | | UUUUAGAGCUAGAAAUAGCA | |
| | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 66 | ACCUCUCCCCUG | 362 | ACCUCUCCCCUGGCCCUCAUG | 658 |
| 002 | | GCCCUCAUGUU | | UUUUAGAGCUAGAAAUAGCA | |
| | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 67 | ACGGUGCCCAU | 363 | ACGGUGCCCAUGAGGGCCAG | 659 |
| 003 | | GAGGGCCAGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 68 | GACGGUGCCCA | 364 | GACGGUGCCCAUGAGGGCCA | 660 |
| 004 | | UGAGGGCCAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 69 | UGACGGUGCCC | 365 | UGACGGUGCCCAUGAGGGCC | 661 |
| 005 | | AUGAGGGCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 70 | UCAUGGGCACC | 366 | UCAUGGGCACCGUCAGCUCC | 662 |
| 006 | | GUCAGCUCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 71 | GGAGCUGACGG | 367 | GGAGCUGACGGUGCCCAUGA | 663 |
| 007 | | UGCCCAUGAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 72 | UGGAGCUGACG | 368 | UGGAGCUGACGGUGCCCAUG | 664 |
| 008 | | GUGCCCAUGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 73 | UGGGCACCGUC | 369 | UGGGCACCGUCAGCUCCAGG | 665 |
| 009 | | AGCUCCAGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 74 | CCGUCAGCUCCA | 370 | CCGUCAGCUCCAGGCGGUCC | 666 |
| hc- | | GGCGGUCCGUU | | GUUUUAGAGCUAGAAAUAGC | |
| 010 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 75 | CCAGGACCGCCU | 371 | CCAGGACCGCCUGGAGCUGA | 667 |
| hc- | | GGAGCUGAGUU | | GUUUUAGAGCUAGAAAUAGC | |
| 011 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 76 | UCAGCUCCAGGC | 372 | UCAGCUCCAGGCGGUCCUGG | 668 |
| hc- | | GGUCCUGGGUU | | GUUUUAGAGCUAGAAAUAGC | |
| 012 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 77 | CAGCGGCCACCA | 373 | CAGCGGCCACCAGGACCGCCG | 669 |
| 013 | | GGACCGCCGUU | | UUUUAGAGCUAGAAAUAGCA | |
| | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 78 | CAGCAGUGGCA | 374 | CAGCAGUGGCAGCGGCCACC | 670 |
| 014 | | GCGGCCACCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 79 | GCUGCUGCUCC | 375 | GCUGCUGCUCCUGGGUCCCG | 671 |
| 015 | | UGGGUCCCGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 80 | CUGCUGCUCCU | 376 | CUGCUGCUCCUGGGUCCCGC | 672 |
| 016 | | GGGUCCCGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 81 | CACGGGCGCCCG | 377 | CACGGGCGCCCGCGGGACCC | 673 |
| 017 | | CGGGACCCGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 82 | UCCCGCGGGCGC | 378 | UCCCGCGGGCGCCCGUGCGC | 674 |
| 018 | | CCGUGCGCGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 83 | CGCGGGCGCCCG | 379 | CGCGGGCGCCCGUGCGCAGG | 675 |
| 019 | | UGCGCAGGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 84 | UCCUGCGCACG | 380 | UCCUGCGCACGGGCGCCCGC | 676 |
| 020 | | GGCGCCCGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 85 | CUCCUGCGCACG | 381 | CUCCUGCGCACGGGCGCCCG | 677 |
| 021 | | GGCGCCCGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 86 | CGCCCGUGCGCA | 382 | CGCCCGUGCGCAGGAGGACG | 678 |
| hc- | | GGAGGACGGUU | | GUUUUAGAGCUAGAAAUAGC | |
| 022 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 87 | CGUGCGCAGGA | 383 | CGUGCGCAGGAGGACGAGGA | 679 |
| hc- | | GGACGAGGAGU | | GUUUUAGAGCUAGAAAUAGC | |
| 023 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 88 | GUCCUCGUCCU | 384 | GUCCUCGUCCUCCUGCGCAC | 680 |
| hc- | | CCUGCGCACGU | | GUUUUAGAGCUAGAAAUAGC | |
| 024 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 89 | CGUCCUCGUCC | 385 | CGUCCUCGUCCUCCUGCGCA | 681 |
| hc- | | UCCUGCGCAGU | | GUUUUAGAGCUAGAAAUAGC | |
| 025 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 90 | GGACGAGGACG | 386 | GGACGAGGACGGCGACUACG | 682 |
| hc- | | GCGACUACGGU | | GUUUUAGAGCUAGAAAUAGC | |
| 026 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 91 | GGACGGCGACU | 387 | GGACGGCGACUACGAGGAGC | 683 |
| hc- | | ACGAGGAGCGU | | GUUUUAGAGCUAGAAAUAGC | |
| 027 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 92 | GGUGCUAGCCU | 388 | GGUGCUAGCCUUGCGUUCCG | 684 |
| hc- | | UGCGUUCCGGU | | GUUUUAGAGCUAGAAAUAGC | |
| 028 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 93 | GCUAGCCUUGC | 389 | GCUAGCCUUGCGUUCCGAGG | 685 |
| hc- | | GUUCCGAGGGU | | GUUUUAGAGCUAGAAAUAGC | |
| 029 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 94 | GCCUUGCGUUC | 390 | GCCUUGCGUUCCGAGGAGGA | 686 |
| hc- | | CGAGGAGGAGU | | GUUUUAGAGCUAGAAAUAGC | |
| 030 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 95 | GCCGUCCUCCUC | 391 | GCCGUCCUCCUCGGAACGCA | 687 |
| hc- | | GGAACGCAGUU | | GUUUUAGAGCUAGAAAUAGC | |
| 031 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 96 | GCGUUCCGAGG | 392 | GCGUUCCGAGGAGGACGGCC | 688 |
| hc- | | AGGACGGCCGU | | GUUUUAGAGCUAGAAAUAGC | |
| 032 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 97 | UUCGGCCAGGC | 393 | UUCGGCCAGGCCGUCCUCCU | 689 |
| 033 | | CGUCCUCCUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 98 | CUGGCCGAAGC | 394 | CUGGCCGAAGCACCCGAGCA | 690 |
| 034 | | ACCCGAGCAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 99 | CGUGCUCGGGU | 395 | CGUGCUCGGGUGCUUCGGCC | 691 |
| 035 | | GCUUCGGCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 100 | GGUUCCGUGCU | 396 | GGUUCCGUGCUCGGGUGCU | 692 |
| 036 | | CGGGUGCUUGU | | UGUUUUAGAGCUAGAAAUA | |
| | | UUUAGAGCUAU | | GCAAGUUAAAAUAAGGCUAG | |
| | | GCUGUUUUG | | UCCGUUAUCAACUUGAAAAA | |
| | | | | GUGGCACCGAGUCGGUGCUU | |
| | | | | UU | |
| P9-h- | 101 | GUGGCUGUGGU | 397 | GUGGCUGUGGUUCCGUGCU | 693 |
| 037 | | UCCGUGCUCGU | | CGUUUUAGAGCUAGAAAUAG | |
| | | UUUAGAGCUAU | | CAAGUUAAAAUAAGGCUAGU | |
| | | GCUGUUUUG | | CCGUUAUCAACUUGAAAAAG | |
| | | | | UGGCACCGAGUCGGUGCUUU | |
| | | | | U | |
| P9-h- | 102 | GGUGGCUGUGG | 398 | GGUGGCUGUGGUUCCGUGC | 694 |
| 038 | | UUCCGUGCUGU | | UGUUUUAGAGCUAGAAAUA | |
| | | UUUAGAGCUAU | | GCAAGUUAAAAUAAGGCUAG | |
| | | GCUGUUUUG | | UCCGUUAUCAACUUGAAAAA | |
| | | | | GUGGCACCGAGUCGGUGCUU | |
| | | | | UU | |
| P9- | 103 | CACCUUCCACCG | 399 | CACCUUCCACCGCUGCGCCAG | 695 |
| hc- | | CUGCGCCAGUU | | UUUUAGAGCUAGAAAUAGCA | |
| 039 | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9- | 104 | CUUGGCGCAGC | 400 | CUUGGCGCAGCGGUGGAAGG | 696 |
| hc- | | GGUGGAAGGGU | | GUUUUAGAGCUAGAAAUAGC | |
| 040 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 105 | UCCACCGCUGCG | 401 | UCCACCGCUGCGCCAAGGUG | 697 |
| hc- | | CCAAGGUGGUU | | GUUUUAGAGCUAGAAAUAGC | |
| 041 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 106 | CACCUUGGCGCA | 402 | CACCUUGGCGCAGCGGUGGA | 698 |
| hc- | | GCGGUGGAGUU | | GUUUUAGAGCUAGAAAUAGC | |
| 042 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 107 | CCACCGCUGCGC | 403 | CCACCGCUGCGCCAAGGUGC | 699 |
| hc- | | CAAGGUGCGUU | | GUUUUAGAGCUAGAAAUAGC | |
| 043 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 108 | CCCGCACCUUGG | 404 | CCCGCACCUUGGCGCAGCGG | 700 |
| hc- | | CGCAGCGGGUU | | GUUUUAGAGCUAGAAAUAGC | |
| 044 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 109 | ACACCCGCACCU | 405 | ACACCCGCACCUUGGCGCAG | 701 |
| 045 | | UGGCGCAGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 110 | CUGCGCCAAGG | 406 | CUGCGCCAAGGUGCGGGUGU | 702 |
| 046 | | UGCGGGUGUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 111 | UGCGCCAAGGU | 407 | UGCGCCAAGGUGCGGGUGUA | 703 |
| 047 | | GCGGGUGUAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 112 | CCAAGGUGCGG | 408 | CCAAGGUGCGGGUGUAGGGA | 704 |
| 048 | | GUGUAGGGAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 113 | CAAGGUGCGGG | 409 | CAAGGUGCGGGUGUAGGGA | 705 |
| 049 | | UGUAGGGAUGU | | UGUUUUAGAGCUAGAAAUA | |
| | | UUUAGAGCUAU | | GCAAGUUAAAAUAAGGCUAG | |
| | | GCUGUUUUG | | UCCGUUAUCAACUUGAAAAA | |
| | | | | GUGGCACCGAGUCGGUGCUU | |
| | | | | UU | |
| P9-h- | 114 | CCAUCCCUACAC | 410 | CCAUCCCUACACCCGCACCUG | 706 |
| 050 | | CCGCACCUGUU | | UUUUAGAGCUAGAAAUAGCA | |
| | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 115 | GAUCCUGGCCCC | 411 | GAUCCUGGCCCCAUGCAAGG | 707 |
| 051 | | AUGCAAGGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 116 | UUGCAUGGGGC | 412 | UUGCAUGGGGCCAGGAUCCG | 708 |
| 052 | | CAGGAUCCGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 117 | ACGGAUCCUGG | 413 | ACGGAUCCUGGCCCCAUGCA | 709 |
| 053 | | CCCCAUGCAGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 118 | CAUGGGGCCAG | 414 | CAUGGGGCCAGGAUCCGUGG | 710 |
| 054 | | GAUCCGUGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 119 | CAGGAUCCGUG | 415 | CAGGAUCCGUGGAGGUUGCC | 711 |
| 055 | | GAGGUUGCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 120 | CAGGCAACCUCC | 416 | CAGGCAACCUCCACGGAUCCG | 712 |
| 056 | | ACGGAUCCGUU | | UUUUAGAGCUAGAAAUAGCA | |
| | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 121 | UAGGUGCCAGG | 417 | UAGGUGCCAGGCAACCUCCA | 713 |
| 057 | | CAACCUCCAGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 122 | GAGGUUGCCUG | 418 | GAGGUUGCCUGGCACCUACG | 714 |
| 058 | | GCACCUACGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 123 | GUUGCCUGGCA | 419 | GUUGCCUGGCACCUACGUGG | 715 |
| 059 | | CCUACGUGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 124 | AGCACCACCACG | 420 | AGCACCACCACGUAGGUGCC | 716 |
| hc- | | UAGGUGCCGUU | | GUUUUAGAGCUAGAAAUAGC | |
| 060 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 125 | CACCUACGUGG | 421 | CACCUACGUGGUGGUGCUGA | 717 |
| hc- | | UGGUGCUGAGU | | GUUUUAGAGCUAGAAAUAGC | |
| 061 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 126 | CUACGUGGUGG | 422 | CUACGUGGUGGUGCUGAAG | 718 |
| hc- | | UGCUGAAGGGU | | GGUUUUAGAGCUAGAAAUA | |
| 062 | | UUUAGAGCUAU | | GCAAGUUAAAAUAAGGCUAG | |
| | | GCUGUUUUG | | UCCGUUAUCAACUUGAAAAA | |
| | | | | GUGGCACCGAGUCGGUGCUU | |
| | | | | UU | |
| P9- | 127 | CUCCUUCAGCAC | 423 | CUCCUUCAGCACCACCACGUG | 719 |
| hc- | | CACCACGUGUU | | UUUUAGAGCUAGAAAUAGCA | |
| 063 | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 128 | GCGCUCUGACU | 424 | GCGCUCUGACUGCGAGAGGU | 720 |
| 064 | | GCGAGAGGUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 129 | UGCGCUCUGAC | 425 | UGCGCUCUGACUGCGAGAGG | 721 |
| 065 | | UGCGAGAGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 130 | CAGUGCGCUCU | 426 | CAGUGCGCUCUGACUGCGAG | 722 |
| hc- | | GACUGCGAGGU | | GUUUUAGAGCUAGAAAUAGC | |
| 066 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 131 | GCGCACUGCCCG | 427 | GCGCACUGCCCGCCGCCUGCG | 723 |
| hc- | | CCGCCUGCGUU | | UUUUAGAGCUAGAAAUAGCA | |
| 067 | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9- | 132 | UGCCCGCCGCCU | 428 | UGCCCGCCGCCUGCAGGCCCG | 724 |
| hc- | | GCAGGCCCGUU | | UUUUAGAGCUAGAAAUAGCA | |
| 068 | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 133 | UGCAGGCCCAG | 429 | UGCAGGCCCAGGCUGCCCGC | 725 |
| 069 | | GCUGCCCGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 134 | CAGGCCCAGGCU | 430 | CAGGCCCAGGCUGCCCGCCG | 726 |
| 070 | | GCCCGCCGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 135 | GUAUCCCCGGC | 431 | GUAUCCCCGGCGGGCAGCCU | 727 |
| 071 | | GGGCAGCCUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 136 | GGUAUCCCCGG | 432 | GGUAUCCCCGGCGGGCAGCC | 728 |
| 072 | | CGGGCAGCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 137 | CUUGGUGAGGU | 433 | CUUGGUGAGGUAUCCCCGGC | 729 |
| hc- | | AUCCCCGGCGU | | GUUUUAGAGCUAGAAAUAGC | |
| 073 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 138 | UCUUGGUGAGG | 434 | UCUUGGUGAGGUAUCCCCGG | 730 |
| hc- | | UAUCCCCGGGU | | GUUUUAGAGCUAGAAAUAGC | |
| 074 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 139 | GGAUCUUGGUG | 435 | GGAUCUUGGUGAGGUAUCCC | 731 |
| hc- | | AGGUAUCCCGU | | GUUUUAGAGCUAGAAAUAGC | |
| 075 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 140 | AGACAUGCAGG | 436 | AGACAUGCAGGAUCUUGGUG | 732 |
| hc- | | AUCUUGGUGGU | | GUUUUAGAGCUAGAAAUAGC | |
| 076 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 141 | AAGAUCCUGCA | 437 | AAGAUCCUGCAUGUCUUCCA | 733 |
| 077 | | UGUCUUCCAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 142 | AUGGAAGACAU | 438 | AUGGAAGACAUGCAGGAUCU | 734 |
| hc- | | GCAGGAUCUGU | | GUUUUAGAGCUAGAAAUAGC | |
| 078 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 143 | GAAGGCCAUGG | 439 | GAAGGCCAUGGAAGACAUGC | 735 |
| 079 | | AAGACAUGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 144 | GUCUUCCAUGG | 440 | GUCUUCCAUGGCCUUCUUCC | 736 |
| 080 | | CCUUCUUCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 145 | CUCAUCUUCACC | 441 | CUCAUCUUCACCAGGAAGCC | 737 |
| hc- | | AGGAAGCCGUU | | GUUUUAGAGCUAGAAAUAGC | |
| 081 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 146 | GGCUUCCUGGU | 442 | GGCUUCCUGGUGAAGAUGA | 738 |
| hc- | | GAAGAUGAGGU | | GGUUUUAGAGCUAGAAAUA | |
| 082 | | UUUAGAGCUAU | | GCAAGUUAAAAUAAGGCUAG | |
| | | GCUGUUUUG | | UCCGUUAUCAACUUGAAAAA | |
| | | | | GUGGCACCGAGUCGGUGCUU | |
| | | | | UU | |
| P9- | 147 | GGUCGCCACUCA | 443 | GGUCGCCACUCAUCUUCACC | 739 |
| hc- | | UCUUCACCGUU | | GUUUUAGAGCUAGAAAUAGC | |
| 083 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 148 | GAAGAUGAGUG | 444 | GAAGAUGAGUGGCGACCUGC | 740 |
| hc- | | GCGACCUGCGU | | GUUUUAGAGCUAGAAAUAGC | |
| 084 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 149 | GAGUGGCGACC | 445 | GAGUGGCGACCUGCUGGAGC | 741 |
| hc- | | UGCUGGAGCGU | | GUUUUAGAGCUAGAAAUAGC | |
| 085 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 150 | GGUGGCUCACC | 446 | GGUGGCUCACCAGCUCCAGC | 742 |
| hc- | | AGCUCCAGCGU | | GUUUUAGAGCUAGAAAUAGC | |
| 086 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 151 | AGCUGGUGAGC | 447 | AGCUGGUGAGCCACCCUUUU | 743 |
| 087 | | CACCCUUUUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 152 | GCUGGUGAGCC | 448 | GCUGGUGAGCCACCCUUUUU | 744 |
| 088 | | ACCCUUUUUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 153 | AAGGCCUGCAG | 449 | AAGGCCUGCAGAAGCCAGAG | 745 |
| 089 | | AAGCCAGAGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 154 | GUCGACAUGGG | 450 | GUCGACAUGGGGCAACUUCA | 746 |
| hc- | | GCAACUUCAGU | | GUUUUAGAGCUAGAAAUAGC | |
| 090 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 155 | GCCCCAUGUCGA | 451 | GCCCCAUGUCGACUACAUCG | 747 |
| hc- | | CUACAUCGGUU | | GUUUUAGAGCUAGAAAUAGC | |
| 091 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 156 | CCAUGUCGACU | 452 | CCAUGUCGACUACAUCGAGG | 748 |
| hc- | | ACAUCGAGGGU | | GUUUUAGAGCUAGAAAUAGC | |
| 092 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 157 | UCCUCGAUGUA | 453 | UCCUCGAUGUAGUCGACAUG | 749 |
| hc- | | GUCGACAUGGU | | GUUUUAGAGCUAGAAAUAGC | |
| 093 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 158 | CUCCUCGAUGU | 454 | CUCCUCGAUGUAGUCGACAU | 750 |
| hc- | | AGUCGACAUGU | | GUUUUAGAGCUAGAAAUAGC | |
| 094 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 159 | CCUCCUCGAUG | 455 | CCUCCUCGAUGUAGUCGACA | 751 |
| hc- | | UAGUCGACAGU | | GUUUUAGAGCUAGAAAUAGC | |
| 095 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 160 | GAUGCUCUGGG | 456 | GAUGCUCUGGGCAAAGACAG | 752 |
| 096 | | CAAAGACAGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 161 | UCUUUGCCCAG | 457 | UCUUUGCCCAGAGCAUCCCG | 753 |
| 097 | | AGCAUCCCGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 162 | CCAGAGCAUCCC | 458 | CCAGAGCAUCCCGUGGAACC | 754 |
| 098 | | GUGGAACCGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 163 | CAGGUUCCACG | 459 | CAGGUUCCACGGGAUGCUCU | 755 |
| 099 | | GGAUGCUCUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 164 | GCAUCCCGUGG | 460 | GCAUCCCGUGGAACCUGGAG | 756 |
| 101 | | AACCUGGAGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 165 | GUAAUCCGCUCC | 461 | GUAAUCCGCUCCAGGUUCCA | 757 |
| 103 | | AGGUUCCAGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 166 | UGGAGCGGAUU | 462 | UGGAGCGGAUUACCCCUCCA | 758 |
| 104 | | ACCCCUCCAGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 167 | GUGGAGGGGUA | 463 | GUGGAGGGGUAAUCCGCUCC | 759 |
| 105 | | AUCCGCUCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 168 | GGAUUACCCCUC | 464 | GGAUUACCCCUCCACGGUAC | 760 |
| 106 | | CACGGUACGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 169 | GAUUACCCCUCC | 465 | GAUUACCCCUCCACGGUACC | 761 |
| 107 | | ACGGUACCGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 170 | UACCCCUCCACG | 466 | UACCCCUCCACGGUACCGGG | 762 |
| 108 | | GUACCGGGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 171 | AUCCGCCCGGUA | 467 | AUCCGCCCGGUACCGUGGAG | 763 |
| 109 | | CCGUGGAGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 172 | CAUCCGCCCGGU | 468 | CAUCCGCCCGGUACCGUGGA | 764 |
| 110 | | ACCGUGGAGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 173 | UCAUCCGCCCGG | 469 | UCAUCCGCCCGGUACCGUGG | 765 |
| 111 | | UACCGUGGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 174 | UAUUCAUCCGCC | 470 | UAUUCAUCCGCCCGGUACCG | 766 |
| 112 | | CGGUACCGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 175 | GGGGCUGGUAU | 471 | GGGGCUGGUAUUCAUCCGCC | 767 |
| hc- | | UCAUCCGCCGU | | GUUUUAGAGCUAGAAAUAGC | |
| 113 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 176 | GCGGAUGAAUA | 472 | GCGGAUGAAUACCAGCCCCC | 768 |
| hc- | | CCAGCCCCCGUU | | GUUUUAGAGCUAGAAAUAGC | |
| 114 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 177 | CAGAUGGGGGU | 473 | CAGAUGGGGGUCUUACCGGG | 769 |
| 116 | | CUUACCGGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 178 | ACAGAUGGGGG | 474 | ACAGAUGGGGGUCUUACCGG | 770 |
| 117 | | UCUUACCGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 179 | CUUUCCAAGGC | 475 | CUUUCCAAGGCGACAUUUGU | 771 |
| 118 | | GACAUUUGUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 180 | UCUUUCCAAGG | 476 | UCUUUCCAAGGCGACAUUUG | 772 |
| 119 | | CGACAUUUGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 181 | CAAAUGUCGCC | 477 | CAAAUGUCGCCUUGGAAAGA | 773 |
| 120 | | UUGGAAAGAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 182 | AUGUCGCCUUG | 478 | AUGUCGCCUUGGAAAGACGG | 774 |
| 121 | | GAAAGACGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 183 | GAAAGACGGAG | 479 | GAAAGACGGAGGCAGCCUGG | 775 |
| 123 | | GCAGCCUGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 184 | CUAGGAGAUAC | 480 | CUAGGAGAUACACCUCCACC | 776 |
| hc- | | ACCUCCACCGUU | | GUUUUAGAGCUAGAAAUAGC | |
| 124 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 185 | CACUCUGUAUG | 481 | CACUCUGUAUGCUGGUGUCU | 777 |
| hc- | | CUGGUGUCUGU | | GUUUUAGAGCUAGAAAUAGC | |
| 125 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 186 | CCAGCAUACAGA | 482 | CCAGCAUACAGAGUGACCAC | 778 |
| hc- | | GUGACCACGUU | | GUUUUAGAGCUAGAAAUAGC | |
| 126 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 187 | GAGUGACCACC | 483 | GAGUGACCACCGGGAAAUCG | 779 |
| hc- | | GGGAAAUCGGU | | GUUUUAGAGCUAGAAAUAGC | |
| 129 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 188 | AGUGACCACCG | 484 | AGUGACCACCGGGAAAUCGA | 780 |
| hc- | | GGAAAUCGAGU | | GUUUUAGAGCUAGAAAUAGC | |
| 130 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 189 | ACCACCGGGAAA | 485 | ACCACCGGGAAAUCGAGGGC | 781 |
| hc- | | UCGAGGGCGUU | | GUUUUAGAGCUAGAAAUAGC | |
| 131 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 190 | CCACCGGGAAAU | 486 | CCACCGGGAAAUCGAGGGCA | 782 |
| hc- | | CGAGGGCAGUU | | GUUUUAGAGCUAGAAAUAGC | |
| 132 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 191 | CCCUGCCCUCGA | 487 | CCCUGCCCUCGAUUUCCCGG | 783 |
| hc- | | UUUCCCGGGUU | | GUUUUAGAGCUAGAAAUAGC | |
| 133 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 192 | UGACCCUGCCCU | 488 | UGACCCUGCCCUCGAUUUCC | 784 |
| hc- | | CGAUUUCCGUU | | GUUUUAGAGCUAGAAAUAGC | |
| 134 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 193 | CGACUUCGAGA | 489 | CGACUUCGAGAAUGUGCCCG | 785 |
| 135 | | AUGUGCCCGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 194 | CUCGGGCACAU | 490 | CUCGGGCACAUUCUCGAAGU | 786 |
| 136 | | UCUCGAAGUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 195 | CUUCGAGAAUG | 491 | CUUCGAGAAUGUGCCCGAGG | 787 |
| 137 | | UGCCCGAGGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 196 | AAGCGGGUCCC | 492 | AAGCGGGUCCCGUCCUCCUC | 788 |
| hc- | | GUCCUCCUCGU | | GUUUUAGAGCUAGAAAUAGC | |
| 139 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 197 | GAAGCGGGUCC | 493 | GAAGCGGGUCCCGUCCUCCU | 789 |
| hc- | | CGUCCUCCUGU | | GUUUUAGAGCUAGAAAUAGC | |
| 140 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 198 | CGGGACCCGCU | 494 | CGGGACCCGCUUCCACAGAC | 790 |
| hc- | | UCCACAGACGU | | GUUUUAGAGCUAGAAAUAGC | |
| 141 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 199 | GCUUACCUGUC | 495 | GCUUACCUGUCUGUGGAAGC | 791 |
| hc- | | UGUGGAAGCGU | | GUUUUAGAGCUAGAAAUAGC | |
| 142 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 200 | CGGCCGUGCUU | 496 | CGGCCGUGCUUACCUGUCUG | 792 |
| 143 | | ACCUGUCUGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 201 | AGGUAAGCACG | 497 | AGGUAAGCACGGCCGUCUGA | 793 |
| 144 | | GCCGUCUGAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 202 | GGUAAGCACGG | 498 | GGUAAGCACGGCCGUCUGAU | 794 |
| 145 | | CCGUCUGAUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 203 | GGUCCUUGUGU | 499 | GGUCCUUGUGUUCGUCGAGC | 795 |
| 146 | | UCGUCGAGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 204 | UGGCCUGCUCG | 500 | UGGCCUGCUCGACGAACACA | 796 |
| 147 | | ACGAACACAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 205 | GCCAGCAAGUG | 501 | GCCAGCAAGUGUGACAGUCA | 797 |
| 148 | | UGACAGUCAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 206 | GCCAUGACUGU | 502 | GCCAUGACUGUCACACUUGC | 798 |
| 149 | | CACACUUGCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 207 | GCUGCGCAUGC | 503 | GCUGCGCAUGCUGGCACCCU | 799 |
| 156 | | UGGCACCCUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 208 | CACGCGCAGGCU | 504 | CACGCGCAGGCUGCGCAUGC | 800 |
| 157 | | GCGCAUGCGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 209 | CUGCGCGUGCU | 505 | CUGCGCGUGCUCAACUGCCA | 801 |
| hc- | | CAACUGCCAGU | | GUUUUAGAGCUAGAAAUAGC | |
| 158 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 210 | UGCGCGUGCUC | 506 | UGCGCGUGCUCAACUGCCAA | 802 |
| hc- | | AACUGCCAAGU | | GUUUUAGAGCUAGAAAUAGC | |
| 159 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 211 | CUUGGCAGUUG | 507 | CUUGGCAGUUGAGCACGCGC | 803 |
| hc- | | AGCACGCGCGU | | GUUUUAGAGCUAGAAAUAGC | |
| 160 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 212 | CGUGCUCAACU | 508 | CGUGCUCAACUGCCAAGGGA | 804 |
| hc- | | GCCAAGGGAGU | | GUUUUAGAGCUAGAAAUAGC | |
| 161 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9 | 213 | GUGCUCAACUG | 509 | GUGCUCAACUGCCAAGGGAA | 805 |
| hc- | | CCAAGGGAAGU | | GUUUUAGAGCUAGAAAUAGC | |
| 162 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 214 | CAAGGGAAGGG | 510 | CAAGGGAAGGGCACGGUUAG | 806 |
| 163 | | CACGGUUAGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 215 | CGCUAACCGUGC | 511 | CGCUAACCGUGCCCUUCCCU | 807 |
| 164 | | CCUUCCCUGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 216 | ACGGUUAGCGG | 512 | ACGGUUAGCGGCACCCUCAU | 808 |
| 165 | | CACCCUCAUGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 217 | GGGCCAUCACU | 513 | GGGCCAUCACUUACCUAUGA | 809 |
| 166 | | UACCUAUGAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 218 | GGGGCCAUCAC | 514 | GGGGCCAUCACUUACCUAUG | 810 |
| 167 | | UUACCUAUGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9 | 219 | UCCGAAUAAAC | 515 | UCCGAAUAAACUCCAGGCCU | 811 |
| hc- | | UCCAGGCCUGU | | GUUUUAGAGCUAGAAAUAGC | |
| 169 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 220 | GGCUUUUCCGA | 516 | GGCUUUUCCGAAUAAACUCC | 812 |
| hc- | | AUAAACUCCGU | | GUUUUAGAGCUAGAAAUAGC | |
| 171 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 221 | GUUUAUUCGGA | 517 | GUUUAUUCGGAAAAGCCAGC | 813 |
| hc- | | AAAGCCAGCGU | | GUUUUAGAGCUAGAAAUAGC | |
| 172 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 222 | GCCAGCUGGUC | 518 | GCCAGCUGGUCCAGCCUGUG | 814 |
| 173 | | CAGCCUGUGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 223 | AGCACCACCAGU | 519 | AGCACCACCAGUGGCCCCACG | 815 |
| 174 | | GGCCCCACGUU | | UUUUAGAGCUAGAAAUAGCA | |
| | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9- | 224 | CGGCUGUACCCA | 520 | CGGCUGUACCCACCCGCCAGG | 816 |
| hc- | | CCCGCCAGGUU | | UUUUAGAGCUAGAAAUAGCA | |
| 175 | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9- | 225 | GUCGUGCUGGU | 521 | GUCGUGCUGGUCACCGCUGC | 817 |
| hc- | | CACCGCUGCGU | | GUUUUAGAGCUAGAAAUAGC | |
| 185 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 226 | UCACCGCUGCCG | 522 | UCACCGCUGCCGGCAACUUC | 818 |
| hc- | | GCAACUUCGUU | | GUUUUAGAGCUAGAAAUAGC | |
| 186 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 227 | CACCGCUGCCGG | 523 | CACCGCUGCCGGCAACUUCCG | 819 |
| hc- | | CAACUUCCGUU | | UUUUAGAGCUAGAAAUAGCA | |
| 187 | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9- | 228 | GUCCCGGAAGU | 524 | GUCCCGGAAGUUGCCGGCAG | 820 |
| hc- | | UGCCGGCAGGU | | GUUUUAGAGCUAGAAAUAGC | |
| 188 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 229 | GGCAUCGUCCC | 525 | GGCAUCGUCCCGGAAGUUGC | 821 |
| hc- | | GGAAGUUGCGU | | GUUUUAGAGCUAGAAAUAGC | |
| 189 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 230 | AGUAGAGGCAG | 526 | AGUAGAGGCAGGCAUCGUCC | 822 |
| hc- | | GCAUCGUCCGU | | GUUUUAGAGCUAGAAAUAGC | |
| 190 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 231 | CCAGCCUCAGCU | 527 | CCAGCCUCAGCUCCCGAGGU | 823 |
| 191 | | CCCGAGGUGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 232 | AGCACCUACCUC | 528 | AGCACCUACCUCGGGAGCUG | 824 |
| 192 | | GGGAGCUGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 233 | CUUUCCAGGUC | 529 | CUUUCCAGGUCAUCACAGUU | 825 |
| hc- | | AUCACAGUUGU | | GUUUUAGAGCUAGAAAUAGC | |
| 194 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 234 | UUUCCAGGUCA | 530 | UUUCCAGGUCAUCACAGUUG | 826 |
| hc- | | UCACAGUUGGU | | GUUUUAGAGCUAGAAAUAGC | |
| 195 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 235 | UGGCCCCAACUG | 531 | UGGCCCCAACUGUGAUGACC | 827 |
| hc- | | UGAUGACCGUU | | GUUUUAGAGCUAGAAAUAGC | |
| 196 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 236 | CGGCUGGUCUU | 532 | CGGCUGGUCUUGGGCAUUG | 828 |
| 197 | | GGGCAUUGGGU | | GGUUUUAGAGCUAGAAAUA | |
| | | UUUAGAGCUAU | | GCAAGUUAAAAUAAGGCUAG | |
| | | GCUGUUUUG | | UCCGUUAUCAACUUGAAAAA | |
| | | | | GUGGCACCGAGUCGGUGCUU | |
| | | | | UU | |
| P9-h- | 237 | CACCGGCUGGU | 533 | CACCGGCUGGUCUUGGGCAU | 829 |
| 198 | | CUUGGGCAUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 238 | CAAGACCAGCCG | 534 | CAAGACCAGCCGGUGACCCU | 830 |
| 199 | | GUGACCCUGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 239 | AAGACCAGCCGG | 535 | AAGACCAGCCGGUGACCCUG | 831 |
| 200 | | UGACCCUGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 240 | CAGGGUCACCG | 536 | CAGGGUCACCGGCUGGUCUU | 832 |
| 201 | | GCUGGUCUUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 241 | GACCAACUUUG | 537 | GACCAACUUUGGCCGCUGUG | 833 |
| hc- | | GCCGCUGUGGU | | GUUUUAGAGCUAGAAAUAGC | |
| 208 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 242 | GUCCACACAGCG | 538 | GUCCACACAGCGGCCAAAGU | 834 |
| hc- | | GCCAAAGUGUU | | GUUUUAGAGCUAGAAAUAGC | |
| 209 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 243 | GGGCAAAGAGG | 539 | GGGCAAAGAGGUCCACACAG | 835 |
| hc- | | UCCACACAGGU | | GUUUUAGAGCUAGAAAUAGC | |
| 210 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 244 | GUGUGGACCUC | 540 | GUGUGGACCUCUUUGCCCCA | 836 |
| hc- | | UUUGCCCCAGU | | GUUUUAGAGCUAGAAAUAGC | |
| 212 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 245 | UGUCCUCCCCU | 541 | UGUCCUCCCCUGGGGCAAAG | 837 |
| hc- | | GGGGCAAAGGU | | GUUUUAGAGCUAGAAAUAGC | |
| 215 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 246 | ACCUGCUUUGU | 542 | ACCUGCUUUGUGUCACAGAG | 838 |
| 218 | | GUCACAGAGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 247 | CCUGCUUUGUG | 543 | CCUGCUUUGUGUCACAGAGU | 839 |
| 219 | | UCACAGAGUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 248 | CCCACUCUGUGA | 544 | CCCACUCUGUGACACAAAGC | 840 |
| 220 | | CACAAAGCGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 249 | GUCACAGAGUG | 545 | GUCACAGAGUGGGACAUCAC | 841 |
| 221 | | GGACAUCACGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 250 | UGGUGACUUAC | 546 | UGGUGACUUACCAGCCACGU | 842 |
| hc- | | CAGCCACGUGU | | GUUUUAGAGCUAGAAAUAGC | |
| 223 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 251 | GUGGUGACUUA | 547 | GUGGUGACUUACCAGCCACG | 843 |
| hc- | | CCAGCCACGGUU | | GUUUUAGAGCUAGAAAUAGC | |
| 224 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 252 | UGCCGAGCCGG | 548 | UGCCGAGCCGGAGCUCACCC | 844 |
| 228 | | AGCUCACCCGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 253 | GGCCAGGGUGA | 549 | GGCCAGGGUGAGCUCCGGCU | 845 |
| 229 | | GCUCCGGCUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 254 | AACUCGGCCAG | 550 | AACUCGGCCAGGGUGAGCUC | 846 |
| 230 | | GGUGAGCUCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 255 | GUCUCUGCCUC | 551 | GUCUCUGCCUCAACUCGGCC | 847 |
| 233 | | AACUCGGCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 256 | GAUCAGUCUCU | 552 | GAUCAGUCUCUGCCUCAACU | 848 |
| hc- | | GCCUCAACUGU | | GUUUUAGAGCUAGAAAUAGC | |
| 234 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 257 | UGACAUCUUUG | 553 | UGACAUCUUUGGCAGAGAAG | 849 |
| hc- | | GCAGAGAAGGU | | GUUUUAGAGCUAGAAAUAGC | |
| 235 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 258 | AAGAUGUCAUC | 554 | AAGAUGUCAUCAAUGAGGCC | 850 |
| hc- | | AAUGAGGCCGU | | GUUUUAGAGCUAGAAAUAGC | |
| 236 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 259 | CAAUGAGGCCU | 555 | CAAUGAGGCCUGGUUCCCUG | 851 |
| hc- | | GGUUCCCUGGU | | GUUUUAGAGCUAGAAAUAGC | |
| 238 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 260 | UCAGUACCCGCU | 556 | UCAGUACCCGCUGGUCCUCA | 852 |
| hc- | | GGUCCUCAGUU | | GUUUUAGAGCUAGAAAUAGC | |
| 239 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 261 | GCGGGUACUGA | 557 | GCGGGUACUGACCCCCAACC | 853 |
| hc- | | CCCCCAACCGUU | | GUUUUAGAGCUAGAAAUAGC | |
| 241 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 262 | GGUUGGGGGUC | 558 | GGUUGGGGGUCAGUACCCGC | 854 |
| hc- | | AGUACCCGCGU | | GUUUUAGAGCUAGAAAUAGC | |
| 242 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 263 | GGUACUGACCCC | 559 | GGUACUGACCCCCAACCUGG | 855 |
| hc- | | CAACCUGGGUU | | GUUUUAGAGCUAGAAAUAGC | |
| 243 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 264 | CCUGCCCCCCAG | 560 | CCUGCCCCCCAGCACCCAUGG | 856 |
| 244 | | CACCCAUGGUU | | UUUUAGAGCUAGAAAUAGCA | |
| | | UUAGAGCUAUG | | AGUUAAAAUAAGGCUAGUCC | |
| | | CUGUUUUG | | GUUAUCAACUUGAAAAAGUG | |
| | | | | GCACCGAGUCGGUGCUUUU | |
| P9-h- | 265 | UUACCUGCCCCA | 561 | UUACCUGCCCCAUGGGUGCU | 857 |
| 246 | | UGGGUGCUGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 266 | GCCAACCUGCAA | 562 | GCCAACCUGCAAAAAGGGCC | 858 |
| 250 | | AAAGGGCCGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 267 | GACUGUAUGGU | 563 | GACUGUAUGGUCAGCACACU | 859 |
| 252 | | CAGCACACUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 268 | ACUGUAUGGUC | 564 | ACUGUAUGGUCAGCACACUC | 860 |
| 253 | | AGCACACUCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 269 | CUGUAUGGUCA | 565 | CUGUAUGGUCAGCACACUCG | 861 |
| 254 | | GCACACUCGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 270 | CAGCACACUCGG | 566 | CAGCACACUCGGGGCCUACA | 862 |
| 255 | | GGCCUACAGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 271 | ACACUCGGGGCC | 567 | ACACUCGGGGCCUACACGGA | 863 |
| 256 | | UACACGGAGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 272 | ACGGCUGUGGC | 568 | ACGGCUGUGGCCAUCCGUGU | 864 |
| hc- | | CAUCCGUGUGU | | GUUUUAGAGCUAGAAAUAGC | |
| 257 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 273 | GCUCCUCAUCU | 569 | GCUCCUCAUCUGGGGCGCAG | 865 |
| 260 | | GGGGCGCAGGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 274 | GCAGACCAGCU | 570 | GCAGACCAGCUUGCCCCCUU | 866 |
| 268 | | UGCCCCCUUGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 275 | UGCCGGGCCCAC | 571 | UGCCGGGCCCACAACGCUUU | 867 |
| hc- | | AACGCUUUGUU | | GUUUUAGAGCUAGAAAUAGC | |
| 272 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 276 | GCCGGGCCCACA | 572 | GCCGGGCCCACAACGCUUUU | 868 |
| hc- | | ACGCUUUUGUU | | GUUUUAGAGCUAGAAAUAGC | |
| 273 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 277 | CCGGGCCCACAA | 573 | CCGGGCCCACAACGCUUUUG | 869 |
| hc- | | CGCUUUUGGUU | | GUUUUAGAGCUAGAAAUAGC | |
| 274 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 278 | CGGGCCCACAAC | 574 | CGGGCCCACAACGCUUUUGG | 870 |
| hc- | | GCUUUUGGGUU | | GUUUUAGAGCUAGAAAUAGC | |
| 275 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 279 | CCACAACGCUUU | 575 | CCACAACGCUUUUGGGGGUG | 871 |
| hc- | | UGGGGGUGGUU | | GUUUUAGAGCUAGAAAUAGC | |
| 277 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 280 | CUCACCCCCAAA | 576 | CUCACCCCCAAAAGCGUUGU | 872 |
| hc- | | AGCGUUGUGUU | | GUUUUAGAGCUAGAAAUAGC | |
| 279 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 281 | AGGGUGUCUAC | 577 | AGGGUGUCUACGCCAUUGCC | 873 |
| hc- | | GCCAUUGCCGU | | GUUUUAGAGCUAGAAAUAGC | |
| 281 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 282 | GUGGACACGGG | 578 | GUGGACACGGGUCCCCAUGC | 874 |
| hc- | | UCCCCAUGCGU | | GUUUUAGAGCUAGAAAUAGC | |
| 282 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9- | 283 | AAGGUCCUCCAC | 579 | AAGGUCCUCCACCUCCCAGU | 875 |
| hc- | | CUCCCAGUGUU | | GUUUUAGAGCUAGAAAUAGC | |
| 283 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 284 | CCUCAGCACAGG | 580 | CCUCAGCACAGGCGGCUUGU | 876 |
| hc- | | CGGCUUGUGUU | | GUUUUAGAGCUAGAAAUAGC | |
| 284 | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9- | 285 | CACUGGUUGGG | 581 | CACUGGUUGGGCUGACCUCG | 877 |
| hc- | | CUGACCUCGGU | | GUUUUAGAGCUAGAAAUAGC | |
| 285 | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 286 | AGGUCAGCCCAA | 582 | AGGUCAGCCCAACCAGUGCG | 878 |
| 286 | | CCAGUGCGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 287 | GGUCAGCCCAAC | 583 | GGUCAGCCCAACCAGUGCGU | 879 |
| 287 | | CAGUGCGUGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 288 | CAACCAGUGCG | 584 | CAACCAGUGCGUGGGCCACA | 880 |
| 288 | | UGGGCCACAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 289 | UAGACAACACG | 585 | UAGACAACACGUGUGUAGUC | 881 |
| 289 | | UGUGUAGUCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 290 | GACUACACACGU | 586 | GACUACACACGUGUUGUCUA | 882 |
| 290 | | GUUGUCUAGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 291 | CACGUGUGUAG | 587 | CACGUGUGUAGUCAGGAGCC | 883 |
| 291 | | UCAGGAGCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 292 | AGCCGGGACGU | 588 | AGCCGGGACGUCAGCACUAC | 884 |
| 292 | | CAGCACUACGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 293 | UGCCUGUAGUG | 589 | UGCCUGUAGUGCUGACGUCC | 885 |
| 293 | | CUGACGUCCGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |
| P9-h- | 294 | ACUACAGGCAGC | 590 | ACUACAGGCAGCACCAGCGA | 886 |
| 294 | | ACCAGCGAGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 295 | UACAGGCAGCAC | 591 | UACAGGCAGCACCAGCGAAG | 887 |
| 295 | | CAGCGAAGGUU | | GUUUUAGAGCUAGAAAUAGC | |
| | | UUAGAGCUAUG | | AAGUUAAAAUAAGGCUAGUC | |
| | | CUGUUUUG | | CGUUAUCAACUUGAAAAAGU | |
| | | | | GGCACCGAGUCGGUGCUUUU | |
| P9-h- | 296 | GCAGAUGGCAA | 592 | GCAGAUGGCAACGGCUGUCA | 888 |
| 296 | | CGGCUGUCAGU | | GUUUUAGAGCUAGAAAUAGC | |
| | | UUUAGAGCUAU | | AAGUUAAAAUAAGGCUAGUC | |
| | | GCUGUUUUG | | CGUUAUCAACUUGAAAAAGU | |
| | | | | GGCACCGAGUCGGUGCUUUU | |

The 296 sgRNA sequences shown in Table 5 (SEQ ID NOs: 593-888) were tested further in in vitro and in vivo assays.

### Example 2. Target Analysis

On-target efficiency analysis: deep amplicon sequencing was used to evaluate on-targeting cutting efficiency. In-house computational tools and workflows were used to enumerate and visualize targeted mutations introduced by gene-editing systems disclosed herein. Editing effects on coding and non-coding elements associated with the selected target regions were evaluated.

Off-targeted cleavage was also evaluated. For instance, a cell-based oligo insertion-based assay was also performed (Tasi et al., 2015) in PHH, Huh7 and HepG2 cell lines. The sites with high dsODN insertion efficiencies were chosen for further analysis using amplicon based next generation sequencing for a more precise evaluation of the off target editing.

Cas9 Protein and sgRNA Delivery In Vitro

HepG2 cell line. The human hepatocellular carcinoma cell line HepG2 was cultured in DMEM media supplemented with 10% fetal bovine serum. Cells were plated at a density of 1,000,000-1,500,000 cells/well in a 6-well plate or 8,000-22,000 cells/well in a 96-well plate 24 hours prior to electroporation. Cells were electroporated with Celetrix electroporator (Celetrix, CTX-1500A) per the manufacturer's protocol. Cells were electroporated with a RNP complex containing Cas9 Nuclease (5-50 pmol), sgRNA (10-500 pmol) and Celetrix buffer.

Cas9 mRNA and sgRNA Delivery In Vitro

HepG2 cell line. The human hepatocellular carcinoma cell line HepG2 was cultured in DMEM media supplemented with 10% fetal bovine serum. Cells were plated at a density of 1,000,000-1,500,000 cells/well in a 6-well plate or 8,000-22,000 cells/well in a 96-well plate 24 hours prior to electroporation. Cells were transfected with Lipofectamine MessengerMAX (ThermoFisher, Cat. LMRNA003) per the manufacturer's protocol. Cells were transfected with a lipoplex containing 1-500 ng Cas9 mRNA, 2-1,000 ng sgRNA and Lipofectamine MessengerMAX.

Huh7 cell line. The human hepatocellular carcinoma cell line Huh7 was cultured in DMEM media supplemented with 10% fetal bovine serum. Cells were plated at a density of 500,000-1,500,000 cells/well in a 6-well plate or 5,000-15,000 cells/well in a 96-well plate 24 hours prior to electroporation. Cells were transfected with Lipofectamine MessengerMAX (ThermoFisher, Cat. LMRNA003) per the manufacturer's protocol. Cells were transfected with a lipoplex containing 1-500 ng Cas9 mRNA, 2-1,000 ng sgRNA and Lipofectamine MessengerMAX.

Cos-7 cell line. The Green Monkey kidney cell line Cos-7 was cultured in DMEM media supplemented with 10% fetal bovine serum. Cells were plated at a density of 5,000-15,000 cells/well in a 96-well plate 24 hours prior to electroporation. Cells were transfected with Lipofectamine MessengerMAX (ThermoFisher, Cat. LMRNA003) per the manufacturer's protocol. Cells were transfected with a lipoplex containing 1-500 ng Cas9 mRNA, 2-1,000 ng sgRNA and Lipofectamine MessengerMAX.

Primary liver hepatocytes. Primary human liver hepatocytes (PHH) and primary cynomolgus liver hepatocytes (PCH) (BioIVT) were cultured per the manufacturer's protocol. In brief, the cells were thawed and resuspended in hepatocyte thawing medium with supplements followed by centrifugation at 100 g for 10 minutes for human and 80 g for 4 minutes for cyno. The supernatant was discarded and the pelleted cells resuspended in hepatocyte plating medium plus supplement pack. Cells were counted and plated on Bio-coat collagen I coated 96-well plates (ThermoFisher, Cat. 877272) at a density of 60,000 cells/well in a 96-well plate or 125,000 cells/well in a 24-well plate or 270,000 cells/well in a 6-well plate. Plated cells were allowed to settle and adhere for 6 or 24 hours in a tissue culture incubator at 37° C. and 5% CO₂ atmosphere.

After incubation cells were checked for monolayer formation and media was replaced with hepatocyte culture medium with serum-free supplement pack.

Genomic DNA isolation. For in vitro study, transfected cells were harvested post-transfection at 72 hours. The genomic DNA was extracted from either each well of a 6-well/24-well/96-well plate using QuickExtract DNA Extraction Solution (LGC Lucigen, Cat. QE09050) per manufacturer's protocol. All DNA samples were subjected to subsequent Sanger sequencing analyses, as described herein.

For in vivo study, the genomic DNA was extracted from mice liver homogenate using FastPure Blood/Cell/Tissue/Bacteria DNA Isolation Mini Kit (Vazyme, Cat. DC112) following manufacture's protocol.

Sanger Sequencing analysis. To quantitatively determine the efficiency of editing at the target location in the genome and quickly shortlist potential gRNAs, Sanger sequencing was utilized to identify the editing efficiency introduced by gene editing.

Primers were designed around the target site within the gene of interest (e.g. PCSK9), and the genomic area of interest was amplified.

Sanger sequencing was performed on 3730xl DNA Analyzer (ThermoFisher, Cat. 3730XL) per manufacturer's protocol. The raw sequencing files (.ab1) were analyzed for determining editing efficiency using online analysis tools.

Next-generation sequencing (NGS) analysis. To quantitatively determine the efficiency and pattern of editing at the target location in the genome, sequencing was utilized to identify the presence of insertions and deletions introduced by gene editing.

Primers were designed around the target site within the gene of interest (e.g. PCSK9), and the genomic area of interest was amplified.

Additional PCR was performed per the manufacturer's protocols (Illumina) to add chemistry for sequencing. The amplicons were sequenced on an Illumina NovaSeq 6000 instrument. The reads were aligned to a reference genome (e.g., the human reference genome (hg38), the cynomolgous reference genome (mf5), the rat reference genome (6), or the mouse reference genome (mm10)) after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated.

The editing percentage (e.g., the “editing efficiency” or “percent editing” or “indel frequency”) is defined as the total number of sequence reads with insertions/deletions (“indels”) or substitutions over the total number of sequence reads, including wild type.

PCSK9 ELISA analysis used in cell studies. Cell (HepG2 or Huh7) lysates were collected and isolated, then the PCSK9 expression levels were determined using a Human PCSK9 ELISA Kit (Abcam, Cat. ab209884), according to manufacturer's protocol. Briefly, samples were serial diluted with kit sample diluent to a final dilution of 5,000-fold when measuring human PCSK9. 100 uL of the prepared standard curve or diluted serum samples were added to the ELISA plate, incubated for 30 minutes at room temperature then washed 3 times with provided wash buffer. 100 uL of detection antibody was then added to each well and incubated for 20 minutes at room temperature followed by 3 washes. 100 uL of substrate is added then incubated for 10 minutes at room temperature before the addition of 100 ul stop solution. The absorbance of the contents was measured on the Spectramax M5 plate reader with analysis using SoftmaxPro version 7.0 software. PCSK9 levels were calculated from the standard curve using 4 parameter logistic fit and expressed as ng/ml of serum or percent knockdown relative control (vehicle treated) cells.

SgRNA synthesis. sgRNA was synthesized on a 192-YiBo solid-phase synthesizer. Controlled-pore glass (CPG) was used as the solid support, TBDMS-modified phosphoramidite were used to add each monomer per cycle. At the end of the synthesis process, sgRNA were cleaved from the CPG for deprotection process. The purification were performed in a AKTA purification machine.

In other experiments, the sgRNA was ordered from vendor such as Genscript, General Biosystem or synthego. SgRNA from the same vendor and of similar purity were used for every experiment if the aim was to compare the potency or off target among sgRNA.

mRNA codon optimization. 004R sequence was optimized by Genscript using its internal algorithm for optimized human protein production and low GC content that facilitates gene synthesis. Seq311 and Seq204 were from U.S. Pat. No. 11,697,806B2 for comparison. K1-1, K4-8, K8-1 and K10-2 were optimized based on high Codon Adaptation Index (CAI) and low minimum free energy (MFE). As the calculation of MFE requires full length mRNA, all tested sequences includes the same 5′ UTR (5′ UTR HSD, TCCCGCAGTCGGCGTCCAGCGGCTCTGCTTGTTCGTGTGTGTGTCGTTGCAGG CCTTATTC, SEQ ID NO: 961), 3′ UTR (3′ UTR ALB, CATCACATTTAAAAGCATCTCAGCCTACCATGAGAATAAGAGAAAGAAAATG AAGATCAATAGCTTATTCATCTCTTTTTCTTTTTCGTTGGTGTAAAGCCAACAC CCTGTCTAAAAAACATAAATTTCTTTAATCATTTTGCCTCTTTTCTCTGTGCTT CAATTAATAAAAAATGGAAAGAA, SEQ ID NO: 962) and Nuclear localization sequences (G3S-NLS). All codon optimized CDS were compared with the same UTR as following.

mRNA plasmid construction and in vitro transcription (IVT). Different sequence elements (e.g. UTR, CDS, polyA, see sequence list) were PCR amplified or de novo synthesized and cloned into the original plasmid (Genscript, General Biosystem or GENEWIZ), which is used for the production of Cas9 mRNA. PolyA length in the plasmid were validated by sanger by the gene synthesis provider with a difference less than 3 from the designed number.

Capped and polyadenylated Cas9 mRNA containing N1-methyl pseudo-U was generated by in vitro transcription using a linearized plasmid template and T7 RNA polymerase. The transcript concentration was determined by measuring the light absorbance at 260 nm (Nanodrop), and the transcript was analyzed by capillary electrophoresis by Bioanalyzer (Agilent).

PolyA length measurement by Mass spectroscopy. For detection of the length of poly A mRNA, the full length of mRNA was cleaved by RNase Tlt to break up the phosphodiester bond between the 3′-phosphate group of the guanine ribonucleotide and the 5′-hydroxyl group of the adjacent ribonucleotide. This process released a short poly A fragment from the parental mRNA molecule. The released poly A fragment was then purified using biotin-avidin magnetic beads. The molecular weight distribution of this polyA fragment is then analyzed by a mass spectrometer.

LNP Delivery In Vivo. Compositions for delivery of the protein and nucleic acid components of CRISPR/Cas to a cell, such as a cell in a patient, are needed. Particularly, compositions with useful properties for in vitro and in vivo delivery that can stabilize and deliver RNA components are of interest.

Herein, we provide lipid nanoparticle (LNP)-based compositions with useful properties, in particular for delivery of CRISPR/Cas gene editing components. The LNP compositions comprise: an RNA component; and a lipid component, wherein the lipid component comprises: (1) 45-55 mol-% amine lipid; (2) 9-11 mol-% neutral lipid; and (3) 1-5 mol-% PEG lipid, wherein the remainder of the lipid component is helper lipid, and wherein the N/P ratio of the LNP composition is 3-8.

Unless otherwise noted, PCSK9-humanized mice, ranging 6-15 weeks of age were used in each study. Animals were weighed and grouped according to body weight for preparing dosing solutions based on group average weight. LNPs were dosed via the tail vein in a volume of 0.2 ml per animal (approximately 10 ml per kilogram body weight). The animals were observed every day to monitor status. Blood samples were collected from saphenous vein or heart puncture at indicated time points. Liver tissues were collected from mice after blood collection and immediately put in −80° C. for further analysis.

PCSK9 ELISA analysis used in animal studies. Blood was collected and the serum was isolated. The total human PCSK9 serum levels were measured using Human PCSK9 ELISA Kit (Abcam, Cat. ab209884), following manufacture's protocol.

### Example 3: Screening of sgRNA Sequences

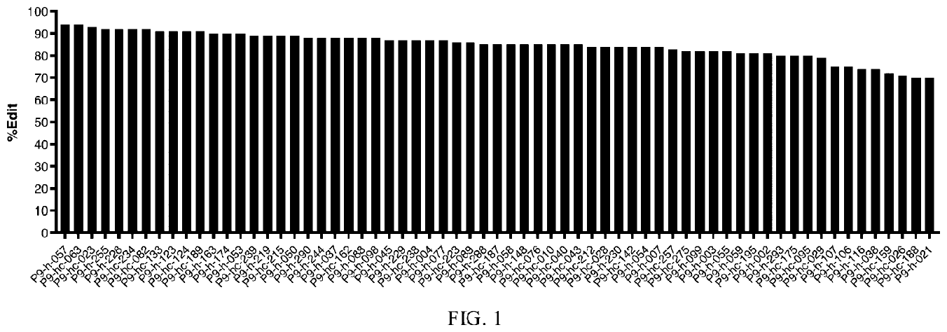

Screening of PCSK9 guide RNAs in HepG2 cells. sgRNAs targeting the human PCSK9 gene were delivered to HepG2 as described in Example 2. Percent editing was determined for sgRNAs comprising each guide sequence and the guide sequences were then rank ordered based on highest % edit. The editing data are listed below in Table 6 below. The data are shown graphically in FIG. 1.

TABLE 6
PCSK9 editing data in HepG2 cells with Cas9 protein and sgRNAs

| Name | % Edit |
|---|---|
| P9-h-057 | 94 |
| P9-hc-063 | 94 |
| P9-hc-023 | 93 |
| P9-h-255 | 92 |
| P9-h-228 | 92 |
| P9-hc-234 | 92 |
| P9-hc-082 | 92 |
| P9-hc-133 | 91 |
| P9-h-123 | 91 |
| P9-hc-124 | 91 |
| P9-hc-189 | 91 |
| P9-h-163 | 90 |
| P9-h-174 | 90 |
| P9-h-053 | 90 |
| P9-hc-239 | 89 |
| P9-h-219 | 89 |
| P9-hc-215 | 89 |
| P9-h-050 | 89 |
| P9-h-290 | 88 |
| P9-h-244 | 88 |
| P9-h-037 | 88 |
| P9-hc-162 | 88 |
| P9-h-088 | 88 |
| P9-h-098 | 88 |
| P9-h-045 | 87 |
| P9-h-229 | 87 |
| P9-hc-238 | 87 |
| P9-h-004 | 87 |
| P9-h-077 | 87 |
| P9-hc-223 | 86 |
| P9-h-089 | 86 |
| P9-h-288 | 85 |
| P9-hc-187 | 85 |
| P9-h-058 | 85 |
| P9-h-148 | 85 |
| P9-hc-076 | 85 |
| P9-hc-010 | 85 |
| P9-hc-040 | 85 |
| P9-hc-043 | 85 |
| P9-hc-212 | 84 |
| P9-hc-028 | 84 |
| P9-h-230 | 84 |
| P9-hc-142 | 84 |
| P9-h-054 | 84 |
| P9-h-007 | 84 |
| P9-hc-257 | 83 |
| P9-hc-275 | 82 |
| P9-h-099 | 82 |
| P9-h-003 | 82 |
| P9-h-055 | 82 |
| P9-h-059 | 81 |
| P9-hc-195 | 81 |
| P9-h-002 | 81 |
| P9-h-293 | 80 |
| P9-hc-175 | 80 |
| P9-hc-095 | 80 |
| P9-hc-029 | 79 |
| P9-h-107 | 75 |
| P9-h-106 | 75 |
| P9-h-116 | 74 |
| P9-h-038 | 74 |
| P9-hc-159 | 72 |
| P9-hc-026 | 71 |
| P9-hc-188 | 70 |
| P9-h-021 | 70 |

### Example 4. Dose Response of sgRNAs in Cos-7 and PCH Cells

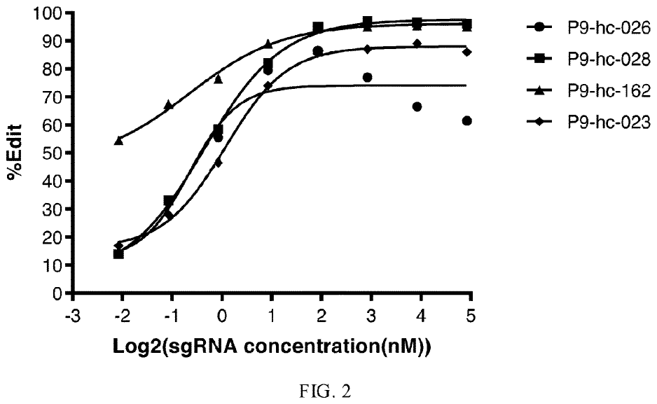

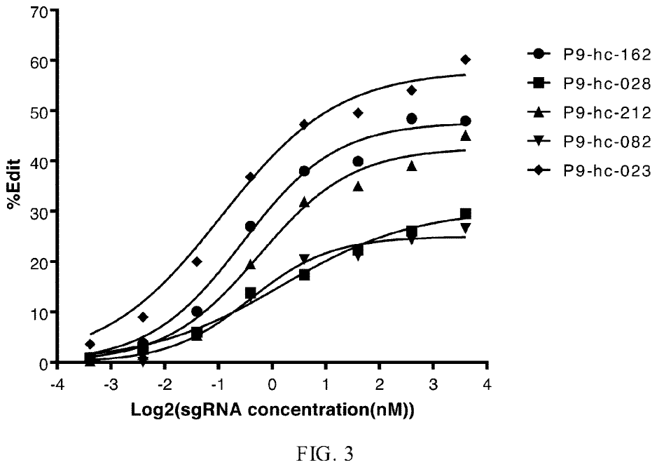

sgRNAs targeting human and monkey PCSK9 and Cas9 mRNA were delivered to Cos-7 and PCH cells as described in Example 2, in an 8 point 2-fold dose response curve. The cells were lysed 72 hours post treatment for editing analysis as described in Example 2. Percent editing was determined for sgRNAs comprising each guide sequence and the guide sequences were then rank ordered based on EC50 values and maximum editing percent. The dose response curve data for the guide sequences in Cos-7 and PCH cells is shown in FIGS. 2 and 3. The EC50 values and maximum editing percent are listed in Tables 7 and 8 below.

Table 7 shows the EC50 and maximum editing of the tested human PCSK9 sgRNAs with Cas9 mRNA on Cos-7 as dose response curves. The data are shown graphically in FIG. 2.

TABLE 7
PCSK9 editing data in Cos-7 cells
treated with Cas9 mRNA and sgRNAs

| Name | EC50 (nM) | Max Edit (%) |
|---|---|---|
| P9-hc-026 | 0.221 | 87 |
| P9-hc-028 | 0.439 | 97 |
| P9-hc-162 | 0.217 | 96 |
| P9-hc-023 | 1.158 | 89 |

Table 8 below shows the EC50 and maximum editing of the tested human PCSK9 sgRNAs with Cas9 mRNA on PCH as dose response curves. The data are shown graphically in FIG. 3.

TABLE 8
PCSK9 editing data in PCH cells
treated with Cas9 mRNA and sgRNAs

| Name | EC50 (nM) | Max Edit (%) |
|---|---|---|
| P9-hc-082 | 0.749 | 27 |
| P9-hc-028 | 1.126 | 30 |
| P9-hc-162 | 0.704 | 48 |
| P9-hc-023 | 0.553 | 60 |
| P9-hc-212 | 0.880 | 45 |

### Example 5. Phenotypic Analysis

ELISA analysis of intracellular PCSK9. HepG2 and Huh7 cells were transfected as described in Example 2 with Cas9 mRNA and sgRNA. The transfected pools of cells were retained in tissue culture and passaged for further analysis. At Day 5 post-transfection, cell lysates were harvested and subjected to analysis by ELISA as previously described.

Percent reduction of PCSK9 protein was calculated. Percent reduction of PCSK9 protein was determined after the PCSK9 level were normalized to scrambled controls. Results are shown in Tables 9 and 10 below.

TABLE 9
Percent reduction of PCSK9 protein in HepG2 cells.

| Name | Percent Reduction |
|---|---|
| P9-hc-026 | 36 |
| P9-h-045 | 91 |
| P9-h-255 | 84 |
| P9-h-288 | 76 |
| P9-hc-212 | 95 |
| P9-h-290 | 98 |
| P9-hc-223 | 79 |
| P9-hc-187 | 80 |
| P9-h-244 | 86 |
| P9-hc-133 | 32 |
| P9-hc-028 | 66 |
| P9-h-123 | 83 |
| P9-h-229 | 91 |
| P9-h-058 | 78 |
| P9-h-148 | 66 |
| P9-h-057 | 44 |
| P9-h-230 | 68 |
| P9-hc-239 | 68 |
| P9-hc-189 | 47 |
| P9-hc-063 | 72 |
| P9-h-163 | 12 |
| P9-h-219 | 79 |
| P9-h-037 | 75 |
| P9-h-174 | 62 |
| P9-hc-076 | 88 |
| P9-hc-215 | 33 |
| P9-hc-162 | 73 |
| P9-hc-238 | 56 |
| P9-h-004 | 75 |
| P9-hc-010 | 91 |
| P9-hc-023 | 2 |
| P9-hc-040 | 90 |
| P9-hc-043 | 92 |
| P9-h-050 | 83 |
| P9-h-053 | 65 |
| P9-h-077 | 94 |
| P9-hc-082 | 79 |
| P9-h-088 | 17 |
| P9-h-089 | 80 |
| P9-hc-095 | 91 |

TABLE 10
Percent reduction of PCSK9 protein in Huh7 cells.

| Name | Percent Reduction |
|---|---|
| P9-hc-026 | 52.18 |
| P9-hc-028 | 74.1 |
| P9-hc-162 | 52.16 |
| P9-hc-275 | 79.89 |
| P9-hc-187 | 44.15 |
| P9-hc-043 | 39.08 |
| P9-hc-082 | 32.27 |
| P9-hc-023 | 35.98 |
| P9-hc-010 | 86.02 |
| P9-hc-040 | 81.27 |
| P9-hc-095 | 71.7 |
| P9-hc-175 | 67.54 |
| P9-hc-195 | 33.84 |
| P9-hc-223 | 50.05 |
| P9-hc-063 | 65.98 |
| P9-hc-238 | 77.63 |
| P9-hc-232 | 59.23 |
| P9-hc-127 | 49.82 |
| P9-hc-213 | 75.12 |
| P9-hc-205 | 80.73 |

### Example 6: Screening of sgRNA Sequences in PHH

Primary human liver hepatocytes (PHH) were thawed in thawing medium (Gibco, Catalog #CM7500). After centrifugation, the supernatant was discarded and the pelleted cells were resuspended in hepatocyte plating medium plus supplement pack (William's E Medium plus Plating Supplements CM3000, Thermofisher) on collagen coated plates (Stem cell technologies Cat #100-0365) at a density of 35,000 cells/well in a 24-well plate. Plated cells were allowed to settle and adhere for 4 to 6 hours in a tissue culture incubator at 37° C. and 5% CO₂ atmosphere. Media were changed for William's E Medium plus Maintenance Supplements CM4000 (Thermofisher) which were used until the end of the experiments. Transfections were performed using RNAiMax reagent (Thermofisher) at 500 ng of cas9 mRNA (Trilink) and 100 ng of sgRNA (Synthego). After three days, the cells were lysed with QuickExtract™ DNA Extraction Solution (Lucigen, Cat #QE0905T) and the targeted genomic regions were amplified for NGS sequencing. The results of the PCSK9 protein reduction and gene editing efficiency were shown in Table 11 below.

TABLE 11
PCSK9 protein reduction and gene editing efficiency
in primary human liver hepatocytes

| | Percentage of PCSK9 | Gene Editing |
|---|---|---|
| Name | Protein reduction | efficiency |
| P9-h-057 | 77.2 | 64 |
| P9-hc-162 | 79.4 | 63 |
| P9-hc-026 | 81.4 | 62 |
| P9-hc-028 | 79.1 | 61 |
| P9-hc-023 | 75.2 | 61 |
| P9-hc-026 | 22.2 | 59 |
| P9-h-045 | 76.6 | 58 |
| P9-hc-124 | 58.1 | 57 |
| P9-h-059 | 86.4 | 55 |
| P9-hc-040 | 77.7 | 55 |
| P9-h-255 | 68.5 | 53 |
| P9-hc-010 | 74.7 | 52 |
| P9-hc-275 | 58.9 | 51 |
| P9-hc-175 | 78 | 50 |
| P9-hc-095 | 73.5 | 47 |
| P9-hc-195 | 72.7 | 47 |
| P9-hc-212 | 61.5 | 47 |
| P9-h-290 | 67.6 | 45 |
| P9-hc-215 | 68.2 | 43 |
| P9-h-106 | 72.4 | 42 |
| P9-h-229 | 59.2 | 40 |
| P9-h-228 | 73.3 | 35 |
| P9-hc-239 | 50.2 | |

### Example 7: SgRNA Editing Efficiency in PHH

Primary human liver hepatocytes (PHH) from another donor were thawed in InvitroGRO CP Medium containing 10% FBS and 1% Pen/Strep medium and plated on collagen coated plates at a density of 27,000 cells/well in a 24-well plate. After 4 to 6 hours, the medium was replaced by InvitroGRO CP medium. Transfection were performed using RNAiMax reagent (Thermofisher) at 500 ng of cas9 mRNA and 250 ng of sgRNA (Genscript). The day after transfection, the medium was replaced by 1% Pen/Strep InvitroGRO CP medium until cell harvest which were 3 days after transfection. Gene editing result were shown in Table 12 below.

TABLE 12
Gene editing efficiency of PCSK9 in
primary human livery hepatocytes

| Name | Gene Editing efficiency |
|---|---|
| P9-hc-212 | 65 |
| P9-hc-095 | 63 |
| P9-hc-162 | 58 |
| P9-hc-023 | 58 |
| P9-hc-028 | 53 |
| P9-hc-026 | 52 |
| P9-hc-223 | 51 |
| P9-hc-010 | 37 |
| P9-hc-026 | 32 |

### Example 8. Off-Target Analysis by dsDNA Insertion Assay in HepG2

A double-stranded (dsDNA) insertion-based assay was used to screen for potential genomic off-target sites cleaved by Cas9 with the corresponding gRNA. HepG2 cells were maintained in MEM (Gibco) supplemented with 10% FBS (OPCEL) at 37° C. and 5% CO₂. 1 million HepG2 cells were electroporated in 4D-Nuclefector (LONZA, X-unit) with 200 pmol of dsDNA, 35 pmol of Cas9 (NEB, EnGen Spy Cas9 NLS) protein and 200 pmol of gRNA (Genscript). Genomic DNA was extracted and processed for a NGS assay (See, e.g., Tsai et al., Nature Biotechnology 33, 187-197; 2015) in a NextSeq 6000 sequencer. The dsDNA incorporation efficiency for each potential off-target site was calculated as the number of reads at this site divided by the reads at the on-target site (PCSK9). The sum of efficiencies from the top 30 off target sites was divided by that of on target sites (top 30 off/on) and were used as semi-quantitative readouts for comparison of off-target potentials between different gRNAs. The number of total off target sites, and the first five sites of highest dsODN incorporation efficiencies were listed in Table 13 and Table 14 below, which represent two independent replicates.

TABLE 13
dsDNS incorporation efficiency and top 5 off target sites in HepG2 cells

| | Total | | | | |
|---|---|---|---|---|---|
| | off | Top30 | | | |
| gRNA | sites | off/on | TOP1 | TOP2 | TOP3 |
| P9-h-123 | 31 | 1.501 | chr1: | chr19: | chr11: |
| | | | 22,765,718-22,765,737 | 46,831,010-46,831,029 | 121,382,188-121,382,207 |
| P9-hc-063 | 49 | 1.099 | chr17: | chr17: | chr8: |
| | | | 81,058,499-81,058,518 | 65,504,271-65,504,290 | 144,096,510-144,096,529 |
| P9-h-099 | 38 | 0.969 | chr8: | chr11: | chr15: |
| | | | 144,096,491-144,096,510 | 70,520,931-70,520,950 | 89,024,786-89,024,805 |
| P9-h-255 | 9 | 0.786 | chr9: | chr11: | chr1: |
| | | | 69,131,769-69,131,788 | 46,204,368-46,204,387 | 37,475,817-37,475,836 |
| P9-h-174 | 91 | 0.758 | chr12: | chr14: | chr16: |
| | | | 113,843,108-113,843,127 | 90,654,842-90,654,861 | 2,032,492-2,032,511 |
| P9-hc-189 | 7 | 0.726 | chr14: | chr15: | chr14: |
| | | | 22,550,601-22,550,620 | 78,153,907-78,153,926 | 53,211,361-53,211,380 |
| P9-h-229 | 14 | 0.401 | chr7: | chr2: | chr16: |
| | | | 101,200,395-101,200,414 | 218,367,716-218,367,735 | 48,114,239-48,114,258 |
| P9-h-058 | 16 | 0.315 | chr1: | chr16: | chr18: |
| | | | 218,392,663-218,392,682 | 14,379,534-14,379,553 | 46,580,581-46,580,600 |
| P9-h-228 | 14 | 0.22 | chr2: | chr2: | chr5: |
| | | | 95,756,289-95,756,308 | 96,068,134-96,068,153 | 7,850,574-7,850,593 |
| P9-hc-023 | 6 | 0.14 | chr18: | chr16: | chr16: |
| | | | 79,605,563-79,605,582 | 68,281,962-68,281,981 | 14,379,520-14,379,539 |
| P9-h-219 | 37 | 0.094 | chr3: | chr18: | chr22: |
| | | | 67,228,766-67,228,785 | 47,911,938-47,911,957 | 44,660,810-44,660,829 |
| P9-h-230 | 98 | 0.088 | chr17: | chr8: | chrX: |
| | | | 75,047,974-75,047,993 | 109,640,524-109,640,543 | 154,020,388-154,020,407 |
| P9-hc-010 | 5 | 0.063 | chr2: | chr22: | chr1: |
| | | | 177,430,130-177,430,149 | 21,916,523-21,916,542 | 37,475,805-37,475,824 |
| P9-hc-195 | 35 | 0.014 | chr14: | chr1: | chr9: |
| | | | 22,550,567-22,550,586 | 97,964,484-97,964,503 | 131,530,200-131,530,219 |
| P9-hc-212 | 1 | 0.003 | chr2: | | |
| | | | 129,165,510-129,165,529 | | |
| P9-h-148 | 20 | 0.002 | chr1: | chr20: | chr4: |
| | | | 55,063,455-55,063,474 | 9,115,013-9,115,032 | 4,607,949-4,607,968 |
| P9-hc-142 | 14 | 0.002 | chr7: | chr21: | chr3: |
| | | | 74,110,470-74,110,489 | 38,884,757-38,884,776 | 30,672,160-30,672,179 |
| P9-hc-028 | 2 | 0.001 | chr10: | chr1: | |
| | | | 124,459,146-124,459,165 | 55,039,894-55,039,913 | |

| gRNA | TOP4 | TOP5 |
|---|---|---|
| P9-h-123 | chr19: | chr5: |
| | 19,492,137-19,492,156 | 7,850,568-7,850,587 |
| P9-hc-063 | chr2: | chr3: |
| | 150,932,057-150,932,076 | 177,458,135-177,458,154 |
| P9-h-099 | chr15: | chr10: |
| | 84,580,606-84,580,625 | 89,929,051-89,929,070 |
| P9-h-255 | chr19: | chr1: |
| | 55,115,763-55,115,782 | 55,058,145-55,058,164 |
| P9-h-174 | chr1: | chr13: |
| | 22,103,632-22,103,651 | 113,962,663-113,962,682 |
| P9-hc-189 | chr1: | chr2: |
| | 237,367,208-237,367,227 | 54,411,850-54,411,869 |
| P9-h-229 | chr1: | chr8: |
| | 37,475,804-37,475,823 | 143,568,643-143,568,662 |
| P9-h-058 | chr10: | chr2: |
| | 126,299,477-126,299,496 | 28,490,182-28,490,201 |
| P9-h-228 | chr7: | chr2: |
| | 101,200,390-101,200,409 | 97,623,954-97,623,973 |
| P9-hc-023 | chr1: | chr1: |
| | 55,039,973-55,039,992 | 16,703,918-16,703,937 |
| P9-h-219 | chr2: | chr14: |
| | 241,554,397-241,554,416 | 104,863,214-104,863,233 |
| P9-h-230 | chr16: | chr4: |
| | 88,487,986-88,488,005 | 1,013,717-1,013,736 |
| P9-hc-010 | chr18: | chr1: |
| | 23,556,283-23,556,302 | 55,058,142-55,058,161 |
| P9-hc-195 | chr5: | chr1: |
| | 39,621,802-39,621,821 | 147,566,167-147,566,186 |
| P9-hc-212 | | |
| P9-h-148 | chr3: | chr5: |
| | 46,861,928-46,861,947 | 131,522,751-131,522,770 |
| P9-hc-142 | chr1: | chr12: |
| | 55,058,195-55,058,214 | 53,065,370-53,065,389 |
| P9-hc-028 | | |

TABLE 14
dsDNS incorporation efficiency and top 5 off target sites in HepG2

| | Total | | | | |
|---|---|---|---|---|---|
| | off | Top30 | | | |
| gRNA | site | off/on | TOP1 | TOP2 | TOP3 |
| P9-h-229 | 145 | 2.68 | chr7: | chr16: | chr2: |
| | | | 101,200,395-101,200,414 | 48,114,239-48,114,258 | 218,367,716-218,367,735 |
| P9-hc-175 | 53 | 2.043 | chr11: | chr15: | chr20: |
| | | | 13,756,014-13,756,033 | 31,393,448-31,393,467 | 25,405,003-25,405,022 |
| P9-hc-023 | 154 | 1.902 | chr18: | chr1: | chr20: |
| | | | 79,605,563-79,605,582 | 28,193,861-28,193,880 | 56,766,886-56,766,905 |
| P9-h-059 | 154 | 1.451 | chr3: | chrX: | chr19: |
| | | | 52,779,925-52,779,944 | 130,014,908-130,014,927 | 6,857,062-6,857,081 |
| P9-hc-010 | 150 | 0.763 | chr2: | chr1: | chr4: |
| | | | 177,430,130-177,430,149 | 70,727,965-70,727,984 | 56,908,797-56,908,816 |
| P9-h-057 | 58 | 0.669 | chr20: | chr11: | chr18: |
| | | | 62,441,472-62,441,491 | 118,897,238-118,897,257 | 597,034-597,053 |
| P9-hc-124 | 105 | 0.557 | chr3: | chrX: | chr10: |
| | | | 42,755,854-42,755,873 | 154,095,388-154,095,407 | 129,629,813-129,629,832 |
| P9-h-228 | 56 | 0.365 | chr2: | chr2: | chr2: |
| | | | 95,756,289-95,756,308 | 96,068,134-96,068,153 | 97,623,954-97,623,973 |
| P9-hc-215 | 45 | 0.282 | chrX: | chr17: | chr4: |
| | | | 39,471,730-39,471,749 | 73,878,027-73,878,046 | 95,729,364-95,729,383 |
| P9-hc-028 | 91 | 0.244 | chr1: | chr2: | chr15: |
| | | | 630,787-630,806 | 192,062,681-192,062,700 | 56,903,939-56,903,958 |
| P9-hc-257 | 154 | 0.19 | chr20: | chr 10: | chr16: |
| | | | 31,935,903-31,935,922 | 42,497,710-42,497,729 | 1,288,125-1,288,144 |
| P9-hc-162 | 70 | 0.11 | chr14: | chr1: | chr19: |
| | | | 37,394,736-37,394,755 | 125,179,806-125,179,825 | 19,486,269-19,486,288 |
| P9-hc-239 | 59 | 0.062 | chrUn_GL000216v2: | chr1: | chr10: |
| | | | 149,557-149,606 | 55,039,819-55,039,838 | 26,446,530-26,446,549 |
| P9-hc-095 | 61 | 0.031 | chr6: | chr5: | chr10: |
| | | | 141,448,222-141,448,241 | 27,796,638-27,796,657 | 115,875,267-115,875,286 |
| P9-h-106 | 22 | 0.021 | chr2: | chr7: | chr1: |
| | | | 239,954,005-239,954,024 | 6,530,957-6,530,976 | 67,367,901-67,367,920 |
| P9-hc-212 | 58 | 0.008 | chr2: | chr16: | chr10: |
| | | | 14,164,443-14,164,462 | 68,040,298-68,040,317 | 31,932,782-31,932,801 |

| gRNA | TOP4 | TOP5 |
|---|---|---|
| P9-h-229 | chr19: | chr1: |
| | 35,059,590-35,059,609 | 198,080,802-198,080,821 |
| P9-hc-175 | chr3: | chr16: |
| | 45,321,777-45,321,796 | 85,587,111-85,587,130 |
| P9-hc-023 | chr16: | chr22: |
| | 68,281,962-68,281,981 | 37,486,121-37,486,140 |
| P9-h-059 | chr18: | chr7: |
| | 58,719,256-58,719,275 | 123,174,165-123,174,184 |
| P9-hc-010 | chr8: | chr20: |
| | 129,939,848-129,939,867 | 52,117,908-52,117,927 |
| P9-h-057 | chr14: | chr9: |
| | 69,234,955-69,234,974 | 83,585,160-83,585,179 |
| P9-hc-124 | chr12: | chr3: |
| | 43,523,153-43,523,172 | 34,013,806-34,013,825 |
| P9-h-228 | chr8: | chr8: |
| | 109,640,534-109,640,553 | 12,952,357-12,952,376 |
| P9-hc-215 | chr11: | chr17: |
| | 47,178,038-47,178,057 | 49,055,714-49,055,733 |
| P9-hc-028 | chr8: | chr2: |
| | 142,366,467-142,366,486 | 20,352,229-20,352,248 |
| P9-hc-257 | chr10: | chr18: |
| | 110,844,041-110,844,060 | 5,957,445-5,957,464 |
| P9-hc-162 | chr20: | chr7: |
| | 31,176,613-31,176,632 | 45,022,331-45,022,350 |
| P9-hc-239 | chr14_GL000225v1_{—} | chr7: |
| | random: 130,493-130,512 | 44,012,939-44,012,958 |
| P9-hc-095 | chr14: | chr2: |
| | 84,551,655-84,551,674 | 210,002,385-210,002,404 |
| P9-h-106 | chr8: | chr2: |
| | 129,599,284-129,599,303 | 71,669,889-71,669,908 |
| P9-hc-212 | chr2: | chr6: |
| | 218,401,936-218,401,955 | 55,812,081-55,812,100 |

### Example 9: Off-Target Analysis by dsDNA Insertion Assay in PHH

Primary human liver hepatocytes (PHH) were thawed in thawing medium (Gibco, Catalog #CM7500). After centrifugation, the supernatant was discarded and the pelleted cells were resuspended in hepatocyte plating medium plus supplement pack (William's E Medium plus Plating Supplements CM3000, Thermofisher) on collagen coated plates (Stem cell technologies Catalog #100-0365) at a density of 35,000 cells/well in a 24-well plate. Plated cells were allowed to settle and adhere for 4 to 6 hours in a tissue culture incubator at 37° C. and 5% CO₂ atmosphere. After that, the medium were changed for William's E Medium plus Maintenance Supplements CM4000 (Thermofisher) which were used until the end of the experiments. Transfections were performed using RNAiMax reagent (Thermofisher) at 500 ng of Cas9 mRNA (Trilink), 5 pmol dsDNA and 100 ng of sgRNA (Synthego). After three days, the genomic DNA was extracted with OceanNano Tech PureBind Genomic DNA Isolation Kit and processed for NGS assay (See, e.g., Tsai et al., Nature Biotechnology 33, 187-197; 2015) in a NextSeq 2000 sequencer. The sum of efficiencies from the top30 off target sites divided by that of on target site (top 30 off/on) were used as semi-quantitative readouts for comparison of off-target potentials between different gRNAs. The number of total off target sites, and the first five sites of highest dsODN incorporation efficiencies were listed in Table 15 below.

TABLE 15
dsDNS incorporation efficiency and top 5 off target sites in PHH

| | Total | | | | |
|---|---|---|---|---|---|
| | off | Top30 | | | |
| gRNA | site | off/on | TOP1 | TOP2 | TOP3 |
| P9-hc- | 20 | 1.383 | chr11: | chr16: | chrX: |
| 215 | | | 47,178,038-47,178,057 | 58,509,568-58,509,587 | 39,471,730-39,471,749 |
| P9-hc- | 9 | 1.291 | chr15: | chr11: | chr3: |
| 175 | | | 31,393,448-31,393,467 | 13,756,014-13,756,033 | 45,321,777-45,321,796 |
| P9-h- | 7 | 1.232 | chr9: | chr3: | chr2: |
| 255 | | | 69,131,769-69,131,788 | 45,598,852-45,598,871 | 73,085,193-73,085,212 |
| P9-hc- | 33 | 1.174 | chr17: | chr17: | chr8: |
| 063 | | | 81,058,499-81,058,518 | 65,504,271-65,504,290 | 144,096,510-144,096,529 |
| P9-hc- | 27 | 1.1 | chr11: | chr7: | chr3: |
| 082 | | | 102,721,655-102,721,674 | 151,819,036-151,819,055 | 99,835,974-99,835,993 |
| P9-h- | 14 | 0.905 | chr11: | chr15: | chr19: |
| 057 | | | 118,897,238-118,897,257 | 69,094,806-69,094,825 | 38,192,714-38,192,733 |
| P9-h- | 17 | 0.549 | chr3: | chr22: | chrX: |
| 059 | | | 52,779,925-52,779,944 | 24,522,132-24,522,151 | 130,014,908-130,014,927 |
| P9-hc- | 35 | 0.321 | chr18: | chr1: | chr1: |
| 023 | | | 79,605,563-79,605,582 | 1,508,714-1,508,733 | 220,528,025-220,528,044 |
| P9-h- | 6 | 0.26 | chr2: | chr2: | chr11: |
| 228 | | | 96,068,134-96,068,153 | 95,756,289-95,756,308 | 794,440-794,459 |
| P9-h- | 12 | 0.236 | chr2: | chr11: | chr8: |
| 228 | | | 95,756,289-95,756,308 | 794,440-794,459 | 12,952,357-12,952,376 |
| P9-hc- | 4 | 0.163 | chr19: | chr6: | chrX: |
| 187 | | | 14,107,101-14,107,120 | 39,901,995-39,902,014 | 5,776,278-5,776,297 |
| P9-hc- | 9 | 0.159 | chr18: | chr1: | chr1: |
| 023 | | | 79,605,563-79,605,582 | 16,703,918-16,703,937 | 4,799,463-4,799,482 |
| P9-hc- | 1 | 0.148 | chr2: | | |
| 010 | | | 177,430,130-177,430,149 | | |
| P9-hc- | 3 | 0.116 | chr14: | chr1: | chr22: |
| 026 | | | 61,280,100-61,280,119 | 9,113,030-9,113,049 | 24,178,166-24,178,185 |
| P9-hc- | 1 | 0.113 | chr2: | | |
| 010 | | | 177,430,130-177,430,149 | | |
| P9-hc- | 4 | 0.107 | chr11: | chr14: | chr17: |
| 043 | | | 61,680,777-61,680,796 | 51,095,021-51,095,040 | 80,699,297-80,699,316 |
| P9-hc- | 7 | 0.08 | chr1: | chr14: | chr7: |
| 026 | | | 9,113,030-9,113,049 | 61,280,100-61,280,119 | 6,447,730-6,447,749 |
| P9-hc- | 9 | 0.078 | chr3: | chr10: | chr1: |
| 124 | | | 42,755,854-42,755,873 | 129,629,813-129,629,832 | 243,461,825-243,461,844 |
| P9-hc- | 3 | 0.056 | chr3: | chr14: | chr1: |
| 124 | | | 42,755,854-42,755,873 | 57,034,199-57,034,218 | 243,461,825-243,461,844 |
| P9-hc- | 3 | 0.037 | chr1: | chr4: | chr18: |
| 275 | | | 111,858,601-111,858,620 | 155,917,675-155,917,694 | 6,962,026-6,962,045 |
| P9-hc- | 3 | 0.035 | chr3: | chr18: | chr1: |
| 162 | | | 29,531,655-29,531,674 | 69,892,913-69,892,932 | 87,453,845-87,453,864 |
| P9-hc- | 7 | 0.031 | chr2: | chr10: | chrX: |
| 212 | | | 14,164,443-14,164,462 | 94,769,353-94,769,372 | 77,928,515-77,928,534 |
| P9-hc- | 3 | 0.028 | chr22: | chr4: | chr12: |
| 026 | | | 24,178,166-24,178,185 | 73,258,296-73,258,315 | 65,170,382-65,170,401 |
| P9-hc- | 2 | 0.028 | chr3: | chr18: | |
| 257 | | | 133,380,685-133,380,704 | 5,957,445-5,957,464 | |
| P9-hc- | 3 | 0.011 | chr2: | chr15: | chr2: |
| 028 | | | 192,062,681-192,062,700 | 56,903,939-56,903,958 | 20,352,229-20,352,248 |
| P9-hc- | 1 | 0.007 | chr3: | | |
| 162 | | | 29,531,655-29,531,674 | | |
| P9-hc- | 3 | 0.007 | chr16: | chr11: | chr7: |
| 223 | | | 86,013,706-86,013,725 | 59,200,083-59,200,102 | 132,416,077-132,416,096 |
| P9-hc- | 2 | 0.006 | chr2: | chr15: | |
| 028 | | | 192,062,681-192,062,700 | 56,903,939-56,903,958 | |
| P9-hc- | 1 | 0.005 | chr10: | | |
| 239 | | | 26,446,530-26,446,549 | | |
| P9-hc- | 1 | 0.003 | chr18: | | |
| 275 | | | 6,962,026-6,962,045 | | |
| P9-h- | Not | Not | | | |
| 106 | detected | detected | | | |
| P9-hc- | Not | Not | | | |
| 095 | detected | detected | | | |

| gRNA | TOP4 | TOP5 |
|---|---|---|
| P9-hc- | chr17: | chr2: |
| 215 | 73,878,027-73,878,046 | 98,296,582-98,296,601 |
| P9-hc- | chr9: | chr16: |
| 175 | 5,832,894-5,832,913 | 85,587,111-85,587,130 |
| P9-h- | chr1: | chr1: |
| 255 | 207,826,595-207,826,614 | 227,323,355-227,323,374 |
| P9-hc- | chr8: | chr15: |
| 063 | 122,522,498-122,522,517 | 59,479,609-59,479,628 |
| P9-hc- | chr16: | chr2: |
| 082 | 77,303,154-77,303,173 | 54,240,691-54,240,710 |
| P9-h- | chr9: | chr14: |
| 057 | 83,585,160-83,585,179 | 69,234,955-69,234,974 |
| P9-h- | chr19: | chr7: |
| 059 | 6,857,062-6,857,081 | 87,876,709-87,876,728 |
| P9-hc- | chr10: | chr16: |
| 023 | 97,037,461-97,037,480 | 3,696,272-3,696,291 |
| P9-h- | chr8: | chr11: |
| 228 | 12,952,357-12,952,376 | 3,443,954-3,443,973 |
| P9-h- | chr2: | chr12: |
| 228 | 96,068,134-96,068,153 | 52,965,624-52,965,643 |
| P9-hc- | chr8: | |
| 187 | 116,755,764-116,755,783 | |
| P9-hc- | chr10: | chr11: |
| 023 | 97,037,461-97,037,480 | 3,231,531-3,231,550 |
| P9-hc- | | |
| 010 | | |
| P9-hc- | | |
| 026 | | |
| P9-hc- | | |
| 010 | | |
| P9-hc- | chr7: | — |
| 043 | 74,544,921-74,544,940 | |
| P9-hc- | chr22: | chr4: |
| 026 | 24,178,166-24,178,185 | 73,258,296-73,258,315 |
| P9-hc- | chrX: | chr12: |
| 124 | 154,095,388-154,095,407 | 43,523,153-43,523,172 |
| P9-hc- | | |
| 124 | | |
| P9-hc- | | |
| 275 | | |
| P9-hc- | | |
| 162 | | |
| P9-hc- | chr14: | chr7: |
| 212 | 49,950,458-49,950,477 | 98,083,464-98,083,483 |
| P9-hc- | | |
| 026 | | |
| P9-hc- | | |
| 257 | | |
| P9-hc- | | |
| 028 | | |
| P9-hc- | | |
| 162 | | |
| P9-hc- | | |
| 223 | | |
| P9-hc- | | |
| 028 | | |
| P9-hc- | | |
| 239 | | |
| P9-hc- | | |
| 275 | | |
| P9-h- | | |
| 106 | | |
| P9-hc- | | |
| 095 | | |

### Example 10: Off-Target Analysis of sgRNA by Amplicon-Based NGS in PHH

Primary human liver hepatocytes (PHH) were thawed in InvitroGRO CP Medium containing 10% FBS and 1% Pen/Strat and plated on collagen coated plates at a density of 270,000 cells/well in a 24-well plate. After 4 to 6 hours, the medium was replaced by InvitroGRO CP medium. Transfection were performed using 1.5 μL RNAiMax reagent (Thermofisher) at 400 ng of cas9 mRNA and 200 ng of sgRNA (Genscript). The day after transfection, the medium was replaced by 1% Pen/Strep InvitroGRO CP medium until cell harvesting 3 days after transfection. The genomic DNA was extracted with QuickExtract DNA extract solution (Lucigen). The editing at the on target and top off target sites were amplified by PCR with Taq Pro Multiplex DNA Polymerase (Vazyme). PCR product was purified with VAHTS DNA Clean Beads (Vazyme) and sequenced at an Illumina Novaseq6000 platform. The off-target site editing efficiency was divided by the on-target efficiency in the same experiment to normalize for different transfection efficiencies. Editing efficiencies of the top off target sites divided by the on-target editing efficiencies are shown in Table 16 below.

TABLE 16
Editing efficiencies of the top off target sites
divided by the on-target editing efficiencies

| gRNA | OT1 | OT2 | OT3 | OT4 | OT5 | OT6 | OT7 |
|---|---|---|---|---|---|---|---|
| P9-hc-063 | 26.20% | 0.10% | 0.20% | 0.10% | 2.30% | 0.00% | 1.20% |
| P9-hc-175 | 13.40% | 3.90% | 0.10% | 0.00% | 0.00% | 0.10% | 0.00% |
| P9-hc-023 | 2.60% | 0.60% | 0.30% | 0.00% | N/A | N/A | N/A |
| P9-hc-082 | 1.60% | 0.10% | 0.10% | 0.20% | 0.10% | N/A | N/A |
| P9-hc-010 | 1.10% | 0.50% | 0.20% | N/A | N/A | N/A | N/A |
| P9-h-057 | 0.50% | 0.30% | 0.60% | 0.30% | 3.20% | 0.50% | N/A |
| P9-hc-212 | 0.30% | 0.30% | N/A | N/A | N/A | N/A | N/A |
| P9-hc-026 | 0.20% | 1.60% | 1.80% | 0.10% | N/A | N/A | N/A |
| P9-hc-223 | 0.20% | 0.00% | 0.00% | 0.00% | 0.00% | N/A | N/A |
| P9-hc-028 | 0.10% | 0.10% | 0.20% | 0.00% | N/A | N/A | N/A |
| P9-hc-043 | 0.10% | 0.00% | 0.00% | N/A | N/A | N/A | N/A |
| P9-hc-162 | 0.10% | 0.10% | 0.10% | N/A | N/A | N/A | N/A |
| P9-hc-095 | 0.00% | 0.00% | 0.00% | N/A | N/A | N/A | N/A |

### Example 11. On/Off Target Editing Efficiency of DNA-RNA Hybrid gRNA in Huh7 Cells

Huh7 cells were plated 8,000 cells/well in a 96-well plate. Transfection was performed using 0.4 μL RNAiMax reagent (Thermofisher) at 100 ng of cas9 mRNA and 100 ng of sgRNA with or without deoxyribonucleotide replacement (General Biosystems). Cells were harvested 3 days after transfection. The genomic DNA was extracted with QuickExtract DNA extract solution (Lucigen). The editing at the on target site and top off target sites were amplified by PCR with Taq Pro Multiplex DNA Polymerase (Vazyme). PCR product was purified with VAHTS DNA Clean Beads (Vazyme) and sequenced at an illumina Novaseq6000 platform. The editing efficiency of on and top off target sites were listed in Table 17 below.

TABLE 17
Editing efficiency of on and top off target sites

| gRNA | ON | OT1 | OT2 | OT3 | OT4 | OT5 |
|---|---|---|---|---|---|---|
| P9-hc-023 | 96.10% | 33.60% | 0.60% | 0.20% | 0.00% | N/A |
| P9-hc-023-seq2 | 95.60% | 24.90% | 0.30% | 0.30% | 0.00% | N/A |
| P9-hc-023-seq3 | 92.90% | 10.80% | 0.30% | 0.20% | 0.00% | N/A |
| P9-hc-023-seq4 | 95.50% | 5.70% | 0.30% | 0.20% | 0.00% | N/A |
| P9-hc-023-seq5 | 94.90% | 3.30% | 0.30% | 0.20% | 0.00% | N/A |
| P9-hc-023-seq6 | 74.20% | 21.50% | 0.30% | 0.20% | 0.00% | N/A |
| P9-hc-023-seq7 | 68.20% | 0.50% | 0.30% | 0.20% | 0.00% | N/A |
| P9-hc-023-seq8 | 48.20% | 0.70% | 0.30% | 0.20% | 0.00% | N/A |
| P9-hc-023-seq9 | 90.40% | 0.50% | 0.30% | 0.20% | 0.00% | N/A |
| P9-hc-023- | 85.20% | 0.30% | 0.30% | 0.20% | 0.00% | N/A |
| seq10 | | | | | | |
| P9-hc-023- | 88.80% | 0.10% | 0.30% | 0.20% | 0.00% | N/A |
| seq11 | | | | | | |
| P9-hc-023- | 83.70% | 0.00% | 0.30% | 0.20% | 0.00% | N/A |
| seq12 | | | | | | |
| P9-hc-028 | 94.80% | 1.20% | 0.20% | 0.30% | 0.00% | N/A |
| P9-hc-028-seq2 | 91.60% | 0.50% | 0.10% | 0.10% | 0.00% | N/A |
| P9-hc-028-seq3 | 90.00% | 0.60% | 0.10% | 0.20% | 0.00% | N/A |
| P9-hc-028-seq4 | 93.80% | 0.30% | 0.00% | 0.10% | 0.00% | N/A |
| P9-hc-028-seq5 | 96.10% | 0.90% | 0.10% | 0.10% | 0.00% | N/A |
| P9-hc-028-seq6 | 90.60% | 0.60% | 0.00% | 0.10% | 0.00% | N/A |
| P9-hc-082 | 91.70% | 4.70% | 0.20% | 0.10% | 0.30% | 1.90% |
| P9-hc-082-seq2 | 90.20% | 0.50% | 0.30% | 0.10% | 0.30% | 2.70% |
| P9-hc-082-seq3 | 84.30% | 0.40% | 0.10% | 0.00% | 0.10% | 1.20% |
| P9-hc-082-seq4 | 92.30% | 0.90% | 0.00% | 0.00% | 0.20% | 1.00% |
| P9-hc-082-seq5 | 61.30% | 0.10% | 0.00% | 0.00% | 0.10% | 0.40% |
| P9-hc-082-seq6 | 93.50% | 0.20% | 0.00% | 0.00% | 0.10% | 0.70% |
| P9-hc-082-seq7 | 94.10% | 0.10% | 0.00% | 0.00% | 0.10% | 0.30% |
| P9-hc-162 | 91.20% | 3.50% | 0.10% | 0.30% | N/A | N/A |
| P9-hc-162-seq2 | 78.30% | 0.20% | 0.20% | 0.10% | N/A | N/A |
| P9-hc-162-seq3 | 86.50% | 0.30% | 0.10% | 0.10% | N/A | N/A |
| P9-hc-162-seq4 | 34.70% | 0.10% | 0.00% | 0.10% | N/A | N/A |
| P9-hc-162-seq5 | 85.60% | 0.20% | 0.10% | 0.10% | N/A | N/A |
| P9-hc-162-seq6 | 90.10% | 0.10% | 0.10% | 0.10% | N/A | N/A |
| P9-hc-162-seq7 | 75.60% | 0.20% | 0.00% | 0.10% | N/A | N/A |
| P9-hc-212 | 92.60% | 1.40% | 0.20% | N/A | N/A | N/A |
| P9-hc-212-seq2 | 93.90% | 0.50% | 0.20% | N/A | N/A | N/A |
| P9-hc-212-seq3 | 93.30% | 0.30% | 0.20% | N/A | N/A | N/A |
| P9-hc-212-seq4 | 93.50% | 0.60% | 0.10% | N/A | N/A | N/A |
| P9-hc-212-seq5 | 91.40% | 0.40% | 0.20% | N/A | N/A | N/A |
| P9-hc-212-seq6 | 91.70% | 0.30% | 0.20% | N/A | N/A | N/A |

### Example 12: On/Off Target Editing Efficiency of DNA-RNA Hybrid gRNA in PHH Cells

Primary human liver hepatocytes (PHH) were thawed in InvitroGRO CP Medium containing 10% FBS and 1% Pen/Strat and plated on collagen coated plates at a density of 130,000 cells/well in a 48-well plate. After 4 to 6 hours, the medium was replaced by InvitroGRO CP medium. Transfection were performed using 0.75 μL RNAiMax reagent (Thermofisher) at 250 ng of cas9 mRNA and 250 ng of sgRNA (General BioL). The day after transfection, the medium was replaced by 1% Pen/Strep InvitroGRO CP medium until cell harvest which were 3 days after transfection. The genomic DNA was extracted with QuickExtract DNA extract solution (Lucigen). The editing at the on target and top off target sites were amplified by PCR with Taq Pro Multiplex DNA Polymerase (Vazyme). PCR product was purified with VAHTS DNA Clean Beads (Vazyme) and sequenced at an illumina Novaseq6000 platform. The editing efficiency of on and top off target sites are listed in Table 18 below.

TABLE 18
The editing efficiency of on and top off target sites

| gRNA | ON | OT1 | OT2 | OT3 | OT4 | OT5 |
|---|---|---|---|---|---|---|
| P9-hc-023 | 65.7% | 2.2% | 0.6% | 0.3% | 0.0% | N/A |
| P9-hc-023-seq5 | 60.0% | 0.3% | 0.3% | 0.3% | 0.0% | N/A |
| P9-hc-023-seq9 | 58.9% | 0.2% | 0.3% | 0.3% | 0.0% | N/A |
| P9-hc-023-seq10 | 36.4% | 0.1% | 0.3% | 0.3% | 0.0% | N/A |
| P9-hc-023-seq11 | 39.3% | 0.1% | 0.3% | 0.3% | 0.0% | N/A |
| P9-hc-028-seq1 | 53.4% | 0.3% | 0.1% | 0.2% | 0.0% | N/A |
| P9-hc-028-seq4 | 33.8% | 0.1% | 0.0% | 0.1% | 0.0% | N/A |
| P9-hc-028-seq5 | 50.2% | 0.3% | 0.0% | 0.1% | 0.0% | N/A |
| P9-hc-028-seq6 | 50.8% | 0.2% | 0.0% | 0.1% | 0.0% | N/A |
| P9-hc-082 | 44.1% | 1.0% | 0.1% | 0.0% | 0.2% | 0.1% |
| P9-hc-082-seq4 | 39.0% | 0.1% | 0.0% | 0.0% | 0.1% | 0.0% |
| P9-hc-082-seq6 | 22.1% | 0.0% | 0.0% | 0.0% | 0.1% | 0.0% |
| P9-hc-082-seq7 | 22.5% | 0.0% | 0.0% | 0.0% | 0.1% | 0.0% |
| P9-hc-162 | 45.6% | 0.2% | 0.1% | 0.2% | N/A | N/A |
| P9-hc-162-seq2 | 45.6% | 0.2% | 0.1% | 0.2% | N/A | N/A |
| P9-hc-162-seq3 | 54.4% | 0.1% | 0.1% | 0.1% | N/A | N/A |
| P9-hc-162-seq5 | 55.3% | 0.1% | 0.1% | 0.1% | N/A | N/A |
| P9-hc-162-seq6 | 54.4% | 0.0% | 0.0% | 0.1% | N/A | N/A |
| P9-hc-212 | 60.8% | 1.2% | 0.2% | N/A | N/A | N/A |
| P9-hc-212-seq2 | 56.6% | 0.4% | 0.1% | N/A | N/A | N/A |
| P9-hc-212-seq3 | 53.8% | 0.3% | 0.2% | N/A | N/A | N/A |
| P9-hc-212-seq6 | 49.3% | 0.3% | 0.3% | N/A | N/A | N/A |

### Example 13. The Effect of Various Cas9 mRNA Elements in Huh7 and PHH Cells

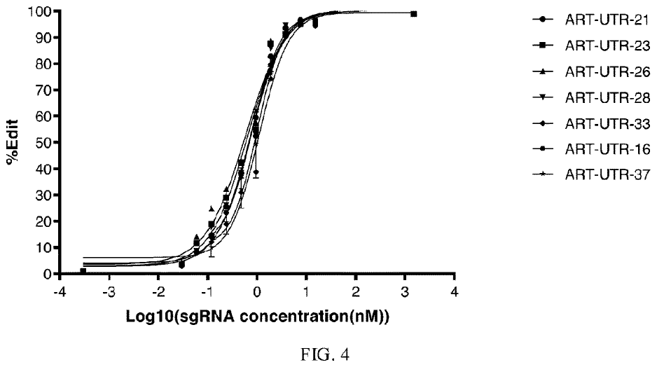

UTR screening in Huh7 cells. sgRNA P9-hc-162 targeting human PCSK9 and Cas9 mRNA composing different UTR were delivered to Huh7 cells as described in Example 2, in an 8-12 point 2-fold dose response curve. The cells were lysed 72 hours post treatment for editing analysis as described in Example 2. The UTR elements were then listed based on EC50 values and maximum editing percent. The dose response curve data for the guide sequences in Huh7 cells is shown in FIG. 4. The EC50 values and maximum editing percent are listed in Table 19 below.

TABLE 19
The efficacy of Cas9 mRNA variants featuring distinct UTRs

| Name | EC50 (nM) | Max Edit (%) |
|---|---|---|
| ART-UTR-16 | 0.797 | 96.83 |
| ART-UTR-37 | 0.994 | 96.66 |
| ART-UTR-21 | 0.721 | 95.51 |
| ART-UTR-23 | 0.628 | 95.42 |
| ART-UTR-26 | 0.576 | 95.68 |
| ART-UTR-28 | 0.725 | 96.17 |
| ART-UTR-33 | 1.094 | 95.32 |

CDS design with MFE and CAI. The CDS was designed considering three factors: high CAI, low MFE and moderate GC content. The same UTRs and nuclear localization signal sequences were used for calculation of MFE and cellular experiments. The characteristics of the designed CDS are listed in Table 20 below. The percentage similarity among the designed CDS are listed in Table 21 below. The sequences of the CDS are provided as SEQ ID NOs 954-960 in Table 22 below.

TABLE 20
The characteristics of designed CDS

| | MFE (kcal/mol) | CAI Score | GC Content(%) |
|---|---|---|---|
| ART-CDS-K1-1 | −1824 | 0.923 | 58.6 |
| ART-CDS-K4-8 | −1776 | 0.968 | 59.5 |
| ART-CDS-S311 | −1745 | 0.992 | 61.8 |
| ART-CDS-K10-2 | −1742 | 0.991 | 60.3 |
| ART-CDS-K8-1 | −1729 | 0.990 | 60.2 |
| ART-CDS-004R | −1412 | 0.910 | 52.6 |
| ART-CDS-S204 | −1169 | 0.865 | 50.4 |

TABLE 21
The percentage similarity among the designed CDS

| | ART- | ART- | ART- | ART- | ART- | ART- |
|---|---|---|---|---|---|---|
| | CDS- | CDS- | CDS- | CDS- | CDS- | CDS- |
| | K1-1 | K4-8 | K8-1 | K10-2 | s311 | S204 |
| 004 | 85.5 | 86.8 | 88.1 | 88.2 | 87.5 | 84.7 |
| ART-CDS-K1-1 | | 93.2 | 93.6 | 93.5 | 93.3 | 83.2 |
| ART-CDS-K4-8 | | | 97.0 | 96.8 | 95.7 | 84.4 |
| ART-CDS-K8-1 | | | | 98.5 | 96.4 | 85.1 |
| ART-CDS-K10-2 | | | | | 96.6 | 85.2 |
| ART-CDS-s311 | | | | | | 82.4 |

TABLE 22
Sequences of CDS designs

| Sequence | Name | SEQ ID NO |
|---|---|---|
| ATGGACAAGAAATATAGCATCGGGCTGGATATCGGCACC | ART-CDS-004R | 954 |
| AACTCTGTCGGCTGGGCGGTAATCACCGACGAGTACAAG | | |
| GTTCCTAGCAAGAAGTTCAAGGTGCTGGGAAACACCGAC | | |
| CGGCACAGCATCAAGAAGAACCTGATCGGCGCTTTGCTG | | |
| TTCGATAGCGGCGAAACCGCCGAAGCCACCAGGCTGAA | | |
| GAGAACCGCCAGGCGGCGTTACACAAGAAGAAAAAACC | | |
| GGATCTGTTACCTGCAGGAGATTTTCAGCAACGAGATGG | | |
| CCAAGGTGGATGACAGCTTCTTCCACAGACTGGAAGAGA | | |
| GCTTCCTGGTGGAGGAAGATAAAAAGCACGAAAGACAC | | |
| CCCATCTTTGGCAATATCGTGGACGAGGTTGCATACCACG | | |
| AGAAGTACCCTACTATCTACCACCTGAGAAAGAAGTTGG | | |
| TGGACTCTACAGACAAGGCTGATCTGCGGCTGATCTACCT | | |
| GGCGCTGGCCCACATGATCAAGTTCCGGGGGCATTTCCT | | |
| GATCGAAGGCGATCTGAACCCCGACAACAGCGACGTGG | | |
| ACAAGCTGTTCATCCAACTGGTTCAGACATATAATCAACT | | |
| GTTCGAGGAGAACCCTATCAACGCCTCAGGCGTGGACGC | | |
| CAAGGCCATCTTAAGCGCCAGACTGTCAAAATCTAGACG | | |
| GCTGGAAAACCTGATCGCCCAGCTGCCCGGCGAAAAGAA | | |
| GAACGGGCTGTTCGGCAACCTGATCGCCCTGAGCCTTGG | | |
| TCTGACACCTAACTTCAAGAGCAATTTCGATCTGGCTGAG | | |
| GATGCCAAGCTCCAGCTCAGCAAAGACACCTACGACGAC | | |
| GATCTGGACAATCTGCTGGCTCAAATCGGCGACCAGTAC | | |
| GCTGACCTGTTCCTGGCTGCCAAGAACCTGTCTGACGCCA | | |
| TCCTGCTCAGCGACATCCTGAGAGTTAACACAGAGATTAC | | |
| AAAGGCCCCTCTGTCTGCCAGCATGATCAAGCGGTACGA | | |
| CGAGCACCACCAGGACCTGACCCTGCTGAAGGCCCTGGT | | |
| GAGACAGCAGCTGCCTGAGAAGTACAAGGAAATCTTTTT | | |
| CGACCAGAGCAAGAACGGATATGCCGGCTATATCGATGG | | |
| CGGCGCCAGTCAGGAAGAGTTCTACAAGTTCATCAAACC | | |
| TATCCTCGAAAAGATGGACGGCACAGAGGAACTGCTAGT | | |
| GAAGCTGAACAGAGAAGATCTGCTGAGAAAGCAGCGGA | | |
| CATTCGATAACGGATCCATTCCTCACCAGATCCACCTGGG | | |
| CGAGCTGCATGCCATCCTTCGAAGGCAAGAAGATTTCTA | | |
| CCCTTTCCTGAAAGATAACAGAGAGAAGATCGAGAAAAT | | |
| CCTGACATTCAGAATCCCCTACTACGTGGGACCTCTGGCC | | |
| AGAGGCAACAGCCGGTTCGCCTGGATGACCCGGAAAAG | | |
| CGAGGAGACAATCACACCTTGGAACTTCGAGGAGGTCGT | | |
| GGACAAAGGCGCCAGCGCTCAGAGCTTCATCGAGAGAA | | |
| TGACCAATTTCGACAAAAACCTGCCCAACGAGAAGGTGC | | |
| TCCCTAAGCACAGCCTGCTGTACGAGTACTTTACCGTCTA | | |
| CAACGAGCTGACCAAGGTCAAGTATGTGACCGAGGGTAT | | |
| GCGGAAGCCTGCTTTCCTGAGCGGCGAGCAGAAGAAGG | | |
| CCATCGTGGACCTGCTGTTCAAGACCAACAGAAAAGTGA | | |
| CCGTGAAGCAGCTGAAAGAGGACTACTTTAAGAAGATCG | | |
| AGTGCTTCGATTCGGTGGAAATCAGCGGCGTGGAGGAC | | |
| AGGTTCAACGCCTCCCTGGGCACATACCACGACCTGCTG | | |
| AAGATCATCAAGGATAAGGATTTTCTCGACAATGAAGAG | | |
| AACGAGGACATCCTGGAGGACATCGTGCTCACACTGACA | | |
| CTGTTTGAAGACAGAGAAATGATCGAGGAAAGACTGAA | | |
| GACCTACGCTCACCTGTTTGACGACAAAGTCATGAAACA | | |
| GCTGAAGCGGAGACGCTACACCGGCTGGGGCCGGCTGA | | |
| GCAGAAAGCTGATCAACGGCATCAGAGACAAGCAAAGC | | |
| GGCAAGACCATTCTGGATTTCCTGAAGAGCGACGGCTTC | | |
| GCCAACCGGAACTTCATGCAGCTGATCCACGACGACTCTC | | |
| TGACCTTCAAAGAAGACATCCAGAAGGCCCAAGTGAGTG | | |
| GCCAGGGCGACTCCCTCCATGAACACATAGCCAACCTGG | | |
| CCGGCTCTCCAGCCATCAAGAAGGGAATCTTGCAGACCG | | |
| TGAAGGTGGTGGATGAACTGGTGAAAGTGATGGGCAGA | | |
| CATAAGCCCGAGAACATCGTGATCGAAATGGCCAGAGA | | |
| GAATCAGACCACCCAGAAGGGCCAGAAGAACTCTCGGG | | |
| AAAGAATGAAGCGGATCGAGGAGGGCATCAAGGAGCTG | | |
| GGCAGCCAGATCCTCAAGGAACATCCCGTGGAAAACACA | | |
| CAGCTGCAGAATGAGAAGCTGTACTTGTACTACCTGCAG | | |
| AACGGTCGAGATATGTACGTGGACCAGGAGCTGGACATC | | |
| AACAGACTGTCCGACTACGATGTGGATCACATCGTGCCTC | | |
| AGAGCTTCCTGAAGGACGATTCCATCGATAACAAGGTGC | | |
| TGACCAGAAGCGATAAGAACAGAGGGAAGAGCGACAAC | | |
| GTGCCATCTGAAGAAGTGGTGAAGAAGATGAAGAATTA | | |
| CTGGAGACAGCTGCTGAACGCCAAACTGATCACACAAAG | | |
| AAAATTCGACAACCTGACGAAGGCCGAACGGGGAGGAT | | |
| TGAGCGAACTTGACAAAGCCGGCTTTATCAAGAGACAGC | | |
| TGGTCGAAACCAGACAGATCACCAAACACGTAGCCCAGA | | |
| TCCTGGACAGCCGGATGAACACCAAGTACGACGAGAATG | | |
| ACAAGCTGATACGGGAGGTGAAGGTTATCACCCTGAAAT | | |
| CTAAGCTGGTGAGCGACTTCAGAAAGGACTTCCAGTTCT | | |
| ACAAAGTGCGGGAGATCAACAACTACCACCACGCCCACG | | |
| ATGCCTACCTCAACGCGGTGGTGGGCACCGCCCTGATCA | | |
| AGAAGTACCCTAAGCTGGAATCTGAGTTCGTGTACGGCG | | |
| ATTACAAGGTCTACGACGTGCGGAAGATGATTGCCAAGT | | |
| CCGAGCAGGAGATCGGCAAGGCCACCGCCAAGTACTTTT | | |
| TCTACTCGAACATCATGAACTTCTTTAAAACCGAGATCAC | | |
| ACTGGCCAATGGAGAGATCAGAAAGCGGCCTCTGATTGA | | |
| AACAAACGGCGAAACAGGCGAGATTGTCTGGGACAAAG | | |
| GCAGGGATTTCGCTACAGTGAGGAAGGTGCTGAGCATG | | |
| CCTCAGGTCAATATCGTGAAGAAGACCGAGGTGCAGACC | | |
| GGCGGATTCAGCAAGGAGTCCATCCTGCCTAAGAGAAAC | | |
| TCTGACAAACTGATCGCCCGGAAAAAGGACTGGGATCCT | | |
| AAGAAGTATGGCGGCTTCGACAGCCCCACCGTGGCCTAC | | |
| AGCGTGCTGGTGGTGGCCAAGGTGGAAAAGGGCAAGAG | | |
| CAAAAAACTGAAGTCTGTGAAAGAGCTGCTGGGCATCAC | | |
| AATCATGGAAAGAAGCAGCTTTGAGAAGAACCCCATAGA | | |
| CTTCCTGGAAGCCAAGGGCTACAAGGAGGTGAAAAAGG | | |
| ATCTGATCATCAAGCTGCCAAAATACAGCCTGTTTGAACT | | |
| GGAGAACGGCAGAAAGCGCATGTTGGCCAGCGCCGGCG | | |
| AGCTGCAAAAAGGAAACGAGCTGGCTCTGCCCAGCAAGT | | |
| ACGTGAACTTCCTGTACCTGGCCAGCCACTACGAGAAGC | | |
| TGAAGGGCTCACCCGAAGATAATGAGCAGAAGCAGTTAT | | |
| TCGTGGAGCAGCACAAGCACTACTTGGACGAGATCATTG | | |
| AACAGATCAGCGAGTTCAGCAAGAGAGTGATCCTGGCCG | | |
| ATGCCAACCTGGACAAGGTGCTGAGCGCCTACAACAAGC | | |
| ACCGGGACAAGCCTATCAGGGAACAGGCCGAAAATATCA | | |
| TCCACCTGTTTACCCTCACCAATCTGGGAGCCCCTGCCGC | | |
| CTTCAAGTATTTCGACACAACCATCGACAGAAAGAGATAT | | |
| ACCTCCACCAAGGAGGTACTCGACGCCACCCTGATCCACC | | |
| AGAGCATTACCGGCCTGTACGAAACCAGAATCGACCTGT | | |
| CTCAGCTGGGAGGCGACGGTGGTGGCAGCCCGAAGAAG | | |
| AAAAGAAAGGTG | | |
| ATGGATAAGAAATATTCCATTGGCCTGGACATCGGGACC | ART-CDS-K1-1 | 955 |
| AACAGCGTGGGCTGGGCCGTGATCACCGACGAGTACAA | | |
| GGTCCCCAGCAAGAAGTTCAAGGTGCTGGGAAACACCG | | |
| ACCGGCACTCCATCAAGAAGAACTTGATCGGGGCCTTGC | | |
| TGTTCGACAGCGGCGAGACCGCCGAGGCCACGCGGTTG | | |
| AAGCGGACCGCCCGCAGGCGGTACACTCGCCGGAAGAA | | |
| CAGGATCTGCTACCTGCAGGAGATCTTCTCAAACGAGAT | | |
| GGCCAAGGTGGACGACTCCTTCTTCCACCGGCTGGAGGA | | |
| GTCGTTCCTGGTCGAGGAGGACAAGAAGCACGAGCGGC | | |
| ACCCCATCTTCGGCAACATCGTGGACGAGGTGGCCTACC | | |
| ACGAGAAGTACCCCACCATCTACCACCTGCGGAAGAAGC | | |
| TGGTGGACTCCACTGACAAGGCCGACCTGCGGCTGATCT | | |
| ACCTGGCCCTGGCCCACATGATCAAGTTCAGGGGCCACT | | |
| TCCTGATCGAGGGCGATCTGAACCCCGATAACAGCGATG | | |
| TGGACAAGCTGTTCATCCAGCTGGTGCAGACCTACAACC | | |
| AGCTGTTCGAGGAGAATCCCATCAACGCCAGCGGCGTGG | | |
| ACGCCAAGGCCATCCTGAGCGCCAGGCTGAGCAAGAGC | | |
| CGGCGCTTAGAGAACCTGATCGCCCAGCTGCCCGGGGAG | | |
| AAGAAGAACGGGCTGTTCGGGAACCTGATCGCCCTCAGC | | |
| CTGGGCCTGACCCCCAACTTCAAGTCAAACTTCGACTTGG | | |
| CCGAGGACGCCAAGTTGCAGTTGTCCAAGGACACCTACG | | |
| ACGACGACTTGGACAACCTCCTGGCACAGATCGGGGATC | | |
| AGTACGCAGACCTGTTTCTGGCCGCCAAGAACTTGAGCG | | |
| ACGCCATCCTGCTGTCAGACATCCTGCGGGTGAACACCG | | |
| AGATCACCAAGGCCCCCCTGTCCGCGTCCATGATCAAGC | | |
| GGTACGACGAGCACCACCAGGACCTGACCCTGCTGAAGG | | |
| CCCTGGTGAGGCAGCAGTTGCCCGAGAAGTACAAGGAG | | |
| ATCTTCTTCGATCAGAGCAAGAACGGCTACGCTGGCTAC | | |
| ATCGACGGCGGGGCCAGCCAGGAGGAGTTCTACAAGTT | | |
| CATCAAGCCCATCCTGGAGAAGATGGACGGCACCGAGG | | |
| AGCTGCTCGTGAAGCTGAACCGGGAGGACCTGCTGCGG | | |
| AAGCAGCGGACCTTCGACAACGGTTCAATCCCCCACCAG | | |
| ATCCACCTGGGGGAGCTGCACGCCATCTTGCGGCGGCAG | | |
| GAGGATTTCTATCCTTTTCTGAAGGATAACCGGGAGAAG | | |
| ATCGAGAAGATCCTCACCTTCCGGATCCCCTACTACGTGG | | |
| GGCCCCTCGCTCGCGGCAACTCCCGGTTCGCCTGGATGA | | |
| CCAGGAAGTCCGAGGAGACCATCACCCCCTGGAACTTCG | | |
| AGGAGGTGGTGGACAAGGGCGCCTCCGCTCAGAGCTTC | | |
| ATCGAGCGGATGACCAACTTCGACAAGAACCTGCCCAAC | | |
| GAGAAGGTGCTGCCAAAGCACAGCTTGTTGTACGAGTAC | | |
| TTCACCGTCTATAACGAGCTGACCAAGGTGAAGTACGTG | | |
| ACCGAGGGGATGCGGAAGCCCGCATTCCTCTCGGGCGA | | |
| GCAGAAGAAGGCTATCGTGGACCTGCTGTTCAAGACCAA | | |
| CCGGAAGGTCACCGTGAAGCAGCTGAAGGAGGACTACT | | |
| TCAAGAAGATCGAGTGCTTCGATTCCGTCGAGATTAGCG | | |
| GAGTCGAGGACCGATTTAACGCTTCACTGGGCACCTACC | | |
| ACGACCTGCTGAAGATCATCAAGGACAAGGACTTCCTGG | | |
| ACAACGAGGAGAACGAGGACATCTTGGAGGACATCGTG | | |
| CTGACCCTGACCTTGTTCGAGGACCGGGAGATGATCGAG | | |
| GAGCGCTTGAAGACCTACGCTCACCTGTTCGACGACAAG | | |
| GTGATGAAGCAGCTGAAGCGGCGCCGGTACACTGGCTG | | |
| GGGCCGCTTGAGCAGGAAGCTGATCAACGGCATCCGCG | | |
| ACAAGCAGAGCGGCAAGACCATCCTGGACTTCCTGAAGT | | |
| CCGATGGTTTTGCCAACCGCAACTTCATGCAGCTGATCCA | | |
| CGACGACAGCCTGACCTTCAAGGAGGACATCCAGAAGGC | | |
| TCAGGTGTCAGGGCAGGGAGACAGCCTGCATGAGCACA | | |
| TCGCTAACCTGGCTGGGAGCCCCGCCATCAAGAAGGGGA | | |
| TCCTGCAGACCGTGAAGGTCGTGGATGAGCTGGTGAAG | | |
| GTGATGGGCCGGCACAAGCCTGAGAACATCGTGATCGA | | |
| AATGGCTCGGGAGAACCAGACCACCCAGAAGGGTCAGA | | |
| AGAACAGCCGGGAGCGGATGAAGCGGATCGAGGAGGG | | |
| CATCAAGGAGCTCGGCTCACAGATCCTGAAGGAGCACCC | | |
| CGTGGAGAACACGCAGCTCCAGAACGAGAAGCTGTATCT | | |
| GTACTACCTGCAGAATGGCCGCGACATGTATGTCGACCA | | |
| GGAGCTGGACATCAACCGGCTGTCCGACTACGATGTGGA | | |
| CCACATCGTGCCCCAGAGCTTCCTGAAGGACGATAGCAT | | |
| CGACAACAAGGTGCTGACGCGGTCCGACAAGAATCGGG | | |
| GCAAGAGCGACAACGTTCCCAGCGAGGAGGTGGTGAAG | | |
| AAGATGAAGAACTACTGGCGGCAGCTGCTGAACGCCAA | | |
| GCTGATCACCCAGCGGAAGTTCGACAACTTGACCAAGGC | | |
| CGAGCGGGGCGGCCTGAGCGAGCTGGACAAGGCCGGCT | | |
| TCATCAAGCGGCAGCTGGTGGAGACCAGGCAGATCACCA | | |
| AGCACGTCGCTCAGATCTTGGACAGCAGGATGAACACCA | | |
| AGTACGACGAAAACGATAAGCTGATCAGGGAGGTGAAG | | |
| GTGATCACGCTGAAGTCAAAGCTGGTGAGCGACTTCAGG | | |
| AAGGACTTCCAGTTCTACAAGGTGAGGGAGATCAACAAC | | |
| TACCACCACGCCCACGACGCTTACCTGAACGCCGTGGTG | | |
| GGCACCGCCTTGATCAAGAAGTACCCCAAGCTGGAGTCC | | |
| GAGTTCGTGTACGGCGACTACAAGGTGTACGACGTGCGG | | |
| AAGATGATCGCCAAGAGCGAGCAGGAGATCGGCAAGGC | | |
| CACCGCCAAGTACTTCTTCTACAGCAACATCATGAACTTC | | |
| TTCAAGACCGAGATCACCTTGGCCAACGGTGAGATCAGG | | |
| AAGCGGCCTCTGATCGAGACCAACGGCGAGACCGGGGA | | |
| GATCGTGTGGGACAAGGGCCGGGACTTCGCCACCGTGA | | |
| GGAAGGTGCTGAGCATGCCCCAGGTCAACATCGTGAAG | | |
| AAGACCGAGGTGCAGACCGGGGGCTTCTCAAAGGAGTC | | |
| AATCCTGCCTAAGCGGAACTCCGACAAGCTGATCGCCAG | | |
| GAAGAAGGACTGGGATCCCAAGAAGTACGGCGGCTTCG | | |
| ACTCCCCCACCGTGGCGTACAGCGTGCTGGTCGTGGCCA | | |
| AGGTGGAGAAGGGCAAGTCGAAGAAGCTGAAGAGCGT | | |
| GAAGGAGCTCTTGGGCATCACCATCATGGAGCGGAGTA | | |
| GCTTCGAGAAGAATCCCATCGATTTTCTCGAGGCTAAGG | | |
| GCTACAAGGAGGTCAAGAAGGACCTCATCATCAAGCTCC | | |
| CCAAGTACAGCTTGTTCGAGCTGGAGAACGGGAGGAAG | | |
| CGCATGCTGGCCTCCGCCGGCGAGCTGCAGAAGGGCAA | | |
| CGAGCTCGCCCTGCCCTCCAAGTACGTGAACTTCCTGTAC | | |
| TTGGCCAGCCACTACGAGAAGCTGAAGGGCAGCCCTGA | | |
| GGACAACGAGCAGAAGCAGTTGTTCGTGGAGCAGCACA | | |
| AGCACTACCTGGACGAGATCATCGAGCAGATCTCAGAGT | | |
| TCAGCAAGCGGGTCATCCTGGCCGACGCGAACCTGGACA | | |
| AGGTGCTGTCCGCCTACAACAAGCACCGGGACAAGCCCA | | |
| TCCGGGAGCAGGCCGAGAACATCATCCACCTGTTCACCC | | |
| TGACCAACCTGGGCGCTCCCGCCGCTTTCAAGTACTTCGA | | |
| CACCACCATCGACAGGAAGCGGTACACCTCCACCAAGGA | | |
| GGTGTTGGACGCCACCTTGATCCACCAGAGCATCACCGG | | |
| GCTGTACGAGACCCGGATCGACCTGAGCCAGCTGGGCG | | |
| GCGACGGCGGGGGCAGCCCCAAGAAGAAGCGGAAGGT | | |
| G | | |
| ATGGATAAGAAGTACAGCATCGGGCTGGACATCGGCACC | ART-CDS-K4-8 | 956 |
| AACAGCGTGGGCTGGGCCGTGATCACCGACGAGTACAA | | |
| GGTGCCCAGCAAGAAGTTCAAGGTGCTGGGCAACACCG | | |
| ACCGGCACTCCATCAAGAAGAACTTGATCGGAGCCCTGC | | |
| TGTTCGACAGCGGGGAGACCGCCGAGGCCACCCGGCTG | | |
| AAGCGGACCGCCCGGCGGCGGTACACCCGGCGGAAGAA | | |
| CCGGATCTGCTACCTGCAGGAGATCTTCAGCAACGAGAT | | |
| GGCCAAGGTGGACGACTCCTTCTTCCACCGGCTGGAGGA | | |
| GTCATTCCTGGTCGAGGAGGACAAGAAGCACGAGCGGC | | |
| ACCCCATCTTCGGCAACATCGTGGATGAGGTGGCCTACC | | |
| ACGAGAAGTACCCCACCATCTACCACCTGCGGAAGAAGC | | |
| TGGTGGACAGCACCGACAAGGCCGACCTGCGGCTGATCT | | |
| ACCTGGCCCTGGCCCACATGATCAAGTTCCGGGGCCACTT | | |
| CCTGATCGAGGGCGACCTGAACCCCGATAACAGCGACGT | | |
| GGACAAGCTGTTCATCCAGCTGGTGCAGACCTACAACCA | | |
| GCTGTTCGAGGAGAACCCCATCAACGCTAGCGGGGTGG | | |
| ACGCCAAGGCCATCCTGAGCGCCAGGCTGAGCAAGAGC | | |
| CGCAGGTTGGAGAACCTGATCGCCCAGCTGCCCGGGGA | | |
| GAAGAAGAACGGCCTGTTCGGCAATCTGATCGCCCTCTC | | |
| CCTGGGCCTGACCCCCAACTTCAAGTCCAACTTCGACCTG | | |
| GCCGAGGACGCCAAGCTGCAGCTGTCCAAGGACACCTAC | | |
| GACGACGACCTGGACAACCTGCTGGCCCAGATCGGCGAC | | |
| CAGTACGCCGATCTGTTCCTGGCCGCCAAGAACCTGTCCG | | |
| ACGCCATCCTGCTGTCAGACATCCTGCGGGTGAACACCG | | |
| AGATCACCAAGGCCCCCCTGAGCGCTAGCATGATCAAGC | | |
| GGTACGACGAGCACCACCAGGACCTGACCCTGCTGAAGG | | |
| CCCTGGTGCGGCAGCAGCTGCCCGAGAAGTACAAGGAG | | |
| ATCTTCTTCGACCAGTCCAAGAACGGATACGCCGGCTACA | | |
| TCGACGGGGGCGCCAGCCAGGAGGAGTTCTACAAGTTC | | |
| ATCAAGCCCATCCTGGAGAAGATGGACGGCACCGAGGA | | |
| GCTGCTCGTGAAGCTGAACCGGGAGGACCTGCTGCGGA | | |
| AGCAGCGGACCTTCGACAACGGCAGCATCCCCCACCAGA | | |
| TCCACCTGGGCGAGCTGCACGCCATCCTGCGGCGGCAGG | | |
| AGGACTTCTACCCCTTCCTGAAGGACAACCGGGAGAAGA | | |
| TCGAGAAGATCTTGACCTTCCGGATCCCCTACTACGTGGG | | |
| GCCCCTGGCCCGGGGCAACTCCCGGTTTGCCTGGATGAC | | |
| CAGGAAGAGCGAGGAGACCATCACCCCCTGGAACTTCGA | | |
| GGAGGTGGTGGACAAGGGGGCCTCCGCCCAGAGCTTCA | | |
| TCGAGCGGATGACCAACTTCGACAAGAACCTGCCCAACG | | |
| AGAAGGTGCTGCCAAAGCACTCCCTGCTGTACGAGTACT | | |
| TCACCGTGTACAACGAGCTGACCAAGGTGAAGTACGTGA | | |
| CAGAGGGAATGCGGAAGCCCGCCTTCCTGAGCGGCGAG | | |
| CAGAAGAAGGCCATCGTGGACCTGCTGTTCAAGACCAAC | | |
| CGGAAGGTCACCGTGAAGCAGCTGAAGGAGGACTACTT | | |
| CAAGAAGATCGAGTGCTTCGACAGCGTGGAGATCAGCG | | |
| GCGTGGAGGACCGCTTCAACGCCAGCCTGGGCACCTACC | | |
| ACGACCTGCTGAAGATCATCAAGGACAAGGACTTCCTGG | | |
| ATAACGAGGAGAACGAGGACATCCTGGAGGACATCGTG | | |
| TTGACCCTGACCCTGTTCGAGGACAGGGAGATGATCGAG | | |
| GAGCGGTTGAAGACCTACGCCCACCTGTTCGATGACAAG | | |
| GTGATGAAGCAGCTGAAGCGGAGGCGGTACACCGGCTG | | |
| GGGGCGGCTGAGCCGGAAGCTGATCAACGGGATCCGGG | | |
| ACAAGCAGTCCGGCAAGACCATCCTGGACTTCCTGAAGA | | |
| GCGACGGCTTCGCCAACCGGAACTTCATGCAGCTGATCC | | |
| ACGACGACAGCCTGACCTTCAAGGAGGACATCCAGAAG | | |
| GCCCAGGTGTCAGGCCAGGGCGACAGCCTGCACGAGCA | | |
| CATCGCCAACCTGGCCGGCAGCCCCGCCATCAAGAAGGG | | |
| CATCTTGCAGACCGTGAAGGTCGTGGACGAGCTGGTGAA | | |
| GGTGATGGGCCGGCACAAGCCCGAGAACATCGTGATCG | | |
| AGATGGCCCGGGAGAACCAGACCACCCAGAAGGGCCAG | | |
| AAGAACAGCAGGGAGCGGATGAAGCGGATCGAGGAGG | | |
| GGATCAAGGAGCTGGGCTCCCAGATCCTGAAGGAGCAC | | |
| CCCGTCGAGAACACCCAGCTGCAGAACGAGAAGCTGTAC | | |
| CTGTACTACCTGCAGAACGGGAGGGACATGTACGTGGAC | | |
| CAGGAGCTGGACATCAACCGGCTCAGCGACTACGACGTG | | |
| GACCACATCGTGCCCCAGTCCTTCCTGAAGGACGACAGC | | |
| ATCGATAACAAGGTGCTGACCCGGAGCGATAAGAACCG | | |
| GGGCAAGAGCGATAACGTGCCCAGCGAGGAGGTGGTGA | | |
| AGAAGATGAAGAACTACTGGCGGCAGCTGCTGAACGCC | | |
| AAGCTGATCACCCAGCGGAAGTTCGACAACCTGACCAAG | | |
| GCCGAGCGGGGCGGCCTGAGCGAGCTGGACAAGGCCG | | |
| GCTTCATCAAGCGGCAGCTGGTGGAGACCCGGCAGATCA | | |
| CCAAGCACGTGGCCCAGATCCTGGACTCCAGGATGAACA | | |
| CCAAGTACGACGAGAACGACAAGCTGATCCGGGAGGTG | | |
| AAGGTGATCACCCTGAAGAGCAAGCTGGTGTCAGACTTC | | |
| CGGAAGGACTTCCAGTTCTACAAGGTGCGGGAGATCAAC | | |
| AACTACCACCACGCCCACGACGCCTACCTGAACGCCGTG | | |
| GTGGGCACCGCCCTGATCAAGAAGTACCCCAAGCTGGAG | | |
| TCCGAGTTCGTGTACGGCGACTACAAGGTGTACGACGTG | | |
| CGGAAGATGATCGCCAAGAGCGAGCAGGAGATCGGCAA | | |
| GGCCACCGCCAAGTACTTCTTCTACAGCAACATCATGAAC | | |
| TTCTTCAAGACCGAGATCACCCTGGCCAACGGGGAGATC | | |
| CGGAAGCGGCCCCTGATCGAGACCAACGGCGAGACCGG | | |
| CGAGATCGTGTGGGACAAGGGCCGGGACTTCGCCACCG | | |
| TGCGGAAGGTGCTGTCCATGCCCCAGGTGAACATCGTGA | | |
| AGAAGACCGAGGTGCAGACCGGCGGGTTCAGCAAGGAG | | |
| TCCATCCTGCCCAAGCGGAACAGCGACAAGCTGATCGCT | | |
| CGGAAGAAGGATTGGGACCCCAAGAAGTACGGCGGCTT | | |
| CGACAGCCCCACCGTGGCCTACAGCGTGCTGGTGGTGGC | | |
| CAAGGTGGAGAAGGGCAAGTCAAAGAAGCTGAAGTCAG | | |
| TGAAGGAGCTGCTGGGCATCACCATCATGGAGCGGAGC | | |
| AGCTTCGAGAAGAACCCCATCGACTTCTTGGAGGCCAAG | | |
| GGCTACAAGGAGGTGAAGAAGGATCTGATCATCAAGCT | | |
| GCCCAAGTACTCACTGTTCGAGCTGGAGAACGGCCGGAA | | |
| GCGGATGCTGGCCTCCGCCGGCGAGCTGCAGAAGGGAA | | |
| ACGAGCTGGCCCTGCCCAGCAAGTACGTGAACTTCTTGT | | |
| ACCTGGCCAGCCACTACGAGAAGTTGAAGGGATCACCCG | | |
| AGGACAACGAGCAGAAGCAGCTGTTCGTGGAGCAGCAC | | |
| AAGCACTACCTGGACGAGATCATCGAGCAGATCTCAGAG | | |
| TTCAGCAAGCGGGTGATCCTGGCCGACGCCAACCTGGAC | | |
| AAGGTGCTCAGCGCCTACAACAAGCACCGGGACAAGCCC | | |
| ATCCGGGAGCAGGCCGAGAACATCATCCACCTGTTCACC | | |
| CTGACCAACCTGGGCGCCCCCGCCGCTTTCAAGTACTTCG | | |
| ACACCACCATCGACCGGAAGCGGTACACCAGCACCAAGG | | |
| AGGTGCTGGACGCCACTCTGATCCACCAGAGCATCACCG | | |
| GGCTGTACGAGACCCGGATCGACCTGAGCCAGCTGGGC | | |
| GGCGACGGCGGGGGCAGCCCCAAGAAGAAGCGGAAGG | | |
| TG | | |
| ATGGATAAGAAATATAGCATCGGCCTGGATATCGGCACA | ART-CDS-K8-1 | 957 |
| AACAGCGTGGGCTGGGCCGTGATCACCGACGAGTACAA | | |
| GGTGCCCAGCAAGAAGTTCAAGGTGCTGGGCAACACCG | | |
| ACCGGCACAGCATCAAGAAGAACCTGATCGGCGCCCTGC | | |
| TGTTCGACAGCGGCGAGACCGCCGAGGCCACCCGGCTG | | |
| AAGCGGACCGCCCGGCGGCGGTACACCCGGCGGAAGAA | | |
| CCGGATCTGCTACCTGCAGGAGATCTTCAGCAACGAGAT | | |
| GGCCAAGGTGGACGACAGCTTCTTCCACCGGCTGGAGGA | | |
| GAGCTTCCTGGTGGAGGAGGACAAGAAGCACGAGCGGC | | |
| ACCCCATCTTCGGCAACATCGTGGACGAGGTGGCCTACC | | |
| ACGAGAAGTACCCCACCATCTACCACCTGCGGAAGAAGC | | |
| TGGTGGACAGCACCGACAAGGCCGACCTGCGGCTGATCT | | |
| ACCTGGCCCTGGCCCACATGATCAAGTTCCGGGGCCACTT | | |
| CCTGATCGAGGGCGACCTGAACCCCGATAACTCCGACGT | | |
| GGACAAGCTGTTCATCCAGCTGGTGCAGACCTACAACCA | | |
| GCTGTTCGAGGAGAACCCCATCAACGCCTCAGGCGTGGA | | |
| CGCCAAGGCCATCTTGAGCGCCCGGCTGAGCAAGAGCCG | | |
| GCGGCTGGAGAACCTGATCGCCCAGCTGCCCGGCGAGA | | |
| AGAAGAACGGCCTGTTCGGCAACCTGATCGCCCTGAGCC | | |
| TGGGCCTGACCCCCAACTTCAAGAGCAACTTCGACCTGG | | |
| CCGAGGACGCCAAGCTGCAGCTGAGCAAGGACACCTAC | | |
| GACGACGACCTGGACAACCTGCTGGCCCAGATCGGCGAC | | |
| CAGTACGCCGATCTGTTCCTGGCCGCCAAGAACCTGAGC | | |
| GACGCCATCCTGCTGAGCGACATCCTGCGGGTGAACACC | | |
| GAGATCACCAAGGCCCCCCTGAGCGCCAGCATGATCAAG | | |
| CGGTACGACGAGCACCACCAGGACCTGACCCTGCTGAAG | | |
| GCCCTGGTGCGGCAGCAGCTGCCCGAGAAGTACAAGGA | | |
| GATCTTCTTCGACCAGAGCAAGAACGGCTACGCCGGCTA | | |
| CATCGACGGGGGGCCAGCCAGGAGGAGTTCTACAAGT | | |
| TCATCAAGCCCATCCTGGAGAAGATGGACGGCACCGAGG | | |
| AGCTGCTGGTGAAGCTGAACCGGGAGGACCTGCTGCGG | | |
| AAGCAGCGGACCTTCGACAACGGCAGCATCCCCCACCAG | | |
| ATCCACCTGGGCGAGCTGCACGCCATCCTGCGGCGGCAG | | |
| GAGGACTTCTACCCCTTCCTGAAGGACAACCGGGAGAAG | | |
| ATCGAGAAGATCCTGACCTTCCGGATCCCCTACTACGTGG | | |
| GCCCCCTGGCCCGGGGCAACTCCCGGTTCGCCTGGATGA | | |
| CCCGGAAGAGCGAGGAGACCATCACCCCCTGGAACTTCG | | |
| AGGAGGTGGTGGACAAGGGCGCCAGCGCCCAGAGCTTC | | |
| ATCGAGCGGATGACCAACTTCGACAAGAACCTGCCCAAC | | |
| GAGAAGGTGCTGCCCAAGCACAGCCTGCTGTACGAGTAC | | |
| TTCACCGTGTACAACGAGCTGACCAAGGTGAAGTACGTG | | |
| ACCGAGGGCATGCGGAAGCCCGCCTTCCTGAGCGGCGA | | |
| GCAGAAGAAGGCCATCGTGGACCTGCTGTTCAAGACCAA | | |
| CCGGAAGGTGACCGTGAAGCAGCTGAAGGAGGACTACT | | |
| TCAAGAAGATCGAGTGCTTCGACAGCGTGGAGATCAGCG | | |
| GCGTGGAGGACCGGTTCAACGCCAGCCTGGGCACCTACC | | |
| ACGACCTGCTGAAGATCATCAAGGACAAGGACTTCCTGG | | |
| ACAACGAGGAGAACGAGGACATCCTGGAGGACATCGTG | | |
| CTGACCCTGACCCTGTTCGAGGATCGGGAGATGATCGAG | | |
| GAGCGGCTGAAGACCTACGCCCACCTGTTCGACGACAAG | | |
| GTGATGAAGCAGCTGAAGCGGCGGCGGTACACCGGCTG | | |
| GGGCCGGCTGTCCCGGAAGCTGATCAACGGCATCCGGG | | |
| ACAAGCAGAGCGGCAAGACCATCCTGGACTTCCTGAAGT | | |
| CCGACGGATTCGCCAACCGGAACTTCATGCAGCTGATCC | | |
| ACGACGACAGCCTGACCTTCAAGGAGGACATCCAGAAG | | |
| GCCCAGGTGTCAGGCCAGGGCGACAGCCTGCACGAGCA | | |
| CATCGCCAACCTGGCCGGCAGCCCCGCCATCAAGAAGGG | | |
| CATCCTGCAGACCGTGAAGGTGGTGGACGAGCTGGTGA | | |
| AGGTGATGGGGCGGCACAAGCCCGAGAACATCGTGATC | | |
| GAGATGGCCAGGGAGAACCAGACCACCCAGAAGGGCCA | | |
| GAAGAACAGCCGGGAGCGGATGAAGCGGATCGAGGAG | | |
| GGCATCAAGGAGCTGGGCTCCCAGATCCTGAAGGAGCA | | |
| CCCCGTGGAGAACACCCAGCTGCAGAACGAGAAGCTGTA | | |
| CCTGTACTACCTGCAGAACGGCCGGGACATGTACGTGGA | | |
| CCAGGAGCTGGACATCAACCGGCTGAGCGACTACGACGT | | |
| GGACCACATCGTGCCCCAGAGCTTCCTGAAGGACGACAG | | |
| CATCGACAACAAGGTGCTGACCCGGTCCGACAAGAACCG | | |
| GGGCAAGAGCGACAACGTGCCCTCCGAGGAGGTGGTGA | | |
| AGAAGATGAAGAACTACTGGCGGCAGCTGCTGAACGCC | | |
| AAGCTGATCACCCAGCGGAAGTTCGACAACCTGACCAAG | | |
| GCCGAGCGGGGCGGCCTGAGCGAGCTGGACAAGGCCG | | |
| GCTTCATCAAGCGGCAGCTGGTGGAGACCCGGCAGATCA | | |
| CCAAGCACGTGGCCCAGATCCTGGACAGCCGGATGAACA | | |
| CCAAGTACGACGAGAACGACAAGCTGATCCGGGAGGTG | | |
| AAGGTGATCACCCTGAAGAGCAAGCTGGTGAGCGACTTC | | |
| CGGAAGGACTTCCAGTTCTACAAGGTGCGGGAGATCAAC | | |
| AACTACCACCACGCCCACGACGCCTACCTGAACGCCGTG | | |
| GTGGGCACCGCCCTGATCAAGAAGTACCCCAAGCTGGAG | | |
| TCCGAGTTCGTGTACGGCGACTACAAGGTGTACGACGTG | | |
| CGGAAGATGATCGCCAAGAGCGAGCAGGAGATCGGCAA | | |
| GGCCACCGCCAAGTACTTCTTCTACAGCAACATCATGAAC | | |
| TTCTTCAAGACCGAGATCACCCTGGCCAACGGGGAGATC | | |
| CGGAAGCGGCCCCTGATCGAGACCAACGGCGAGACCGG | | |
| CGAGATCGTGTGGGACAAGGGCCGGGACTTCGCCACCG | | |
| TGCGGAAGGTGCTGTCCATGCCCCAGGTGAACATCGTGA | | |
| AGAAGACCGAGGTGCAGACCGGCGGGTTCAGCAAGGAG | | |
| AGCATCCTGCCCAAGCGGAACAGCGACAAGCTGATCGCC | | |
| CGGAAGAAGGACTGGGACCCCAAGAAGTACGGCGGCTT | | |
| CGACAGCCCCACCGTGGCCTACAGCGTGCTGGTGGTGGC | | |
| CAAGGTGGAGAAGGGCAAGTCAAAGAAGCTGAAGAGCG | | |
| TGAAGGAGCTGCTGGGCATCACCATCATGGAGCGGAGC | | |
| AGCTTCGAGAAGAACCCCATCGACTTCTTGGAGGCCAAG | | |
| GGCTACAAGGAGGTGAAGAAGGACCTGATCATCAAGCT | | |
| GCCCAAGTACTCACTGTTCGAGCTGGAGAACGGCCGGAA | | |
| GCGGATGCTGGCCAGCGCCGGCGAGCTGCAGAAGGGCA | | |
| ACGAGCTGGCCCTGCCCAGCAAGTACGTGAACTTCCTGT | | |
| ACCTGGCCAGCCACTACGAGAAGCTGAAGGGCAGCCCC | | |
| GAGGACAACGAGCAGAAGCAGCTGTTCGTGGAGCAGCA | | |
| CAAGCACTACCTGGACGAGATCATCGAGCAGATCTCAGA | | |
| GTTCAGCAAGCGGGTGATCCTGGCCGACGCCAACCTGGA | | |
| CAAGGTGCTGAGCGCCTACAACAAGCACCGGGACAAGC | | |
| CCATCCGGGAGCAGGCCGAGAACATCATCCACCTGTTCA | | |
| CCCTGACCAACCTGGGCGCCCCCGCCGCCTTCAAGTACTT | | |
| CGACACCACCATCGACCGGAAGCGGTACACCAGCACCAA | | |
| GGAGGTGCTGGACGCCACCCTGATCCACCAGAGCATCAC | | |
| CGGCCTGTACGAGACCCGGATCGACCTGAGCCAGCTGGG | | |
| CGGCGACGGCGGGGGCAGCCCCAAGAAGAAGCGGAAG | | |
| GTG | | |
| ATGGATAAGAAGTACTCCATCGGCCTGGACATCGGCACC | ART-CDS-K10- | 958 |
| AACAGCGTGGGCTGGGCCGTGATCACCGACGAGTACAA | 2 | |
| GGTGCCCAGCAAGAAGTTCAAGGTGCTGGGCAACACCG | | |
| ACCGGCACAGCATCAAGAAGAACCTGATCGGCGCCCTGC | | |
| TGTTCGACAGCGGCGAGACCGCCGAGGCCACCCGGCTG | | |
| AAGCGGACCGCCCGGCGGCGGTACACCCGGCGGAAGAA | | |
| CCGGATCTGCTACCTGCAGGAGATCTTCAGCAACGAGAT | | |
| GGCCAAGGTGGACGACAGCTTCTTCCACCGGCTGGAGGA | | |
| GAGCTTCCTGGTGGAGGAGGACAAGAAGCACGAGCGGC | | |
| ACCCCATCTTCGGCAACATCGTGGACGAGGTGGCCTACC | | |
| ACGAGAAGTACCCCACCATCTACCACCTGCGGAAGAAGC | | |
| TGGTGGACAGCACCGACAAGGCCGACCTGCGGCTGATCT | | |
| ACCTGGCCCTGGCCCACATGATCAAGTTCCGGGGCCACTT | | |
| CCTGATCGAGGGCGACCTGAACCCCGATAACAGCGACGT | | |
| GGACAAGCTGTTCATCCAGCTGGTGCAGACCTACAACCA | | |
| GCTGTTCGAGGAGAACCCCATCAACGCCAGCGGCGTGGA | | |
| CGCCAAGGCCATCCTGAGCGCCCGGCTGAGCAAGAGCC | | |
| GGCGGCTGGAGAACCTGATCGCCCAGCTGCCCGGCGAG | | |
| AAGAAGAACGGCCTGTTCGGCAACCTGATCGCCCTGAGC | | |
| CTGGGCCTGACCCCCAACTTCAAGAGCAACTTCGACCTG | | |
| GCCGAGGACGCCAAGCTGCAGCTGAGCAAGGACACCTA | | |
| CGACGACGACCTGGACAACCTGCTGGCCCAGATCGGCGA | | |
| CCAGTACGCCGATCTGTTCCTGGCCGCCAAGAACCTGTCC | | |
| GACGCCATCCTGCTGAGCGACATCCTGCGGGTGAACACC | | |
| GAGATCACCAAGGCCCCCCTGAGCGCCAGCATGATCAAG | | |
| CGGTACGACGAGCACCACCAGGACCTGACCCTGCTGAAG | | |
| GCCCTGGTGCGGCAGCAGCTGCCCGAGAAGTACAAGGA | | |
| GATCTTCTTCGACCAGTCCAAGAATGGCTACGCCGGCTAC | | |
| ATCGACGGCGGCGCCAGCCAGGAGGAGTTCTACAAGTTC | | |
| ATCAAGCCCATCCTGGAGAAGATGGACGGCACCGAGGA | | |
| GCTGCTGGTGAAGCTGAACCGGGAGGACCTGCTGCGGA | | |
| AGCAGCGGACCTTCGACAACGGCTCCATCCCCCACCAGA | | |
| TCCACCTGGGCGAGCTGCACGCCATCCTGCGGCGGCAGG | | |
| AGGACTTCTACCCCTTCCTGAAGGACAACCGGGAGAAGA | | |
| TCGAGAAGATCCTGACCTTCCGGATCCCCTACTACGTGGG | | |
| CCCCCTGGCCCGGGGCAACAGCCGGTTCGCCTGGATGAC | | |
| CCGGAAGAGCGAGGAGACCATCACCCCCTGGAACTTCGA | | |
| GGAGGTGGTGGACAAGGGCGCCAGCGCCCAGTCCTTCA | | |
| TCGAGCGGATGACCAACTTCGACAAGAACCTGCCCAACG | | |
| AGAAGGTGCTGCCCAAGCACAGCCTGCTGTACGAGTACT | | |
| TCACCGTGTACAACGAGCTGACCAAGGTGAAGTACGTGA | | |
| CCGAGGGCATGCGGAAGCCCGCCTTCCTGTCAGGCGAGC | | |
| AGAAGAAGGCCATCGTGGACCTGCTGTTCAAGACCAACC | | |
| GGAAGGTGACCGTGAAGCAGCTGAAGGAGGACTACTTC | | |
| AAGAAGATCGAGTGCTTCGACAGCGTGGAGATCAGCGG | | |
| CGTGGAGGACCGGTTCAACGCCAGCCTGGGCACCTACCA | | |
| CGACCTGCTGAAGATCATCAAGGACAAGGACTTCCTGGA | | |
| CAACGAGGAGAACGAGGACATCCTGGAGGATATCGTGC | | |
| TGACCCTGACCCTGTTCGAGGACCGGGAGATGATCGAGG | | |
| AGCGGCTGAAGACCTACGCCCACCTGTTCGACGACAAGG | | |
| TGATGAAGCAGCTGAAGCGGCGGCGGTACACCGGCTGG | | |
| GGCCGGCTGAGCCGGAAGCTGATCAACGGCATCCGGGA | | |
| CAAGCAGTCAGGCAAGACCATCCTGGACTTCCTGAAGAG | | |
| CGACGGCTTCGCCAACCGGAACTTCATGCAGCTGATCCA | | |
| CGATGACAGCCTGACCTTCAAGGAGGACATCCAGAAGGC | | |
| CCAGGTGAGCGGCCAGGGCGACAGCCTGCACGAGCACA | | |
| TCGCCAACCTGGCCGGCAGCCCCGCCATCAAGAAGGGCA | | |
| TCCTGCAGACCGTGAAGGTGGTGGATGAGCTGGTGAAG | | |
| GTGATGGGCCGGCACAAGCCCGAGAACATCGTGATCGA | | |
| GATGGCCCGGGAGAACCAGACCACCCAGAAGGGCCAGA | | |
| AGAACAGCCGGGAGCGGATGAAGCGGATCGAGGAGGG | | |
| CATCAAGGAGCTGGGCAGCCAGATCCTGAAGGAGCACC | | |
| CCGTGGAGAACACCCAGCTGCAGAACGAGAAGCTGTACC | | |
| TGTACTACCTGCAGAACGGCCGGGACATGTACGTGGACC | | |
| AGGAGCTGGACATCAACCGGCTGAGCGACTACGACGTG | | |
| GACCACATCGTGCCCCAGTCATTCCTGAAGGACGACAGC | | |
| ATCGACAACAAGGTGCTGACCCGGAGCGACAAGAACCG | | |
| GGGCAAGAGCGACAACGTGCCCTCCGAGGAGGTGGTGA | | |
| AGAAGATGAAGAACTACTGGCGGCAGCTGCTGAACGCC | | |
| AAGCTGATCACCCAGCGGAAGTTCGACAACCTGACCAAG | | |
| GCCGAGCGGGGCGGCCTGAGCGAGCTGGACAAGGCCG | | |
| GCTTCATCAAGCGGCAGCTGGTGGAGACCCGGCAGATCA | | |
| CCAAGCACGTGGCCCAGATCCTGGACAGCCGGATGAACA | | |
| CCAAGTACGACGAGAACGACAAGCTGATCCGGGAGGTG | | |
| AAGGTGATCACCCTGAAGAGCAAGCTGGTGAGCGACTTC | | |
| CGGAAGGACTTCCAGTTCTACAAGGTGCGGGAGATCAAC | | |
| AACTACCACCACGCCCACGACGCCTACCTGAACGCCGTG | | |
| GTGGGCACCGCCCTGATCAAGAAGTACCCCAAGCTGGAG | | |
| TCCGAGTTCGTGTACGGCGACTACAAGGTGTACGACGTG | | |
| CGGAAGATGATCGCCAAGAGCGAGCAGGAGATCGGCAA | | |
| GGCCACCGCCAAGTACTTCTTCTACAGCAACATCATGAAC | | |
| TTCTTCAAGACCGAGATCACCCTGGCCAACGGCGAGATC | | |
| CGGAAGCGGCCCCTGATCGAGACCAACGGCGAGACCGG | | |
| CGAGATCGTGTGGGACAAGGGCCGGGACTTCGCCACCG | | |
| TGCGGAAGGTGCTGTCCATGCCCCAGGTGAACATCGTGA | | |
| AGAAGACCGAGGTGCAGACCGGCGGCTTCAGCAAGGAG | | |
| AGCATCCTGCCCAAGCGGAACAGCGACAAGCTGATCGCC | | |
| CGGAAGAAGGACTGGGACCCCAAGAAGTACGGCGGCTT | | |
| CGACAGCCCCACCGTGGCCTACAGCGTGCTGGTGGTGGC | | |
| CAAGGTGGAGAAGGGCAAGAGCAAGAAGCTGAAGAGC | | |
| GTGAAGGAGCTGCTGGGCATCACCATCATGGAGCGGAG | | |
| CAGCTTCGAGAAGAACCCCATCGACTTCCTGGAGGCCAA | | |
| GGGCTACAAGGAGGTGAAGAAGGACCTGATCATCAAGC | | |
| TGCCCAAGTACAGCCTGTTCGAGCTGGAGAACGGCCGGA | | |
| AGCGGATGCTGGCCAGCGCCGGCGAGCTGCAGAAGGGC | | |
| AACGAGCTGGCCCTGCCCAGCAAGTACGTGAACTTCCTG | | |
| TACCTGGCCAGCCACTACGAGAAGCTGAAGGGCAGCCCC | | |
| GAGGATAACGAGCAGAAGCAGCTGTTCGTGGAGCAGCA | | |
| CAAGCACTACCTGGACGAGATCATCGAGCAGATCTCAGA | | |
| GTTCAGCAAGCGGGTGATCCTGGCCGACGCCAACCTGGA | | |
| CAAGGTGCTGTCCGCCTACAACAAGCACCGGGACAAGCC | | |
| CATCCGGGAGCAGGCCGAGAACATCATCCACCTGTTCAC | | |
| CCTGACCAACCTGGGCGCCCCCGCCGCCTTCAAGTACTTC | | |
| GACACCACCATCGACCGGAAGCGGTACACCAGCACCAAG | | |
| GAGGTGCTGGACGCCACCCTGATCCACCAGAGCATCACC | | |
| GGCCTGTACGAGACCCGGATCGACCTGAGCCAGCTGGGC | | |
| GGCGACGGCGGCGGCAGCCCCAAGAAGAAGCGGAAGG | | |
| TG | | |
| ATGGACAAGAAGTACTCCATCGGCCTGGACATCGGCACC | ART-CDS-S311 | 959 |
| AACTCCGTGGGCTGGGCCGTGATCACCGACGAGTACAAG | | |
| GTGCCCTCCAAGAAGTTCAAGGTGCTGGGCAACACCGAC | | |
| CGGCACTCCATCAAGAAGAACCTGATCGGCGCCCTGCTG | | |
| TTCGACTCCGGCGAGACCGCCGAGGCCACCCGGCTGAAG | | |
| CGGACCGCCCGGCGGCGGTACACCCGGCGGAAGAACCG | | |
| GATCTGCTACCTGCAGGAGATCTTCTCCAACGAGATGGC | | |
| CAAGGTGGACGACTCCTTCTTCCACCGGCTGGAGGAGTC | | |
| CTTCCTGGTGGAGGAGGACAAGAAGCACGAGCGGCACC | | |
| CCATCTTCGGCAACATCGTGGACGAGGTGGCCTACCACG | | |
| AGAAGTACCCCACCATCTACCACCTGCGGAAGAAGCTGG | | |
| TGGACTCCACCGACAAGGCCGACCTGCGGCTGATCTACC | | |
| TGGCCCTGGCCCACATGATCAAGTTCCGGGGCCACTTCCT | | |
| GATCGAGGGCGACCTGAACCCCGACAACTCCGACGTGGA | | |
| CAAGCTGTTCATCCAGCTGGTGCAGACCTACAACCAGCT | | |
| GTTCGAGGAGAACCCCATCAACGCCTCCGGCGTGGACGC | | |
| CAAGGCCATCCTGTCCGCCCGGCTGTCCAAGTCCCGGCG | | |
| GCTGGAGAACCTGATCGCCCAGCTGCCCGGCGAGAAGA | | |
| AGAACGGCCTGTTCGGCAACCTGATCGCCCTGTCCCTGG | | |
| GCCTGACCCCCAACTTCAAGTCCAACTTCGACCTGGCCGA | | |
| GGACGCCAAGCTGCAGCTGTCCAAGGACACCTACGACGA | | |
| CGACCTGGACAACCTGCTGGCCCAGATCGGCGACCAGTA | | |
| CGCCGACCTGTTCCTGGCCGCCAAGAACCTGTCCGACGC | | |
| CATCCTGCTGTCCGACATCCTGCGGGTGAACACCGAGAT | | |
| CACCAAGGCCCCCCTGTCCGCCTCCATGATCAAGCGGTAC | | |
| GACGAGCACCACCAGGACCTGACCCTGCTGAAGGCCCTG | | |
| GTGCGGCAGCAGCTGCCCGAGAAGTACAAGGAGATCTTC | | |
| TTCGACCAGTCCAAGAACGGCTACGCCGGCTACATCGAC | | |
| GGCGGCGCCTCCCAGGAGGAGTTCTACAAGTTCATCAAG | | |
| CCCATCCTGGAGAAGATGGACGGCACCGAGGAGCTGCT | | |
| GGTGAAGCTGAACCGGGAGGACCTGCTGCGGAAGCAGC | | |
| GGACCTTCGACAACGGCTCCATCCCCCACCAGATCCACCT | | |
| GGGCGAGCTGCACGCCATCCTGCGGCGGCAGGAGGACT | | |
| TCTACCCCTTCCTGAAGGACAACCGGGAGAAGATCGAGA | | |
| AGATCCTGACCTTCCGGATCCCCTACTACGTGGGCCCCCT | | |
| GGCCCGGGGCAACTCCCGGTTCGCCTGGATGACCCGGAA | | |
| GTCCGAGGAGACCATCACCCCCTGGAACTTCGAGGAGGT | | |
| GGTGGACAAGGGCGCCTCCGCCCAGTCCTTCATCGAGCG | | |
| GATGACCAACTTCGACAAGAACCTGCCCAACGAGAAGGT | | |
| GCTGCCCAAGCACTCCCTGCTGTACGAGTACTTCACCGTG | | |
| TACAACGAGCTGACCAAGGTGAAGTACGTGACCGAGGG | | |
| CATGCGGAAGCCCGCCTTCCTGTCCGGCGAGCAGAAGAA | | |
| GGCCATCGTGGACCTGCTGTTCAAGACCAACCGGAAGGT | | |
| GACCGTGAAGCAGCTGAAGGAGGACTACTTCAAGAAGA | | |
| TCGAGTGCTTCGACTCCGTGGAGATCTCCGGCGTGGAGG | | |
| ACCGGTTCAACGCCTCCCTGGGCACCTACCACGACCTGCT | | |
| GAAGATCATCAAGGACAAGGACTTCCTGGACAACGAGG | | |
| AGAACGAGGACATCCTGGAGGACATCGTGCTGACCCTGA | | |
| CCCTGTTCGAGGACCGGGAGATGATCGAGGAGCGGCTG | | |
| AAGACCTACGCCCACCTGTTCGACGACAAGGTGATGAAG | | |
| CAGCTGAAGCGGCGGCGGTACACCGGCTGGGGCCGGCT | | |
| GTCCCGGAAGCTGATCAACGGCATCCGGGACAAGCAGTC | | |
| CGGCAAGACCATCCTGGACTTCCTGAAGTCCGACGGCTT | | |
| CGCCAACCGGAACTTCATGCAGCTGATCCACGACGACTC | | |
| CCTGACCTTCAAGGAGGACATCCAGAAGGCCCAGGTGTC | | |
| CGGCCAGGGCGACTCCCTGCACGAGCACATCGCCAACCT | | |
| GGCCGGCTCCCCCGCCATCAAGAAGGGCATCCTGCAGAC | | |
| CGTGAAGGTGGTGGACGAGCTGGTGAAGGTGATGGGCC | | |
| GGCACAAGCCCGAGAACATCGTGATCGAGATGGCCCGG | | |
| GAGAACCAGACCACCCAGAAGGGCCAGAAGAACTCCCG | | |
| GGAGCGGATGAAGCGGATCGAGGAGGGCATCAAGGAG | | |
| CTGGGCTCCCAGATCCTGAAGGAGCACCCCGTGGAGAAC | | |
| ACCCAGCTGCAGAACGAGAAGCTGTACCTGTACTACCTG | | |
| CAGAACGGCCGGGACATGTACGTGGACCAGGAGCTGGA | | |
| CATCAACCGGCTGTCCGACTACGACGTGGACCACATCGT | | |
| GCCCCAGTCCTTCCTGAAGGACGACTCCATCGACAACAA | | |
| GGTGCTGACCCGGTCCGACAAGAACCGGGGCAAGTCCG | | |
| ACAACGTGCCCTCCGAGGAGGTGGTGAAGAAGATGAAG | | |
| AACTACTGGCGGCAGCTGCTGAACGCCAAGCTGATCACC | | |
| CAGCGGAAGTTCGACAACCTGACCAAGGCCGAGCGGGG | | |
| CGGCCTGTCCGAGCTGGACAAGGCCGGCTTCATCAAGCG | | |
| GCAGCTGGTGGAGACCCGGCAGATCACCAAGCACGTGG | | |
| CCCAGATCCTGGACTCCCGGATGAACACCAAGTACGACG | | |
| AGAACGACAAGCTGATCCGGGAGGTGAAGGTGATCACC | | |
| CTGAAGTCCAAGCTGGTGTCCGACTTCCGGAAGGACTTC | | |
| CAGTTCTACAAGGTGCGGGAGATCAACAACTACCACCAC | | |
| GCCCACGACGCCTACCTGAACGCCGTGGTGGGCACCGCC | | |
| CTGATCAAGAAGTACCCCAAGCTGGAGTCCGAGTTCGTG | | |
| TACGGCGACTACAAGGTGTACGACGTGCGGAAGATGATC | | |
| GCCAAGTCCGAGCAGGAGATCGGCAAGGCCACCGCCAA | | |
| GTACTTCTTCTACTCCAACATCATGAACTTCTTCAAGACCG | | |
| AGATCACCCTGGCCAACGGCGAGATCCGGAAGCGGCCCC | | |
| TGATCGAGACCAACGGCGAGACCGGCGAGATCGTGTGG | | |
| GACAAGGGCCGGGACTTCGCCACCGTGCGGAAGGTGCT | | |
| GTCCATGCCCCAGGTGAACATCGTGAAGAAGACCGAGGT | | |
| GCAGACCGGCGGCTTCTCCAAGGAGTCCATCCTGCCCAA | | |
| GCGGAACTCCGACAAGCTGATCGCCCGGAAGAAGGACT | | |
| GGGACCCCAAGAAGTACGGCGGCTTCGACTCCCCCACCG | | |
| TGGCCTACTCCGTGCTGGTGGTGGCCAAGGTGGAGAAG | | |
| GGCAAGTCCAAGAAGCTGAAGTCCGTGAAGGAGCTGCT | | |
| GGGCATCACCATCATGGAGCGGTCCTCCTTCGAGAAGAA | | |
| CCCCATCGACTTCCTGGAGGCCAAGGGCTACAAGGAGGT | | |
| GAAGAAGGACCTGATCATCAAGCTGCCCAAGTACTCCCT | | |
| GTTCGAGCTGGAGAACGGCCGGAAGCGGATGCTGGCCT | | |
| CCGCCGGCGAGCTGCAGAAGGGCAACGAGCTGGCCCTG | | |
| CCCTCCAAGTACGTGAACTTCCTGTACCTGGCCTCCCACT | | |
| ACGAGAAGCTGAAGGGCTCCCCCGAGGACAACGAGCAG | | |
| AAGCAGCTGTTCGTGGAGCAGCACAAGCACTACCTGGAC | | |
| GAGATCATCGAGCAGATCTCCGAGTTCTCCAAGCGGGTG | | |
| ATCCTGGCCGACGCCAACCTGGACAAGGTGCTGTCCGCC | | |
| TACAACAAGCACCGGGACAAGCCCATCCGGGAGCAGGC | | |
| CGAGAACATCATCCACCTGTTCACCCTGACCAACCTGGGC | | |
| GCCCCCGCCGCCTTCAAGTACTTCGACACCACCATCGACC | | |
| GGAAGCGGTACACCTCCACCAAGGAGGTGCTGGACGCC | | |
| ACCCTGATCCACCAGTCCATCACCGGCCTGTACGAGACCC | | |
| GGATCGACCTGTCCCAGCTGGGCGGCGACGGCGGCGGC | | |
| TCCCCCAAGAAGAAGCGGAAGGTG | | |
| ATGGACAAGAAGTACAGCATCGGACTGGACATCGGAAC | ART-CDS-S204 | 960 |
| AAACAGCGTCGGATGGGCAGTCATCACAGACGAATACAA | | |
| GGTCCCGAGCAAGAAGTTCAAGGTCCTGGGAAACACAG | | |
| ACAGACACAGCATCAAGAAGAACCTGATCGGAGCACTGC | | |
| TGTTCGACAGCGGAGAAACAGCAGAAGCAACAAGACTG | | |
| AAGAGAACAGCAAGAAGAAGATACACAAGAAGAAAGAA | | |
| CAGAATCTGCTACCTGCAGGAAATCTTCAGCAACGAAAT | | |
| GGCAAAGGTCGACGACAGCTTCTTCCACAGACTGGAAGA | | |
| AAGCTTCCTGGTCGAAGAAGACAAGAAGCACGAAAGAC | | |
| ACCCGATCTTCGGAAACATCGTCGACGAAGTCGCATACC | | |
| ACGAAAAGTACCCGACAATCTACCACCTGAGAAAGAAGC | | |
| TGGTCGACAGCACAGACAAGGCAGACCTGAGACTGATCT | | |
| ACCTGGCACTGGCACACATGATCAAGTTCAGAGGACACT | | |
| TCCTGATCGAAGGAGACCTGAACCCGGACAACAGCGACG | | |
| TCGACAAGCTGTTCATCCAGCTGGTCCAGACATACAACCA | | |
| GCTGTTCGAAGAAAACCCGATCAACGCAAGCGGAGTCGA | | |
| CGCAAAGGCAATCCTGAGCGCAAGACTGAGCAAGAGCA | | |
| GAAGACTGGAAAACCTGATCGCACAGCTGCCGGGAGAA | | |
| AAGAAGAACGGACTGTTCGGAAACCTGATCGCACTGAGC | | |
| CTGGGACTGACACCGAACTTCAAGAGCAACTTCGACCTG | | |
| GCAGAAGACGCAAAGCTGCAGCTGAGCAAGGACACATA | | |
| CGACGACGACCTGGACAACCTGCTGGCACAGATCGGAG | | |
| ACCAGTACGCAGACCTGTTCCTGGCAGCAAAGAACCTGA | | |
| GCGACGCAATCCTGCTGAGCGACATCCTGAGAGTCAACA | | |
| CAGAAATCACAAAGGCACCGCTGAGCGCAAGCATGATCA | | |
| AGAGATACGACGAACACCACCAGGACCTGACACTGCTGA | | |
| AGGCACTGGTCAGACAGCAGCTGCCGGAAAAGTACAAG | | |
| GAAATCTTCTTCGACCAGAGCAAGAACGGATACGCAGGA | | |
| TACATCGACGGAGGAGCAAGCCAGGAAGAATTCTACAA | | |
| GTTCATCAAGCCGATCCTGGAAAAGATGGACGGAACAGA | | |
| AGAACTGCTGGTCAAGCTGAACAGAGAAGACCTGCTGA | | |
| GAAAGCAGAGAACATTCGACAACGGAAGCATCCCGCACC | | |
| AGATCCACCTGGGAGAACTGCACGCAATCCTGAGAAGAC | | |
| AGGAAGACTTCTACCCGTTCCTGAAGGACAACAGAGAAA | | |
| AGATCGAAAAGATCCTGACATTCAGAATCCCGTACTACGT | | |
| CGGACCGCTGGCAAGAGGAAACAGCAGATTCGCATGGA | | |
| TGACAAGAAAGAGCGAAGAAACAATCACACCGTGGAAC | | |
| TTCGAAGAAGTCGTCGACAAGGGAGCAAGCGCACAGAG | | |
| CTTCATCGAAAGAATGACAAACTTCGACAAGAACCTGCC | | |
| GAACGAAAAGGTCCTGCCGAAGCACAGCCTGCTGTACGA | | |
| ATACTTCACAGTCTACAACGAACTGACAAAGGTCAAGTA | | |
| CGTCACAGAAGGAATGAGAAAGCCGGCATTCCTGAGCG | | |
| GAGAACAGAAGAAGGCAATCGTCGACCTGCTGTTCAAGA | | |
| CAAACAGAAAGGTCACAGTCAAGCAGCTGAAGGAAGAC | | |
| TACTTCAAGAAGATCGAATGCTTCGACAGCGTCGAAATC | | |
| AGCGGAGTCGAAGACAGATTCAACGCAAGCCTGGGAAC | | |
| ATACCACGACCTGCTGAAGATCATCAAGGACAAGGACTT | | |
| CCTGGACAACGAAGAAAACGAAGACATCCTGGAAGACAT | | |
| CGTCCTGACACTGACACTGTTCGAAGACAGAGAAATGAT | | |
| CGAAGAAAGACTGAAGACATACGCACACCTGTTCGACGA | | |
| CAAGGTCATGAAGCAGCTGAAGAGAAGAAGATACACAG | | |
| GATGGGGAAGACTGAGCAGAAAGCTGATCAACGGAATC | | |
| AGAGACAAGCAGAGCGGAAAGACAATCCTGGACTTCCT | | |
| GAAGAGCGACGGATTCGCAAACAGAAACTTCATGCAGCT | | |
| GATCCACGACGACAGCCTGACATTCAAGGAAGACATCCA | | |
| GAAGGCACAGGTCAGCGGACAGGGAGACAGCCTGCACG | | |
| AACACATCGCAAACCTGGCAGGAAGCCCGGCAATCAAGA | | |
| AGGGAATCCTGCAGACAGTCAAGGTCGTCGACGAACTG | | |
| GTCAAGGTCATGGGAAGACACAAGCCGGAAAACATCGT | | |
| CATCGAAATGGCAAGAGAAAACCAGACAACACAGAAGG | | |
| GACAGAAGAACAGCAGAGAAAGAATGAAGAGAATCGAA | | |
| GAAGGAATCAAGGAACTGGGAAGCCAGATCCTGAAGGA | | |
| ACACCCGGTCGAAAACACACAGCTGCAGAACGAAAAGCT | | |
| GTACCTGTACTACCTGCAGAACGGAAGAGACATGTACGT | | |
| CGACCAGGAACTGGACATCAACAGACTGAGCGACTACGA | | |
| CGTCGACCACATCGTCCCGCAGAGCTTCCTGAAGGACGA | | |
| CAGCATCGACAACAAGGTCCTGACAAGAAGCGACAAGA | | |
| ACAGAGGAAAGAGCGACAACGTCCCGAGCGAAGAAGTC | | |
| GTCAAGAAGATGAAGAACTACTGGAGACAGCTGCTGAA | | |
| CGCAAAGCTGATCACACAGAGAAAGTTCGACAACCTGAC | | |
| AAAGGCAGAGAGAGGAGGACTGAGCGAACTGGACAAG | | |
| GCAGGATTCATCAAGAGACAGCTGGTCGAAACAAGACA | | |
| GATCACAAAGCACGTCGCACAGATCCTGGACAGCAGAAT | | |
| GAACACAAAGTACGACGAAAACGACAAGCTGATCAGAG | | |
| AAGTCAAGGTCATCACACTGAAGAGCAAGCTGGTCAGCG | | |
| ACTTCAGAAAGGACTTCCAGTTCTACAAGGTCAGAGAAA | | |
| TCAACAACTACCACCACGCACACGACGCATACCTGAACGC | | |
| AGTCGTCGGAACAGCACTGATCAAGAAGTACCCGAAGCT | | |
| GGAAAGCGAATTCGTCTACGGAGACTACAAGGTCTACGA | | |
| CGTCAGAAAGATGATCGCAAAGAGCGAACAGGAAATCG | | |
| GAAAGGCAACAGCAAAGTACTTCTTCTACAGCAACATCA | | |
| TGAACTTCTTCAAGACAGAAATCACACTGGCAAACGGAG | | |
| AAATCAGAAAGAGACCGCTGATCGAAACAAACGGAGAA | | |
| ACAGGAGAAATCGTCTGGGACAAGGGAAGAGACTTCGC | | |
| AACAGTCAGAAAGGTCCTGAGCATGCCGCAGGTCAACAT | | |
| CGTCAAGAAGACAGAAGTCCAGACAGGAGGATTCAGCA | | |
| AGGAAAGCATCCTGCCGAAGAGAAACAGCGACAAGCTG | | |
| ATCGCAAGAAAGAAGGACTGGGACCCGAAGAAGTACGG | | |
| AGGATTCGACAGCCCGACAGTCGCATACAGCGTCCTGGT | | |
| CGTCGCAAAGGTCGAAAAGGGAAAGAGCAAGAAGCTGA | | |
| AGAGCGTCAAGGAACTGCTGGGAATCACAATCATGGAA | | |
| AGAAGCAGCTTCGAAAAGAACCCGATCGACTTCCTGGAA | | |
| GCAAAGGGATACAAGGAAGTCAAGAAGGACCTGATCAT | | |
| CAAGCTGCCGAAGTACAGCCTGTTCGAACTGGAAAACGG | | |
| AAGAAAGAGAATGCTGGCAAGCGCAGGAGAACTGCAGA | | |
| AGGGAAACGAACTGGCACTGCCGAGCAAGTACGTCAACT | | |
| TCCTGTACCTGGCAAGCCACTACGAAAAGCTGAAGGGAA | | |
| GCCCGGAAGACAACGAACAGAAGCAGCTGTTCGTCGAA | | |
| CAGCACAAGCACTACCTGGACGAAATCATCGAACAGATC | | |
| AGCGAATTCAGCAAGAGAGTCATCCTGGCAGACGCAAAC | | |
| CTGGACAAGGTCCTGAGCGCATACAACAAGCACAGAGAC | | |
| AAGCCGATCAGAGAACAGGCAGAAAACATCATCCACCTG | | |
| TTCACACTGACAAACCTGGGAGCACCGGCAGCATTCAAG | | |
| TACTTCGACACAACAATCGACAGAAAGAGATACACAAGC | | |
| ACAAAGGAAGTCCTGGACGCAACACTGATCCACCAGAGC | | |
| ATCACAGGACTGTACGAAACAAGAATCGACCTGAGCCAG | | |
| CTGGGAGGAGACGGCGGCGGCAGCCCCAAGAAGAAGC | | |
| GGAAGGTG | | |

The efficacy of CDS variants in Huh7 cells. sgRNA with seed sequence NTLA-2001 (AAAGGCUGCUGAUGACACCU, SEQ ID NO: 973) and Cas9 mRNA composing different CDS were delivered to Huh7 cells as described in Example 2, in a 4 point 4-fold duplicates. The cells were lysed 72 hours post treatment for editing analysis as described in Example 2. The editing efficiencies are listed in the following Table 23 below.

TABLE 23
Editing efficacy of cas9 mRNA variants featuring different CDS

| gRNA(nM) | 15.2 | 3.8 | 0.9 | 0.2 |
|---|---|---|---|---|
| K1-1 | 91 | 88 | 66 | 30 |
| | 90 | 90 | 70 | 40 |
| K4-8 | 88 | 91 | 66 | 43 |
| | 89 | 90 | 69 | 46 |
| K8-1 | 90 | 90 | 71 | 42 |
| | 92 | 90 | 72 | 41 |
| K10-2 | 92 | 91 | 73 | 40 |
| | 90 | 91 | 74 | 39 |
| 004R | 86 | 84 | 57 | 21 |
| | 86 | 85 | 55 | 21 |
| S311 | 86 | 85 | 58 | 27 |
| | 83 | 85 | 57 | 25 |
| S204 | 76 | 74 | 45 | 14 |
| | 77 | 74 | 45 | 14 |

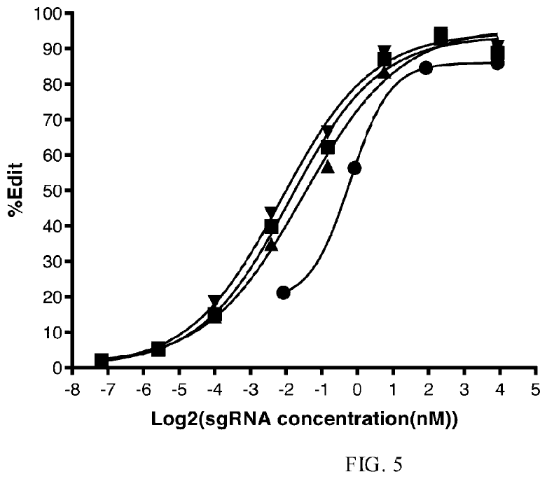

The efficacy of selected CDS variants in Huh7 cells. sgRNA P9-hc-162 targeting human PCSK9 and Cas9 mRNA composing different CDS were delivered to Huh7 cells as described in Example 2, in a 4 point 4-fold or 8-10 point 2-fold dose response curve. The cells were lysed 72 hours post treatment for editing analysis as described in Example 2. The CDS elements were then listed based on EC50 values and maximum editing percent. The dose response curve data for the guide sequences in Huh7 cells is shown in FIG. 5. The EC50 values and maximum editing percent are listed in Table 24 below.

TABLE 24
Editing efficacy of Cas9 mRNA variants
featuring selected CDS in Huh7

| Name | EC50 | Max % Edit |
|---|---|---|
| ART-CDS-004R | 0.865 | 86.2 |
| ART-CDS-K1-1 | 0.266 | 93.8 |
| ART-CDS-K4-8 | 0.348 | 92.9 |
| ART-CDS-K10-2 | 0.226 | 94.1 |

The efficacy of selected CDS variants in PHH cells. Primary human liver hepatocytes (PHH) were thawed in InvitroGRO CP Medium containing 10% FBS and 1% Pen/Strat and plated on collagen coated plates at a density of 13,000 cells/well in a 48-well plate. After 4 to 6 hours, the medium was replaced by InvitroGRO CP medium. Transfections were performed using RNAiMax reagent (Thermofisher) at two fold dilution starting from 200 ng of cas9 mRNA (Levostar) and 100 ng of ART-001-g091 sgRNA per well. The day after transfection, the medium was replaced by 10% FBS, 1% Pen/Strep InvitroGRO CP medium. Another two days later, the cells were harvested for gene editing efficiency as a read-out of expression as shown in Table 25 below.

TABLE 25
Editing efficacy of Cas9 mRNA variants
featuring selected CDS in PHH

| Total | ART-CDS- | ART-CDS- | ART-CDS- | ART-CDS- |
|---|---|---|---|---|
| RNA ng | K1-1 | K10-2 | K4-8 | 004R |
| 37.5 | 31.3 | 39.8 | 37.7 | 34.4 |
| 75 | 58.1 | 60.0 | 59.1 | 53.6 |
| 150 | 71.7 | 71.7 | 74.4 | 70.7 |
| 300 | 74.7 | 71.3 | 76.8 | 67.0 |

Rational Design of polyA Tail for its More Precise Size Distribution

Evaluation of rational designs of PolyA sequences in Huh7 cells. Huh7 cells were plated 8,000 cells/well in a 96-well plate. Transfection were performed using 0.4 uL RNAiMax reagent (Thermofisher) at 100 ng of cas9 mRNA and 100 ng of sgRNA (NTLA-2001) (Genscript). Cells were harvested 3 days after transfection. The genomic DNA was extracted with QuickExtract DNA extract solution (Lucigen). The editing at the on-target sites were amplified by PCR with Taq Pro Multiplex DNA Polymerase (Vazyme). PCR product was purified with VAHTS DNA Clean Beads (Vazyme) and sequenced at an Illumina Novaseq6000 platform. Editing efficacy of cas9 mRNA variants featuring different poly A tail designs in Huh7 cells is shown in Table 26 below. Sequences of the different poly A tail designs are provided as SEQ ID NOs: 963-972 in

Table 27 below.

TABLE 26
Editing efficacy of cas9 mRNA variants featuring different polyA tail designs in Huh7 cells

| GRNA | | | | | | | | pA- | pA- | pA- |
|---|---|---|---|---|---|---|---|---|---|---|
| (nM) | pA31C8A | pA51C8A | pA60C8A | pA60C20A | pA79C20A | pA100 | pA120 | 3Seg | 3SegG | 3SegGG |
| 0.12 | 21 | 31 | 27 | 26 | 32 | 27 | 31 | 35 | 26 | 31 |
| 0.24 | 34 | 43 | 39 | 43 | 42 | 41 | 41 | 44 | 32 | 42 |
| 0.47 | 44 | 58 | 54 | 55 | 53 | 47 | 53 | 55 | 45 | 54 |
| 0.95 | 54 | 63 | 60 | 63 | 65 | 52 | 56 | 63 | 60 | 65 |
| 1.89 | 76 | 73 | 69 | 74 | 76 | 75 | 77 | 72 | 75 | 71 |
| 3.79 | 89 | 91 | 90 | 88 | 91 | 88 | 89 | 89 | 87 | 84 |
| 7.58 | 89 | 93 | 92 | 94 | 92 | 93 | 91 | 93 | 87 | 92 |
| 15.15 | 89 | 92 | 93 | 92 | 92 | 90 | 90 | 92 | 87 | 92 |

TABLE 27
Sequences of polyA tail designs

| Sequence | Name | SEQ ID NO |
|---|---|---|
| AAAAAAAAAAAAAAAAAAAAAAAA | pA31C8A | 963 |
| AAAAAAACCCCCCCCA | | |
| AAAAAAAAAAAAAAAAAAAAAAAA | pA51C8A | 964 |
| AAAAAAAAAAAAAAAAAAAAAAAA | | |
| AAACCCCCCCCA | | |
| AAAAAAAAAAAAAAAAAAAAAAAA | pA60C8A | 965 |
| AAAAAAAAAAAAAAAAAAAAAAAA | | |
| AAAAAAAAAAAACCCCCCCCA | | |
| AAAAAAAAAAAAAAAAAAAAAAAA | pA60C20A | 966 |
| AAAAAAAAAAAAAAAAAAAAAAAA | | |
| AAAAAAAAAAAACCCCCCCCCCCCC | | |
| CCCCCCCA | | |
| AAAAAAAAAAAAAAAAAAAAAAAA | pA79C20A | 967 |
| AAAAAAAAAAAAAAAAAAAAAAAA | | |
| AAAAAAAAAAAAAAAAAAAAAAAA | | |
| AAAAAAACCCCCCCCCCCCCCCCCC | | |
| CCA | | |
| AAAAAAAAAAAAAAAAAAAAAAAA | pA100 | 968 |
| AAAAAAAAAAAAAAAAAAAAAAAA | | |
| AAAAAAAAAAAAAAAAAAAAAAAA | | |
| AAAAAAAAAAAAAAAAAAAAAAAA | | |
| AAAA | | |
| AAAAAAAAAAAAAAAAAAAAAAAA | pA120 | 969 |
| AAAAAAAAAAAAAAAAAAAAAAAA | | |
| AAAAAAAAAAAAAAAAAAAAAAAA | | |
| AAAAAAAAAAAAAAAAAAAAAAAA | | |
| AAAAAAAAAAAAAAAAAAAAAAAA | | |
| AAAAAAAAAAAAAAAAAAAAAAAA | pA-3Seg | 970 |
| AAAAAAGCGAAAAAAAAAAAAAAA | | |
| AAAAAAAAAAAAAAACCGAAAAAA | | |
| AAAAAAAAAAAAAAAAAAAAAAAA | | |
| AAAAAAAAAAAA | | |
| AAAAAAAAAAAAAAAAAAAAAAAA | pA-3SegG | 971 |
| AAAAAAGCGAAAAAAAAAAAAAAA | | |
| AAAAAAAAAAAAAAACCGAAAAAA | | |
| AAAAAAAAAAAAAAAAAAAAAAAA | | |
| AAAAAAAAAAAAG | | |
| AAAAAAAAAAAAAAAAAAAAAAAA | pA-3SegGG | 972 |
| AAAAAAGCGAAAAAAAAAAAAAAA | | |
| AAAAAAAAAAAAAAACCGAAAAAA | | |
| AAAAAAAAAAAAAAAAAAAAAAAA | | |
| AAAAAAAAAAAAGG | | |

Determine the Length Distribution of polyA in mRNA Tail by Mass Spectrometry

The polyA tail of mRNA of different designed were analyzed as described in the methods. The distribution of polyA length deviated from expected size in plasmid are shown in Table 28 below, as percentage of total detected events. Adding G or GG at the end reduces the size distribution widths and deviation of the peak value from expected peak.

TABLE 28
Length distribution of polyA in mRNAs

| Deviation from expected size | pA-3Seg | pA-3SegG | pA-3SegGG |
|---|---|---|---|
| −2 | | | 1.7 |
| −1 | 3.5 | 8.6 | 5.8 |
| 0 | 8.5 | 17.3 | 15.6 |
| +1 | 14.5 | 23.1 | 24.0 |
| +2 | 18.4 | 21.4 | 19.6 |
| +3 | 16.9 | 14.8 | 14.7 |
| +4 | 13.8 | 10.0 | 14.1 |
| +5 | 10.7 | 7.7 | 4.6 |
| +6 | 8.8 | | |
| +7 | 2.5 | | |
| +8 | 2.4 | | |

Evaluation of rational designs of PolyA sequences in PHH cells. Primary human liver hepatocytes (PHH) were thawed in InvitroGRO CP Medium containing 10% FBS and 1% Pen/Strat and plated on collagen coated plates at a density of 13,000 cells/well in a 48-well plate. After 4 to 6 hours, the medium was replaced by InvitroGRO CP medium. Transfection was performed using RNAiMax reagent (Thermofisher) at 250 ng of cas9 mRNA and 250 ng of sgRNA (General Biosystem). The day after transfection, the medium was replaced by 1% Pen/Strep InvitroGRO CP medium supplemented with 10% FBS until cell harvest which was 3 days after transfection. Editing efficacy of Cas9 mRNA variants featuring different polyA tail designs in PHH cells is shown in Table 29 below.

TABLE 29
Editing efficacy of Cas9 mRNA variants featuring
different polyA tail designs in PHH cells

| | | | | | | | pA- |
|---|---|---|---|---|---|---|---|
| gRNA(ng) | pA51C8A | pA60C8A | pA60C20A | pA100 | pA120 | pA-3SegG | 3SegGG |
| 7.8 | 16.3 | 15.6 | 17.7 | 13.7 | 12.7 | Not tested | 19.1 |
| 15.6 | 27.1 | 26.2 | 30.4 | 27.3 | 26.3 | Not tested | 29.3 |
| 31.3 | 38.7 | 40.6 | 39.8 | 32.7 | 37.6 | Not tested | 36.9 |
| 62.5 | 44.7 | 42.3 | 45.3 | 46.2 | 38.9 | 38.1 | 41.5 |
| 125.0 | 53.5 | 49.2 | 51 | 53.8 | 39.7 | 45.1 | 44 |
| 250.0 | 53.2 | 52.5 | 22.3 | 56.6 | 34 | 44.1 | 51.2 |

### Example 14: In Vivo Evaluation of sgRNAs in Humanized PCSK9 Mice

Humanized PCSK9 mice were engineered such that a region of the endogenous murine Pcsk9 locus was deleted and replaced with an orthologous human PCSK9 sequence so that the locus encodes a human PCSK9 protein. These mice humanized with respect to the PCSK9 gene were dosed with LNP formulation containing Cas9 mRNA (SEQ ID NO: 902) in a 2:1 ratio by weight to the sgRNA, as indicated in Table 30 below. The LNPs contained ALC-0315, DSPC, Cholesterol, and PEG2k-DMG. Dosing level was at 1 mg/kg or 0.3 mg/kg (by total RNA content) via intravenous injection. Mice dosed with vehicle alone (20 mM Tris buffer containing 7.5% sucrose) served as the negative controls.

TABLE 30
Experimental design for in vivo evaluation
of sgRNAs in humanized PCSK9 mice

| | Dose | Days post treatment for | |
|---|---|---|---|
| sgRNA | (mg/kg RNA) | liver and serum collection | N |
| P9-hc-212 | 1 | 7 | 1 |
| P9-h-057 | 1 | 7 | 1 |
| P9-hc-162 | 1 | 7 | 2 |
| P9-hc-275 | 1 | 7 | 1 |
| P9-hc-175 | 1 | 7 | 1 |
| P9-hc-010 | 1 | 7 | 1 |
| P9-hc-212 | 0.3 | 7 | 1 |
| P9-h-057 | 0.3 | 7 | 1 |
| P9-hc-162 | 0.3 | 7 | 2 |
| P9-hc-026 | 0.3 | 11 | 2 |
| P9-hc-082 | 0.3 | 11 | 1 |
| P9-hc-043 | 0.3 | 11 | 1 |
| P9-hc-162 | 0.3 | 11 | 4 |
| P9-hc-223 | 0.3 | 11 | 1 |
| P9-hc-026 | 1 | 11 | 2 |
| P9-hc-082 | 1 | 11 | 1 |
| P9-hc-043 | 1 | 11 | 1 |
| P9-hc-162 | 1 | 11 | 4 |
| P9-hc-223 | 1 | 11 | 1 |
| Vehicle | NA | 11 | 1 |
| P9-hc-028 | 0.3 | 11 | 1 |
| P9-hc-095 | 0.3 | 11 | 1 |
| P9-hc-010 | 0.3 | 11 | 1 |
| P9-hc-063 | 0.3 | 11 | 1 |
| P9-hc-028 | 1 | 11 | 1 |
| P9-hc-095 | 1 | 11 | 1 |
| P9-hc-010 | 1 | 11 | 1 |
| P9-hc-063 | 1 | 11 | 1 |
| P9-hc-023 | 1 | 14 | 1 |
| P9-hc-028 | 1 | 14 | 1 |
| P9-hc-175 | 1 | 14 | 1 |
| P9-hc-026 | 1 | 14 | 1 |
| P9-hc-023 | 0.3 | 14 | 1 |
| P9-hc-028 | 0.3 | 14 | 1 |
| P9-hc-175 | 0.3 | 14 | 1 |
| P9-hc-026 | 0.3 | 14 | 1 |
| Vehicle | NA | 14 | 1 |
| P9-hc-162 | 1 | 14 | 1 |
| P9-hc-162 | 0.3 | 14 | 1 |

Liver editing results were determined using primers designed to amplify the region of interest for NGS analysis, and knockdown of serum human PCSK9 protein was detected using a specific human PCSK9 ELISA kit as described above. Results of liver gene editing and knockdown of serum PCSK9 protein for each group are shown in Table 31 below. Editing of PCSK9 gene and subsequent protein knockdown were demonstrated with a series of sgRNAs, including P9-hc-162, P9-hc-212, P9-h-057, P9-hc-082 and P9-hc-023. A clear dose response is observed for both liver gene editing and reduction of serum PCSK9 protein with each of sgRNAs. Some data are not available due to the low number of sequencing reads.

TABLE 31
Liver PCSK9 gene editing and serum PCSK9
(% KD) results for sgRNAs screening

| | | Liver | |
|---|---|---|---|
| | | PCSK9 | Knockdown of |
| | Dose | gene editing | Serum PCSK9 |
| sgRNA | (mg/kg RNA) | (%) | (% vs predose) |
| P9-hc-212 | 1 | 64.1 | 99.2 |
| P9-h-057 | 1 | 65.2 | 99.8 |
| P9-hc-162 | 1 | 57.8 | 99.1 |
| P9-hc-275 | 1 | No available | 66.2 |
| | | data | |
| P9-hc-175 | 1 | No available | 56.7 |
| | | data | |
| P9-hc-010 | 1 | No available | 65.6 |
| | | data | |
| P9-hc-212 | 0.3 | 18.8 | 43.8 |
| P9-h-057 | 0.3 | 22.8 | 51.9 |
| P9-hc-162 | 0.3 | 34.8 | 71.5 |
| P9-hc-026 | 0.3 | 16.0 | 42.2 |
| P9-hc-082 | 0.3 | 17.4 | 51.5 |
| P9-hc-043 | 0.3 | 3.4 | −56.0 |
| P9-hc-162 | 0.3 | 28.7 | 71.6 |
| P9-hc-223 | 0.3 | 2.5 | −32.8 |
| P9-hc-026 | 1 | 50.3 | 92.2 |
| P9-hc-082 | 1 | 54.4 | 94.3 |
| P9-hc-043 | 1 | 15.0 | 71.2 |
| P9-hc-162 | 1 | 54.9 | 95.3 |
| P9-hc-223 | 1 | 24.5 | 74.5 |
| Vehicle | NA | 0.1 | 13.8 |
| P9-hc-028 | 0.3 | 2.2 | 53.1 |
| P9-hc-095 | 0.3 | 2.9 | 6.1 |
| P9-hc-010 | 0.3 | 1.6 | 33.9 |
| P9-hc-063 | 0.3 | 3.0 | 25.0 |
| P9-hc-028 | 1 | 40.6 | 77.5 |
| P9-hc-095 | 1 | 17.2 | 54.2 |
| P9-hc-010 | 1 | 10.2 | 46.5 |
| P9-hc-063 | 1 | 24.9 | 76.3 |
| P9-hc-023 | 1 | 48.1 | 95.5 |
| P9-hc-028 | 1 | 13.8 | 74.4 |
| P9-hc-175 | 1 | 2.2 | 84.8 |
| P9-hc-026 | 1 | 53.6 | 96.5 |
| P9-hc-023 | 0.3 | 11.7 | 70.3 |
| P9-hc-028 | 0.3 | 1.0 | 29.6 |
| P9-hc-175 | 0.3 | 0.1 | 57.9 |
| P9-hc-026 | 0.3 | 18.0 | 47.6 |
| Vehicle | NA | 0.0 | 18.8 |
| P9-hc-162 | 1 | No available | 92.1 |
| | | data | |
| P9-hc-162 | 0.3 | No available | 66.4 |
| | | data | |

### Example 15: In Vivo Assessment of DNA/RNA Hybrid sgRNA Designs in Humanized PCSK9 Mice

Humanized PCSK9 mice were engineered such that a region of the endogenous murine Pcsk9 locus was deleted and replaced with an orthologous human PCSK9 sequence so that the locus encodes a human PCSK9 protein. These mice humanized with respect to the PCSK9 gene were dosed with LNP formulation containing Cas9 mRNA (SEQ ID NO: 902) in a 2:1 ratio by weight to the sgRNA, as indicated in Table 32 below. The LNPs contained ALC-0315, DSPC, Cholesterol, and PEG2k-DMG. Dosing level was at 0.6 mg/kg or 0.2 mg/kg (by total RNA content) and administration was via 10 intravenous injection. As negative controls, mice of the corresponding genotype were dosed with vehicle alone (20 mM Tris buffer containing 7.5% sucrose).

TABLE 32
Experimental design for in vivo assessment of
DNA/RNA hybrid sgRNAs in humanized PCSK9 mice

| | | Days post | |
|---|---|---|---|
| | | treatment for | |
| | Dose | liver and serum | |
| sgRNA | (mg/kg RNA) | collection | N |
| Vehicle | 0 | 7 | 1 |
| P9-hc-023 | 0.6 | 7 | 1 |
| P9-hc-023-seq5 | 0.6 | 7 | 1 |
| P9-hc-023-seq9 | 0.6 | 7 | 1 |
| P9-hc-162 | 0.6 | 7 | 1 |
| P9-hc-162-seq5 | 0.6 | 7 | 1 |
| P9-hc-162-seq6 | 0.6 | 7 | 1 |
| P9-hc-028-seq5 | 0.6 | 7 | 1 |
| P9-hc-212-seq2 | 0.6 | 7 | 1 |
| P9-hc-023 | 0.2 | 7 | 1 |
| P9-hc-023-seq5 | 0.2 | 7 | 1 |
| P9-hc-023-seq9 | 0.2 | 7 | 1 |
| P9-hc-162 | 0.2 | 7 | 1 |
| P9-hc-162-seq5 | 0.2 | 7 | 1 |
| P9-hc-162-seq6 | 0.2 | 7 | 1 |
| P9-hc-028-seq5 | 0.2 | 7 | 1 |
| P9-hc-212-seq2 | 0.2 | 7 | 1 |

Knockdown of serum human PCSK9 protein was detected using a specific human PCSK9 ELISA kit as described above. Serum PCSK9 protein reduction for each group is shown in Table 33 below. Efficacy of protein knockdown was shown with a range of sgRNAs including P9-hc-162, P9-hc-162-seq5, P9-hc-162-seq6, P9-hc-023, and P9-hc-023-seq9.

TABLE 33
Serum PCSK9 (% KD) results for sgRNAs screening.

| | | Knockdown of |
|---|---|---|
| | Dose | Serum PCSK9 |
| sgRNA | (mg/kg RNA) | (% vs predose) |
| Vehicle | 0 | 0.9 |
| P9-hc-023 | 0.6 | 63.6 |
| P9-hc-023-seq5 | 0.6 | 43.7 |
| P9-hc-023-seq9 | 0.6 | 58.2 |
| P9-hc-162 | 0.6 | 80.3 |
| P9-hc-162-seq5 | 0.6 | 79.5 |
| P9-hc-162-seq6 | 0.6 | 81.9 |
| P9-hc-028-seq5 | 0.6 | 47.5 |
| P9-hc-212-seq2 | 0.6 | 46.3 |
| P9-hc-023 | 0.2 | 56.0 |
| P9-hc-023-seq5 | 0.2 | 54.3 |
| P9-hc-023-seq9 | 0.2 | 65.2 |
| P9-hc-162 | 0.2 | 69.0 |
| P9-hc-162-seq5 | 0.2 | 44.9 |
| P9-hc-162-seq6 | 0.2 | 55.6 |
| P9-hc-028-seq5 | 0.2 | 48.0 |
| P9-hc-212-seq2 | 0.2 | 26.5 |

### Example 16: In Vivo Evaluation of UTR Designs in Humanized PCSK9 Mice

Humanized PCSK9 mice were engineered such that a region of the endogenous murine Pcsk9 locus was deleted and replaced with an orthologous human PCSK9 sequence so that the locus encodes a human PCSK9 protein. These mice humanized with respect to the PCSK9 gene were dosed with LNP formulation containing Cas9 mRNA comprising distinct UTR sequences (SEQ ID NOs 941-953 provided in Table 34 below) in a 2:1 ratio by weight to the sgRNA P9-hc-162 (SEQ ID NO: 805), as indicated in Table 35 below. The LNPs contained ALC-0315, DSPC, Cholesterol, and PEG2k-DMG. Dosing was at 1 mg/kg or 0.3 mg/kg (by total RNA content) via intravenous injection (N=1/group). As negative controls, mice of the corresponding genotype were dosed with vehicle alone (20 mM Tris buffer containing 7.5% sucrose).

TABLE 34
UTR sequences of Cas9 mRNAs

| | | SEQ |
|---|---|---|
| | | ID |
| Sequence | Name | NO |
| AGGGACCCGCAGCTCAGCTACAGCAC | ART-UTR-16-5′ | 941 |
| AGATCAGCACC | | |
| AGCAATCCTTTCTTTCAGCTGGAGTGC | ART-UTR-37-5′ | 942 |
| TCCTCAGGAGCCAGCCCCACCCTTAGA | | |
| AAAG | | |
| AGTATATTAGTGCTAATTTCCCTCCGTT | ART-UTR-21-5′ | 943 |
| TGTCCTAGCTTTTCTCTTCTGTCAACCC | | |
| CACACGCCTTTGGCACA | | |
| AGCACTGCCTGGCTCCACGTGCCTCCT | ART-UTR-23-5′ | 944 |
| GGTCTCAGT | | |
| ACTATAAATAGCAGCCACCTCTCCCTG | ART-UTR-26-5′ | 945 |
| GCAGACAGGGACCCGCAGCTCAGCTA | | |
| CAGCACAGATCAGCACC | | |
| AGGCACAGACACCAAGGACAGAGACG | ART-UTR-28-5′ | 946 |
| CTGGCTAGGCCGCCCTCCCCACTGTTA | | |
| CCAAC | | |
| TCGGGGAGCTCGGCTCTTGAGACAGG | ART-UTR-33-5′ | 947 |
| AATCTTGCCCATTCCCCGAACGAATAA | | |
| ACCCCTTCCTTAACTCAGCGTCTGAGG | | |
| AATTTTGTCTGCGGCTCCTCCTGCTACA | | |
| TTCTGAGTGGGGAAAGGGACTAAGGT | | |
| GGTCTGAGGACCCCACAGAGTCAGGA | | |
| AGATTGAGAGCCTGATAAAGGTCCTG | | |
| CGGGCAGGACAGGACCTCCCAACCAA | | |
| GCCCTCCAGCAAGGATTCAGAGTGCCC | | |
| CTCCGGCCTCGCC | | |
| GCTTCCTCTTCACTCTGCTCTCAGGAGA | ART-UTR-16-3′ | 948 |
| TCTGGCTGTGAGGCCCTCAGGGCAGG | | |
| GATACAAAGCGGGGAGAGGGTACACA | | |
| ATGGGTATCTAATAAATACTTAAGAGG | | |
| TGGAA | | |
| ACTAAGTTAAATATTTCTGCACAGTGT | ART-UTR-37-3′ | 949 |
| TCCCATGGCCCCTTGCATTTCCTTCTTA | | |
| ACTCTCTGTTACACGTCATTGAAACTAC | | |
| ACTTTTTTGGTCTGTTTTTGTGCTAGAC | | |
| TGTAAGTTCCTTGGGGGCAGGGCCTTT | | |
| GTCTGTCTCATCTCTGTATTCCCAAATG | | |
| CCTAACAGTACAGAGCCATGACTCAAT | | |
| AAATACATGTTAAATGGATGAATG | | |
| CAGGACACAGCCTTGGATCAGGACAG | ART-UTR-23-3′ | 950 |
| AGACTTGGGGGCCATCCTGCCCCTCCA | | |
| ACCCGACATGTGTACCTCAGCTTTTTCC | | |
| CTCACTTGCATCAATAAAGCTTCTGTGT | | |
| TTGGAACAGCT | | |
| GCTTCCTCTTCACTCTGCTCTCAGGAGA | ART-UTR-26-3′ | 951 |
| CCTGGCTATGAGGCCCTCGGGGCAGG | | |
| GATACAAAGTTAGTGAGGTCTATGTCC | | |
| AGAGAAGCTGAGATATGGCATATAAT | | |
| AGGCATCTAATAAATGCTTAAGAGGT | | |
| GG | | |
| AGTGTCCAGACCATTGTCTTCCAACCC | ART-UTR-28-3′ | 952 |
| CAGCTGGCCTCTAGAACACCCACTGGC | | |
| CAGTCCTAGAGCTCCTGTCCCTACCCA | | |
| CTCTTTGCTACAATAAATGCTGAATGA | | |
| ATCC | | |
| GGACCTGAAGGGTGACATCCCAGGAG | ART-UTR-33-3′ | 953 |
| GGGCCTCTGAAATTTCCCACACCCCAG | | |
| CGCCTGTGCTGAGGACTCCCTCCATGT | | |
| GGCCCCAGGTGCCACCAATAAAAATCC | | |
| TACAG | | |

TABLE 35
Experimental design for in vivo screening
of UTRs in humanized PCSK9 mice

| | | Days post treatment | |
|---|---|---|---|
| | Dose | for liver and serum | |
| UTR | (mg/kg RNA) | collection | N |
| Vehicle | NA | 7 | 1 |
| ART-UTR-16 | 0.3 | 7 | 1 |
| ART-UTR-21 | 0.3 | 7 | 1 |
| ART-UTR-23 | 0.3 | 7 | 1 |
| ART-UTR-26 | 0.3 | 7 | 1 |
| ART-UTR-28 | 0.3 | 7 | 1 |
| ART-UTR-33 | 0.3 | 7 | 1 |
| ART-UTR-37 | 0.3 | 7 | 1 |
| ART-UTR-16 | 1 | 7 | 1 |
| ART-UTR-21 | 1 | 7 | 1 |
| ART-UTR-23 | 1 | 7 | 1 |
| ART-UTR-26 | 1 | 7 | 1 |
| ART-UTR-28 | 1 | 7 | 1 |
| ART-UTR-33 | 1 | 7 | 1 |
| ART-UTR-37 | 1 | 7 | 1 |

Liver editing results were determined using primers designed to amplify the region of interest for NGS analysis, and knockdown of serum human PCSK9 protein was detected using a specific human PCSK9 ELISA kit as described above. Results of liver gene editing and knockdown of serum PCSK9 protein at 7 days post treatment for each group are shown in Table 36 below. Efficient editing of PCSK9 sequence and protein knockdown were shown in tested LNPs with Cas9 mRNAs comprising different UTR sequences including ART-UTR-21, ART-UTR-26, ART-UTR-28, ART-UTR-28, and ART-UTR-37.

TABLE 36
Efficacy of gene editing and serum PCSK9 protein
reduction of different UTR designs

| | Dose | Liver PCSK9 | Knockdown of |
|---|---|---|---|
| | (mg/kg | gene editing | Serum PCSK9 |
| UTR | RNA) | (%) | (% vs predose) |
| Vehicle | NA | 0.0 | 36.7 |
| ART-UTR-16 | 0.3 | 13.7 | 62.5 |
| ART-UTR-21 | 0.3 | 9.9 | 42.5 |
| ART-UTR-23 | 0.3 | 10.2 | 53.5 |
| ART-UTR-26 | 0.3 | 14.2 | 37.5 |
| ART-UTR-28 | 0.3 | 17.0 | 50.4 |
| ART-UTR-33 | 0.3 | 12.6 | 52.0 |
| ART-UTR-37 | 0.3 | 7.6 | 64.4 |
| ART-UTR-16 | 1 | 21.6 | 69.5 |
| ART-UTR-21 | 1 | 49.9 | 94.1 |
| ART-UTR-23 | 1 | 30.9 | 73.4 |
| ART-UTR-26 | 1 | 42.8 | 96.7 |
| ART-UTR-28 | 1 | 47.9 | 90.0 |
| ART-UTR-33 | 1 | 40.9 | 83.5 |
| ART-UTR-37 | 1 | 53.7 | 96.2 |

### Example 17: In Vivo Assessment of Different CDS Designs in Humanized PCSK9 Mice

Humanized PCSK9 mice were engineered such that a region of the endogenous murine Pcsk9 locus was deleted and replaced with an orthologous human PCSK9 sequence so that the locus encodes a human PCSK9 protein. These mice humanized with respect to the PCSK9 gene were dosed with LNP formulation containing Cas9 mRNA comprising a range of CDS designs (provided in Table 22 as SEQ ID NOs: 954-960) in a 2:1 ratio by weight to the sgRNA P9-hc-162 (SEQ ID NO: 805), as indicated in Table 37 below. The LNPs contained ALC-0315, DSPC, Cholesterol, and PEG2k-DMG in a 49.5:9. 5:38.5:2.5 molar ratio. Dosing was at 0.3 mg/kg or 0.1 mg/kg (by total RNA content) via intravenous injection (N=1/group). As negative controls, mice of the corresponding genotype were dosed with vehicle alone (20 mM Tris buffer containing 7.5% sucrose).

TABLE 37
Experimental design of in vivo evaluation of
different CDS designs in humanized PCSK9 mice

| | | Days post | |
|---|---|---|---|
| | | treatment for | |
| | Dose | liver and serum | |
| CDS | (mg/kg RNA) | collection | N |
| Vehicle | NA | 7 | 1 |
| ART-CDS-004R | 0.3 | 7 | 1 |
| ART-CDS-K1-1 | 0.3 | 7 | 1 |
| ART-CDS-K4-8 | 0.3 | 7 | 1 |
| ART-CDS-K10-2 | 0.3 | 7 | 1 |
| ART-CDS-004R | 0.1 | 7 | 1 |
| ART-CDS-K1-1 | 0.1 | 7 | 1 |
| ART-CDS-K4-8 | 0.1 | 7 | 1 |
| ART-CDS-K10-2 | 0.1 | 7 | 1 |

Liver editing was assessed by using primers designed to amplify the region of interest for subsequent NGS analysis. Additionally, the reduction of serum human PCSK9 protein was detected using a specific human PCSK9 ELISA kit, as described above. Results of liver gene editing and knockdown of serum PCSK9 protein at 7 days post treatment for each group are shown in Table 38 below. Efficient editing of PCSK9 sequence and protein knockdown were shown in all tested CDS sequences.

TABLE 38
The efficacy of gene editing and serum
PCSK9 reduction of different CDS design

| | Dose | Liver PCSK9 | Knockdown of |
|---|---|---|---|
| | (mg/kg | gene editing | Serum PCSK9 |
| CDS | RNA) | (%) | (% vs predose) |
| Vehicle | NA | 1.1 | 15.0 |
| ART-CDS-004R | 0.3 | 41.3 | 78.8 |
| ART-CDS-K1-1 | 0.3 | 38.4 | 84.3 |
| ART-CDS-K4-8 | 0.3 | 42.4 | 86.3 |
| ART-CDS-K10-2 | 0.3 | 40.0 | 80.4 |
| ART-CDS-004R | 0.1 | 14.7 | 72.5 |
| ART-CDS-K1-1 | 0.1 | 16.5 | 73.4 |
| ART-CDS-K4-8 | 0.1 | 14.9 | 51.6 |
| ART-CDS-K10-2 | 0.1 | 16.7 | 65.4 |

### Other Embodiments

It is to be understood that the foregoing description is intended to illustrate and not limit the scope of the appended claims. Other aspects, advantages, and modifications are within the scope of the following claims.

1. A guide RNA comprising:
a. a sequence selected from SEQ ID NOs: 911, 915, 933, 934, 1-296, 908-910, 912-914, 916-932, and 935-940;
b. at least 15, 16, 17, 18, 19, or 20 contiguous nucleotides of a sequence selected from SEQ ID NOs: 911, 915, 933, 934, 1-296, 908-910, 912-914, 916-932, and 935-940; or
c. a sequence that is at least 90%, 91%, 92%, 93%, 94%, 95%, 96%, 97%, 98%, or 99% identical to a sequence selected from SEQ ID NOs: 911, 915, 933, 934, 1-296, 908-910, 912-914, 916-932, and 935-940.

2. A vector comprising one of more nucleic acids encoding one or more guide RNAs, wherein the one or more guide RNAs comprise:
a. one or more sequences selected from SEQ ID NOs: 911, 915, 933, 934, 1-296, 908-910, 912-914, 916-932, and 935-940;
b. at least 15, 16, 17, 18, 19, or 20 contiguous nucleotides of one or more sequences selected from SEQ ID NOs: 911, 915, 933, 934, 1-296, 908-910, 912-914, 916-932, and 935-940; or
c. one or more sequences that is at least 90%, 91%, 92%, 93%, 94%, 95%, 96%, 97%, 98%, or 99% identical to a sequence selected from SEQ ID NOs: 911, 915, 933, 934, 1-296, 908-910, 912-914, 916-932, and 935-940.

3. A composition comprising
(i) a nucleic acid encoding a guide RNA, or a vector comprising the nucleic acid encoding a guide RNA, wherein the guide RNA comprises
a. a sequence selected from SEQ ID NOs: 911, 915, 933, 934, 1-296, 908-910, 912-914, 916-932, and 935-940;
b. at least 15, 16, 17, 18, 19, or 20 contiguous nucleotides of a sequence selected from SEQ ID NOs: 911, 915, 933, 934, 1-296, 908-910, 912-914, 916-932, and 935-940; or c. a sequence that is at least 90%, 91%, 92%, 93%, 94%, 95%, 96%, 97%, 98%, or 99% identical to a sequence selected from SEQ ID NOs: 911, 915, 933, 934, 1-296, 908-910, 912-914, 916-932, and 935-940; and
(ii) an RNA-guided DNA binding agent, a nucleic acid encoding an RNA-guided DNA binding agent, or a vector comprising the nucleic acid encoding an RNA-guided DNA binding agent.

4. The composition of claim 3, wherein the RNA-guided DNA binding agent comprises a Cas nuclease or a Cas nickase.

5. The composition of claim 3, wherein the nucleic acid encoding the RNA-guided DNA binding agent is a Cas9-encoding nucleic acid comprising the polynucleotide sequence set forth in one or more of SEQ ID NOs: 956, 955, 957, 958, 954 959, 960, 902, 903, 941-953, 961, 962 and 963-972.

6.-20. (canceled)

21. The composition of claim 3, wherein the guide RNA is at least partially complementary to a target sequence present in the human PCSK9 gene, wherein the target sequence is in exon 1, 2, 3, 4, 5, 6, 7, 8, 9, 10, 11, or 12 of the human PCSK9 gene.

22.-24. (canceled)

25. The composition of claim 21, wherein the guide RNA sequence comprises a first guide sequence, wherein the first guide sequence is complementary to a first target sequence in the positive strand of the PCSK9 gene, and wherein the composition further comprises a second guide sequence that is complementary to a second target sequence in the negative strand of the PCSK9 gene.

26. The composition of claim 3, wherein the guide RNA comprises a crRNA and further comprises a tracrRNA or a portion thereof, wherein the tracrRNA (trRNA) comprises the nucleotide sequence set forth in SEQ ID NO: 904 wherein the trRNA is operably linked to the crRNA.

27. The composition of claim 3, wherein the guide RNA is a dual guide RNA (dgRNA) or a single guide RNA (sgRNA).

28. (canceled)

29. The composition of claim 3, wherein guide RNA comprises at least one modification.

30.-34. (canceled)

35. The composition of claim 27, wherein the sgRNA comprises a nucleotide sequence that is at least 90% identical to the nucleotide sequence set forth in any one of SEQ ID NOs: 593-888.

36. The composition of claim 3, wherein the guide RNA is associated with a lipid nanoparticle (LNP).

37. The composition of claim 3, wherein the composition is a pharmaceutical formulation and further comprises a pharmaceutically acceptable carrier.

38.-54. (canceled)

55. A method of reducing LDL levels in the blood and/or treating hypercholesterolemia and/or cardiovascular disease in a subject, comprising administering the composition of claim 3 to the subject in need thereof, wherein the composition recognizes and cleaves a PCSK9 target sequence, thereby reducing LDL levels in the blood and/or treating hypercholesterolemia and/or cardiovascular disease in a subject.

56.-57. (canceled)

58. The method of claim 55, wherein the nucleic acid encoding the RNA-guided DNA binding agent is a Cas9-encoding nucleic acid comprising the nucleic acid sequence set forth in one or more of SEQ ID NOs: 956, 955, 957, 958, 954, 959, 960, 902, 903, 941-953, 961, 962 and 963-972.

59.-73. (canceled)

74. The method of claim 55, wherein the composition is administered or delivered at least once.

75.-101. (canceled)

102. The method of claim 55, wherein the subject has familial hypercholesterolemia or exhibits symptoms of atherosclerosis.

103.-106. (canceled)

107. The method of claim 55, wherein the composition or pharmaceutical formulation is administered via a viral vector or via lipid nanoparticles.

108. (canceled)

109. A Cas9-encoding nucleic acid comprising a polynucleotide sequence set forth in any one of SEQ ID NOs: 956, 955, 957 and 958.

110. The Cas9-encoding nucleic acid of claim 109, wherein the nucleic acid further comprises the polynucleotide sequence set forth in one or more of SEQ ID NOs: 961, 962, 972, 971, 941-953, and 963-970.

111. The Cas9-encoding nucleic acid of claim 110, wherein the nucleic acid comprises: from 5′ to 3′, SEQ ID NOs: 961, 956 and 962, or wherein the nucleic acid comprises: from 5′ to 3′, SEQ ID NOs: 961, 956, 962 and 972.

112. The composition of claim 3, wherein the composition comprises:
(i) a nucleic acid encoding a guide RNA, or a vector comprising the nucleic acid encoding a guide RNA, wherein the guide RNA comprises a sequence selected from SEQ ID NOs: 911 or 915; and
(ii) a nucleic acid encoding an RNA-guided DNA binding agent, or a vector comprising the nucleic acid encoding an RNA-guided DNA binding agent, wherein the nucleic acid encoding an RNA-guided DNA binding agent comprises: from 5′ to 3′, SEQ ID NOs: 961, 956 and 962.


SEQUENCE LISTING (974 sequences)

SEQ ID NO: 1 - RNA, 20 nt, synthetic construct
  source 1..20: mol_type = other RNA
  ncRNA 1..20: ncRNA_class = guide_RNA
ccaggttccacgggatgctc

SEQ ID NO: 2 - RNA, 20 nt, synthetic construct
  source 1..20: mol_type = other RNA
  ncRNA 1..20: ncRNA_class = guide_RNA
taatccgctccaggttccac

SEQ ID NO: 3 - RNA, 20 nt, synthetic construct
  source 1..20: mol_type = other RNA
  ncRNA 1..20: ncRNA_class = guide_RNA
tgggggtcttaccggggggc

SEQ ID NO: 4 - RNA, 20 nt, synthetic construct
  source 1..20: mol_type = other RNA
  ncRNA 1..20: ncRNA_class = guide_RNA
ttggaaagacggaggcagcc

SEQ ID NO: 5 - RNA, 20 nt, synthetic construct
  source 1..20: mol_type = other RNA
  ncRNA 1..20: ncRNA_class = guide_RNA
cagcatacagagtgaccacc

SEQ ID NO: 6 - RNA, 20 nt, synthetic construct
  source 1..20: mol_type = other RNA
  ncRNA 1..20: ncRNA_class = guide_RNA
ccggtggtcactctgtatgc

SEQ ID NO: 7 - RNA, 20 nt, synthetic construct
  source 1..20: mol_type = other RNA
  ncRNA 1..20: ncRNA_class = guide_RNA
agaatgtgcccgaggaggac

SEQ ID NO: 8 - RNA, 20 nt, synthetic construct
  source 1..20: mol_type = other RNA
  ncRNA 1..20: ncRNA_class = guide_RNA
agtcatggcacccacctggc

SEQ ID NO: 9 - RNA, 20 nt, synthetic construct
  source 1..20: mol_type = other RNA
  ncRNA 1..20: ncRNA_class = guide_RNA
gtcatggcacccacctggca

SEQ ID NO: 10 - RNA, 20 nt, synthetic construct
  source 1..20: mol_type = other RNA
  ncRNA 1..20: ncRNA_class = guide_RNA
ccgggatgccggcgtggcca

SEQ ID NO: 11 - RNA, 20 nt, synthetic construct
  source 1..20: mol_type = other RNA
  ncRNA 1..20: ncRNA_class = guide_RNA
cgggatgccggcgtggccaa

SEQ ID NO: 12 - RNA, 20 nt, synthetic construct
  source 1..20: mol_type = other RNA
  ncRNA 1..20: ncRNA_class = guide_RNA
ccttggccacgccggcatcc

SEQ ID NO: 13 - RNA, 20 nt, synthetic construct
  source 1..20: mol_type = other RNA
  ncRNA 1..20: ncRNA_class = guide_RNA
gctggcacccttggccacgc

SEQ ID NO: 14 - RNA, 20 nt, synthetic construct
  source 1..20: mol_type = other RNA
  ncRNA 1..20: ncRNA_class = guide_RNA
tcccaggcctggagtttatt

SEQ ID NO: 15 - RNA, 20 nt, synthetic construct
  source 1..20: mol_type = other RNA
  ncRNA 1..20: ncRNA_class = guide_RNA
ttccgaataaactccaggcc

SEQ ID NO: 16 - RNA, 20 nt, synthetic construct
  source 1..20: mol_type = other RNA
  ncRNA 1..20: ncRNA_class = guide_RNA
gcggctgtacccacccgcca

SEQ ID NO: 17 - RNA, 20 nt, synthetic construct
  source 1..20: mol_type = other RNA
  ncRNA 1..20: ncRNA_class = guide_RNA
cgcggctgtacccacccgcc

SEQ ID NO: 18 - RNA, 20 nt, synthetic construct
  source 1..20: mol_type = other RNA
  ncRNA 1..20: ncRNA_class = guide_RNA
ggcaggcggcgttgaggacg

SEQ ID NO: 19 - RNA, 20 nt, synthetic construct
  source 1..20: mol_type = other RNA
  ncRNA 1..20: ncRNA_class = guide_RNA
caacgccgcctgccagcgcc

SEQ ID NO: 20 - RNA, 20 nt, synthetic construct
  source 1..20: mol_type = other RNA
  ncRNA 1..20: ncRNA_class = guide_RNA
ggcgctggcaggcggcgttg

SEQ ID NO: 21 - RNA, 20 nt, synthetic construct
  source 1..20: mol_type = other RNA
  ncRNA 1..20: ncRNA_class = guide_RNA
ccgcctgccagcgcctggcg

SEQ ID NO: 22 - RNA, 20 nt, synthetic construct
  source 1..20: mol_type = other RNA
  ncRNA 1..20: ncRNA_class = guide_RNA
cgcctgccagcgcctggcga

SEQ ID NO: 23 - RNA, 20 nt, synthetic construct
  source 1..20: mol_type = other RNA
  ncRNA 1..20: ncRNA_class = guide_RNA
agccctcgccaggcgctggc

SEQ ID NO: 24 - RNA, 20 nt, synthetic construct
  source 1..20: mol_type = other RNA
  ncRNA 1..20: ncRNA_class = guide_RNA
gcacgaccccagccctcgcc

SEQ ID NO: 25 - RNA, 20 nt, synthetic construct
  source 1..20: mol_type = other RNA
  ncRNA 1..20: ncRNA_class = guide_RNA
cctttccaggtcatcacagt

SEQ ID NO: 26 - RNA, 20 nt, synthetic construct
  source 1..20: mol_type = other RNA
  ncRNA 1..20: ncRNA_class = guide_RNA
gccggtgaccctggggactt

SEQ ID NO: 27 - RNA, 20 nt, synthetic construct
  source 1..20: mol_type = other RNA
  ncRNA 1..20: ncRNA_class = guide_RNA
aagtccccagggtcaccggc

SEQ ID NO: 28 - RNA, 20 nt, synthetic construct
  source 1..20: mol_type = other RNA
  ncRNA 1..20: ncRNA_class = guide_RNA
ccggtgaccctggggacttt

SEQ ID NO: 29 - RNA, 20 nt, synthetic construct
  source 1..20: mol_type = other RNA
  ncRNA 1..20: ncRNA_class = guide_RNA
gttggtccccaaagtcccca

SEQ ID NO: 30 - RNA, 20 nt, synthetic construct
  source 1..20: mol_type = other RNA
  ncRNA 1..20: ncRNA_class = guide_RNA
agttggtccccaaagtcccc

SEQ ID NO: 31 - RNA, 20 nt, synthetic construct
  source 1..20: mol_type = other RNA
  ncRNA 1..20: ncRNA_class = guide_RNA
gggactttggggaccaactt

SEQ ID NO: 32 - RNA, 20 nt, synthetic construct
  source 1..20: mol_type = other RNA
  ncRNA 1..20: ncRNA_class = guide_RNA
tgtgtggacctctttgcccc

SEQ ID NO: 33 - RNA, 20 nt, synthetic construct
  source 1..20: mol_type = other RNA
  ncRNA 1..20: ncRNA_class = guide_RNA
tgtggacctctttgccccag

SEQ ID NO: 34 - RNA, 20 nt, synthetic construct
  source 1..20: mol_type = other RNA
  ncRNA 1..20: ncRNA_class = guide_RNA
ggacctctttgccccagggg

SEQ ID NO: 35 - RNA, 20 nt, synthetic construct
  source 1..20: mol_type = other RNA
  ncRNA 1..20: ncRNA_class = guide_RNA
gcaccaatgatgtcctcccc

SEQ ID NO: 36 - RNA, 20 nt, synthetic construct
  source 1..20: mol_type = other RNA
  ncRNA 1..20: ncRNA_class = guide_RNA
aaagcaggtgctgcagtcgc

SEQ ID NO: 37 - RNA, 20 nt, synthetic construct
  source 1..20: mol_type = other RNA
  ncRNA 1..20: ncRNA_class = guide_RNA
atcacaggctgctgcccacg

SEQ ID NO: 38 - RNA, 20 nt, synthetic construct
  source 1..20: mol_type = other RNA
  ncRNA 1..20: ncRNA_class = guide_RNA
agcatcatggctgcaatgcc

SEQ ID NO: 39 - RNA, 20 nt, synthetic construct
  source 1..20: mol_type = other RNA
  ncRNA 1..20: ncRNA_class = guide_RNA
catgatgctgtctgccgagc

SEQ ID NO: 40 - RNA, 20 nt, synthetic construct
  source 1..20: mol_type = other RNA
  ncRNA 1..20: ncRNA_class = guide_RNA
cggctcggcagacagcatca

SEQ ID NO: 41 - RNA, 20 nt, synthetic construct
  source 1..20: mol_type = other RNA
  ncRNA 1..20: ncRNA_class = guide_RNA
agctcaccctggccgagttg

SEQ ID NO: 42 - RNA, 20 nt, synthetic construct
  source 1..20: mol_type = other RNA
  ncRNA 1..20: ncRNA_class = guide_RNA
tctctgcctcaactcggcca

SEQ ID NO: 43 - RNA, 20 nt, synthetic construct
  source 1..20: mol_type = other RNA
  ncRNA 1..20: ncRNA_class = guide_RNA
ggcctcattgatgacatctt

SEQ ID NO: 44 - RNA, 20 nt, synthetic construct
  source 1..20: mol_type = other RNA
  ncRNA 1..20: ncRNA_class = guide_RNA
gtcagtacccgctggtcctc

SEQ ID NO: 45 - RNA, 20 nt, synthetic construct
  source 1..20: mol_type = other RNA
  ncRNA 1..20: ncRNA_class = guide_RNA
tacctgccccatgggtgctg

SEQ ID NO: 46 - RNA, 20 nt, synthetic construct
  source 1..20: mol_type = other RNA
  ncRNA 1..20: ncRNA_class = guide_RNA
cttacctgccccatgggtgc

SEQ ID NO: 47 - RNA, 20 nt, synthetic construct
  source 1..20: mol_type = other RNA
  ncRNA 1..20: ncRNA_class = guide_RNA
atcctgcttacctgccccat

SEQ ID NO: 48 - RNA, 20 nt, synthetic construct
  source 1..20: mol_type = other RNA
  ncRNA 1..20: ncRNA_class = guide_RNA
cccaggccctttttgcaggt

SEQ ID NO: 49 - RNA, 20 nt, synthetic construct
  source 1..20: mol_type = other RNA
  ncRNA 1..20: ncRNA_class = guide_RNA
caggttggcagctgttttgc

SEQ ID NO: 50 - RNA, 20 nt, synthetic construct
  source 1..20: mol_type = other RNA
  ncRNA 1..20: ncRNA_class = guide_RNA
cgcccgctgcgccccagatg

SEQ ID NO: 51 - RNA, 20 nt, synthetic construct
  source 1..20: mol_type = other RNA
  ncRNA 1..20: ncRNA_class = guide_RNA
ctcctcatctggggcgcagc

SEQ ID NO: 52 - RNA, 20 nt, synthetic construct
  source 1..20: mol_type = other RNA
  ncRNA 1..20: ncRNA_class = guide_RNA
tccaggagtgggaagcggcg

SEQ ID NO: 53 - RNA, 20 nt, synthetic construct
  source 1..20: mol_type = other RNA
  ncRNA 1..20: ncRNA_class = guide_RNA
gaagcggcggggcgagcgca

SEQ ID NO: 54 - RNA, 20 nt, synthetic construct
  source 1..20: mol_type = other RNA
  ncRNA 1..20: ncRNA_class = guide_RNA
gcggcggggcgagcgcatgg

SEQ ID NO: 55 - RNA, 20 nt, synthetic construct
  source 1..20: mol_type = other RNA
  ncRNA 1..20: ncRNA_class = guide_RNA
gtctttgactctaaggccca

SEQ ID NO: 56 - RNA, 20 nt, synthetic construct
  source 1..20: mol_type = other RNA
  ncRNA 1..20: ncRNA_class = guide_RNA
tctttgactctaaggcccaa

SEQ ID NO: 57 - RNA, 20 nt, synthetic construct
  source 1..20: mol_type = other RNA
  ncRNA 1..20: ncRNA_class = guide_RNA
tttgactctaaggcccaagg

SEQ ID NO: 58 - RNA, 20 nt, synthetic construct
  source 1..20: mol_type = other RNA
  ncRNA 1..20: ncRNA_class = guide_RNA
taaggcccaagggggcaagc

SEQ ID NO: 59 - RNA, 20 nt, synthetic construct
  source 1..20: mol_type = other RNA
  ncRNA 1..20: ncRNA_class = guide_RNA
aagggggcaagctggtctgc

SEQ ID NO: 60 - RNA, 20 nt, synthetic construct
  source 1..20: mol_type = other RNA
  ncRNA 1..20: ncRNA_class = guide_RNA
ggcagaccagcttgccccct

SEQ ID NO: 61 - RNA, 20 nt, synthetic construct
  source 1..20: mol_type = other RNA
  ncRNA 1..20: ncRNA_class = guide_RNA
agggggcaagctggtctgcc

SEQ ID NO: 62 - RNA, 20 nt, synthetic construct
  source 1..20: mol_type = other RNA
  ncRNA 1..20: ncRNA_class = guide_RNA
ccccaaaagcgttgtgggcc

SEQ ID NO: 63 - RNA, 20 nt, synthetic construct
  source 1..20: mol_type = other RNA
  ncRNA 1..20: ncRNA_class = guide_RNA
cacaacgcttttgggggtga

SEQ ID NO: 64 - RNA, 20 nt, synthetic construct
  source 1..20: mol_type = other RNA
  ncRNA 1..20: ncRNA_class = guide_RNA
cctcacccccaaaagcgttg

SEQ ID NO: 65 - RNA, 20 nt, synthetic construct
  source 1..20: mol_type = other RNA
  ncRNA 1..20: ncRNA_class = guide_RNA
aacctctcccctggccctca

SEQ ID NO: 66 - RNA, 20 nt, synthetic construct
  source 1..20: mol_type = other RNA
  ncRNA 1..20: ncRNA_class = guide_RNA
acctctcccctggccctcat

SEQ ID NO: 67 - RNA, 20 nt, synthetic construct
  source 1..20: mol_type = other RNA
  ncRNA 1..20: ncRNA_class = guide_RNA
acggtgcccatgagggccag

SEQ ID NO: 68 - RNA, 20 nt, synthetic construct
  source 1..20: mol_type = other RNA
  ncRNA 1..20: ncRNA_class = guide_RNA
gacggtgcccatgagggcca

SEQ ID NO: 69 - RNA, 20 nt, synthetic construct
  source 1..20: mol_type = other RNA
  ncRNA 1..20: ncRNA_class = guide_RNA
tgacggtgcccatgagggcc

SEQ ID NO: 70 - RNA, 20 nt, synthetic construct
  source 1..20: mol_type = other RNA
  ncRNA 1..20: ncRNA_class = guide_RNA
tcatgggcaccgtcagctcc

SEQ ID NO: 71 - RNA, 20 nt, synthetic construct
  source 1..20: mol_type = other RNA
  ncRNA 1..20: ncRNA_class = guide_RNA
ggagctgacggtgcccatga

SEQ ID NO: 72 - RNA, 20 nt, synthetic construct
  source 1..20: mol_type = other RNA
  ncRNA 1..20: ncRNA_class = guide_RNA
tggagctgacggtgcccatg

SEQ ID NO: 73 - RNA, 20 nt, synthetic construct
  source 1..20: mol_type = other RNA
  ncRNA 1..20: ncRNA_class = guide_RNA
tgggcaccgtcagctccagg

SEQ ID NO: 74 - RNA, 20 nt, synthetic construct
  source 1..20: mol_type = other RNA
  ncRNA 1..20: ncRNA_class = guide_RNA
ccgtcagctccaggcggtcc

SEQ ID NO: 75 - RNA, 20 nt, synthetic construct
  source 1..20: mol_type = other RNA
  ncRNA 1..20: ncRNA_class = guide_RNA
ccaggaccgcctggagctga

SEQ ID NO: 76 - RNA, 20 nt, synthetic construct
  source 1..20: mol_type = other RNA
  ncRNA 1..20: ncRNA_class = guide_RNA
tcagctccaggcggtcctgg

SEQ ID NO: 77 - RNA, 20 nt, synthetic construct
  source 1..20: mol_type = other RNA
  ncRNA 1..20: ncRNA_class = guide_RNA
cagcggccaccaggaccgcc

SEQ ID NO: 78 - RNA, 20 nt, synthetic construct
  source 1..20: mol_type = other RNA
  ncRNA 1..20: ncRNA_class = guide_RNA
cagcagtggcagcggccacc

SEQ ID NO: 79 - RNA, 20 nt, synthetic construct
  source 1..20: mol_type = other RNA
  ncRNA 1..20: ncRNA_class = guide_RNA
gctgctgctcctgggtcccg

SEQ ID NO: 80 - RNA, 20 nt, synthetic construct
  source 1..20: mol_type = other RNA
  ncRNA 1..20: ncRNA_class = guide_RNA
ctgctgctcctgggtcccgc

SEQ ID NO: 81 - RNA, 20 nt, synthetic construct
  source 1..20: mol_type = other RNA
  ncRNA 1..20: ncRNA_class = guide_RNA
cacgggcgcccgcgggaccc

SEQ ID NO: 82 - RNA, 20 nt, synthetic construct
  source 1..20: mol_type = other RNA
  ncRNA 1..20: ncRNA_class = guide_RNA
tcccgcgggcgcccgtgcgc

SEQ ID NO: 83 - RNA, 20 nt, synthetic construct
  source 1..20: mol_type = other RNA
  ncRNA 1..20: ncRNA_class = guide_RNA
cgcgggcgcccgtgcgcagg

SEQ ID NO: 84 - RNA, 20 nt, synthetic construct
  source 1..20: mol_type = other RNA
  ncRNA 1..20: ncRNA_class = guide_RNA
tcctgcgcacgggcgcccgc

SEQ ID NO: 85 - RNA, 20 nt, synthetic construct
  source 1..20: mol_type = other RNA
  ncRNA 1..20: ncRNA_class = guide_RNA
ctcctgcgcacgggcgcccg

SEQ ID NO: 86 - RNA, 20 nt, synthetic construct
  source 1..20: mol_type = other RNA
  ncRNA 1..20: ncRNA_class = guide_RNA
cgcccgtgcgcaggaggacg

SEQ ID NO: 87 - RNA, 20 nt, synthetic construct
  source 1..20: mol_type = other RNA
  ncRNA 1..20: ncRNA_class = guide_RNA
cgtgcgcaggaggacgagga

SEQ ID NO: 88 - RNA, 20 nt, synthetic construct
  source 1..20: mol_type = other RNA
  ncRNA 1..20: ncRNA_class = guide_RNA
gtcctcgtcctcctgcgcac

SEQ ID NO: 89 - RNA, 20 nt, synthetic construct
  source 1..20: mol_type = other RNA
  ncRNA 1..20: ncRNA_class = guide_RNA
cgtcctcgtcctcctgcgca

SEQ ID NO: 90 - RNA, 20 nt, synthetic construct
  source 1..20: mol_type = other RNA
  ncRNA 1..20: ncRNA_class = guide_RNA
ggacgaggacggcgactacg

SEQ ID NO: 91 - RNA, 20 nt, synthetic construct
  source 1..20: mol_type = other RNA
  ncRNA 1..20: ncRNA_class = guide_RNA
ggacggcgactacgaggagc

SEQ ID NO: 92 - RNA, 20 nt, synthetic construct
  source 1..20: mol_type = other RNA
  ncRNA 1..20: ncRNA_class = guide_RNA
ggtgctagccttgcgttccg

SEQ ID NO: 93 - RNA, 20 nt, synthetic construct
  source 1..20: mol_type = other RNA
  ncRNA 1..20: ncRNA_class = guide_RNA
gctagccttgcgttccgagg

SEQ ID NO: 94 - RNA, 20 nt, synthetic construct
  source 1..20: mol_type = other RNA
  ncRNA 1..20: ncRNA_class = guide_RNA
gccttgcgttccgaggagga

SEQ ID NO: 95 - RNA, 20 nt, synthetic construct
  source 1..20: mol_type = other RNA
  ncRNA 1..20: ncRNA_class = guide_RNA
gccgtcctcctcggaacgca

SEQ ID NO: 96 - RNA, 20 nt, synthetic construct
  source 1..20: mol_type = other RNA
  ncRNA 1..20: ncRNA_class = guide_RNA
gcgttccgaggaggacggcc

SEQ ID NO: 97 - RNA, 20 nt, synthetic construct
  source 1..20: mol_type = other RNA
  ncRNA 1..20: ncRNA_class = guide_RNA
ttcggccaggccgtcctcct

SEQ ID NO: 98 - RNA, 20 nt, synthetic construct
  source 1..20: mol_type = other RNA
  ncRNA 1..20: ncRNA_class = guide_RNA
ctggccgaagcacccgagca

SEQ ID NO: 99 - RNA, 20 nt, synthetic construct
  source 1..20: mol_type = other RNA
  ncRNA 1..20: ncRNA_class = guide_RNA
cgtgctcgggtgcttcggcc

SEQ ID NO: 100 - RNA, 20 nt, synthetic construct
  source 1..20: mol_type = other RNA
  ncRNA 1..20: ncRNA_class = guide_RNA
ggttccgtgctcgggtgctt

SEQ ID NO: 101 - RNA, 20 nt, synthetic construct
  source 1..20: mol_type = other RNA
  ncRNA 1..20: ncRNA_class = guide_RNA
gtggctgtggttccgtgctc

SEQ ID NO: 102 - RNA, 20 nt, synthetic construct
  source 1..20: mol_type = other RNA
  ncRNA 1..20: ncRNA_class = guide_RNA
ggtggctgtggttccgtgct

SEQ ID NO: 103 - RNA, 20 nt, synthetic construct
  source 1..20: mol_type = other RNA
  ncRNA 1..20: ncRNA_class = guide_RNA
caccttccaccgctgcgcca

SEQ ID NO: 104 - RNA, 20 nt, synthetic construct
  source 1..20: mol_type = other RNA
  ncRNA 1..20: ncRNA_class = guide_RNA
cttggcgcagcggtggaagg

SEQ ID NO: 105 - RNA, 20 nt, synthetic construct
  source 1..20: mol_type = other RNA
  ncRNA 1..20: ncRNA_class = guide_RNA
tccaccgctgcgccaaggtg

SEQ ID NO: 106 - RNA, 20 nt, synthetic construct
  source 1..20: mol_type = other RNA
  ncRNA 1..20: ncRNA_class = guide_RNA
caccttggcgcagcggtgga

SEQ ID NO: 107 - RNA, 20 nt, synthetic construct
  source 1..20: mol_type = other RNA
  ncRNA 1..20: ncRNA_class = guide_RNA
ccaccgctgcgccaaggtgc

SEQ ID NO: 108 - RNA, 20 nt, synthetic construct
  source 1..20: mol_type = other RNA
  ncRNA 1..20: ncRNA_class = guide_RNA
cccgcaccttggcgcagcgg

SEQ ID NO: 109 - RNA, 20 nt, synthetic construct
  source 1..20: mol_type = other RNA
  ncRNA 1..20: ncRNA_class = guide_RNA
acacccgcaccttggcgcag

SEQ ID NO: 110 - RNA, 20 nt, synthetic construct
  source 1..20: mol_type = other RNA
  ncRNA 1..20: ncRNA_class = guide_RNA
ctgcgccaaggtgcgggtgt

SEQ ID NO: 111 - RNA, 20 nt, synthetic construct
  source 1..20: mol_type = other RNA
  ncRNA 1..20: ncRNA_class = guide_RNA
tgcgccaaggtgcgggtgta

SEQ ID NO: 112 - RNA, 20 nt, synthetic construct
  source 1..20: mol_type = other RNA
  ncRNA 1..20: ncRNA_class = guide_RNA
ccaaggtgcgggtgtaggga

SEQ ID NO: 113 - RNA, 20 nt, synthetic construct
  source 1..20: mol_type = other RNA
  ncRNA 1..20: ncRNA_class = guide_RNA
caaggtgcgggtgtagggat

SEQ ID NO: 114 - RNA, 20 nt, synthetic construct
  source 1..20: mol_type = other RNA
  ncRNA 1..20: ncRNA_class = guide_RNA
ccatccctacacccgcacct

SEQ ID NO: 115 - RNA, 20 nt, synthetic construct
  source 1..20: mol_type = other RNA
  ncRNA 1..20: ncRNA_class = guide_RNA
gatcctggccccatgcaagg

SEQ ID NO: 116 - RNA, 20 nt, synthetic construct
  source 1..20: mol_type = other RNA
  ncRNA 1..20: ncRNA_class = guide_RNA
ttgcatggggccaggatccg

SEQ ID NO: 117 - RNA, 20 nt, synthetic construct
  source 1..20: mol_type = other RNA
  ncRNA 1..20: ncRNA_class = guide_RNA
acggatcctggccccatgca

SEQ ID NO: 118 - RNA, 20 nt, synthetic construct
  source 1..20: mol_type = other RNA
  ncRNA 1..20: ncRNA_class = guide_RNA
catggggccaggatccgtgg

SEQ ID NO: 119 - RNA, 20 nt, synthetic construct
  source 1..20: mol_type = other RNA
  ncRNA 1..20: ncRNA_class = guide_RNA
caggatccgtggaggttgcc

SEQ ID NO: 120 - RNA, 20 nt, synthetic construct
  source 1..20: mol_type = other RNA
  ncRNA 1..20: ncRNA_class = guide_RNA
caggcaacctccacggatcc

SEQ ID NO: 121 - RNA, 20 nt, synthetic construct
  source 1..20: mol_type = other RNA
  ncRNA 1..20: ncRNA_class = guide_RNA
taggtgccaggcaacctcca

SEQ ID NO: 122 - RNA, 20 nt, synthetic construct
  source 1..20: mol_type = other RNA
  ncRNA 1..20: ncRNA_class = guide_RNA
gaggttgcctggcacctacg

SEQ ID NO: 123 - RNA, 20 nt, synthetic construct
  source 1..20: mol_type = other RNA
  ncRNA 1..20: ncRNA_class = guide_RNA
gttgcctggcacctacgtgg

SEQ ID NO: 124 - RNA, 20 nt, synthetic construct
  source 1..20: mol_type = other RNA
  ncRNA 1..20: ncRNA_class = guide_RNA
agcaccaccacgtaggtgcc

SEQ ID NO: 125 - RNA, 20 nt, synthetic construct
  source 1..20: mol_type = other RNA
  ncRNA 1..20: ncRNA_class = guide_RNA
cacctacgtggtggtgctga

SEQ ID NO: 126 - RNA, 20 nt, synthetic construct
  source 1..20: mol_type = other RNA
  ncRNA 1..20: ncRNA_class = guide_RNA
ctacgtggtggtgctgaagg

SEQ ID NO: 127 - RNA, 20 nt, synthetic construct
  source 1..20: mol_type = other RNA
  ncRNA 1..20: ncRNA_class = guide_RNA
ctccttcagcaccaccacgt

SEQ ID NO: 128 - RNA, 20 nt, synthetic construct
  source 1..20: mol_type = other RNA
  ncRNA 1..20: ncRNA_class = guide_RNA
gcgctctgactgcgagaggt

SEQ ID NO: 129 - RNA, 20 nt, synthetic construct
  source 1..20: mol_type = other RNA
  ncRNA 1..20: ncRNA_class = guide_RNA
tgcgctctgactgcgagagg

SEQ ID NO: 130 - RNA, 20 nt, synthetic construct
  source 1..20: mol_type = other RNA
  ncRNA 1..20: ncRNA_class = guide_RNA
cagtgcgctctgactgcgag

SEQ ID NO: 131 - RNA, 20 nt, synthetic construct
  source 1..20: mol_type = other RNA
  ncRNA 1..20: ncRNA_class = guide_RNA
gcgcactgcccgccgcctgc

SEQ ID NO: 132 - RNA, 20 nt, synthetic construct
  source 1..20: mol_type = other RNA
  ncRNA 1..20: ncRNA_class = guide_RNA
tgcccgccgcctgcaggccc

SEQ ID NO: 133 - RNA, 20 nt, synthetic construct
  source 1..20: mol_type = other RNA
  ncRNA 1..20: ncRNA_class = guide_RNA
tgcaggcccaggctgcccgc

SEQ ID NO: 134 - RNA, 20 nt, synthetic construct
  source 1..20: mol_type = other RNA
  ncRNA 1..20: ncRNA_class = guide_RNA
caggcccaggctgcccgccg

SEQ ID NO: 135 - RNA, 20 nt, synthetic construct
  source 1..20: mol_type = other RNA
  ncRNA 1..20: ncRNA_class = guide_RNA
gtatccccggcgggcagcct

SEQ ID NO: 136 - RNA, 20 nt, synthetic construct
  source 1..20: mol_type = other RNA
  ncRNA 1..20: ncRNA_class = guide_RNA
ggtatccccggcgggcagcc

SEQ ID NO: 137 - RNA, 20 nt, synthetic construct
  source 1..20: mol_type = other RNA
  ncRNA 1..20: ncRNA_class = guide_RNA
cttggtgaggtatccccggc

SEQ ID NO: 138 - RNA, 20 nt, synthetic construct
  source 1..20: mol_type = other RNA
  ncRNA 1..20: ncRNA_class = guide_RNA
tcttggtgaggtatccccgg

SEQ ID NO: 139 - RNA, 20 nt, synthetic construct
  source 1..20: mol_type = other RNA
  ncRNA 1..20: ncRNA_class = guide_RNA
ggatcttggtgaggtatccc

SEQ ID NO: 140 - RNA, 20 nt, synthetic construct
  source 1..20: mol_type = other RNA
  ncRNA 1..20: ncRNA_class = guide_RNA
agacatgcaggatcttggtg

SEQ ID NO: 141 - RNA, 20 nt, synthetic construct
  source 1..20: mol_type = other RNA
  ncRNA 1..20: ncRNA_class = guide_RNA
aagatcctgcatgtcttcca

SEQ ID NO: 142 - RNA, 20 nt, synthetic construct
  source 1..20: mol_type = other RNA
  ncRNA 1..20: ncRNA_class = guide_RNA
atggaagacatgcaggatct

SEQ ID NO: 143 - RNA, 20 nt, synthetic construct
  source 1..20: mol_type = other RNA
  ncRNA 1..20: ncRNA_class = guide_RNA
gaaggccatggaagacatgc

SEQ ID NO: 144 - RNA, 20 nt, synthetic construct
  source 1..20: mol_type = other RNA
  ncRNA 1..20: ncRNA_class = guide_RNA
gtcttccatggccttcttcc

SEQ ID NO: 145 - RNA, 20 nt, synthetic construct
  source 1..20: mol_type = other RNA
  ncRNA 1..20: ncRNA_class = guide_RNA
ctcatcttcaccaggaagcc

SEQ ID NO: 146 - RNA, 20 nt, synthetic construct
  source 1..20: mol_type = other RNA
  ncRNA 1..20: ncRNA_class = guide_RNA
ggcttcctggtgaagatgag

SEQ ID NO: 147 - RNA, 20 nt, synthetic construct
  source 1..20: mol_type = other RNA
  ncRNA 1..20: ncRNA_class = guide_RNA
ggtcgccactcatcttcacc

SEQ ID NO: 148 - RNA, 20 nt, synthetic construct
  source 1..20: mol_type = other RNA
  ncRNA 1..20: ncRNA_class = guide_RNA
gaagatgagtggcgacctgc

SEQ ID NO: 149 - RNA, 20 nt, synthetic construct
  source 1..20: mol_type = other RNA
  ncRNA 1..20: ncRNA_class = guide_RNA
gagtggcgacctgctggagc

SEQ ID NO: 150 - RNA, 20 nt, synthetic construct
  source 1..20: mol_type = other RNA
  ncRNA 1..20: ncRNA_class = guide_RNA
ggtggctcaccagctccagc

SEQ ID NO: 151 - RNA, 20 nt, synthetic construct
  source 1..20: mol_type = other RNA
  ncRNA 1..20: ncRNA_class = guide_RNA
agctggtgagccaccctttt

SEQ ID NO: 152 - RNA, 20 nt, synthetic construct
  source 1..20: mol_type = other RNA
  ncRNA 1..20: ncRNA_class = guide_RNA
gctggtgagccacccttttt

SEQ ID NO: 153 - RNA, 20 nt, synthetic construct
  source 1..20: mol_type = other RNA
  ncRNA 1..20: ncRNA_class = guide_RNA
aaggcctgcagaagccagag

SEQ ID NO: 154 - RNA, 20 nt, synthetic construct
  source 1..20: mol_type = other RNA
  ncRNA 1..20: ncRNA_class = guide_RNA
gtcgacatggggcaacttca

SEQ ID NO: 155 - RNA, 20 nt, synthetic construct
  source 1..20: mol_type = other RNA
  ncRNA 1..20: ncRNA_class = guide_RNA
gccccatgtcgactacatcg

SEQ ID NO: 156 - RNA, 20 nt, synthetic construct
  source 1..20: mol_type = other RNA
  ncRNA 1..20: ncRNA_class = guide_RNA
ccatgtcgactacatcgagg

SEQ ID NO: 157 - RNA, 20 nt, synthetic construct
  source 1..20: mol_type = other RNA
  ncRNA 1..20: ncRNA_class = guide_RNA
tcctcgatgtagtcgacatg

SEQ ID NO: 158 - RNA, 20 nt, synthetic construct
  source 1..20: mol_type = other RNA
  ncRNA 1..20: ncRNA_class = guide_RNA
ctcctcgatgtagtcgacat

SEQ ID NO: 159 - RNA, 20 nt, synthetic construct
  source 1..20: mol_type = other RNA
  ncRNA 1..20: ncRNA_class = guide_RNA
cctcctcgatgtagtcgaca

SEQ ID NO: 160 - RNA, 20 nt, synthetic construct
  source 1..20: mol_type = other RNA
  ncRNA 1..20: ncRNA_class = guide_RNA
gatgctctgggcaaagacag

SEQ ID NO: 161 - RNA, 20 nt, synthetic construct
  source 1..20: mol_type = other RNA
  ncRNA 1..20: ncRNA_class = guide_RNA
tctttgcccagagcatcccg

SEQ ID NO: 162 - RNA, 20 nt, synthetic construct
  source 1..20: mol_type = other RNA
  ncRNA 1..20: ncRNA_class = guide_RNA
ccagagcatcccgtggaacc

SEQ ID NO: 163 - RNA, 20 nt, synthetic construct
  source 1..20: mol_type = other RNA
  ncRNA 1..20: ncRNA_class = guide_RNA
caggttccacgggatgctct

SEQ ID NO: 164 - RNA, 20 nt, synthetic construct
  source 1..20: mol_type = other RNA
  ncRNA 1..20: ncRNA_class = guide_RNA
gcatcccgtggaacctggag

SEQ ID NO: 165 - RNA, 20 nt, synthetic construct
  source 1..20: mol_type = other RNA
  ncRNA 1..20: ncRNA_class = guide_RNA
gtaatccgctccaggttcca

SEQ ID NO: 166 - RNA, 20 nt, synthetic construct
  source 1..20: mol_type = other RNA
  ncRNA 1..20: ncRNA_class = guide_RNA
tggagcggattacccctcca

SEQ ID NO: 167 - RNA, 20 nt, synthetic construct
  source 1..20: mol_type = other RNA
  ncRNA 1..20: ncRNA_class = guide_RNA
gtggaggggtaatccgctcc

SEQ ID NO: 168 - RNA, 20 nt, synthetic construct
  source 1..20: mol_type = other RNA
  ncRNA 1..20: ncRNA_class = guide_RNA
ggattacccctccacggtac

SEQ ID NO: 169 - RNA, 20 nt, synthetic construct
  source 1..20: mol_type = other RNA
  ncRNA 1..20: ncRNA_class = guide_RNA
gattacccctccacggtacc

SEQ ID NO: 170 - RNA, 20 nt, synthetic construct
  source 1..20: mol_type = other RNA
  ncRNA 1..20: ncRNA_class = guide_RNA
tacccctccacggtaccggg

SEQ ID NO: 171 - RNA, 20 nt, synthetic construct
  source 1..20: mol_type = other RNA
  ncRNA 1..20: ncRNA_class = guide_RNA
atccgcccggtaccgtggag

SEQ ID NO: 172 - RNA, 20 nt, synthetic construct
  source 1..20: mol_type = other RNA
  ncRNA 1..20: ncRNA_class = guide_RNA
catccgcccggtaccgtgga

SEQ ID NO: 173 - RNA, 20 nt, synthetic construct
  source 1..20: mol_type = other RNA
  ncRNA 1..20: ncRNA_class = guide_RNA
  ncRNA 1..20: ncRNA_class = guide_RNA
tcatccgcccggtaccgtgg

SEQ ID NO: 174 - RNA, 20 nt, synthetic construct
  source 1..20: mol_type = other RNA
  ncRNA 1..20: ncRNA_class = guide_RNA
tattcatccgcccggtaccg

SEQ ID NO: 175 - RNA, 20 nt, synthetic construct
  source 1..20: mol_type = other RNA
  ncRNA 1..20: ncRNA_class = guide_RNA
ggggctggtattcatccgcc

SEQ ID NO: 176 - RNA, 20 nt, synthetic construct
  source 1..20: mol_type = other RNA
  ncRNA 1..20: ncRNA_class = guide_RNA
gcggatgaataccagccccc

SEQ ID NO: 177 - RNA, 20 nt, synthetic construct
  source 1..20: mol_type = other RNA
  ncRNA 1..20: ncRNA_class = guide_RNA
cagatgggggtcttaccggg

SEQ ID NO: 178 - RNA, 20 nt, synthetic construct
  source 1..20: mol_type = other RNA
  ncRNA 1..20: ncRNA_class = guide_RNA
acagatgggggtcttaccgg

SEQ ID NO: 179 - RNA, 20 nt, synthetic construct
  source 1..20: mol_type = other RNA
  ncRNA 1..20: ncRNA_class = guide_RNA
ctttccaaggcgacatttgt

SEQ ID NO: 180 - RNA, 20 nt, synthetic construct
  source 1..20: mol_type = other RNA
  ncRNA 1..20: ncRNA_class = guide_RNA
tctttccaaggcgacatttg

SEQ ID NO: 181 - RNA, 20 nt, synthetic construct
  source 1..20: mol_type = other RNA
  ncRNA 1..20: ncRNA_class = guide_RNA
caaatgtcgccttggaaaga

SEQ ID NO: 182 - RNA, 20 nt, synthetic construct
  source 1..20: mol_type = other RNA
  ncRNA 1..20: ncRNA_class = guide_RNA
atgtcgccttggaaagacgg

SEQ ID NO: 183 - RNA, 20 nt, synthetic construct
  source 1..20: mol_type = other RNA
  ncRNA 1..20: ncRNA_class = guide_RNA
gaaagacggaggcagcctgg

SEQ ID NO: 184 - RNA, 20 nt, synthetic construct
  source 1..20: mol_type = other RNA
  ncRNA 1..20: ncRNA_class = guide_RNA
ctaggagatacacctccacc

SEQ ID NO: 185 - RNA, 20 nt, synthetic construct
  source 1..20: mol_type = other RNA
  ncRNA 1..20: ncRNA_class = guide_RNA
cactctgtatgctggtgtct

SEQ ID NO: 186 - RNA, 20 nt, synthetic construct
  source 1..20: mol_type = other RNA
  ncRNA 1..20: ncRNA_class = guide_RNA
ccagcatacagagtgaccac

SEQ ID NO: 187 - RNA, 20 nt, synthetic construct
  source 1..20: mol_type = other RNA
  ncRNA 1..20: ncRNA_class = guide_RNA
gagtgaccaccgggaaatcg

SEQ ID NO: 188 - RNA, 20 nt, synthetic construct
  source 1..20: mol_type = other RNA
  ncRNA 1..20: ncRNA_class = guide_RNA
agtgaccaccgggaaatcga

SEQ ID NO: 189 - RNA, 20 nt, synthetic construct
  source 1..20: mol_type = other RNA
  ncRNA 1..20: ncRNA_class = guide_RNA
accaccgggaaatcgagggc

SEQ ID NO: 190 - RNA, 20 nt, synthetic construct
  source 1..20: mol_type = other RNA
  ncRNA 1..20: ncRNA_class = guide_RNA
ccaccgggaaatcgagggca

SEQ ID NO: 191 - RNA, 20 nt, synthetic construct
  source 1..20: mol_type = other RNA
  ncRNA 1..20: ncRNA_class = guide_RNA
ccctgccctcgatttcccgg

SEQ ID NO: 192 - RNA, 20 nt, synthetic construct
  source 1..20: mol_type = other RNA
  ncRNA 1..20: ncRNA_class = guide_RNA
tgaccctgccctcgatttcc

SEQ ID NO: 193 - RNA, 20 nt, synthetic construct
  source 1..20: mol_type = other RNA
  ncRNA 1..20: ncRNA_class = guide_RNA
cgacttcgagaatgtgcccg

SEQ ID NO: 194 - RNA, 20 nt, synthetic construct
  source 1..20: mol_type = other RNA
  ncRNA 1..20: ncRNA_class = guide_RNA
ctcgggcacattctcgaagt

SEQ ID NO: 195 - RNA, 20 nt, synthetic construct
  source 1..20: mol_type = other RNA
  ncRNA 1..20: ncRNA_class = guide_RNA
cttcgagaatgtgcccgagg

SEQ ID NO: 196 - RNA, 20 nt, synthetic construct
  source 1..20: mol_type = other RNA
  ncRNA 1..20: ncRNA_class = guide_RNA
aagcgggtcccgtcctcctc

SEQ ID NO: 197 - RNA, 20 nt, synthetic construct
  source 1..20: mol_type = other RNA
  ncRNA 1..20: ncRNA_class = guide_RNA
gaagcgggtcccgtcctcct

SEQ ID NO: 198 - RNA, 20 nt, synthetic construct
  source 1..20: mol_type = other RNA
  ncRNA 1..20: ncRNA_class = guide_RNA
cgggacccgcttccacagac

SEQ ID NO: 199 - RNA, 20 nt, synthetic construct
  source 1..20: mol_type = other RNA
  ncRNA 1..20: ncRNA_class = guide_RNA
gcttacctgtctgtggaagc

SEQ ID NO: 200 - RNA, 20 nt, synthetic construct
  source 1..20: mol_type = other RNA
  ncRNA 1..20: ncRNA_class = guide_RNA
cggccgtgcttacctgtctg

SEQ ID NO: 201 - RNA, 20 nt, synthetic construct
  source 1..20: mol_type = other RNA
  ncRNA 1..20: ncRNA_class = guide_RNA
aggtaagcacggccgtctga

SEQ ID NO: 202 - RNA, 20 nt, synthetic construct
  source 1..20: mol_type = other RNA
  ncRNA 1..20: ncRNA_class = guide_RNA
ggtaagcacggccgtctgat

SEQ ID NO: 203 - RNA, 20 nt, synthetic construct
  source 1..20: mol_type = other RNA
  ncRNA 1..20: ncRNA_class = guide_RNA
ggtccttgtgttcgtcgagc

SEQ ID NO: 204 - RNA, 20 nt, synthetic construct
  source 1..20: mol_type = other RNA
  ncRNA 1..20: ncRNA_class = guide_RNA
tggcctgctcgacgaacaca

SEQ ID NO: 205 - RNA, 20 nt, synthetic construct
  source 1..20: mol_type = other RNA
  ncRNA 1..20: ncRNA_class = guide_RNA
gccagcaagtgtgacagtca

SEQ ID NO: 206 - RNA, 20 nt, synthetic construct
  source 1..20: mol_type = other RNA
  ncRNA 1..20: ncRNA_class = guide_RNA
gccatgactgtcacacttgc

SEQ ID NO: 207 - RNA, 20 nt, synthetic construct
  source 1..20: mol_type = other RNA
  ncRNA 1..20: ncRNA_class = guide_RNA
gctgcgcatgctggcaccct

SEQ ID NO: 208 - RNA, 20 nt, synthetic construct
  source 1..20: mol_type = other RNA
  ncRNA 1..20: ncRNA_class = guide_RNA
cacgcgcaggctgcgcatgc

SEQ ID NO: 209 - RNA, 20 nt, synthetic construct
  source 1..20: mol_type = other RNA
  ncRNA 1..20: ncRNA_class = guide_RNA
ctgcgcgtgctcaactgcca

SEQ ID NO: 210 - RNA, 20 nt, synthetic construct
  source 1..20: mol_type = other RNA
  ncRNA 1..20: ncRNA_class = guide_RNA
tgcgcgtgctcaactgccaa

SEQ ID NO: 211 - RNA, 20 nt, synthetic construct
  source 1..20: mol_type = other RNA
  ncRNA 1..20: ncRNA_class = guide_RNA
cttggcagttgagcacgcgc

SEQ ID NO: 212 - RNA, 20 nt, synthetic construct
  source 1..20: mol_type = other RNA
  ncRNA 1..20: ncRNA_class = guide_RNA
cgtgctcaactgccaaggga

SEQ ID NO: 213 - RNA, 20 nt, synthetic construct
  source 1..20: mol_type = other RNA
  ncRNA 1..20: ncRNA_class = guide_RNA
gtgctcaactgccaagggaa

SEQ ID NO: 214 - RNA, 20 nt, synthetic construct
  source 1..20: mol_type = other RNA
  ncRNA 1..20: ncRNA_class = guide_RNA
caagggaagggcacggttag

SEQ ID NO: 215 - RNA, 20 nt, synthetic construct
  source 1..20: mol_type = other RNA
  ncRNA 1..20: ncRNA_class = guide_RNA
cgctaaccgtgcccttccct

SEQ ID NO: 216 - RNA, 20 nt, synthetic construct
  source 1..20: mol_type = other RNA
  ncRNA 1..20: ncRNA_class = guide_RNA
acggttagcggcaccctcat

SEQ ID NO: 217 - RNA, 20 nt, synthetic construct
  source 1..20: mol_type = other RNA
  ncRNA 1..20: ncRNA_class = guide_RNA
gggccatcacttacctatga

SEQ ID NO: 218 - RNA, 20 nt, synthetic construct
  source 1..20: mol_type = other RNA
  ncRNA 1..20: ncRNA_class = guide_RNA
ggggccatcacttacctatg

SEQ ID NO: 219 - RNA, 20 nt, synthetic construct
  source 1..20: mol_type = other RNA
  ncRNA 1..20: ncRNA_class = guide_RNA
tccgaataaactccaggcct

SEQ ID NO: 220 - RNA, 20 nt, synthetic construct
  source 1..20: mol_type = other RNA
  ncRNA 1..20: ncRNA_class = guide_RNA
ggcttttccgaataaactcc

SEQ ID NO: 221 - RNA, 20 nt, synthetic construct
  source 1..20: mol_type = other RNA
  ncRNA 1..20: ncRNA_class = guide_RNA
gtttattcggaaaagccagc

SEQ ID NO: 222 - RNA, 20 nt, synthetic construct
  source 1..20: mol_type = other RNA
  ncRNA 1..20: ncRNA_class = guide_RNA
gccagctggtccagcctgtg

SEQ ID NO: 223 - RNA, 20 nt, synthetic construct
  source 1..20: mol_type = other RNA
  ncRNA 1..20: ncRNA_class = guide_RNA
agcaccaccagtggccccac

SEQ ID NO: 224 - RNA, 20 nt, synthetic construct
  source 1..20: mol_type = other RNA
  ncRNA 1..20: ncRNA_class = guide_RNA
cggctgtacccacccgccag

SEQ ID NO: 225 - RNA, 20 nt, synthetic construct
  source 1..20: mol_type = other RNA
  ncRNA 1..5: ncRNA_class = guide_RNA
gtcgtgctggtcaccgctgc

SEQ ID NO: 226 - RNA, 20 nt, synthetic construct
  source 1..20: mol_type = other RNA
  ncRNA 1..20: ncRNA_class = guide_RNA
tcaccgctgccggcaacttc

SEQ ID NO: 227 - RNA, 20 nt, synthetic construct
  source 1..20: mol_type = other RNA
  ncRNA 1..20: ncRNA_class = guide_RNA
caccgctgccggcaacttcc

SEQ ID NO: 228 - RNA, 20 nt, synthetic construct
  source 1..20: mol_type = other RNA
  ncRNA 1..20: ncRNA_class = guide_RNA
gtcccggaagttgccggcag

SEQ ID NO: 229 - RNA, 20 nt, synthetic construct
  source 1..20: mol_type = other RNA
  ncRNA 1..20: ncRNA_class = guide_RNA
ggcatcgtcccggaagttgc

SEQ ID NO: 230 - RNA, 20 nt, synthetic construct
  source 1..20: mol_type = other RNA
  ncRNA 1..20: ncRNA_class = guide_RNA
agtagaggcaggcatcgtcc

SEQ ID NO: 231 - RNA, 20 nt, synthetic construct
  source 1..20: mol_type = other RNA
  ncRNA 1..20: ncRNA_class = guide_RNA
ccagcctcagctcccgaggt

SEQ ID NO: 232 - RNA, 20 nt, synthetic construct
  source 1..20: mol_type = other RNA
  ncRNA 1..20: ncRNA_class = guide_RNA
agcacctacctcgggagctg

SEQ ID NO: 233 - RNA, 20 nt, synthetic construct
  source 1..20: mol_type = other RNA
  ncRNA 1..20: ncRNA_class = guide_RNA
ctttccaggtcatcacagtt

SEQ ID NO: 234 - RNA, 20 nt, synthetic construct
  source 1..20: mol_type = other RNA
  ncRNA 1..20: ncRNA_class = guide_RNA
tttccaggtcatcacagttg

SEQ ID NO: 235 - RNA, 20 nt, synthetic construct
  source 1..20: mol_type = other RNA
  ncRNA 1..20: ncRNA_class = guide_RNA
tggccccaactgtgatgacc

SEQ ID NO: 236 - RNA, 20 nt, synthetic construct
  source 1..20: mol_type = other RNA
  ncRNA 1..20: ncRNA_class = guide_RNA
cggctggtcttgggcattgg

SEQ ID NO: 237 - RNA, 20 nt, synthetic construct
  source 1..20: mol_type = other RNA
  ncRNA 1..20: ncRNA_class = guide_RNA
caccggctggtcttgggcat

SEQ ID NO: 238 - RNA, 20 nt, synthetic construct
  source 1..20: mol_type = other RNA
  ncRNA 1..20: ncRNA_class = guide_RNA
caagaccagccggtgaccct

SEQ ID NO: 239 - RNA, 20 nt, synthetic construct
  source 1..20: mol_type = other RNA
  ncRNA 1..20: ncRNA_class = guide_RNA
aagaccagccggtgaccctg

SEQ ID NO: 240 - RNA, 20 nt, synthetic construct
  source 1..20: mol_type = other RNA
  ncRNA 1..20: ncRNA_class = guide_RNA
cagggtcaccggctggtctt

SEQ ID NO: 241 - RNA, 20 nt, synthetic construct
  source 1..20: mol_type = other RNA
  ncRNA 1..20: ncRNA_class = guide_RNA
gaccaactttggccgctgtg

SEQ ID NO: 242 - RNA, 20 nt, synthetic construct
  source 1..20: mol_type = other RNA
  ncRNA 1..20: ncRNA_class = guide_RNA
gtccacacagcggccaaagt

SEQ ID NO: 243 - RNA, 20 nt, synthetic construct
  source 1..20: mol_type = other RNA
  ncRNA 1..20: ncRNA_class = guide_RNA
gggcaaagaggtccacacag

SEQ ID NO: 244 - RNA, 20 nt, synthetic construct
  source 1..20: mol_type = other RNA
  ncRNA 1..20: ncRNA_class = guide_RNA
gtgtggacctctttgcccca

SEQ ID NO: 245 - RNA, 20 nt, synthetic construct
  source 1..20: mol_type = other RNA
  ncRNA 1..20: ncRNA_class = guide_RNA
tgtcctcccctggggcaaag

SEQ ID NO: 246 - RNA, 20 nt, synthetic construct
  source 1..20: mol_type = other RNA
  ncRNA 1..20: ncRNA_class = guide_RNA
acctgctttgtgtcacagag

SEQ ID NO: 247 - RNA, 20 nt, synthetic construct
  source 1..20: mol_type = other RNA
  ncRNA 1..20: ncRNA_class = guide_RNA
cctgctttgtgtcacagagt

SEQ ID NO: 248 - RNA, 20 nt, synthetic construct
  source 1..20: mol_type = other RNA
  ncRNA 1..20: ncRNA_class = guide_RNA
cccactctgtgacacaaagc

SEQ ID NO: 249 - RNA, 20 nt, synthetic construct
  source 1..20: mol_type = other RNA
  ncRNA 1..20: ncRNA_class = guide_RNA
gtcacagagtgggacatcac

SEQ ID NO: 250 - RNA, 20 nt, synthetic construct
  source 1..20: mol_type = other RNA
  ncRNA 1..20: ncRNA_class = guide_RNA
tggtgacttaccagccacgt

SEQ ID NO: 251 - RNA, 20 nt, synthetic construct
  source 1..20: mol_type = other RNA
  ncRNA 1..20: ncRNA_class = guide_RNA
gtggtgacttaccagccacg

SEQ ID NO: 252 - RNA, 20 nt, synthetic construct
  source 1..20: mol_type = other RNA
  ncRNA 1..20: ncRNA_class = guide_RNA
tgccgagccggagctcaccc

SEQ ID NO: 253 - RNA, 20 nt, synthetic construct
  source 1..20: mol_type = other RNA
  ncRNA 1..20: ncRNA_class = guide_RNA
ggccagggtgagctccggct

SEQ ID NO: 254 - RNA, 20 nt, synthetic construct
  source 1..20: mol_type = other RNA
  ncRNA 1..20: ncRNA_class = guide_RNA
aactcggccagggtgagctc

SEQ ID NO: 255 - RNA, 20 nt, synthetic construct
  source 1..20: mol_type = other RNA
  ncRNA 1..20: ncRNA_class = guide_RNA
gtctctgcctcaactcggcc

SEQ ID NO: 256 - RNA, 20 nt, synthetic construct
  source 1..20: mol_type = other RNA
  ncRNA 1..20: ncRNA_class = guide_RNA
gatcagtctctgcctcaact

SEQ ID NO: 257 - RNA, 20 nt, synthetic construct
  source 1..20: mol_type = other RNA
  ncRNA 1..20: ncRNA_class = guide_RNA
tgacatctttggcagagaag

SEQ ID NO: 258 - RNA, 20 nt, synthetic construct
  source 1..20: mol_type = other RNA
  ncRNA 1..20: ncRNA_class = guide_RNA
aagatgtcatcaatgaggcc

SEQ ID NO: 259 - RNA, 20 nt, synthetic construct
  source 1..20: mol_type = other RNA
  ncRNA 1..20: ncRNA_class = guide_RNA
caatgaggcctggttccctg

SEQ ID NO: 260 - RNA, 20 nt, synthetic construct
  source 1..20: mol_type = other RNA
  ncRNA 1..20: ncRNA_class = guide_RNA
tcagtacccgctggtcctca

SEQ ID NO: 261 - RNA, 20 nt, synthetic construct
  source 1..20: mol_type = other RNA
  ncRNA 1..20: ncRNA_class = guide_RNA
gcgggtactgacccccaacc

SEQ ID NO: 262 - RNA, 20 nt, synthetic construct
  source 1..20: mol_type = other RNA
  ncRNA 1..20: ncRNA_class = guide_RNA
ggttgggggtcagtacccgc

SEQ ID NO: 263 - RNA, 20 nt, synthetic construct
  source 1..20: mol_type = other RNA
  ncRNA 1..20: ncRNA_class = guide_RNA
ggtactgacccccaacctgg

SEQ ID NO: 264 - RNA, 20 nt, synthetic construct
  source 1..20: mol_type = other RNA
  ncRNA 1..20: ncRNA_class = guide_RNA
cctgccccccagcacccatg

SEQ ID NO: 265 - RNA, 20 nt, synthetic construct
  source 1..20: mol_type = other RNA
  ncRNA 1..20: ncRNA_class = guide_RNA
ttacctgccccatgggtgct

SEQ ID NO: 266 - RNA, 20 nt, synthetic construct
  source 1..20: mol_type = other RNA
  ncRNA 1..20: ncRNA_class = guide_RNA
gccaacctgcaaaaagggcc

SEQ ID NO: 267 - RNA, 20 nt, synthetic construct
  source 1..20: mol_type = other RNA
  ncRNA 1..20: ncRNA_class = guide_RNA
gactgtatggtcagcacact

SEQ ID NO: 268 - RNA, 20 nt, synthetic construct
  source 1..20: mol_type = other RNA
  ncRNA 1..20: ncRNA_class = guide_RNA
actgtatggtcagcacactc

SEQ ID NO: 269 - RNA, 20 nt, synthetic construct
  source 1..20: mol_type = other RNA
  ncRNA 1..20: ncRNA_class = guide_RNA
ctgtatggtcagcacactcg

SEQ ID NO: 270 - RNA, 20 nt, synthetic construct
  source 1..20: mol_type = other RNA
  ncRNA 1..20: ncRNA_class = guide_RNA
cagcacactcggggcctaca

SEQ ID NO: 271 - RNA, 20 nt, synthetic construct
  source 1..20: mol_type = other RNA
  ncRNA 1..20: ncRNA_class = guide_RNA
acactcggggcctacacgga

SEQ ID NO: 272 - RNA, 20 nt, synthetic construct
  source 1..20: mol_type = other RNA
  ncRNA 1..20: ncRNA_class = guide_RNA
acggctgtggccatccgtgt

SEQ ID NO: 273 - RNA, 20 nt, synthetic construct
  source 1..20: mol_type = other RNA
  ncRNA 1..20: ncRNA_class = guide_RNA
gctcctcatctggggcgcag

SEQ ID NO: 274 - RNA, 20 nt, synthetic construct
  source 1..20: mol_type = other RNA
  ncRNA 1..20: ncRNA_class = guide_RNA
gcagaccagcttgccccctt

SEQ ID NO: 275 - RNA, 20 nt, synthetic construct
  source 1..20: mol_type = other RNA
  ncRNA 1..20: ncRNA_class = guide_RNA
tgccgggcccacaacgcttt

SEQ ID NO: 276 - RNA, 20 nt, synthetic construct
  source 1..20: mol_type = other RNA
  ncRNA 1..20: ncRNA_class = guide_RNA
gccgggcccacaacgctttt

SEQ ID NO: 277 - RNA, 20 nt, synthetic construct
  source 1..20: mol_type = other RNA
  ncRNA 1..20: ncRNA_class = guide_RNA
ccgggcccacaacgcttttg

SEQ ID NO: 278 - RNA, 20 nt, synthetic construct
  source 1..20: mol_type = other RNA
  ncRNA 1..20: ncRNA_class = guide_RNA
cgggcccacaacgcttttgg

SEQ ID NO: 279 - RNA, 20 nt, synthetic construct
  source 1..20: mol_type = other RNA
  ncRNA 1..20: ncRNA_class = guide_RNA
ccacaacgcttttgggggtg

SEQ ID NO: 280 - RNA, 20 nt, synthetic construct
  source 1..20: mol_type = other RNA
  ncRNA 1..20: ncRNA_class = guide_RNA
ctcacccccaaaagcgttgt

SEQ ID NO: 281 - RNA, 20 nt, synthetic construct
  source 1..20: mol_type = other RNA
  ncRNA 1..20: ncRNA_class = guide_RNA
agggtgtctacgccattgcc

SEQ ID NO: 282 - RNA, 20 nt, synthetic construct
  source 1..20: mol_type = other RNA
  ncRNA 1..20: ncRNA_class = guide_RNA
gtggacacgggtccccatgc

SEQ ID NO: 283 - RNA, 20 nt, synthetic construct
  source 1..20: mol_type = other RNA
  ncRNA 1..20: ncRNA_class = guide_RNA
aaggtcctccacctcccagt

SEQ ID NO: 284 - RNA, 20 nt, synthetic construct
  source 1..20: mol_type = other RNA
  ncRNA 1..20: ncRNA_class = guide_RNA
cctcagcacaggcggcttgt

SEQ ID NO: 285 - RNA, 20 nt, synthetic construct
  source 1..20: mol_type = other RNA
  ncRNA 1..20: ncRNA_class = guide_RNA
cactggttgggctgacctcg

SEQ ID NO: 286 - RNA, 20 nt, synthetic construct
  source 1..20: mol_type = other RNA
  ncRNA 1..20: ncRNA_class = guide_RNA
aggtcagcccaaccagtgcg

SEQ ID NO: 287 - RNA, 20 nt, synthetic construct
  source 1..20: mol_type = other RNA
  ncRNA 1..20: ncRNA_class = guide_RNA
ggtcagcccaaccagtgcgt

SEQ ID NO: 288 - RNA, 20 nt, synthetic construct
  source 1..20: mol_type = other RNA
  ncRNA 1..20: ncRNA_class = guide_RNA
caaccagtgcgtgggccaca

SEQ ID NO: 289 - RNA, 20 nt, synthetic construct
  source 1..20: mol_type = other RNA
  ncRNA 1..20: ncRNA_class = guide_RNA
tagacaacacgtgtgtagtc

SEQ ID NO: 290 - RNA, 20 nt, synthetic construct
  source 1..20: mol_type = other RNA
  ncRNA 1..20: ncRNA_class = guide_RNA
gactacacacgtgttgtcta

SEQ ID NO: 291 - RNA, 20 nt, synthetic construct
  source 1..20: mol_type = other RNA
  ncRNA 1..20: ncRNA_class = guide_RNA
cacgtgtgtagtcaggagcc

SEQ ID NO: 292 - RNA, 20 nt, synthetic construct
  source 1..20: mol_type = other RNA
  ncRNA 1..20: ncRNA_class = guide_RNA
agccgggacgtcagcactac

SEQ ID NO: 293 - RNA, 20 nt, synthetic construct
  source 1..20: mol_type = other RNA
  ncRNA 1..20: ncRNA_class = guide_RNA
tgcctgtagtgctgacgtcc

SEQ ID NO: 294 - RNA, 20 nt, synthetic construct
  source 1..20: mol_type = other RNA
  ncRNA 1..20: ncRNA_class = guide_RNA
actacaggcagcaccagcga

SEQ ID NO: 295 - RNA, 20 nt, synthetic construct
  source 1..20: mol_type = other RNA
  ncRNA 1..20: ncRNA_class = guide_RNA
tacaggcagcaccagcgaag

SEQ ID NO: 296 - RNA, 20 nt, synthetic construct
  source 1..20: mol_type = other RNA
  ncRNA 1..20: ncRNA_class = guide_RNA
gcagatggcaacggctgtca

SEQ ID NO: 297 - RNA, 42 nt, synthetic construct
  source 1..42: mol_type = other RNA
ccaggttccacgggatgctcgttttagagctatgctgttttg

SEQ ID NO: 298 - RNA, 42 nt, synthetic construct
  source 1..42: mol_type = other RNA
taatccgctccaggttccacgttttagagctatgctgttttg

SEQ ID NO: 299 - RNA, 42 nt, synthetic construct
  source 1..42: mol_type = other RNA
tgggggtcttaccggggggcgttttagagctatgctgttttg

SEQ ID NO: 300 - RNA, 42 nt, synthetic construct
  source 1..42: mol_type = other RNA
ttggaaagacggaggcagccgttttagagctatgctgttttg

SEQ ID NO: 301 - RNA, 42 nt, synthetic construct
  source 1..42: mol_type = other RNA
cagcatacagagtgaccaccgttttagagctatgctgttttg

SEQ ID NO: 302 - RNA, 42 nt, synthetic construct
  source 1..42: mol_type = other RNA
ccggtggtcactctgtatgcgttttagagctatgctgttttg

SEQ ID NO: 303 - RNA, 42 nt, synthetic construct
  source 1..42: mol_type = other RNA
agaatgtgcccgaggaggacgttttagagctatgctgttttg

SEQ ID NO: 304 - RNA, 42 nt, synthetic construct
  source 1..42: mol_type = other RNA
agtcatggcacccacctggcgttttagagctatgctgttttg

SEQ ID NO: 305 - RNA, 42 nt, synthetic construct
  source 1..42: mol_type = other RNA
gtcatggcacccacctggcagttttagagctatgctgttttg

SEQ ID NO: 306 - RNA, 42 nt, synthetic construct
  source 1..42: mol_type = other RNA
ccgggatgccggcgtggccagttttagagctatgctgttttg

SEQ ID NO: 307 - RNA, 42 nt, synthetic construct
  source 1..42: mol_type = other RNA
cgggatgccggcgtggccaagttttagagctatgctgttttg

SEQ ID NO: 308 - RNA, 42 nt, synthetic construct
  source 1..42: mol_type = other RNA
ccttggccacgccggcatccgttttagagctatgctgttttg

SEQ ID NO: 309 - RNA, 42 nt, synthetic construct
  source 1..42: mol_type = other RNA
gctggcacccttggccacgcgttttagagctatgctgttttg

SEQ ID NO: 310 - RNA, 42 nt, synthetic construct
  source 1..42: mol_type = other RNA
tcccaggcctggagtttattgttttagagctatgctgttttg

SEQ ID NO: 311 - RNA, 42 nt, synthetic construct
  source 1..42: mol_type = other RNA
ttccgaataaactccaggccgttttagagctatgctgttttg

SEQ ID NO: 312 - RNA, 42 nt, synthetic construct
  source 1..42: mol_type = other RNA
gcggctgtacccacccgccagttttagagctatgctgttttg

SEQ ID NO: 313 - RNA, 42 nt, synthetic construct
  source 1..42: mol_type = other RNA
cgcggctgtacccacccgccgttttagagctatgctgttttg

SEQ ID NO: 314 - RNA, 42 nt, synthetic construct
  source 1..42: mol_type = other RNA
ggcaggcggcgttgaggacggttttagagctatgctgttttg

SEQ ID NO: 315 - RNA, 42 nt, synthetic construct
  source 1..42: mol_type = other RNA
caacgccgcctgccagcgccgttttagagctatgctgttttg

SEQ ID NO: 316 - RNA, 42 nt, synthetic construct
  source 1..42: mol_type = other RNA
ggcgctggcaggcggcgttggttttagagctatgctgttttg

SEQ ID NO: 317 - RNA, 42 nt, synthetic construct
  source 1..42: mol_type = other RNA
ccgcctgccagcgcctggcggttttagagctatgctgttttg

SEQ ID NO: 318 - RNA, 42 nt, synthetic construct
  source 1..42: mol_type = other RNA
cgcctgccagcgcctggcgagttttagagctatgctgttttg

SEQ ID NO: 319 - RNA, 42 nt, synthetic construct
  source 1..42: mol_type = other RNA
agccctcgccaggcgctggcgttttagagctatgctgttttg

SEQ ID NO: 320 - RNA, 42 nt, synthetic construct
  source 1..42: mol_type = other RNA
gcacgaccccagccctcgccgttttagagctatgctgttttg

SEQ ID NO: 321 - RNA, 42 nt, synthetic construct
  source 1..42: mol_type = other RNA
cctttccaggtcatcacagtgttttagagctatgctgttttg

SEQ ID NO: 322 - RNA, 42 nt, synthetic construct
  source 1..42: mol_type = other RNA
gccggtgaccctggggacttgttttagagctatgctgttttg

SEQ ID NO: 323 - RNA, 42 nt, synthetic construct
  source 1..42: mol_type = other RNA
aagtccccagggtcaccggcgttttagagctatgctgttttg

SEQ ID NO: 324 - RNA, 42 nt, synthetic construct
  source 1..42: mol_type = other RNA
ccggtgaccctggggactttgttttagagctatgctgttttg

SEQ ID NO: 325 - RNA, 42 nt, synthetic construct
  source 1..42: mol_type = other RNA
gttggtccccaaagtccccagttttagagctatgctgttttg

SEQ ID NO: 326 - RNA, 42 nt, synthetic construct
  source 1..42: mol_type = other RNA
agttggtccccaaagtccccgttttagagctatgctgttttg

SEQ ID NO: 327 - RNA, 42 nt, synthetic construct
  source 1..42: mol_type = other RNA
gggactttggggaccaacttgttttagagctatgctgttttg

SEQ ID NO: 328 - RNA, 42 nt, synthetic construct
  source 1..42: mol_type = other RNA
tgtgtggacctctttgccccgttttagagctatgctgttttg

SEQ ID NO: 329 - RNA, 42 nt, synthetic construct
  source 1..42: mol_type = other RNA
tgtggacctctttgccccaggttttagagctatgctgttttg

SEQ ID NO: 330 - RNA, 42 nt, synthetic construct
  source 1..42: mol_type = other RNA
ggacctctttgccccagggggttttagagctatgctgttttg

SEQ ID NO: 331 - RNA, 42 nt, synthetic construct
  source 1..42: mol_type = other RNA
gcaccaatgatgtcctccccgttttagagctatgctgttttg

SEQ ID NO: 332 - RNA, 42 nt, synthetic construct
  source 1..42: mol_type = other RNA
aaagcaggtgctgcagtcgcgttttagagctatgctgttttg

SEQ ID NO: 333 - RNA, 42 nt, synthetic construct
  source 1..42: mol_type = other RNA
atcacaggctgctgcccacggttttagagctatgctgttttg

SEQ ID NO: 334 - RNA, 42 nt, synthetic construct
  source 1..42: mol_type = other RNA
agcatcatggctgcaatgccgttttagagctatgctgttttg

SEQ ID NO: 335 - RNA, 42 nt, synthetic construct
  source 1..42: mol_type = other RNA
catgatgctgtctgccgagcgttttagagctatgctgttttg

SEQ ID NO: 336 - RNA, 42 nt, synthetic construct
  source 1..42: mol_type = other RNA
cggctcggcagacagcatcagttttagagctatgctgttttg

SEQ ID NO: 337 - RNA, 42 nt, synthetic construct
  source 1..42: mol_type = other RNA
agctcaccctggccgagttggttttagagctatgctgttttg

SEQ ID NO: 338 - RNA, 42 nt, synthetic construct
  source 1..42: mol_type = other RNA
tctctgcctcaactcggccagttttagagctatgctgttttg

SEQ ID NO: 339 - RNA, 42 nt, synthetic construct
  source 1..42: mol_type = other RNA
ggcctcattgatgacatcttgttttagagctatgctgttttg

SEQ ID NO: 340 - RNA, 42 nt, synthetic construct
  source 1..42: mol_type = other RNA
gtcagtacccgctggtcctcgttttagagctatgctgttttg

SEQ ID NO: 341 - RNA, 42 nt, synthetic construct
  source 1..42: mol_type = other RNA
tacctgccccatgggtgctggttttagagctatgctgttttg

SEQ ID NO: 342 - RNA, 42 nt, synthetic construct
  source 1..42: mol_type = other RNA
cttacctgccccatgggtgcgttttagagctatgctgttttg

SEQ ID NO: 343 - RNA, 42 nt, synthetic construct
  source 1..42: mol_type = other RNA
atcctgcttacctgccccatgttttagagctatgctgttttg

SEQ ID NO: 344 - RNA, 42 nt, synthetic construct
  source 1..42: mol_type = other RNA
cccaggccctttttgcaggtgttttagagctatgctgttttg

SEQ ID NO: 345 - RNA, 42 nt, synthetic construct
  source 1..42: mol_type = other RNA
caggttggcagctgttttgcgttttagagctatgctgttttg

SEQ ID NO: 346 - RNA, 42 nt, synthetic construct
  source 1..42: mol_type = other RNA
cgcccgctgcgccccagatggttttagagctatgctgttttg

SEQ ID NO: 347 - RNA, 42 nt, synthetic construct
  source 1..42: mol_type = other RNA
ctcctcatctggggcgcagcgttttagagctatgctgttttg

SEQ ID NO: 348 - RNA, 42 nt, synthetic construct
  source 1..42: mol_type = other RNA
tccaggagtgggaagcggcggttttagagctatgctgttttg

SEQ ID NO: 349 - RNA, 42 nt, synthetic construct
  source 1..42: mol_type = other RNA
gaagcggcggggcgagcgcagttttagagctatgctgttttg

SEQ ID NO: 350 - RNA, 42 nt, synthetic construct
  source 1..42: mol_type = other RNA
gcggcggggcgagcgcatgggttttagagctatgctgttttg

SEQ ID NO: 351 - RNA, 42 nt, synthetic construct
  source 1..42: mol_type = other RNA
gtctttgactctaaggcccagttttagagctatgctgttttg

SEQ ID NO: 352 - RNA, 42 nt, synthetic construct
  source 1..42: mol_type = other RNA
tctttgactctaaggcccaagttttagagctatgctgttttg

SEQ ID NO: 353 - RNA, 42 nt, synthetic construct
  source 1..42: mol_type = other RNA
tttgactctaaggcccaagggttttagagctatgctgttttg

SEQ ID NO: 354 - RNA, 42 nt, synthetic construct
  source 1..42: mol_type = other RNA
taaggcccaagggggcaagcgttttagagctatgctgttttg

SEQ ID NO: 355 - RNA, 42 nt, synthetic construct
  source 1..42: mol_type = other RNA
aagggggcaagctggtctgcgttttagagctatgctgttttg

SEQ ID NO: 356 - RNA, 42 nt, synthetic construct
  source 1..42: mol_type = other RNA
ggcagaccagcttgccccctgttttagagctatgctgttttg

SEQ ID NO: 357 - RNA, 42 nt, synthetic construct
  source 1..42: mol_type = other RNA
agggggcaagctggtctgccgttttagagctatgctgttttg

SEQ ID NO: 358 - RNA, 42 nt, synthetic construct
  source 1..42: mol_type = other RNA
ccccaaaagcgttgtgggccgttttagagctatgctgttttg

SEQ ID NO: 359 - RNA, 42 nt, synthetic construct
  source 1..42: mol_type = other RNA
cacaacgcttttgggggtgagttttagagctatgctgttttg

SEQ ID NO: 360 - RNA, 42 nt, synthetic construct
  source 1..42: mol_type = other RNA
cctcacccccaaaagcgttggttttagagctatgctgttttg

SEQ ID NO: 361 - RNA, 42 nt, synthetic construct
  source 1..42: mol_type = other RNA
aacctctcccctggccctcagttttagagctatgctgttttg

SEQ ID NO: 362 - RNA, 42 nt, synthetic construct
  source 1..42: mol_type = other RNA
acctctcccctggccctcatgttttagagctatgctgttttg

SEQ ID NO: 363 - RNA, 42 nt, synthetic construct
  source 1..42: mol_type = other RNA
acggtgcccatgagggccaggttttagagctatgctgttttg

SEQ ID NO: 364 - RNA, 42 nt, synthetic construct
  source 1..42: mol_type = other RNA
gacggtgcccatgagggccagttttagagctatgctgttttg

SEQ ID NO: 365 - RNA, 42 nt, synthetic construct
  source 1..42: mol_type = other RNA
tgacggtgcccatgagggccgttttagagctatgctgttttg

SEQ ID NO: 366 - RNA, 42 nt, synthetic construct
  source 1..42: mol_type = other RNA
tcatgggcaccgtcagctccgttttagagctatgctgttttg

SEQ ID NO: 367 - RNA, 42 nt, synthetic construct
  source 1..42: mol_type = other RNA
ggagctgacggtgcccatgagttttagagctatgctgttttg

SEQ ID NO: 368 - RNA, 42 nt, synthetic construct
  source 1..42: mol_type = other RNA
tggagctgacggtgcccatggttttagagctatgctgttttg

SEQ ID NO: 369 - RNA, 42 nt, synthetic construct
  source 1..42: mol_type = other RNA
tgggcaccgtcagctccagggttttagagctatgctgttttg

SEQ ID NO: 370 - RNA, 42 nt, synthetic construct
  source 1..42: mol_type = other RNA
ccgtcagctccaggcggtccgttttagagctatgctgttttg

SEQ ID NO: 371 - RNA, 42 nt, synthetic construct
  source 1..42: mol_type = other RNA
ccaggaccgcctggagctgagttttagagctatgctgttttg

SEQ ID NO: 372 - RNA, 42 nt, synthetic construct
  source 1..42: mol_type = other RNA
tcagctccaggcggtcctgggttttagagctatgctgttttg

SEQ ID NO: 373 - RNA, 42 nt, synthetic construct
  source 1..42: mol_type = other RNA
cagcggccaccaggaccgccgttttagagctatgctgttttg

SEQ ID NO: 374 - RNA, 42 nt, synthetic construct
  source 1..42: mol_type = other RNA
cagcagtggcagcggccaccgttttagagctatgctgttttg

SEQ ID NO: 375 - RNA, 42 nt, synthetic construct
  source 1..42: mol_type = other RNA
gctgctgctcctgggtcccggttttagagctatgctgttttg

SEQ ID NO: 376 - RNA, 42 nt, synthetic construct
  source 1..42: mol_type = other RNA
ctgctgctcctgggtcccgcgttttagagctatgctgttttg

SEQ ID NO: 377 - RNA, 42 nt, synthetic construct
  source 1..42: mol_type = other RNA
cacgggcgcccgcgggacccgttttagagctatgctgttttg

SEQ ID NO: 378 - RNA, 42 nt, synthetic construct
  source 1..42: mol_type = other RNA
tcccgcgggcgcccgtgcgcgttttagagctatgctgttttg

SEQ ID NO: 379 - RNA, 42 nt, synthetic construct
  source 1..42: mol_type = other RNA
cgcgggcgcccgtgcgcagggttttagagctatgctgttttg

SEQ ID NO: 380 - RNA, 42 nt, synthetic construct
  source 1..42: mol_type = other RNA
tcctgcgcacgggcgcccgcgttttagagctatgctgttttg

SEQ ID NO: 381 - RNA, 42 nt, synthetic construct
  source 1..42: mol_type = other RNA
ctcctgcgcacgggcgcccggttttagagctatgctgttttg

SEQ ID NO: 382 - RNA, 42 nt, synthetic construct
  source 1..42: mol_type = other RNA
cgcccgtgcgcaggaggacggttttagagctatgctgttttg

SEQ ID NO: 383 - RNA, 42 nt, synthetic construct
  source 1..42: mol_type = other RNA
cgtgcgcaggaggacgaggagttttagagctatgctgttttg

SEQ ID NO: 384 - RNA, 42 nt, synthetic construct
  source 1..42: mol_type = other RNA
gtcctcgtcctcctgcgcacgttttagagctatgctgttttg

SEQ ID NO: 385 - RNA, 42 nt, synthetic construct
  source 1..42: mol_type = other RNA
cgtcctcgtcctcctgcgcagttttagagctatgctgttttg

SEQ ID NO: 386 - RNA, 42 nt, synthetic construct
  source 1..42: mol_type = other RNA
ggacgaggacggcgactacggttttagagctatgctgttttg

SEQ ID NO: 387 - RNA, 42 nt, synthetic construct
  source 1..42: mol_type = other RNA
ggacggcgactacgaggagcgttttagagctatgctgttttg

SEQ ID NO: 388 - RNA, 42 nt, synthetic construct
  source 1..42: mol_type = other RNA
ggtgctagccttgcgttccggttttagagctatgctgttttg

SEQ ID NO: 389 - RNA, 42 nt, synthetic construct
  source 1..42: mol_type = other RNA
gctagccttgcgttccgagggttttagagctatgctgttttg

SEQ ID NO: 390 - RNA, 42 nt, synthetic construct
  source 1..42: mol_type = other RNA
gccttgcgttccgaggaggagttttagagctatgctgttttg

SEQ ID NO: 391 - RNA, 42 nt, synthetic construct
  source 1..42: mol_type = other RNA
gccgtcctcctcggaacgcagttttagagctatgctgttttg

SEQ ID NO: 392 - RNA, 42 nt, synthetic construct
  source 1..42: mol_type = other RNA
gcgttccgaggaggacggccgttttagagctatgctgttttg

SEQ ID NO: 393 - RNA, 42 nt, synthetic construct
  source 1..42: mol_type = other RNA
ttcggccaggccgtcctcctgttttagagctatgctgttttg

SEQ ID NO: 394 - RNA, 42 nt, synthetic construct
  source 1..42: mol_type = other RNA
ctggccgaagcacccgagcagttttagagctatgctgttttg

SEQ ID NO: 395 - RNA, 42 nt, synthetic construct
  source 1..42: mol_type = other RNA
cgtgctcgggtgcttcggccgttttagagctatgctgttttg

SEQ ID NO: 396 - RNA, 42 nt, synthetic construct
  source 1..42: mol_type = other RNA
ggttccgtgctcgggtgcttgttttagagctatgctgttttg

SEQ ID NO: 397 - RNA, 42 nt, synthetic construct
  source 1..42: mol_type = other RNA
gtggctgtggttccgtgctcgttttagagctatgctgttttg

SEQ ID NO: 398 - RNA, 42 nt, synthetic construct
  source 1..42: mol_type = other RNA
ggtggctgtggttccgtgctgttttagagctatgctgttttg

SEQ ID NO: 399 - RNA, 42 nt, synthetic construct
  source 1..42: mol_type = other RNA
caccttccaccgctgcgccagttttagagctatgctgttttg

SEQ ID NO: 400 - RNA, 42 nt, synthetic construct
  source 1..42: mol_type = other RNA
cttggcgcagcggtggaagggttttagagctatgctgttttg

SEQ ID NO: 401 - RNA, 42 nt, synthetic construct
  source 1..42: mol_type = other RNA
tccaccgctgcgccaaggtggttttagagctatgctgttttg

SEQ ID NO: 402 - RNA, 42 nt, synthetic construct
  source 1..42: mol_type = other RNA
caccttggcgcagcggtggagttttagagctatgctgttttg

SEQ ID NO: 403 - RNA, 42 nt, synthetic construct
  source 1..42: mol_type = other RNA
ccaccgctgcgccaaggtgcgttttagagctatgctgttttg

SEQ ID NO: 404 - RNA, 42 nt, synthetic construct
  source 1..42: mol_type = other RNA
cccgcaccttggcgcagcgggttttagagctatgctgttttg

SEQ ID NO: 405 - RNA, 42 nt, synthetic construct
  source 1..42: mol_type = other RNA
acacccgcaccttggcgcaggttttagagctatgctgttttg

SEQ ID NO: 406 - RNA, 42 nt, synthetic construct
  source 1..42: mol_type = other RNA
ctgcgccaaggtgcgggtgtgttttagagctatgctgttttg

SEQ ID NO: 407 - RNA, 42 nt, synthetic construct
  source 1..42: mol_type = other RNA
tgcgccaaggtgcgggtgtagttttagagctatgctgttttg

SEQ ID NO: 408 - RNA, 42 nt, synthetic construct
  source 1..42: mol_type = other RNA
ccaaggtgcgggtgtagggagttttagagctatgctgttttg

SEQ ID NO: 409 - RNA, 42 nt, synthetic construct
  source 1..42: mol_type = other RNA
caaggtgcgggtgtagggatgttttagagctatgctgttttg

SEQ ID NO: 410 - RNA, 42 nt, synthetic construct
  source 1..42: mol_type = other RNA
ccatccctacacccgcacctgttttagagctatgctgttttg

SEQ ID NO: 411 - RNA, 42 nt, synthetic construct
  source 1..42: mol_type = other RNA
gatcctggccccatgcaagggttttagagctatgctgttttg

SEQ ID NO: 412 - RNA, 42 nt, synthetic construct
  source 1..42: mol_type = other RNA
ttgcatggggccaggatccggttttagagctatgctgttttg

SEQ ID NO: 413 - RNA, 42 nt, synthetic construct
  source 1..42: mol_type = other RNA
acggatcctggccccatgcagttttagagctatgctgttttg

SEQ ID NO: 414 - RNA, 42 nt, synthetic construct
  source 1..42: mol_type = other RNA
catggggccaggatccgtgggttttagagctatgctgttttg

SEQ ID NO: 415 - RNA, 42 nt, synthetic construct
  source 1..42: mol_type = other RNA
caggatccgtggaggttgccgttttagagctatgctgttttg

SEQ ID NO: 416 - RNA, 42 nt, synthetic construct
  source 1..42: mol_type = other RNA
caggcaacctccacggatccgttttagagctatgctgttttg

SEQ ID NO: 417 - RNA, 42 nt, synthetic construct
  source 1..42: mol_type = other RNA
taggtgccaggcaacctccagttttagagctatgctgttttg

SEQ ID NO: 418 - RNA, 42 nt, synthetic construct
  source 1..42: mol_type = other RNA
gaggttgcctggcacctacggttttagagctatgctgttttg

SEQ ID NO: 419 - RNA, 42 nt, synthetic construct
  source 1..42: mol_type = other RNA
gttgcctggcacctacgtgggttttagagctatgctgttttg

SEQ ID NO: 420 - RNA, 42 nt, synthetic construct
  source 1..42: mol_type = other RNA
agcaccaccacgtaggtgccgttttagagctatgctgttttg

SEQ ID NO: 421 - RNA, 42 nt, synthetic construct
  source 1..42: mol_type = other RNA
cacctacgtggtggtgctgagttttagagctatgctgttttg

SEQ ID NO: 422 - RNA, 42 nt, synthetic construct
  source 1..42: mol_type = other RNA
ctacgtggtggtgctgaagggttttagagctatgctgttttg

SEQ ID NO: 423 - RNA, 42 nt, synthetic construct
  source 1..42: mol_type = other RNA
ctccttcagcaccaccacgtgttttagagctatgctgttttg

SEQ ID NO: 424 - RNA, 42 nt, synthetic construct
  source 1..42: mol_type = other RNA
gcgctctgactgcgagaggtgttttagagctatgctgttttg

SEQ ID NO: 425 - RNA, 42 nt, synthetic construct
  source 1..42: mol_type = other RNA
tgcgctctgactgcgagagggttttagagctatgctgttttg

SEQ ID NO: 426 - RNA, 42 nt, synthetic construct
  source 1..42: mol_type = other RNA
cagtgcgctctgactgcgaggttttagagctatgctgttttg

SEQ ID NO: 427 - RNA, 42 nt, synthetic construct
  source 1..42: mol_type = other RNA
gcgcactgcccgccgcctgcgttttagagctatgctgttttg

SEQ ID NO: 428 - RNA, 42 nt, synthetic construct
  source 1..42: mol_type = other RNA
tgcccgccgcctgcaggcccgttttagagctatgctgttttg

SEQ ID NO: 429 - RNA, 42 nt, synthetic construct
  source 1..42: mol_type = other RNA
tgcaggcccaggctgcccgcgttttagagctatgctgttttg

SEQ ID NO: 430 - RNA, 42 nt, synthetic construct
  source 1..42: mol_type = other RNA
caggcccaggctgcccgccggttttagagctatgctgttttg

SEQ ID NO: 431 - RNA, 42 nt, synthetic construct
  source 1..42: mol_type = other RNA
gtatccccggcgggcagcctgttttagagctatgctgttttg

SEQ ID NO: 432 - RNA, 42 nt, synthetic construct
  source 1..42: mol_type = other RNA
ggtatccccggcgggcagccgttttagagctatgctgttttg

SEQ ID NO: 433 - RNA, 42 nt, synthetic construct
  source 1..42: mol_type = other RNA
cttggtgaggtatccccggcgttttagagctatgctgttttg

SEQ ID NO: 434 - RNA, 42 nt, synthetic construct
  source 1..42: mol_type = other RNA
tcttggtgaggtatccccgggttttagagctatgctgttttg

SEQ ID NO: 435 - RNA, 42 nt, synthetic construct
  source 1..42: mol_type = other RNA
ggatcttggtgaggtatcccgttttagagctatgctgttttg

SEQ ID NO: 436 - RNA, 42 nt, synthetic construct
  source 1..42: mol_type = other RNA
agacatgcaggatcttggtggttttagagctatgctgttttg

SEQ ID NO: 437 - RNA, 42 nt, synthetic construct
  source 1..42: mol_type = other RNA
aagatcctgcatgtcttccagttttagagctatgctgttttg

SEQ ID NO: 438 - RNA, 42 nt, synthetic construct
  source 1..42: mol_type = other RNA
atggaagacatgcaggatctgttttagagctatgctgttttg

SEQ ID NO: 439 - RNA, 42 nt, synthetic construct
  source 1..42: mol_type = other RNA
gaaggccatggaagacatgcgttttagagctatgctgttttg

SEQ ID NO: 440 - RNA, 42 nt, synthetic construct
  source 1..42: mol_type = other RNA
gtcttccatggccttcttccgttttagagctatgctgttttg

SEQ ID NO: 441 - RNA, 42 nt, synthetic construct
  source 1..42: mol_type = other RNA
ctcatcttcaccaggaagccgttttagagctatgctgttttg

SEQ ID NO: 442 - RNA, 42 nt, synthetic construct
  source 1..42: mol_type = other RNA
ggcttcctggtgaagatgaggttttagagctatgctgttttg

SEQ ID NO: 443 - RNA, 42 nt, synthetic construct
  source 1..42: mol_type = other RNA
ggtcgccactcatcttcaccgttttagagctatgctgttttg

SEQ ID NO: 444 - RNA, 42 nt, synthetic construct
  source 1..42: mol_type = other RNA
gaagatgagtggcgacctgcgttttagagctatgctgttttg

SEQ ID NO: 445 - RNA, 42 nt, synthetic construct
  source 1..42: mol_type = other RNA
gagtggcgacctgctggagcgttttagagctatgctgttttg

SEQ ID NO: 446 - RNA, 42 nt, synthetic construct
  source 1..42: mol_type = other RNA
ggtggctcaccagctccagcgttttagagctatgctgttttg

SEQ ID NO: 447 - RNA, 42 nt, synthetic construct
  source 1..42: mol_type = other RNA
agctggtgagccacccttttgttttagagctatgctgttttg

SEQ ID NO: 448 - RNA, 42 nt, synthetic construct
  source 1..42: mol_type = other RNA
gctggtgagccaccctttttgttttagagctatgctgttttg

SEQ ID NO: 449 - RNA, 42 nt, synthetic construct
  source 1..42: mol_type = other RNA
aaggcctgcagaagccagaggttttagagctatgctgttttg

SEQ ID NO: 450 - RNA, 42 nt, synthetic construct
  source 1..42: mol_type = other RNA
gtcgacatggggcaacttcagttttagagctatgctgttttg

SEQ ID NO: 451 - RNA, 42 nt, synthetic construct
  source 1..42: mol_type = other RNA
gccccatgtcgactacatcggttttagagctatgctgttttg

SEQ ID NO: 452 - RNA, 42 nt, synthetic construct
  source 1..42: mol_type = other RNA
ccatgtcgactacatcgagggttttagagctatgctgttttg

SEQ ID NO: 453 - RNA, 42 nt, synthetic construct
  source 1..42: mol_type = other RNA
tcctcgatgtagtcgacatggttttagagctatgctgttttg

SEQ ID NO: 454 - RNA, 42 nt, synthetic construct
  source 1..42: mol_type = other RNA
ctcctcgatgtagtcgacatgttttagagctatgctgttttg

SEQ ID NO: 455 - RNA, 42 nt, synthetic construct
  source 1..42: mol_type = other RNA
cctcctcgatgtagtcgacagttttagagctatgctgttttg

SEQ ID NO: 456 - RNA, 42 nt, synthetic construct
  source 1..42: mol_type = other RNA
gatgctctgggcaaagacaggttttagagctatgctgttttg

SEQ ID NO: 457 - RNA, 42 nt, synthetic construct
  source 1..42: mol_type = other RNA
tctttgcccagagcatcccggttttagagctatgctgttttg

SEQ ID NO: 458 - RNA, 42 nt, synthetic construct
  source 1..42: mol_type = other RNA
ccagagcatcccgtggaaccgttttagagctatgctgttttg

SEQ ID NO: 459 - RNA, 42 nt, synthetic construct
  source 1..42: mol_type = other RNA
caggttccacgggatgctctgttttagagctatgctgttttg

SEQ ID NO: 460 - RNA, 42 nt, synthetic construct
  source 1..42: mol_type = other RNA
gcatcccgtggaacctggaggttttagagctatgctgttttg

SEQ ID NO: 461 - RNA, 42 nt, synthetic construct
  source 1..42: mol_type = other RNA
gtaatccgctccaggttccagttttagagctatgctgttttg

SEQ ID NO: 462 - RNA, 42 nt, synthetic construct
  source 1..42: mol_type = other RNA
tggagcggattacccctccagttttagagctatgctgttttg

SEQ ID NO: 463 - RNA, 42 nt, synthetic construct
  source 1..42: mol_type = other RNA
gtggaggggtaatccgctccgttttagagctatgctgttttg

SEQ ID NO: 464 - RNA, 42 nt, synthetic construct
  source 1..42: mol_type = other RNA
ggattacccctccacggtacgttttagagctatgctgttttg

SEQ ID NO: 465 - RNA, 42 nt, synthetic construct
  source 1..42: mol_type = other RNA
gattacccctccacggtaccgttttagagctatgctgttttg

SEQ ID NO: 466 - RNA, 42 nt, synthetic construct
  source 1..42: mol_type = other RNA
tacccctccacggtaccggggttttagagctatgctgttttg

SEQ ID NO: 467 - RNA, 42 nt, synthetic construct
  source 1..42: mol_type = other RNA
atccgcccggtaccgtggaggttttagagctatgctgttttg

SEQ ID NO: 468 - RNA, 42 nt, synthetic construct
  source 1..42: mol_type = other RNA
catccgcccggtaccgtggagttttagagctatgctgttttg

SEQ ID NO: 469 - RNA, 42 nt, synthetic construct
  source 1..42: mol_type = other RNA
tcatccgcccggtaccgtgggttttagagctatgctgttttg

SEQ ID NO: 470 - RNA, 42 nt, synthetic construct
  source 1..42: mol_type = other RNA
tattcatccgcccggtaccggttttagagctatgctgttttg

SEQ ID NO: 471 - RNA, 42 nt, synthetic construct
  source 1..42: mol_type = other RNA
ggggctggtattcatccgccgttttagagctatgctgttttg

SEQ ID NO: 472 - RNA, 42 nt, synthetic construct
  source 1..42: mol_type = other RNA
gcggatgaataccagcccccgttttagagctatgctgttttg

SEQ ID NO: 473 - RNA, 42 nt, synthetic construct
  source 1..42: mol_type = other RNA
cagatgggggtcttaccggggttttagagctatgctgttttg

SEQ ID NO: 474 - RNA, 42 nt, synthetic construct
  source 1..42: mol_type = other RNA
acagatgggggtcttaccgggttttagagctatgctgttttg

SEQ ID NO: 475 - RNA, 42 nt, synthetic construct
  source 1..42: mol_type = other RNA
ctttccaaggcgacatttgtgttttagagctatgctgttttg

SEQ ID NO: 476 - RNA, 42 nt, synthetic construct
  source 1..42: mol_type = other RNA
tctttccaaggcgacatttggttttagagctatgctgttttg

SEQ ID NO: 477 - RNA, 42 nt, synthetic construct
  source 1..42: mol_type = other RNA
caaatgtcgccttggaaagagttttagagctatgctgttttg

SEQ ID NO: 478 - RNA, 42 nt, synthetic construct
  source 1..42: mol_type = other RNA
atgtcgccttggaaagacgggttttagagctatgctgttttg

SEQ ID NO: 479 - RNA, 42 nt, synthetic construct
  source 1..42: mol_type = other RNA
gaaagacggaggcagcctgggttttagagctatgctgttttg

SEQ ID NO: 480 - RNA, 42 nt, synthetic construct
  source 1..42: mol_type = other RNA
ctaggagatacacctccaccgttttagagctatgctgttttg

SEQ ID NO: 481 - RNA, 42 nt, synthetic construct
  source 1..42: mol_type = other RNA
cactctgtatgctggtgtctgttttagagctatgctgttttg

SEQ ID NO: 482 - RNA, 42 nt, synthetic construct
  source 1..42: mol_type = other RNA
ccagcatacagagtgaccacgttttagagctatgctgttttg

SEQ ID NO: 483 - RNA, 42 nt, synthetic construct
  source 1..42: mol_type = other RNA
gagtgaccaccgggaaatcggttttagagctatgctgttttg

SEQ ID NO: 484 - RNA, 42 nt, synthetic construct
  source 1..42: mol_type = other RNA
agtgaccaccgggaaatcgagttttagagctatgctgttttg

SEQ ID NO: 485 - RNA, 42 nt, synthetic construct
  source 1..42: mol_type = other RNA
accaccgggaaatcgagggcgttttagagctatgctgttttg

SEQ ID NO: 486 - RNA, 42 nt, synthetic construct
  source 1..42: mol_type = other RNA
ccaccgggaaatcgagggcagttttagagctatgctgttttg

SEQ ID NO: 487 - RNA, 42 nt, synthetic construct
  source 1..42: mol_type = other RNA
ccctgccctcgatttcccgggttttagagctatgctgttttg

SEQ ID NO: 488 - RNA, 42 nt, synthetic construct
  source 1..42: mol_type = other RNA
tgaccctgccctcgatttccgttttagagctatgctgttttg

SEQ ID NO: 489 - RNA, 42 nt, synthetic construct
  source 1..42: mol_type = other RNA
cgacttcgagaatgtgcccggttttagagctatgctgttttg

SEQ ID NO: 490 - RNA, 42 nt, synthetic construct
  source 1..42: mol_type = other RNA
ctcgggcacattctcgaagtgttttagagctatgctgttttg

SEQ ID NO: 491 - RNA, 42 nt, synthetic construct
  source 1..42: mol_type = other RNA
cttcgagaatgtgcccgagggttttagagctatgctgttttg

SEQ ID NO: 492 - RNA, 42 nt, synthetic construct
  source 1..42: mol_type = other RNA
aagcgggtcccgtcctcctcgttttagagctatgctgttttg

SEQ ID NO: 493 - RNA, 42 nt, synthetic construct
  source 1..42: mol_type = other RNA
gaagcgggtcccgtcctcctgttttagagctatgctgttttg

SEQ ID NO: 494 - RNA, 42 nt, synthetic construct
  source 1..42: mol_type = other RNA
cgggacccgcttccacagacgttttagagctatgctgttttg

SEQ ID NO: 495 - RNA, 42 nt, synthetic construct
  source 1..42: mol_type = other RNA
gcttacctgtctgtggaagcgttttagagctatgctgttttg

SEQ ID NO: 496 - RNA, 42 nt, synthetic construct
  source 1..42: mol_type = other RNA
cggccgtgcttacctgtctggttttagagctatgctgttttg

SEQ ID NO: 497 - RNA, 42 nt, synthetic construct
  source 1..42: mol_type = other RNA
aggtaagcacggccgtctgagttttagagctatgctgttttg

SEQ ID NO: 498 - RNA, 42 nt, synthetic construct
  source 1..42: mol_type = other RNA
ggtaagcacggccgtctgatgttttagagctatgctgttttg

SEQ ID NO: 499 - RNA, 42 nt, synthetic construct
  source 1..42: mol_type = other RNA
ggtccttgtgttcgtcgagcgttttagagctatgctgttttg

SEQ ID NO: 500 - RNA, 42 nt, synthetic construct
  source 1..42: mol_type = other RNA
tggcctgctcgacgaacacagttttagagctatgctgttttg

SEQ ID NO: 501 - RNA, 42 nt, synthetic construct
  source 1..42: mol_type = other RNA
gccagcaagtgtgacagtcagttttagagctatgctgttttg

SEQ ID NO: 502 - RNA, 42 nt, synthetic construct
  source 1..42: mol_type = other RNA
gccatgactgtcacacttgcgttttagagctatgctgttttg

SEQ ID NO: 503 - RNA, 42 nt, synthetic construct
  source 1..42: mol_type = other RNA
gctgcgcatgctggcaccctgttttagagctatgctgttttg

SEQ ID NO: 504 - RNA, 42 nt, synthetic construct
  source 1..42: mol_type = other RNA
cacgcgcaggctgcgcatgcgttttagagctatgctgttttg

SEQ ID NO: 505 - RNA, 42 nt, synthetic construct
  source 1..42: mol_type = other RNA
ctgcgcgtgctcaactgccagttttagagctatgctgttttg

SEQ ID NO: 506 - RNA, 42 nt, synthetic construct
  source 1..42: mol_type = other RNA
tgcgcgtgctcaactgccaagttttagagctatgctgttttg

SEQ ID NO: 507 - RNA, 42 nt, synthetic construct
  source 1..42: mol_type = other RNA
cttggcagttgagcacgcgcgttttagagctatgctgttttg

SEQ ID NO: 508 - RNA, 42 nt, synthetic construct
  source 1..42: mol_type = other RNA
cgtgctcaactgccaagggagttttagagctatgctgttttg

SEQ ID NO: 509 - RNA, 42 nt, synthetic construct
  source 1..42: mol_type = other RNA
gtgctcaactgccaagggaagttttagagctatgctgttttg

SEQ ID NO: 510 - RNA, 42 nt, synthetic construct
  source 1..42: mol_type = other RNA
caagggaagggcacggttaggttttagagctatgctgttttg

SEQ ID NO: 511 - RNA, 42 nt, synthetic construct
  source 1..42: mol_type = other RNA
cgctaaccgtgcccttccctgttttagagctatgctgttttg

SEQ ID NO: 512 - RNA, 42 nt, synthetic construct
  source 1..42: mol_type = other RNA
acggttagcggcaccctcatgttttagagctatgctgttttg

SEQ ID NO: 513 - RNA, 42 nt, synthetic construct
  source 1..42: mol_type = other RNA
gggccatcacttacctatgagttttagagctatgctgttttg

SEQ ID NO: 514 - RNA, 42 nt, synthetic construct
  source 1..42: mol_type = other RNA
ggggccatcacttacctatggttttagagctatgctgttttg

SEQ ID NO: 515 - RNA, 42 nt, synthetic construct
  source 1..42: mol_type = other RNA
tccgaataaactccaggcctgttttagagctatgctgttttg

SEQ ID NO: 516 - RNA, 42 nt, synthetic construct
  source 1..42: mol_type = other RNA
ggcttttccgaataaactccgttttagagctatgctgttttg

SEQ ID NO: 517 - RNA, 42 nt, synthetic construct
  source 1..42: mol_type = other RNA
gtttattcggaaaagccagcgttttagagctatgctgttttg

SEQ ID NO: 518 - RNA, 42 nt, synthetic construct
  source 1..42: mol_type = other RNA
gccagctggtccagcctgtggttttagagctatgctgttttg

SEQ ID NO: 519 - RNA, 42 nt, synthetic construct
  source 1..42: mol_type = other RNA
agcaccaccagtggccccacgttttagagctatgctgttttg

SEQ ID NO: 520 - RNA, 42 nt, synthetic construct
  source 1..42: mol_type = other RNA
cggctgtacccacccgccaggttttagagctatgctgttttg

SEQ ID NO: 521 - RNA, 42 nt, synthetic construct
  source 1..42: mol_type = other RNA
gtcgtgctggtcaccgctgcgttttagagctatgctgttttg

SEQ ID NO: 522 - RNA, 42 nt, synthetic construct
  source 1..42: mol_type = other RNA
tcaccgctgccggcaacttcgttttagagctatgctgttttg

SEQ ID NO: 523 - RNA, 42 nt, synthetic construct
  source 1..42: mol_type = other RNA
caccgctgccggcaacttccgttttagagctatgctgttttg

SEQ ID NO: 524 - RNA, 42 nt, synthetic construct
  source 1..42: mol_type = other RNA
gtcccggaagttgccggcaggttttagagctatgctgttttg

SEQ ID NO: 525 - RNA, 42 nt, synthetic construct
  source 1..42: mol_type = other RNA
ggcatcgtcccggaagttgcgttttagagctatgctgttttg

SEQ ID NO: 526 - RNA, 42 nt, synthetic construct
  source 1..42: mol_type = other RNA
agtagaggcaggcatcgtccgttttagagctatgctgttttg

SEQ ID NO: 527 - RNA, 42 nt, synthetic construct
  source 1..42: mol_type = other RNA
ccagcctcagctcccgaggtgttttagagctatgctgttttg

SEQ ID NO: 528 - RNA, 42 nt, synthetic construct
  source 1..42: mol_type = other RNA
agcacctacctcgggagctggttttagagctatgctgttttg

SEQ ID NO: 529 - RNA, 42 nt, synthetic construct
  source 1..42: mol_type = other RNA
ctttccaggtcatcacagttgttttagagctatgctgttttg

SEQ ID NO: 530 - RNA, 42 nt, synthetic construct
  source 1..42: mol_type = other RNA
tttccaggtcatcacagttggttttagagctatgctgttttg

SEQ ID NO: 531 - RNA, 42 nt, synthetic construct
  source 1..42: mol_type = other RNA
tggccccaactgtgatgaccgttttagagctatgctgttttg

SEQ ID NO: 532 - RNA, 42 nt, synthetic construct
  source 1..42: mol_type = other RNA
cggctggtcttgggcattgggttttagagctatgctgttttg

SEQ ID NO: 533 - RNA, 42 nt, synthetic construct
  source 1..42: mol_type = other RNA
caccggctggtcttgggcatgttttagagctatgctgttttg

SEQ ID NO: 534 - RNA, 42 nt, synthetic construct
  source 1..42: mol_type = other RNA
caagaccagccggtgaccctgttttagagctatgctgttttg

SEQ ID NO: 535 - RNA, 42 nt, synthetic construct
  source 1..42: mol_type = other RNA
aagaccagccggtgaccctggttttagagctatgctgttttg

SEQ ID NO: 536 - RNA, 42 nt, synthetic construct
  source 1..42: mol_type = other RNA
cagggtcaccggctggtcttgttttagagctatgctgttttg

SEQ ID NO: 537 - RNA, 42 nt, synthetic construct
  source 1..42: mol_type = other RNA
gaccaactttggccgctgtggttttagagctatgctgttttg

SEQ ID NO: 538 - RNA, 42 nt, synthetic construct
  source 1..42: mol_type = other RNA
gtccacacagcggccaaagtgttttagagctatgctgttttg

SEQ ID NO: 539 - RNA, 42 nt, synthetic construct
  source 1..42: mol_type = other RNA
gggcaaagaggtccacacaggttttagagctatgctgttttg

SEQ ID NO: 540 - RNA, 42 nt, synthetic construct
  source 1..42: mol_type = other RNA
gtgtggacctctttgccccagttttagagctatgctgttttg

SEQ ID NO: 541 - RNA, 42 nt, synthetic construct
  source 1..42: mol_type = other RNA
tgtcctcccctggggcaaaggttttagagctatgctgttttg

SEQ ID NO: 542 - RNA, 42 nt, synthetic construct
  source 1..42: mol_type = other RNA
acctgctttgtgtcacagaggttttagagctatgctgttttg

SEQ ID NO: 543 - RNA, 42 nt, synthetic construct
  source 1..42: mol_type = other RNA
cctgctttgtgtcacagagtgttttagagctatgctgttttg

SEQ ID NO: 544 - RNA, 42 nt, synthetic construct
  source 1..42: mol_type = other RNA
cccactctgtgacacaaagcgttttagagctatgctgttttg

SEQ ID NO: 545 - RNA, 42 nt, synthetic construct
  source 1..42: mol_type = other RNA
gtcacagagtgggacatcacgttttagagctatgctgttttg

SEQ ID NO: 546 - RNA, 42 nt, synthetic construct
  source 1..42: mol_type = other RNA
tggtgacttaccagccacgtgttttagagctatgctgttttg

SEQ ID NO: 547 - RNA, 42 nt, synthetic construct
  source 1..42: mol_type = other RNA
gtggtgacttaccagccacggttttagagctatgctgttttg

SEQ ID NO: 548 - RNA, 42 nt, synthetic construct
  source 1..42: mol_type = other RNA
tgccgagccggagctcacccgttttagagctatgctgttttg

SEQ ID NO: 549 - RNA, 42 nt, synthetic construct
  source 1..42: mol_type = other RNA
ggccagggtgagctccggctgttttagagctatgctgttttg

SEQ ID NO: 550 - RNA, 42 nt, synthetic construct
  source 1..42: mol_type = other RNA
aactcggccagggtgagctcgttttagagctatgctgttttg

SEQ ID NO: 551 - RNA, 42 nt, synthetic construct
  source 1..42: mol_type = other RNA
gtctctgcctcaactcggccgttttagagctatgctgttttg

SEQ ID NO: 552 - RNA, 42 nt, synthetic construct
  source 1..42: mol_type = other RNA
gatcagtctctgcctcaactgttttagagctatgctgttttg

SEQ ID NO: 553 - RNA, 42 nt, synthetic construct
  source 1..42: mol_type = other RNA
tgacatctttggcagagaaggttttagagctatgctgttttg

SEQ ID NO: 554 - RNA, 42 nt, synthetic construct
  source 1..42: mol_type = other RNA
aagatgtcatcaatgaggccgttttagagctatgctgttttg

SEQ ID NO: 555 - RNA, 42 nt, synthetic construct
  source 1..42: mol_type = other RNA
caatgaggcctggttccctggttttagagctatgctgttttg

SEQ ID NO: 556 - RNA, 42 nt, synthetic construct
  source 1..42: mol_type = other RNA
tcagtacccgctggtcctcagttttagagctatgctgttttg

SEQ ID NO: 557 - RNA, 42 nt, synthetic construct
  source 1..42: mol_type = other RNA
gcgggtactgacccccaaccgttttagagctatgctgttttg

SEQ ID NO: 558 - RNA, 42 nt, synthetic construct
  source 1..42: mol_type = other RNA
ggttgggggtcagtacccgcgttttagagctatgctgttttg

SEQ ID NO: 559 - RNA, 42 nt, synthetic construct
  source 1..42: mol_type = other RNA
ggtactgacccccaacctgggttttagagctatgctgttttg

SEQ ID NO: 560 - RNA, 42 nt, synthetic construct
  source 1..42: mol_type = other RNA
cctgccccccagcacccatggttttagagctatgctgttttg

SEQ ID NO: 561 - RNA, 42 nt, synthetic construct
  source 1..42: mol_type = other RNA
ttacctgccccatgggtgctgttttagagctatgctgttttg

SEQ ID NO: 562 - RNA, 42 nt, synthetic construct
  source 1..42: mol_type = other RNA
gccaacctgcaaaaagggccgttttagagctatgctgttttg

SEQ ID NO: 563 - RNA, 42 nt, synthetic construct
  source 1..42: mol_type = other RNA
gactgtatggtcagcacactgttttagagctatgctgttttg

SEQ ID NO: 564 - RNA, 42 nt, synthetic construct
  source 1..42: mol_type = other RNA
actgtatggtcagcacactcgttttagagctatgctgttttg

SEQ ID NO: 565 - RNA, 42 nt, synthetic construct
  source 1..42: mol_type = other RNA
ctgtatggtcagcacactcggttttagagctatgctgttttg

SEQ ID NO: 566 - RNA, 42 nt, synthetic construct
  source 1..42: mol_type = other RNA
cagcacactcggggcctacagttttagagctatgctgttttg

SEQ ID NO: 567 - RNA, 42 nt, synthetic construct
  source 1..42: mol_type = other RNA
acactcggggcctacacggagttttagagctatgctgttttg

SEQ ID NO: 568 - RNA, 42 nt, synthetic construct
  source 1..42: mol_type = other RNA
acggctgtggccatccgtgtgttttagagctatgctgttttg

SEQ ID NO: 569 - RNA, 42 nt, synthetic construct
  source 1..42: mol_type = other RNA
gctcctcatctggggcgcaggttttagagctatgctgttttg

SEQ ID NO: 570 - RNA, 42 nt, synthetic construct
  source 1..42: mol_type = other RNA
gcagaccagcttgcccccttgttttagagctatgctgttttg

SEQ ID NO: 571 - RNA, 42 nt, synthetic construct
  source 1..42: mol_type = other RNA
tgccgggcccacaacgctttgttttagagctatgctgttttg

SEQ ID NO: 572 - RNA, 42 nt, synthetic construct
  source 1..42: mol_type = other RNA
gccgggcccacaacgcttttgttttagagctatgctgttttg

SEQ ID NO: 573 - RNA, 42 nt, synthetic construct
  source 1..42: mol_type = other RNA
ccgggcccacaacgcttttggttttagagctatgctgttttg

SEQ ID NO: 574 - RNA, 42 nt, synthetic construct
  source 1..42: mol_type = other RNA
cgggcccacaacgcttttgggttttagagctatgctgttttg

SEQ ID NO: 575 - RNA, 42 nt, synthetic construct
  source 1..42: mol_type = other RNA
ccacaacgcttttgggggtggttttagagctatgctgttttg

SEQ ID NO: 576 - RNA, 42 nt, synthetic construct
  source 1..42: mol_type = other RNA
ctcacccccaaaagcgttgtgttttagagctatgctgttttg

SEQ ID NO: 577 - RNA, 42 nt, synthetic construct
  source 1..42: mol_type = other RNA
agggtgtctacgccattgccgttttagagctatgctgttttg

SEQ ID NO: 578 - RNA, 42 nt, synthetic construct
  source 1..42: mol_type = other RNA
gtggacacgggtccccatgcgttttagagctatgctgttttg

SEQ ID NO: 579 - RNA, 42 nt, synthetic construct
  source 1..42: mol_type = other RNA
aaggtcctccacctcccagtgttttagagctatgctgttttg

SEQ ID NO: 580 - RNA, 42 nt, synthetic construct
  source 1..42: mol_type = other RNA
cctcagcacaggcggcttgtgttttagagctatgctgttttg

SEQ ID NO: 581 - RNA, 42 nt, synthetic construct
  source 1..42: mol_type = other RNA
cactggttgggctgacctcggttttagagctatgctgttttg

SEQ ID NO: 582 - RNA, 42 nt, synthetic construct
  source 1..42: mol_type = other RNA
aggtcagcccaaccagtgcggttttagagctatgctgttttg

SEQ ID NO: 583 - RNA, 42 nt, synthetic construct
  source 1..42: mol_type = other RNA
ggtcagcccaaccagtgcgtgttttagagctatgctgttttg

SEQ ID NO: 584 - RNA, 42 nt, synthetic construct
  source 1..42: mol_type = other RNA
caaccagtgcgtgggccacagttttagagctatgctgttttg

SEQ ID NO: 585 - RNA, 42 nt, synthetic construct
  source 1..42: mol_type = other RNA
tagacaacacgtgtgtagtcgttttagagctatgctgttttg

SEQ ID NO: 586 - RNA, 42 nt, synthetic construct
  source 1..42: mol_type = other RNA
gactacacacgtgttgtctagttttagagctatgctgttttg

SEQ ID NO: 587 - RNA, 42 nt, synthetic construct
  source 1..42: mol_type = other RNA
cacgtgtgtagtcaggagccgttttagagctatgctgttttg

SEQ ID NO: 588 - RNA, 42 nt, synthetic construct
  source 1..42: mol_type = other RNA
agccgggacgtcagcactacgttttagagctatgctgttttg

SEQ ID NO: 589 - RNA, 42 nt, synthetic construct
  source 1..42: mol_type = other RNA
tgcctgtagtgctgacgtccgttttagagctatgctgttttg

SEQ ID NO: 590 - RNA, 42 nt, synthetic construct
  source 1..42: mol_type = other RNA
actacaggcagcaccagcgagttttagagctatgctgttttg

SEQ ID NO: 591 - RNA, 42 nt, synthetic construct
  source 1..42: mol_type = other RNA
tacaggcagcaccagcgaaggttttagagctatgctgttttg

SEQ ID NO: 592 - RNA, 42 nt, synthetic construct
  source 1..42: mol_type = other RNA
gcagatggcaacggctgtcagttttagagctatgctgttttg

SEQ ID NO: 593 - RNA, 100 nt, synthetic construct
  source 1..100: mol_type = other RNA
  ncRNA 1..100: ncRNA_class = sgRNA
ccaggttccacgggatgctcgttttagagctagaaatagcaagttaaaataaggctagtccgttatcaacttgaaaaagtggcaccgagtcggtgctttt

SEQ ID NO: 594 - RNA, 100 nt, synthetic construct
  source 1..100: mol_type = other RNA
  ncRNA 1..100: ncRNA_class = sgRNA
taatccgctccaggttccacgttttagagctagaaatagcaagttaaaataaggctagtccgttatcaacttgaaaaagtggcaccgagtcggtgctttt

SEQ ID NO: 595 - RNA, 100 nt, synthetic construct
  source 1..100: mol_type = other RNA
  ncRNA 1..100: ncRNA_class = sgRNA
tgggggtcttaccggggggcgttttagagctagaaatagcaagttaaaataaggctagtccgttatcaacttgaaaaagtggcaccgagtcggtgctttt

SEQ ID NO: 596 - RNA, 100 nt, synthetic construct
  source 1..100: mol_type = other RNA
  ncRNA 1..100: ncRNA_class = sgRNA
ttggaaagacggaggcagccgttttagagctagaaatagcaagttaaaataaggctagtccgttatcaacttgaaaaagtggcaccgagtcggtgctttt

SEQ ID NO: 597 - RNA, 100 nt, synthetic construct
  source 1..100: mol_type = other RNA
  ncRNA 1..100: ncRNA_class = sgRNA
cagcatacagagtgaccaccgttttagagctagaaatagcaagttaaaataaggctagtccgttatcaacttgaaaaagtggcaccgagtcggtgctttt

SEQ ID NO: 598 - RNA, 100 nt, synthetic construct
  source 1..100: mol_type = other RNA
  ncRNA 1..100: ncRNA_class = sgRNA
ccggtggtcactctgtatgcgttttagagctagaaatagcaagttaaaataaggctagtccgttatcaacttgaaaaagtggcaccgagtcggtgctttt

SEQ ID NO: 599 - RNA, 100 nt, synthetic construct
  source 1..100: mol_type = other RNA
  ncRNA 1..100: ncRNA_class = sgRNA
agaatgtgcccgaggaggacgttttagagctagaaatagcaagttaaaataaggctagtccgttatcaacttgaaaaagtggcaccgagtcggtgctttt

SEQ ID NO: 600 - RNA, 100 nt, synthetic construct
  source 1..100: mol_type = other RNA
  ncRNA 1..100: ncRNA_class = sgRNA
agtcatggcacccacctggcgttttagagctagaaatagcaagttaaaataaggctagtccgttatcaacttgaaaaagtggcaccgagtcggtgctttt

SEQ ID NO: 601 - RNA, 100 nt, synthetic construct
  source 1..100: mol_type = other RNA
  ncRNA 1..100: ncRNA_class = sgRNA
gtcatggcacccacctggcagttttagagctagaaatagcaagttaaaataaggctagtccgttatcaacttgaaaaagtggcaccgagtcggtgctttt

SEQ ID NO: 602 - RNA, 100 nt, synthetic construct
  source 1..100: mol_type = other RNA
  ncRNA 1..100: ncRNA_class = sgRNA
ccgggatgccggcgtggccagttttagagctagaaatagcaagttaaaataaggctagtccgttatcaacttgaaaaagtggcaccgagtcggtgctttt

SEQ ID NO: 603 - RNA, 100 nt, synthetic construct
  source 1..100: mol_type = other RNA
  ncRNA 1..100: ncRNA_class = sgRNA
cgggatgccggcgtggccaagttttagagctagaaatagcaagttaaaataaggctagtccgttatcaacttgaaaaagtggcaccgagtcggtgctttt

SEQ ID NO: 604 - RNA, 100 nt, synthetic construct
  source 1..100: mol_type = other RNA
  ncRNA 1..100: ncRNA_class = sgRNA
ccttggccacgccggcatccgttttagagctagaaatagcaagttaaaataaggctagtccgttatcaacttgaaaaagtggcaccgagtcggtgctttt

SEQ ID NO: 605 - RNA, 100 nt, synthetic construct
  source 1..100: mol_type = other RNA
  ncRNA 1..100: ncRNA_class = sgRNA
gctggcacccttggccacgcgttttagagctagaaatagcaagttaaaataaggctagtccgttatcaacttgaaaaagtggcaccgagtcggtgctttt

SEQ ID NO: 606 - RNA, 100 nt, synthetic construct
  source 1..100: mol_type = other RNA
  ncRNA 1..100: ncRNA_class = sgRNA
tcccaggcctggagtttattgttttagagctagaaatagcaagttaaaataaggctagtccgttatcaacttgaaaaagtggcaccgagtcggtgctttt

SEQ ID NO: 607 - RNA, 100 nt, synthetic construct
  source 1..100: mol_type = other RNA
  ncRNA 1..100: ncRNA_class = sgRNA
ttccgaataaactccaggccgttttagagctagaaatagcaagttaaaataaggctagtccgttatcaacttgaaaaagtggcaccgagtcggtgctttt

SEQ ID NO: 608 - RNA, 100 nt, synthetic construct
  source 1..100: mol_type = other RNA
  ncRNA 1..100: ncRNA_class = sgRNA
gcggctgtacccacccgccagttttagagctagaaatagcaagttaaaataaggctagtccgttatcaacttgaaaaagtggcaccgagtcggtgctttt

SEQ ID NO: 609 - RNA, 100 nt, synthetic construct
  source 1..100: mol_type = other RNA
  ncRNA 1..100: ncRNA_class = sgRNA
cgcggctgtacccacccgccgttttagagctagaaatagcaagttaaaataaggctagtccgttatcaacttgaaaaagtggcaccgagtcggtgctttt

SEQ ID NO: 610 - RNA, 100 nt, synthetic construct
  source 1..100: mol_type = other RNA
  ncRNA 1..100: ncRNA_class = sgRNA
ggcaggcggcgttgaggacggttttagagctagaaatagcaagttaaaataaggctagtccgttatcaacttgaaaaagtggcaccgagtcggtgctttt

SEQ ID NO: 611 - RNA, 100 nt, synthetic construct
  source 1..100: mol_type = other RNA
  ncRNA 1..100: ncRNA_class = sgRNA
caacgccgcctgccagcgccgttttagagctagaaatagcaagttaaaataaggctagtccgttatcaacttgaaaaagtggcaccgagtcggtgctttt

SEQ ID NO: 612 - RNA, 100 nt, synthetic construct
  source 1..100: mol_type = other RNA
  ncRNA 1..100: ncRNA_class = sgRNA
ggcgctggcaggcggcgttggttttagagctagaaatagcaagttaaaataaggctagtccgttatcaacttgaaaaagtggcaccgagtcggtgctttt

SEQ ID NO: 613 - RNA, 100 nt, synthetic construct
  source 1..100: mol_type = other RNA
  ncRNA 1..100: ncRNA_class = sgRNA
ccgcctgccagcgcctggcggttttagagctagaaatagcaagttaaaataaggctagtccgttatcaacttgaaaaagtggcaccgagtcggtgctttt

SEQ ID NO: 614 - RNA, 100 nt, synthetic construct
  source 1..100: mol_type = other RNA
  ncRNA 1..100: ncRNA_class = sgRNA
cgcctgccagcgcctggcgagttttagagctagaaatagcaagttaaaataaggctagtccgttatcaacttgaaaaagtggcaccgagtcggtgctttt

SEQ ID NO: 615 - RNA, 100 nt, synthetic construct
  source 1..100: mol_type = other RNA
  ncRNA 1..100: ncRNA_class = sgRNA
agccctcgccaggcgctggcgttttagagctagaaatagcaagttaaaataaggctagtccgttatcaacttgaaaaagtggcaccgagtcggtgctttt

SEQ ID NO: 616 - RNA, 100 nt, synthetic construct
  source 1..100: mol_type = other RNA
  ncRNA 1..100: ncRNA_class = sgRNA
gcacgaccccagccctcgccgttttagagctagaaatagcaagttaaaataaggctagtccgttatcaacttgaaaaagtggcaccgagtcggtgctttt

SEQ ID NO: 617 - RNA, 100 nt, synthetic construct
  source 1..100: mol_type = other RNA
  ncRNA 1..100: ncRNA_class = sgRNA
cctttccaggtcatcacagtgttttagagctagaaatagcaagttaaaataaggctagtccgttatcaacttgaaaaagtggcaccgagtcggtgctttt

SEQ ID NO: 618 - RNA, 100 nt, synthetic construct
  source 1..100: mol_type = other RNA
  ncRNA 1..100: ncRNA_class = sgRNA
gccggtgaccctggggacttgttttagagctagaaatagcaagttaaaataaggctagtccgttatcaacttgaaaaagtggcaccgagtcggtgctttt

SEQ ID NO: 619 - RNA, 100 nt, synthetic construct
  source 1..100: mol_type = other RNA
  ncRNA 1..100: ncRNA_class = sgRNA
aagtccccagggtcaccggcgttttagagctagaaatagcaagttaaaataaggctagtccgttatcaacttgaaaaagtggcaccgagtcggtgctttt

SEQ ID NO: 620 - RNA, 100 nt, synthetic construct
  source 1..100: mol_type = other RNA
  ncRNA 1..100: ncRNA_class = sgRNA
ccggtgaccctggggactttgttttagagctagaaatagcaagttaaaataaggctagtccgttatcaacttgaaaaagtggcaccgagtcggtgctttt

SEQ ID NO: 621 - RNA, 100 nt, synthetic construct
  source 1..100: mol_type = other RNA
  ncRNA 1..100: ncRNA_class = sgRNA
gttggtccccaaagtccccagttttagagctagaaatagcaagttaaaataaggctagtccgttatcaacttgaaaaagtggcaccgagtcggtgctttt

SEQ ID NO: 622 - RNA, 100 nt, synthetic construct
  source 1..100: mol_type = other RNA
  ncRNA 1..100: ncRNA_class = sgRNA
agttggtccccaaagtccccgttttagagctagaaatagcaagttaaaataaggctagtccgttatcaacttgaaaaagtggcaccgagtcggtgctttt

SEQ ID NO: 623 - RNA, 100 nt, synthetic construct
  source 1..100: mol_type = other RNA
  ncRNA 1..100: ncRNA_class = sgRNA
gggactttggggaccaacttgttttagagctagaaatagcaagttaaaataaggctagtccgttatcaacttgaaaaagtggcaccgagtcggtgctttt

SEQ ID NO: 624 - RNA, 100 nt, synthetic construct
  source 1..100: mol_type = other RNA
  ncRNA 1..100: ncRNA_class = sgRNA
tgtgtggacctctttgccccgttttagagctagaaatagcaagttaaaataaggctagtccgttatcaacttgaaaaagtggcaccgagtcggtgctttt

SEQ ID NO: 625 - RNA, 100 nt, synthetic construct
  source 1..100: mol_type = other RNA
  ncRNA 1..100: ncRNA_class = sgRNA
tgtggacctctttgccccaggttttagagctagaaatagcaagttaaaataaggctagtccgttatcaacttgaaaaagtggcaccgagtcggtgctttt

SEQ ID NO: 626 - RNA, 100 nt, synthetic construct
  source 1..100: mol_type = other RNA
  ncRNA 1..100: ncRNA_class = sgRNA
ggacctctttgccccagggggttttagagctagaaatagcaagttaaaataaggctagtccgttatcaacttgaaaaagtggcaccgagtcggtgctttt

SEQ ID NO: 627 - RNA, 100 nt, synthetic construct
  source 1..100: mol_type = other RNA
  ncRNA 1..100: ncRNA_class = sgRNA
gcaccaatgatgtcctccccgttttagagctagaaatagcaagttaaaataaggctagtccgttatcaacttgaaaaagtggcaccgagtcggtgctttt

SEQ ID NO: 628 - RNA, 100 nt, synthetic construct
  source 1..100: mol_type = other RNA
  ncRNA 1..100: ncRNA_class = sgRNA
aaagcaggtgctgcagtcgcgttttagagctagaaatagcaagttaaaataaggctagtccgttatcaacttgaaaaagtggcaccgagtcggtgctttt

SEQ ID NO: 629 - RNA, 100 nt, synthetic construct
  source 1..100: mol_type = other RNA
  ncRNA 1..100: ncRNA_class = sgRNA
atcacaggctgctgcccacggttttagagctagaaatagcaagttaaaataaggctagtccgttatcaacttgaaaaagtggcaccgagtcggtgctttt

SEQ ID NO: 630 - RNA, 100 nt, synthetic construct
  source 1..100: mol_type = other RNA
  ncRNA 1..100: ncRNA_class = sgRNA
agcatcatggctgcaatgccgttttagagctagaaatagcaagttaaaataaggctagtccgttatcaacttgaaaaagtggcaccgagtcggtgctttt

SEQ ID NO: 631 - RNA, 100 nt, synthetic construct
  source 1..100: mol_type = other RNA
  ncRNA 1..100: ncRNA_class = sgRNA
catgatgctgtctgccgagcgttttagagctagaaatagcaagttaaaataaggctagtccgttatcaacttgaaaaagtggcaccgagtcggtgctttt

SEQ ID NO: 632 - RNA, 100 nt, synthetic construct
  source 1..100: mol_type = other RNA
  ncRNA 1..100: ncRNA_class = sgRNA
cggctcggcagacagcatcagttttagagctagaaatagcaagttaaaataaggctagtccgttatcaacttgaaaaagtggcaccgagtcggtgctttt

SEQ ID NO: 633 - RNA, 100 nt, synthetic construct
  source 1..100: mol_type = other RNA
  ncRNA 1..100: ncRNA_class = sgRNA
agctcaccctggccgagttggttttagagctagaaatagcaagttaaaataaggctagtccgttatcaacttgaaaaagtggcaccgagtcggtgctttt

SEQ ID NO: 634 - RNA, 100 nt, synthetic construct
  source 1..100: mol_type = other RNA
  ncRNA 1..100: ncRNA_class = sgRNA
tctctgcctcaactcggccagttttagagctagaaatagcaagttaaaataaggctagtccgttatcaacttgaaaaagtggcaccgagtcggtgctttt

SEQ ID NO: 635 - RNA, 100 nt, synthetic construct
  source 1..100: mol_type = other RNA
  ncRNA 1..100: ncRNA_class = sgRNA
ggcctcattgatgacatcttgttttagagctagaaatagcaagttaaaataaggctagtccgttatcaacttgaaaaagtggcaccgagtcggtgctttt

SEQ ID NO: 636 - RNA, 100 nt, synthetic construct
  source 1..100: mol_type = other RNA
  ncRNA 1..100: ncRNA_class = sgRNA
gtcagtacccgctggtcctcgttttagagctagaaatagcaagttaaaataaggctagtccgttatcaacttgaaaaagtggcaccgagtcggtgctttt

SEQ ID NO: 637 - RNA, 100 nt, synthetic construct
  source 1..100: mol_type = other RNA
  ncRNA 1..100: ncRNA_class = sgRNA
tacctgccccatgggtgctggttttagagctagaaatagcaagttaaaataaggctagtccgttatcaacttgaaaaagtggcaccgagtcggtgctttt

SEQ ID NO: 638 - RNA, 100 nt, synthetic construct
  source 1..100: mol_type = other RNA
  ncRNA 1..100: ncRNA_class = sgRNA
cttacctgccccatgggtgcgttttagagctagaaatagcaagttaaaataaggctagtccgttatcaacttgaaaaagtggcaccgagtcggtgctttt

SEQ ID NO: 639 - RNA, 100 nt, synthetic construct
  source 1..100: mol_type = other RNA
  ncRNA 1..100: ncRNA_class = sgRNA
atcctgcttacctgccccatgttttagagctagaaatagcaagttaaaataaggctagtccgttatcaacttgaaaaagtggcaccgagtcggtgctttt

SEQ ID NO: 640 - RNA, 100 nt, synthetic construct
  source 1..100: mol_type = other RNA
  ncRNA 1..100: ncRNA_class = sgRNA
cccaggccctttttgcaggtgttttagagctagaaatagcaagttaaaataaggctagtccgttatcaacttgaaaaagtggcaccgagtcggtgctttt

SEQ ID NO: 641 - RNA, 100 nt, synthetic construct
  source 1..100: mol_type = other RNA
  ncRNA 1..100: ncRNA_class = sgRNA
caggttggcagctgttttgcgttttagagctagaaatagcaagttaaaataaggctagtccgttatcaacttgaaaaagtggcaccgagtcggtgctttt

SEQ ID NO: 642 - RNA, 100 nt, synthetic construct
  source 1..100: mol_type = other RNA
  ncRNA 1..100: ncRNA_class = sgRNA
cgcccgctgcgccccagatggttttagagctagaaatagcaagttaaaataaggctagtccgttatcaacttgaaaaagtggcaccgagtcggtgctttt

SEQ ID NO: 643 - RNA, 100 nt, synthetic construct
  source 1..100: mol_type = other RNA
  ncRNA 1..100: ncRNA_class = sgRNA
ctcctcatctggggcgcagcgttttagagctagaaatagcaagttaaaataaggctagtccgttatcaacttgaaaaagtggcaccgagtcggtgctttt

SEQ ID NO: 644 - RNA, 100 nt, synthetic construct
  source 1..100: mol_type = other RNA
  ncRNA 1..100: ncRNA_class = sgRNA
tccaggagtgggaagcggcggttttagagctagaaatagcaagttaaaataaggctagtccgttatcaacttgaaaaagtggcaccgagtcggtgctttt

SEQ ID NO: 645 - RNA, 100 nt, synthetic construct
  source 1..100: mol_type = other RNA
  ncRNA 1..100: ncRNA_class = sgRNA
gaagcggcggggcgagcgcagttttagagctagaaatagcaagttaaaataaggctagtccgttatcaacttgaaaaagtggcaccgagtcggtgctttt

SEQ ID NO: 646 - RNA, 100 nt, synthetic construct
  source 1..100: mol_type = other RNA
  ncRNA 1..100: ncRNA_class = sgRNA
gcggcggggcgagcgcatgggttttagagctagaaatagcaagttaaaataaggctagtccgttatcaacttgaaaaagtggcaccgagtcggtgctttt

SEQ ID NO: 647 - RNA, 100 nt, synthetic construct
  source 1..100: mol_type = other RNA
  ncRNA 1..100: ncRNA_class = sgRNA
gtctttgactctaaggcccagttttagagctagaaatagcaagttaaaataaggctagtccgttatcaacttgaaaaagtggcaccgagtcggtgctttt

SEQ ID NO: 648 - RNA, 100 nt, synthetic construct
  source 1..100: mol_type = other RNA
  ncRNA 1..100: ncRNA_class = sgRNA
tctttgactctaaggcccaagttttagagctagaaatagcaagttaaaataaggctagtccgttatcaacttgaaaaagtggcaccgagtcggtgctttt

SEQ ID NO: 649 - RNA, 100 nt, synthetic construct
  source 1..100: mol_type = other RNA
  ncRNA 1..100: ncRNA_class = sgRNA
tttgactctaaggcccaagggttttagagctagaaatagcaagttaaaataaggctagtccgttatcaacttgaaaaagtggcaccgagtcggtgctttt

SEQ ID NO: 650 - RNA, 100 nt, synthetic construct
  source 1..100: mol_type = other RNA
  ncRNA 1..100: ncRNA_class = sgRNA
taaggcccaagggggcaagcgttttagagctagaaatagcaagttaaaataaggctagtccgttatcaacttgaaaaagtggcaccgagtcggtgctttt

SEQ ID NO: 651 - RNA, 100 nt, synthetic construct
  source 1..100: mol_type = other RNA
  ncRNA 1..100: ncRNA_class = sgRNA
aagggggcaagctggtctgcgttttagagctagaaatagcaagttaaaataaggctagtccgttatcaacttgaaaaagtggcaccgagtcggtgctttt

SEQ ID NO: 652 - RNA, 100 nt, synthetic construct
  source 1..100: mol_type = other RNA
  ncRNA 1..100: ncRNA_class = sgRNA
ggcagaccagcttgccccctgttttagagctagaaatagcaagttaaaataaggctagtccgttatcaacttgaaaaagtggcaccgagtcggtgctttt

SEQ ID NO: 653 - RNA, 100 nt, synthetic construct
  source 1..100: mol_type = other RNA
  ncRNA 1..100: ncRNA_class = sgRNA
agggggcaagctggtctgccgttttagagctagaaatagcaagttaaaataaggctagtccgttatcaacttgaaaaagtggcaccgagtcggtgctttt

SEQ ID NO: 654 - RNA, 100 nt, synthetic construct
  source 1..100: mol_type = other RNA
  ncRNA 1..100: ncRNA_class = sgRNA
ccccaaaagcgttgtgggccgttttagagctagaaatagcaagttaaaataaggctagtccgttatcaacttgaaaaagtggcaccgagtcggtgctttt

SEQ ID NO: 655 - RNA, 100 nt, synthetic construct
  source 1..100: mol_type = other RNA
  ncRNA 1..100: ncRNA_class = sgRNA
cacaacgcttttgggggtgagttttagagctagaaatagcaagttaaaataaggctagtccgttatcaacttgaaaaagtggcaccgagtcggtgctttt

SEQ ID NO: 656 - RNA, 100 nt, synthetic construct
  source 1..100: mol_type = other RNA
  ncRNA 1..100: ncRNA_class = sgRNA
cctcacccccaaaagcgttggttttagagctagaaatagcaagttaaaataaggctagtccgttatcaacttgaaaaagtggcaccgagtcggtgctttt

SEQ ID NO: 657 - RNA, 100 nt, synthetic construct
  source 1..100: mol_type = other RNA
  ncRNA 1..100: ncRNA_class = sgRNA
aacctctcccctggccctcagttttagagctagaaatagcaagttaaaataaggctagtccgttatcaacttgaaaaagtggcaccgagtcggtgctttt

SEQ ID NO: 658 - RNA, 100 nt, synthetic construct
  source 1..100: mol_type = other RNA
  ncRNA 1..100: ncRNA_class = sgRNA
acctctcccctggccctcatgttttagagctagaaatagcaagttaaaataaggctagtccgttatcaacttgaaaaagtggcaccgagtcggtgctttt

SEQ ID NO: 659 - RNA, 100 nt, synthetic construct
  source 1..100: mol_type = other RNA
  ncRNA 1..100: ncRNA_class = sgRNA
acggtgcccatgagggccaggttttagagctagaaatagcaagttaaaataaggctagtccgttatcaacttgaaaaagtggcaccgagtcggtgctttt

SEQ ID NO: 660 - RNA, 100 nt, synthetic construct
  source 1..100: mol_type = other RNA
  ncRNA 1..100: ncRNA_class = sgRNA
gacggtgcccatgagggccagttttagagctagaaatagcaagttaaaataaggctagtccgttatcaacttgaaaaagtggcaccgagtcggtgctttt

SEQ ID NO: 661 - RNA, 100 nt, synthetic construct
  source 1..100: mol_type = other RNA
  ncRNA 1..100: ncRNA_class = sgRNA
tgacggtgcccatgagggccgttttagagctagaaatagcaagttaaaataaggctagtccgttatcaacttgaaaaagtggcaccgagtcggtgctttt

SEQ ID NO: 662 - RNA, 100 nt, synthetic construct
  source 1..100: mol_type = other RNA
  ncRNA 1..100: ncRNA_class = sgRNA
tcatgggcaccgtcagctccgttttagagctagaaatagcaagttaaaataaggctagtccgttatcaacttgaaaaagtggcaccgagtcggtgctttt

SEQ ID NO: 663 - RNA, 100 nt, synthetic construct
  source 1..100: mol_type = other RNA
  ncRNA 1..100: ncRNA_class = sgRNA
ggagctgacggtgcccatgagttttagagctagaaatagcaagttaaaataaggctagtccgttatcaacttgaaaaagtggcaccgagtcggtgctttt

SEQ ID NO: 664 - RNA, 100 nt, synthetic construct
  source 1..100: mol_type = other RNA
  ncRNA 1..100: ncRNA_class = sgRNA
tggagctgacggtgcccatggttttagagctagaaatagcaagttaaaataaggctagtccgttatcaacttgaaaaagtggcaccgagtcggtgctttt

SEQ ID NO: 665 - RNA, 100 nt, synthetic construct
  source 1..100: mol_type = other RNA
  ncRNA 1..100: ncRNA_class = sgRNA
tgggcaccgtcagctccagggttttagagctagaaatagcaagttaaaataaggctagtccgttatcaacttgaaaaagtggcaccgagtcggtgctttt

SEQ ID NO: 666 - RNA, 100 nt, synthetic construct
  source 1..100: mol_type = other RNA
  ncRNA 1..100: ncRNA_class = sgRNA
ccgtcagctccaggcggtccgttttagagctagaaatagcaagttaaaataaggctagtccgttatcaacttgaaaaagtggcaccgagtcggtgctttt

SEQ ID NO: 667 - RNA, 100 nt, synthetic construct
  source 1..100: mol_type = other RNA
  ncRNA 1..100: ncRNA_class = sgRNA
ccaggaccgcctggagctgagttttagagctagaaatagcaagttaaaataaggctagtccgttatcaacttgaaaaagtggcaccgagtcggtgctttt

SEQ ID NO: 668 - RNA, 100 nt, synthetic construct
  source 1..100: mol_type = other RNA
  ncRNA 1..100: ncRNA_class = sgRNA
tcagctccaggcggtcctgggttttagagctagaaatagcaagttaaaataaggctagtccgttatcaacttgaaaaagtggcaccgagtcggtgctttt

SEQ ID NO: 669 - RNA, 100 nt, synthetic construct
  source 1..100: mol_type = other RNA
  ncRNA 1..100: ncRNA_class = sgRNA
cagcggccaccaggaccgccgttttagagctagaaatagcaagttaaaataaggctagtccgttatcaacttgaaaaagtggcaccgagtcggtgctttt

SEQ ID NO: 670 - RNA, 100 nt, synthetic construct
  source 1..100: mol_type = other RNA
  ncRNA 1..100: ncRNA_class = sgRNA
cagcagtggcagcggccaccgttttagagctagaaatagcaagttaaaataaggctagtccgttatcaacttgaaaaagtggcaccgagtcggtgctttt

SEQ ID NO: 671 - RNA, 100 nt, synthetic construct
  source 1..100: mol_type = other RNA
  ncRNA 1..100: ncRNA_class = sgRNA
gctgctgctcctgggtcccggttttagagctagaaatagcaagttaaaataaggctagtccgttatcaacttgaaaaagtggcaccgagtcggtgctttt

SEQ ID NO: 672 - RNA, 100 nt, synthetic construct
  source 1..100: mol_type = other RNA
  ncRNA 1..100: ncRNA_class = sgRNA
ctgctgctcctgggtcccgcgttttagagctagaaatagcaagttaaaataaggctagtccgttatcaacttgaaaaagtggcaccgagtcggtgctttt

SEQ ID NO: 673 - RNA, 100 nt, synthetic construct
  source 1..100: mol_type = other RNA
  ncRNA 1..100: ncRNA_class = sgRNA
cacgggcgcccgcgggacccgttttagagctagaaatagcaagttaaaataaggctagtccgttatcaacttgaaaaagtggcaccgagtcggtgctttt

SEQ ID NO: 674 - RNA, 100 nt, synthetic construct
  source 1..100: mol_type = other RNA
  ncRNA 1..100: ncRNA_class = sgRNA
tcccgcgggcgcccgtgcgcgttttagagctagaaatagcaagttaaaataaggctagtccgttatcaacttgaaaaagtggcaccgagtcggtgctttt

SEQ ID NO: 675 - RNA, 100 nt, synthetic construct
  source 1..100: mol_type = other RNA
  ncRNA 1..100: ncRNA_class = sgRNA
cgcgggcgcccgtgcgcagggttttagagctagaaatagcaagttaaaataaggctagtccgttatcaacttgaaaaagtggcaccgagtcggtgctttt

SEQ ID NO: 676 - RNA, 100 nt, synthetic construct
  source 1..100: mol_type = other RNA
  ncRNA 1..100: ncRNA_class = sgRNA
tcctgcgcacgggcgcccgcgttttagagctagaaatagcaagttaaaataaggctagtccgttatcaacttgaaaaagtggcaccgagtcggtgctttt

SEQ ID NO: 677 - RNA, 100 nt, synthetic construct
  source 1..100: mol_type = other RNA
  ncRNA 1..100: ncRNA_class = sgRNA
ctcctgcgcacgggcgcccggttttagagctagaaatagcaagttaaaataaggctagtccgttatcaacttgaaaaagtggcaccgagtcggtgctttt

SEQ ID NO: 678 - RNA, 100 nt, synthetic construct
  source 1..100: mol_type = other RNA
  ncRNA 1..100: ncRNA_class = sgRNA
cgcccgtgcgcaggaggacggttttagagctagaaatagcaagttaaaataaggctagtccgttatcaacttgaaaaagtggcaccgagtcggtgctttt

SEQ ID NO: 679 - RNA, 100 nt, synthetic construct
  source 1..100: mol_type = other RNA
  ncRNA 1..100: ncRNA_class = sgRNA
cgtgcgcaggaggacgaggagttttagagctagaaatagcaagttaaaataaggctagtccgttatcaacttgaaaaagtggcaccgagtcggtgctttt

SEQ ID NO: 680 - RNA, 100 nt, synthetic construct
  source 1..100: mol_type = other RNA
  ncRNA 1..100: ncRNA_class = sgRNA
gtcctcgtcctcctgcgcacgttttagagctagaaatagcaagttaaaataaggctagtccgttatcaacttgaaaaagtggcaccgagtcggtgctttt

SEQ ID NO: 681 - RNA, 100 nt, synthetic construct
  source 1..100: mol_type = other RNA
  ncRNA 1..100: ncRNA_class = sgRNA
cgtcctcgtcctcctgcgcagttttagagctagaaatagcaagttaaaataaggctagtccgttatcaacttgaaaaagtggcaccgagtcggtgctttt

SEQ ID NO: 682 - RNA, 100 nt, synthetic construct
  source 1..100: mol_type = other RNA
  ncRNA 1..100: ncRNA_class = sgRNA
ggacgaggacggcgactacggttttagagctagaaatagcaagttaaaataaggctagtccgttatcaacttgaaaaagtggcaccgagtcggtgctttt

SEQ ID NO: 683 - RNA, 100 nt, synthetic construct
  source 1..100: mol_type = other RNA
  ncRNA 1..100: ncRNA_class = sgRNA
ggacggcgactacgaggagcgttttagagctagaaatagcaagttaaaataaggctagtccgttatcaacttgaaaaagtggcaccgagtcggtgctttt

SEQ ID NO: 684 - RNA, 100 nt, synthetic construct
  source 1..100: mol_type = other RNA
  ncRNA 1..100: ncRNA_class = sgRNA
ggtgctagccttgcgttccggttttagagctagaaatagcaagttaaaataaggctagtccgttatcaacttgaaaaagtggcaccgagtcggtgctttt

SEQ ID NO: 685 - RNA, 100 nt, synthetic construct
  source 1..100: mol_type = other RNA
  ncRNA 1..100: ncRNA_class = sgRNA
gctagccttgcgttccgagggttttagagctagaaatagcaagttaaaataaggctagtccgttatcaacttgaaaaagtggcaccgagtcggtgctttt

SEQ ID NO: 686 - RNA, 100 nt, synthetic construct
  source 1..100: mol_type = other RNA
  ncRNA 1..100: ncRNA_class = sgRNA
gccttgcgttccgaggaggagttttagagctagaaatagcaagttaaaataaggctagtccgttatcaacttgaaaaagtggcaccgagtcggtgctttt

SEQ ID NO: 687 - RNA, 100 nt, synthetic construct
  source 1..100: mol_type = other RNA
  ncRNA 1..100: ncRNA_class = sgRNA
gccgtcctcctcggaacgcagttttagagctagaaatagcaagttaaaataaggctagtccgttatcaacttgaaaaagtggcaccgagtcggtgctttt

SEQ ID NO: 688 - RNA, 100 nt, synthetic construct
  source 1..100: mol_type = other RNA
  ncRNA 1..100: ncRNA_class = sgRNA
gcgttccgaggaggacggccgttttagagctagaaatagcaagttaaaataaggctagtccgttatcaacttgaaaaagtggcaccgagtcggtgctttt

SEQ ID NO: 689 - RNA, 100 nt, synthetic construct
  source 1..100: mol_type = other RNA
  ncRNA 1..100: ncRNA_class = sgRNA
ttcggccaggccgtcctcctgttttagagctagaaatagcaagttaaaataaggctagtccgttatcaacttgaaaaagtggcaccgagtcggtgctttt

SEQ ID NO: 690 - RNA, 100 nt, synthetic construct
  source 1..100: mol_type = other RNA
  ncRNA 1..100: ncRNA_class = sgRNA
ctggccgaagcacccgagcagttttagagctagaaatagcaagttaaaataaggctagtccgttatcaacttgaaaaagtggcaccgagtcggtgctttt

SEQ ID NO: 691 - RNA, 100 nt, synthetic construct
  source 1..100: mol_type = other RNA
  ncRNA 1..100: ncRNA_class = sgRNA
cgtgctcgggtgcttcggccgttttagagctagaaatagcaagttaaaataaggctagtccgttatcaacttgaaaaagtggcaccgagtcggtgctttt

SEQ ID NO: 692 - RNA, 100 nt, synthetic construct
  source 1..100: mol_type = other RNA
  ncRNA 1..100: ncRNA_class = sgRNA
ggttccgtgctcgggtgcttgttttagagctagaaatagcaagttaaaataaggctagtccgttatcaacttgaaaaagtggcaccgagtcggtgctttt

SEQ ID NO: 693 - RNA, 100 nt, synthetic construct
  source 1..100: mol_type = other RNA
  ncRNA 1..100: ncRNA_class = sgRNA
gtggctgtggttccgtgctcgttttagagctagaaatagcaagttaaaataaggctagtccgttatcaacttgaaaaagtggcaccgagtcggtgctttt

SEQ ID NO: 694 - RNA, 100 nt, synthetic construct
  source 1..100: mol_type = other RNA
  ncRNA 1..100: ncRNA_class = sgRNA
ggtggctgtggttccgtgctgttttagagctagaaatagcaagttaaaataaggctagtccgttatcaacttgaaaaagtggcaccgagtcggtgctttt

SEQ ID NO: 695 - RNA, 100 nt, synthetic construct
  source 1..100: mol_type = other RNA
  ncRNA 1..100: ncRNA_class = sgRNA
caccttccaccgctgcgccagttttagagctagaaatagcaagttaaaataaggctagtccgttatcaacttgaaaaagtggcaccgagtcggtgctttt

SEQ ID NO: 696 - RNA, 100 nt, synthetic construct
  source 1..100: mol_type = other RNA
  ncRNA 1..100: ncRNA_class = sgRNA
cttggcgcagcggtggaagggttttagagctagaaatagcaagttaaaataaggctagtccgttatcaacttgaaaaagtggcaccgagtcggtgctttt

SEQ ID NO: 697 - RNA, 100 nt, synthetic construct
  source 1..100: mol_type = other RNA
  ncRNA 1..100: ncRNA_class = sgRNA
tccaccgctgcgccaaggtggttttagagctagaaatagcaagttaaaataaggctagtccgttatcaacttgaaaaagtggcaccgagtcggtgctttt

SEQ ID NO: 698 - RNA, 100 nt, synthetic construct
  source 1..100: mol_type = other RNA
  ncRNA 1..100: ncRNA_class = sgRNA
caccttggcgcagcggtggagttttagagctagaaatagcaagttaaaataaggctagtccgttatcaacttgaaaaagtggcaccgagtcggtgctttt

SEQ ID NO: 699 - RNA, 100 nt, synthetic construct
  source 1..100: mol_type = other RNA
  ncRNA 1..100: ncRNA_class = sgRNA
ccaccgctgcgccaaggtgcgttttagagctagaaatagcaagttaaaataaggctagtccgttatcaacttgaaaaagtggcaccgagtcggtgctttt

SEQ ID NO: 700 - RNA, 100 nt, synthetic construct
  source 1..100: mol_type = other RNA
  ncRNA 1..100: ncRNA_class = sgRNA
cccgcaccttggcgcagcgggttttagagctagaaatagcaagttaaaataaggctagtccgttatcaacttgaaaaagtggcaccgagtcggtgctttt

SEQ ID NO: 701 - RNA, 100 nt, synthetic construct
  source 1..100: mol_type = other RNA
  ncRNA 1..100: ncRNA_class = sgRNA
acacccgcaccttggcgcaggttttagagctagaaatagcaagttaaaataaggctagtccgttatcaacttgaaaaagtggcaccgagtcggtgctttt

SEQ ID NO: 702 - RNA, 100 nt, synthetic construct
  source 1..100: mol_type = other RNA
  ncRNA 1..100: ncRNA_class = sgRNA
ctgcgccaaggtgcgggtgtgttttagagctagaaatagcaagttaaaataaggctagtccgttatcaacttgaaaaagtggcaccgagtcggtgctttt

SEQ ID NO: 703 - RNA, 100 nt, synthetic construct
  source 1..100: mol_type = other RNA
  ncRNA 1..100: ncRNA_class = sgRNA
tgcgccaaggtgcgggtgtagttttagagctagaaatagcaagttaaaataaggctagtccgttatcaacttgaaaaagtggcaccgagtcggtgctttt

SEQ ID NO: 704 - RNA, 100 nt, synthetic construct
  source 1..100: mol_type = other RNA
  ncRNA 1..100: ncRNA_class = sgRNA
ccaaggtgcgggtgtagggagttttagagctagaaatagcaagttaaaataaggctagtccgttatcaacttgaaaaagtggcaccgagtcggtgctttt

SEQ ID NO: 705 - RNA, 100 nt, synthetic construct
  source 1..100: mol_type = other RNA
  ncRNA 1..100: ncRNA_class = sgRNA
caaggtgcgggtgtagggatgttttagagctagaaatagcaagttaaaataaggctagtccgttatcaacttgaaaaagtggcaccgagtcggtgctttt

SEQ ID NO: 706 - RNA, 100 nt, synthetic construct
  source 1..100: mol_type = other RNA
  ncRNA 1..100: ncRNA_class = sgRNA
ccatccctacacccgcacctgttttagagctagaaatagcaagttaaaataaggctagtccgttatcaacttgaaaaagtggcaccgagtcggtgctttt

SEQ ID NO: 707 - RNA, 100 nt, synthetic construct
  source 1..100: mol_type = other RNA
  ncRNA 1..100: ncRNA_class = sgRNA
gatcctggccccatgcaagggttttagagctagaaatagcaagttaaaataaggctagtccgttatcaacttgaaaaagtggcaccgagtcggtgctttt

SEQ ID NO: 708 - RNA, 100 nt, synthetic construct
  source 1..100: mol_type = other RNA
  ncRNA 1..100: ncRNA_class = sgRNA
ttgcatggggccaggatccggttttagagctagaaatagcaagttaaaataaggctagtccgttatcaacttgaaaaagtggcaccgagtcggtgctttt

SEQ ID NO: 709 - RNA, 100 nt, synthetic construct
  source 1..100: mol_type = other RNA
  ncRNA 1..100: ncRNA_class = sgRNA
acggatcctggccccatgcagttttagagctagaaatagcaagttaaaataaggctagtccgttatcaacttgaaaaagtggcaccgagtcggtgctttt

SEQ ID NO: 710 - RNA, 100 nt, synthetic construct
  source 1..100: mol_type = other RNA
  ncRNA 1..100: ncRNA_class = sgRNA
catggggccaggatccgtgggttttagagctagaaatagcaagttaaaataaggctagtccgttatcaacttgaaaaagtggcaccgagtcggtgctttt

SEQ ID NO: 711 - RNA, 100 nt, synthetic construct
  source 1..100: mol_type = other RNA
  ncRNA 1..100: ncRNA_class = sgRNA
caggatccgtggaggttgccgttttagagctagaaatagcaagttaaaataaggctagtccgttatcaacttgaaaaagtggcaccgagtcggtgctttt

SEQ ID NO: 712 - RNA, 100 nt, synthetic construct
  source 1..100: mol_type = other RNA
  ncRNA 1..100: ncRNA_class = sgRNA
caggcaacctccacggatccgttttagagctagaaatagcaagttaaaataaggctagtccgttatcaacttgaaaaagtggcaccgagtcggtgctttt

SEQ ID NO: 713 - RNA, 100 nt, synthetic construct
  source 1..100: mol_type = other RNA
  ncRNA 1..100: ncRNA_class = sgRNA
taggtgccaggcaacctccagttttagagctagaaatagcaagttaaaataaggctagtccgttatcaacttgaaaaagtggcaccgagtcggtgctttt

SEQ ID NO: 714 - RNA, 100 nt, synthetic construct
  source 1..100: mol_type = other RNA
  ncRNA 1..100: ncRNA_class = sgRNA
gaggttgcctggcacctacggttttagagctagaaatagcaagttaaaataaggctagtccgttatcaacttgaaaaagtggcaccgagtcggtgctttt

SEQ ID NO: 715 - RNA, 100 nt, synthetic construct
  source 1..100: mol_type = other RNA
  ncRNA 1..100: ncRNA_class = sgRNA
gttgcctggcacctacgtgggttttagagctagaaatagcaagttaaaataaggctagtccgttatcaacttgaaaaagtggcaccgagtcggtgctttt

SEQ ID NO: 716 - RNA, 100 nt, synthetic construct
  source 1..100: mol_type = other RNA
  ncRNA 1..100: ncRNA_class = sgRNA
agcaccaccacgtaggtgccgttttagagctagaaatagcaagttaaaataaggctagtccgttatcaacttgaaaaagtggcaccgagtcggtgctttt

SEQ ID NO: 717 - RNA, 100 nt, synthetic construct
  source 1..100: mol_type = other RNA
  ncRNA 1..100: ncRNA_class = sgRNA
cacctacgtggtggtgctgagttttagagctagaaatagcaagttaaaataaggctagtccgttatcaacttgaaaaagtggcaccgagtcggtgctttt

SEQ ID NO: 718 - RNA, 100 nt, synthetic construct
  source 1..100: mol_type = other RNA
  ncRNA 1..100: ncRNA_class = sgRNA
ctacgtggtggtgctgaagggttttagagctagaaatagcaagttaaaataaggctagtccgttatcaacttgaaaaagtggcaccgagtcggtgctttt

SEQ ID NO: 719 - RNA, 100 nt, synthetic construct
  source 1..100: mol_type = other RNA
  ncRNA 1..100: ncRNA_class = sgRNA
ctccttcagcaccaccacgtgttttagagctagaaatagcaagttaaaataaggctagtccgttatcaacttgaaaaagtggcaccgagtcggtgctttt

SEQ ID NO: 720 - RNA, 100 nt, synthetic construct
  source 1..100: mol_type = other RNA
  ncRNA 1..100: ncRNA_class = sgRNA
gcgctctgactgcgagaggtgttttagagctagaaatagcaagttaaaataaggctagtccgttatcaacttgaaaaagtggcaccgagtcggtgctttt

SEQ ID NO: 721 - RNA, 100 nt, synthetic construct
  source 1..100: mol_type = other RNA
  ncRNA 1..100: ncRNA_class = sgRNA
tgcgctctgactgcgagagggttttagagctagaaatagcaagttaaaataaggctagtccgttatcaacttgaaaaagtggcaccgagtcggtgctttt

SEQ ID NO: 722 - RNA, 100 nt, synthetic construct
  source 1..100: mol_type = other RNA
  ncRNA 1..100: ncRNA_class = sgRNA
cagtgcgctctgactgcgaggttttagagctagaaatagcaagttaaaataaggctagtccgttatcaacttgaaaaagtggcaccgagtcggtgctttt

SEQ ID NO: 723 - RNA, 100 nt, synthetic construct
  source 1..100: mol_type = other RNA
  ncRNA 1..100: ncRNA_class = sgRNA
gcgcactgcccgccgcctgcgttttagagctagaaatagcaagttaaaataaggctagtccgttatcaacttgaaaaagtggcaccgagtcggtgctttt

SEQ ID NO: 724 - RNA, 100 nt, synthetic construct
  source 1..100: mol_type = other RNA
  ncRNA 1..100: ncRNA_class = sgRNA
tgcccgccgcctgcaggcccgttttagagctagaaatagcaagttaaaataaggctagtccgttatcaacttgaaaaagtggcaccgagtcggtgctttt

SEQ ID NO: 725 - RNA, 100 nt, synthetic construct
  source 1..100: mol_type = other RNA
  ncRNA 1..100: ncRNA_class = sgRNA
tgcaggcccaggctgcccgcgttttagagctagaaatagcaagttaaaataaggctagtccgttatcaacttgaaaaagtggcaccgagtcggtgctttt

SEQ ID NO: 726 - RNA, 100 nt, synthetic construct
  source 1..100: mol_type = other RNA
  ncRNA 1..100: ncRNA_class = sgRNA
caggcccaggctgcccgccggttttagagctagaaatagcaagttaaaataaggctagtccgttatcaacttgaaaaagtggcaccgagtcggtgctttt

SEQ ID NO: 727 - RNA, 100 nt, synthetic construct
  source 1..100: mol_type = other RNA
  ncRNA 1..100: ncRNA_class = sgRNA
gtatccccggcgggcagcctgttttagagctagaaatagcaagttaaaataaggctagtccgttatcaacttgaaaaagtggcaccgagtcggtgctttt

SEQ ID NO: 728 - RNA, 100 nt, synthetic construct
  source 1..100: mol_type = other RNA
  ncRNA 1..100: ncRNA_class = sgRNA
ggtatccccggcgggcagccgttttagagctagaaatagcaagttaaaataaggctagtccgttatcaacttgaaaaagtggcaccgagtcggtgctttt

SEQ ID NO: 729 - RNA, 100 nt, synthetic construct
  source 1..100: mol_type = other RNA
  ncRNA 1..100: ncRNA_class = sgRNA
cttggtgaggtatccccggcgttttagagctagaaatagcaagttaaaataaggctagtccgttatcaacttgaaaaagtggcaccgagtcggtgctttt

SEQ ID NO: 730 - RNA, 100 nt, synthetic construct
  source 1..100: mol_type = other RNA
  ncRNA 1..100: ncRNA_class = sgRNA
tcttggtgaggtatccccgggttttagagctagaaatagcaagttaaaataaggctagtccgttatcaacttgaaaaagtggcaccgagtcggtgctttt

SEQ ID NO: 731 - RNA, 100 nt, synthetic construct
  source 1..100: mol_type = other RNA
  ncRNA 1..100: ncRNA_class = sgRNA
ggatcttggtgaggtatcccgttttagagctagaaatagcaagttaaaataaggctagtccgttatcaacttgaaaaagtggcaccgagtcggtgctttt

SEQ ID NO: 732 - RNA, 100 nt, synthetic construct
  source 1..100: mol_type = other RNA
  ncRNA 1..100: ncRNA_class = sgRNA
agacatgcaggatcttggtggttttagagctagaaatagcaagttaaaataaggctagtccgttatcaacttgaaaaagtggcaccgagtcggtgctttt

SEQ ID NO: 733 - RNA, 100 nt, synthetic construct
  source 1..100: mol_type = other RNA
  ncRNA 1..100: ncRNA_class = sgRNA
aagatcctgcatgtcttccagttttagagctagaaatagcaagttaaaataaggctagtccgttatcaacttgaaaaagtggcaccgagtcggtgctttt

SEQ ID NO: 734 - RNA, 100 nt, synthetic construct
  source 1..100: mol_type = other RNA
  ncRNA 1..100: ncRNA_class = sgRNA
atggaagacatgcaggatctgttttagagctagaaatagcaagttaaaataaggctagtccgttatcaacttgaaaaagtggcaccgagtcggtgctttt

SEQ ID NO: 735 - RNA, 100 nt, synthetic construct
  source 1..100: mol_type = other RNA
  ncRNA 1..100: ncRNA_class = sgRNA
gaaggccatggaagacatgcgttttagagctagaaatagcaagttaaaataaggctagtccgttatcaacttgaaaaagtggcaccgagtcggtgctttt

SEQ ID NO: 736 - RNA, 100 nt, synthetic construct
  source 1..100: mol_type = other RNA
  ncRNA 1..100: ncRNA_class = sgRNA
gtcttccatggccttcttccgttttagagctagaaatagcaagttaaaataaggctagtccgttatcaacttgaaaaagtggcaccgagtcggtgctttt

SEQ ID NO: 737 - RNA, 100 nt, synthetic construct
  source 1..100: mol_type = other RNA
  ncRNA 1..100: ncRNA_class = sgRNA
ctcatcttcaccaggaagccgttttagagctagaaatagcaagttaaaataaggctagtccgttatcaacttgaaaaagtggcaccgagtcggtgctttt

SEQ ID NO: 738 - RNA, 100 nt, synthetic construct
  source 1..100: mol_type = other RNA
  ncRNA 1..100: ncRNA_class = sgRNA
ggcttcctggtgaagatgaggttttagagctagaaatagcaagttaaaataaggctagtccgttatcaacttgaaaaagtggcaccgagtcggtgctttt

SEQ ID NO: 739 - RNA, 100 nt, synthetic construct
  source 1..100: mol_type = other RNA
  ncRNA 1..100: ncRNA_class = sgRNA
ggtcgccactcatcttcaccgttttagagctagaaatagcaagttaaaataaggctagtccgttatcaacttgaaaaagtggcaccgagtcggtgctttt

SEQ ID NO: 740 - RNA, 100 nt, synthetic construct
  source 1..100: mol_type = other RNA
  ncRNA 1..100: ncRNA_class = sgRNA
gaagatgagtggcgacctgcgttttagagctagaaatagcaagttaaaataaggctagtccgttatcaacttgaaaaagtggcaccgagtcggtgctttt

SEQ ID NO: 741 - RNA, 100 nt, synthetic construct
  source 1..100: mol_type = other RNA
  ncRNA 1..100: ncRNA_class = sgRNA
gagtggcgacctgctggagcgttttagagctagaaatagcaagttaaaataaggctagtccgttatcaacttgaaaaagtggcaccgagtcggtgctttt

SEQ ID NO: 742 - RNA, 100 nt, synthetic construct
  source 1..100: mol_type = other RNA
  ncRNA 1..100: ncRNA_class = sgRNA
ggtggctcaccagctccagcgttttagagctagaaatagcaagttaaaataaggctagtccgttatcaacttgaaaaagtggcaccgagtcggtgctttt

SEQ ID NO: 743 - RNA, 100 nt, synthetic construct
  source 1..100: mol_type = other RNA
  ncRNA 1..100: ncRNA_class = sgRNA
agctggtgagccacccttttgttttagagctagaaatagcaagttaaaataaggctagtccgttatcaacttgaaaaagtggcaccgagtcggtgctttt

SEQ ID NO: 744 - RNA, 100 nt, synthetic construct
  source 1..100: mol_type = other RNA
  ncRNA 1..100: ncRNA_class = sgRNA
gctggtgagccaccctttttgttttagagctagaaatagcaagttaaaataaggctagtccgttatcaacttgaaaaagtggcaccgagtcggtgctttt

SEQ ID NO: 745 - RNA, 100 nt, synthetic construct
  source 1..100: mol_type = other RNA
  ncRNA 1..100: ncRNA_class = sgRNA
aaggcctgcagaagccagaggttttagagctagaaatagcaagttaaaataaggctagtccgttatcaacttgaaaaagtggcaccgagtcggtgctttt

SEQ ID NO: 746 - RNA, 100 nt, synthetic construct
  source 1..100: mol_type = other RNA
  ncRNA 1..100: ncRNA_class = sgRNA
gtcgacatggggcaacttcagttttagagctagaaatagcaagttaaaataaggctagtccgttatcaacttgaaaaagtggcaccgagtcggtgctttt

SEQ ID NO: 747 - RNA, 100 nt, synthetic construct
  source 1..100: mol_type = other RNA
  ncRNA 1..100: ncRNA_class = sgRNA
gccccatgtcgactacatcggttttagagctagaaatagcaagttaaaataaggctagtccgttatcaacttgaaaaagtggcaccgagtcggtgctttt

SEQ ID NO: 748 - RNA, 100 nt, synthetic construct
  source 1..100: mol_type = other RNA
  ncRNA 1..100: ncRNA_class = sgRNA
ccatgtcgactacatcgagggttttagagctagaaatagcaagttaaaataaggctagtccgttatcaacttgaaaaagtggcaccgagtcggtgctttt

SEQ ID NO: 749 - RNA, 100 nt, synthetic construct
  source 1..100: mol_type = other RNA
  ncRNA 1..100: ncRNA_class = sgRNA
tcctcgatgtagtcgacatggttttagagctagaaatagcaagttaaaataaggctagtccgttatcaacttgaaaaagtggcaccgagtcggtgctttt

SEQ ID NO: 750 - RNA, 100 nt, synthetic construct
  source 1..100: mol_type = other RNA
  ncRNA 1..100: ncRNA_class = sgRNA
ctcctcgatgtagtcgacatgttttagagctagaaatagcaagttaaaataaggctagtccgttatcaacttgaaaaagtggcaccgagtcggtgctttt

SEQ ID NO: 751 - RNA, 100 nt, synthetic construct
  source 1..100: mol_type = other RNA
  ncRNA 1..100: ncRNA_class = sgRNA
cctcctcgatgtagtcgacagttttagagctagaaatagcaagttaaaataaggctagtccgttatcaacttgaaaaagtggcaccgagtcggtgctttt

SEQ ID NO: 752 - RNA, 100 nt, synthetic construct
  source 1..100: mol_type = other RNA
  ncRNA 1..100: ncRNA_class = sgRNA
gatgctctgggcaaagacaggttttagagctagaaatagcaagttaaaataaggctagtccgttatcaacttgaaaaagtggcaccgagtcggtgctttt

SEQ ID NO: 753 - RNA, 100 nt, synthetic construct
  source 1..100: mol_type = other RNA
  ncRNA 1..100: ncRNA_class = sgRNA
tctttgcccagagcatcccggttttagagctagaaatagcaagttaaaataaggctagtccgttatcaacttgaaaaagtggcaccgagtcggtgctttt

SEQ ID NO: 754 - RNA, 100 nt, synthetic construct
  source 1..100: mol_type = other RNA
  ncRNA 1..100: ncRNA_class = sgRNA
ccagagcatcccgtggaaccgttttagagctagaaatagcaagttaaaataaggctagtccgttatcaacttgaaaaagtggcaccgagtcggtgctttt

SEQ ID NO: 755 - RNA, 100 nt, synthetic construct
  source 1..100: mol_type = other RNA
  ncRNA 1..100: ncRNA_class = sgRNA
caggttccacgggatgctctgttttagagctagaaatagcaagttaaaataaggctagtccgttatcaacttgaaaaagtggcaccgagtcggtgctttt

SEQ ID NO: 756 - RNA, 100 nt, synthetic construct
  source 1..100: mol_type = other RNA
  ncRNA 1..100: ncRNA_class = sgRNA
gcatcccgtggaacctggaggttttagagctagaaatagcaagttaaaataaggctagtccgttatcaacttgaaaaagtggcaccgagtcggtgctttt

SEQ ID NO: 757 - RNA, 100 nt, synthetic construct
  source 1..100: mol_type = other RNA
  ncRNA 1..100: ncRNA_class = sgRNA
gtaatccgctccaggttccagttttagagctagaaatagcaagttaaaataaggctagtccgttatcaacttgaaaaagtggcaccgagtcggtgctttt

SEQ ID NO: 758 - RNA, 100 nt, synthetic construct
  source 1..100: mol_type = other RNA
  ncRNA 1..100: ncRNA_class = sgRNA
tggagcggattacccctccagttttagagctagaaatagcaagttaaaataaggctagtccgttatcaacttgaaaaagtggcaccgagtcggtgctttt

SEQ ID NO: 759 - RNA, 100 nt, synthetic construct
  source 1..100: mol_type = other RNA
  ncRNA 1..100: ncRNA_class = sgRNA
gtggaggggtaatccgctccgttttagagctagaaatagcaagttaaaataaggctagtccgttatcaacttgaaaaagtggcaccgagtcggtgctttt

SEQ ID NO: 760 - RNA, 100 nt, synthetic construct
  source 1..100: mol_type = other RNA
  ncRNA 1..100: ncRNA_class = sgRNA
ggattacccctccacggtacgttttagagctagaaatagcaagttaaaataaggctagtccgttatcaacttgaaaaagtggcaccgagtcggtgctttt

SEQ ID NO: 761 - RNA, 100 nt, synthetic construct
  source 1..100: mol_type = other RNA
  ncRNA 1..100: ncRNA_class = sgRNA
gattacccctccacggtaccgttttagagctagaaatagcaagttaaaataaggctagtccgttatcaacttgaaaaagtggcaccgagtcggtgctttt

SEQ ID NO: 762 - RNA, 100 nt, synthetic construct
  source 1..100: mol_type = other RNA
  ncRNA 1..100: ncRNA_class = sgRNA
tacccctccacggtaccggggttttagagctagaaatagcaagttaaaataaggctagtccgttatcaacttgaaaaagtggcaccgagtcggtgctttt

SEQ ID NO: 763 - RNA, 100 nt, synthetic construct
  source 1..100: mol_type = other RNA
  ncRNA 1..100: ncRNA_class = sgRNA
atccgcccggtaccgtggaggttttagagctagaaatagcaagttaaaataaggctagtccgttatcaacttgaaaaagtggcaccgagtcggtgctttt

SEQ ID NO: 764 - RNA, 100 nt, synthetic construct
  source 1..100: mol_type = other RNA
  ncRNA 1..100: ncRNA_class = sgRNA
catccgcccggtaccgtggagttttagagctagaaatagcaagttaaaataaggctagtccgttatcaacttgaaaaagtggcaccgagtcggtgctttt

SEQ ID NO: 765 - RNA, 100 nt, synthetic construct
  source 1..100: mol_type = other RNA
  ncRNA 1..100: ncRNA_class = sgRNA
tcatccgcccggtaccgtgggttttagagctagaaatagcaagttaaaataaggctagtccgttatcaacttgaaaaagtggcaccgagtcggtgctttt

SEQ ID NO: 766 - RNA, 100 nt, synthetic construct
  source 1..100: mol_type = other RNA
  ncRNA 1..100: ncRNA_class = sgRNA
tattcatccgcccggtaccggttttagagctagaaatagcaagttaaaataaggctagtccgttatcaacttgaaaaagtggcaccgagtcggtgctttt

SEQ ID NO: 767 - RNA, 100 nt, synthetic construct
  source 1..100: mol_type = other RNA
  ncRNA 1..100: ncRNA_class = sgRNA
ggggctggtattcatccgccgttttagagctagaaatagcaagttaaaataaggctagtccgttatcaacttgaaaaagtggcaccgagtcggtgctttt

SEQ ID NO: 768 - RNA, 100 nt, synthetic construct
  source 1..100: mol_type = other RNA
  ncRNA 1..100: ncRNA_class = sgRNA
gcggatgaataccagcccccgttttagagctagaaatagcaagttaaaataaggctagtccgttatcaacttgaaaaagtggcaccgagtcggtgctttt

SEQ ID NO: 769 - RNA, 100 nt, synthetic construct
  source 1..100: mol_type = other RNA
  ncRNA 1..100: ncRNA_class = sgRNA
cagatgggggtcttaccggggttttagagctagaaatagcaagttaaaataaggctagtccgttatcaacttgaaaaagtggcaccgagtcggtgctttt

SEQ ID NO: 770 - RNA, 100 nt, synthetic construct
  source 1..100: mol_type = other RNA
  ncRNA 1..100: ncRNA_class = sgRNA
acagatgggggtcttaccgggttttagagctagaaatagcaagttaaaataaggctagtccgttatcaacttgaaaaagtggcaccgagtcggtgctttt

SEQ ID NO: 771 - RNA, 100 nt, synthetic construct
  source 1..100: mol_type = other RNA
  ncRNA 1..100: ncRNA_class = sgRNA
ctttccaaggcgacatttgtgttttagagctagaaatagcaagttaaaataaggctagtccgttatcaacttgaaaaagtggcaccgagtcggtgctttt

SEQ ID NO: 772 - RNA, 100 nt, synthetic construct
  source 1..100: mol_type = other RNA
  ncRNA 1..100: ncRNA_class = sgRNA
tctttccaaggcgacatttggttttagagctagaaatagcaagttaaaataaggctagtccgttatcaacttgaaaaagtggcaccgagtcggtgctttt

SEQ ID NO: 773 - RNA, 100 nt, synthetic construct
  source 1..100: mol_type = other RNA
  ncRNA 1..100: ncRNA_class = sgRNA
caaatgtcgccttggaaagagttttagagctagaaatagcaagttaaaataaggctagtccgttatcaacttgaaaaagtggcaccgagtcggtgctttt

SEQ ID NO: 774 - RNA, 100 nt, synthetic construct
  source 1..100: mol_type = other RNA
  ncRNA 1..100: ncRNA_class = sgRNA
atgtcgccttggaaagacgggttttagagctagaaatagcaagttaaaataaggctagtccgttatcaacttgaaaaagtggcaccgagtcggtgctttt

SEQ ID NO: 775 - RNA, 100 nt, synthetic construct
  source 1..100: mol_type = other RNA
  ncRNA 1..100: ncRNA_class = sgRNA
gaaagacggaggcagcctgggttttagagctagaaatagcaagttaaaataaggctagtccgttatcaacttgaaaaagtggcaccgagtcggtgctttt

SEQ ID NO: 776 - RNA, 100 nt, synthetic construct
  source 1..100: mol_type = other RNA
  ncRNA 1..100: ncRNA_class = sgRNA
ctaggagatacacctccaccgttttagagctagaaatagcaagttaaaataaggctagtccgttatcaacttgaaaaagtggcaccgagtcggtgctttt

SEQ ID NO: 777 - RNA, 100 nt, synthetic construct
  source 1..100: mol_type = other RNA
  ncRNA 1..100: ncRNA_class = sgRNA
cactctgtatgctggtgtctgttttagagctagaaatagcaagttaaaataaggctagtccgttatcaacttgaaaaagtggcaccgagtcggtgctttt

SEQ ID NO: 778 - RNA, 100 nt, synthetic construct
  source 1..100: mol_type = other RNA
  ncRNA 1..100: ncRNA_class = sgRNA
ccagcatacagagtgaccacgttttagagctagaaatagcaagttaaaataaggctagtccgttatcaacttgaaaaagtggcaccgagtcggtgctttt

SEQ ID NO: 779 - RNA, 100 nt, synthetic construct
  source 1..100: mol_type = other RNA
  ncRNA 1..100: ncRNA_class = sgRNA
gagtgaccaccgggaaatcggttttagagctagaaatagcaagttaaaataaggctagtccgttatcaacttgaaaaagtggcaccgagtcggtgctttt

SEQ ID NO: 780 - RNA, 100 nt, synthetic construct
  source 1..100: mol_type = other RNA
  ncRNA 1..100: ncRNA_class = sgRNA
agtgaccaccgggaaatcgagttttagagctagaaatagcaagttaaaataaggctagtccgttatcaacttgaaaaagtggcaccgagtcggtgctttt

SEQ ID NO: 781 - RNA, 100 nt, synthetic construct
  source 1..100: mol_type = other RNA
  ncRNA 1..100: ncRNA_class = sgRNA
accaccgggaaatcgagggcgttttagagctagaaatagcaagttaaaataaggctagtccgttatcaacttgaaaaagtggcaccgagtcggtgctttt

SEQ ID NO: 782 - RNA, 100 nt, synthetic construct
  source 1..100: mol_type = other RNA
  ncRNA 1..100: ncRNA_class = sgRNA
ccaccgggaaatcgagggcagttttagagctagaaatagcaagttaaaataaggctagtccgttatcaacttgaaaaagtggcaccgagtcggtgctttt

SEQ ID NO: 783 - RNA, 100 nt, synthetic construct
  source 1..100: mol_type = other RNA
  ncRNA 1..100: ncRNA_class = sgRNA
ccctgccctcgatttcccgggttttagagctagaaatagcaagttaaaataaggctagtccgttatcaacttgaaaaagtggcaccgagtcggtgctttt

SEQ ID NO: 784 - RNA, 100 nt, synthetic construct
  source 1..100: mol_type = other RNA
  ncRNA 1..100: ncRNA_class = sgRNA
tgaccctgccctcgatttccgttttagagctagaaatagcaagttaaaataaggctagtccgttatcaacttgaaaaagtggcaccgagtcggtgctttt

SEQ ID NO: 785 - RNA, 100 nt, synthetic construct
  source 1..100: mol_type = other RNA
  ncRNA 1..100: ncRNA_class = sgRNA
cgacttcgagaatgtgcccggttttagagctagaaatagcaagttaaaataaggctagtccgttatcaacttgaaaaagtggcaccgagtcggtgctttt

SEQ ID NO: 786 - RNA, 100 nt, synthetic construct
  source 1..100: mol_type = other RNA
  ncRNA 1..100: ncRNA_class = sgRNA
ctcgggcacattctcgaagtgttttagagctagaaatagcaagttaaaataaggctagtccgttatcaacttgaaaaagtggcaccgagtcggtgctttt

SEQ ID NO: 787 - RNA, 100 nt, synthetic construct
  source 1..100: mol_type = other RNA
  ncRNA 1..100: ncRNA_class = sgRNA
cttcgagaatgtgcccgagggttttagagctagaaatagcaagttaaaataaggctagtccgttatcaacttgaaaaagtggcaccgagtcggtgctttt

SEQ ID NO: 788 - RNA, 100 nt, synthetic construct
  source 1..100: mol_type = other RNA
  ncRNA 1..100: ncRNA_class = sgRNA
aagcgggtcccgtcctcctcgttttagagctagaaatagcaagttaaaataaggctagtccgttatcaacttgaaaaagtggcaccgagtcggtgctttt

SEQ ID NO: 789 - RNA, 100 nt, synthetic construct
  source 1..100: mol_type = other RNA
  ncRNA 1..100: ncRNA_class = sgRNA
gaagcgggtcccgtcctcctgttttagagctagaaatagcaagttaaaataaggctagtccgttatcaacttgaaaaagtggcaccgagtcggtgctttt

SEQ ID NO: 790 - RNA, 100 nt, synthetic construct
  source 1..100: mol_type = other RNA
  ncRNA 1..100: ncRNA_class = sgRNA
cgggacccgcttccacagacgttttagagctagaaatagcaagttaaaataaggctagtccgttatcaacttgaaaaagtggcaccgagtcggtgctttt

SEQ ID NO: 791 - RNA, 100 nt, synthetic construct
  source 1..100: mol_type = other RNA
  ncRNA 1..100: ncRNA_class = sgRNA
gcttacctgtctgtggaagcgttttagagctagaaatagcaagttaaaataaggctagtccgttatcaacttgaaaaagtggcaccgagtcggtgctttt

SEQ ID NO: 792 - RNA, 100 nt, synthetic construct
  source 1..100: mol_type = other RNA
  ncRNA 1..100: ncRNA_class = sgRNA
cggccgtgcttacctgtctggttttagagctagaaatagcaagttaaaataaggctagtccgttatcaacttgaaaaagtggcaccgagtcggtgctttt

SEQ ID NO: 793 - RNA, 100 nt, synthetic construct
  source 1..100: mol_type = other RNA
  ncRNA 1..100: ncRNA_class = sgRNA
aggtaagcacggccgtctgagttttagagctagaaatagcaagttaaaataaggctagtccgttatcaacttgaaaaagtggcaccgagtcggtgctttt

SEQ ID NO: 794 - RNA, 100 nt, synthetic construct
  source 1..100: mol_type = other RNA
  ncRNA 1..100: ncRNA_class = sgRNA
ggtaagcacggccgtctgatgttttagagctagaaatagcaagttaaaataaggctagtccgttatcaacttgaaaaagtggcaccgagtcggtgctttt

SEQ ID NO: 795 - RNA, 100 nt, synthetic construct
  source 1..100: mol_type = other RNA
  ncRNA 1..100: ncRNA_class = sgRNA
ggtccttgtgttcgtcgagcgttttagagctagaaatagcaagttaaaataaggctagtccgttatcaacttgaaaaagtggcaccgagtcggtgctttt

SEQ ID NO: 796 - RNA, 100 nt, synthetic construct
  source 1..100: mol_type = other RNA
  ncRNA 1..100: ncRNA_class = sgRNA
tggcctgctcgacgaacacagttttagagctagaaatagcaagttaaaataaggctagtccgttatcaacttgaaaaagtggcaccgagtcggtgctttt

SEQ ID NO: 797 - RNA, 100 nt, synthetic construct
  source 1..100: mol_type = other RNA
  ncRNA 1..100: ncRNA_class = sgRNA
gccagcaagtgtgacagtcagttttagagctagaaatagcaagttaaaataaggctagtccgttatcaacttgaaaaagtggcaccgagtcggtgctttt

SEQ ID NO: 798 - RNA, 100 nt, synthetic construct
  source 1..100: mol_type = other RNA
  ncRNA 1..100: ncRNA_class = sgRNA
gccatgactgtcacacttgcgttttagagctagaaatagcaagttaaaataaggctagtccgttatcaacttgaaaaagtggcaccgagtcggtgctttt

SEQ ID NO: 799 - RNA, 100 nt, synthetic construct
  source 1..100: mol_type = other RNA
  ncRNA 1..100: ncRNA_class = sgRNA
gctgcgcatgctggcaccctgttttagagctagaaatagcaagttaaaataaggctagtccgttatcaacttgaaaaagtggcaccgagtcggtgctttt

SEQ ID NO: 800 - RNA, 100 nt, synthetic construct
  source 1..100: mol_type = other RNA
  ncRNA 1..100: ncRNA_class = sgRNA
cacgcgcaggctgcgcatgcgttttagagctagaaatagcaagttaaaataaggctagtccgttatcaacttgaaaaagtggcaccgagtcggtgctttt

SEQ ID NO: 801 - RNA, 100 nt, synthetic construct
  source 1..100: mol_type = other RNA
  ncRNA 1..100: ncRNA_class = sgRNA
ctgcgcgtgctcaactgccagttttagagctagaaatagcaagttaaaataaggctagtccgttatcaacttgaaaaagtggcaccgagtcggtgctttt

SEQ ID NO: 802 - RNA, 100 nt, synthetic construct
  source 1..100: mol_type = other RNA
  ncRNA 1..100: ncRNA_class = sgRNA
tgcgcgtgctcaactgccaagttttagagctagaaatagcaagttaaaataaggctagtccgttatcaacttgaaaaagtggcaccgagtcggtgctttt

SEQ ID NO: 803 - RNA, 100 nt, synthetic construct
  source 1..100: mol_type = other RNA
  ncRNA 1..100: ncRNA_class = sgRNA
cttggcagttgagcacgcgcgttttagagctagaaatagcaagttaaaataaggctagtccgttatcaacttgaaaaagtggcaccgagtcggtgctttt

SEQ ID NO: 804 - RNA, 100 nt, synthetic construct
  source 1..100: mol_type = other RNA
  ncRNA 1..100: ncRNA_class = sgRNA
cgtgctcaactgccaagggagttttagagctagaaatagcaagttaaaataaggctagtccgttatcaacttgaaaaagtggcaccgagtcggtgctttt

SEQ ID NO: 805 - RNA, 100 nt, synthetic construct
  source 1..100: mol_type = other RNA
  ncRNA 1..100: ncRNA_class = sgRNA
gtgctcaactgccaagggaagttttagagctagaaatagcaagttaaaataaggctagtccgttatcaacttgaaaaagtggcaccgagtcggtgctttt

SEQ ID NO: 806 - RNA, 100 nt, synthetic construct
  source 1..100: mol_type = other RNA
  ncRNA 1..100: ncRNA_class = sgRNA
caagggaagggcacggttaggttttagagctagaaatagcaagttaaaataaggctagtccgttatcaacttgaaaaagtggcaccgagtcggtgctttt

SEQ ID NO: 807 - RNA, 100 nt, synthetic construct
  source 1..100: mol_type = other RNA
  ncRNA 1..100: ncRNA_class = sgRNA
cgctaaccgtgcccttccctgttttagagctagaaatagcaagttaaaataaggctagtccgttatcaacttgaaaaagtggcaccgagtcggtgctttt

SEQ ID NO: 808 - RNA, 100 nt, synthetic construct
  source 1..100: mol_type = other RNA
  ncRNA 1..100: ncRNA_class = sgRNA
acggttagcggcaccctcatgttttagagctagaaatagcaagttaaaataaggctagtccgttatcaacttgaaaaagtggcaccgagtcggtgctttt

SEQ ID NO: 809 - RNA, 100 nt, synthetic construct
  source 1..100: mol_type = other RNA
  ncRNA 1..100: ncRNA_class = sgRNA
gggccatcacttacctatgagttttagagctagaaatagcaagttaaaataaggctagtccgttatcaacttgaaaaagtggcaccgagtcggtgctttt

SEQ ID NO: 810 - RNA, 100 nt, synthetic construct
  source 1..100: mol_type = other RNA
  ncRNA 1..100: ncRNA_class = sgRNA
ggggccatcacttacctatggttttagagctagaaatagcaagttaaaataaggctagtccgttatcaacttgaaaaagtggcaccgagtcggtgctttt

SEQ ID NO: 811 - RNA, 100 nt, synthetic construct
  source 1..100: mol_type = other RNA
  ncRNA 1..100: ncRNA_class = sgRNA
tccgaataaactccaggcctgttttagagctagaaatagcaagttaaaataaggctagtccgttatcaacttgaaaaagtggcaccgagtcggtgctttt

SEQ ID NO: 812 - RNA, 100 nt, synthetic construct
  source 1..100: mol_type = other RNA
  ncRNA 1..100: ncRNA_class = sgRNA
ggcttttccgaataaactccgttttagagctagaaatagcaagttaaaataaggctagtccgttatcaacttgaaaaagtggcaccgagtcggtgctttt

SEQ ID NO: 813 - RNA, 100 nt, synthetic construct
  source 1..100: mol_type = other RNA
  ncRNA 1..100: ncRNA_class = sgRNA
gtttattcggaaaagccagcgttttagagctagaaatagcaagttaaaataaggctagtccgttatcaacttgaaaaagtggcaccgagtcggtgctttt

SEQ ID NO: 814 - RNA, 100 nt, synthetic construct
  source 1..100: mol_type = other RNA
  ncRNA 1..100: ncRNA_class = sgRNA
gccagctggtccagcctgtggttttagagctagaaatagcaagttaaaataaggctagtccgttatcaacttgaaaaagtggcaccgagtcggtgctttt

SEQ ID NO: 815 - RNA, 100 nt, synthetic construct
  source 1..100: mol_type = other RNA
  ncRNA 1..100: ncRNA_class = sgRNA
agcaccaccagtggccccacgttttagagctagaaatagcaagttaaaataaggctagtccgttatcaacttgaaaaagtggcaccgagtcggtgctttt

SEQ ID NO: 816 - RNA, 100 nt, synthetic construct
  source 1..100: mol_type = other RNA
  ncRNA 1..100: ncRNA_class = sgRNA
cggctgtacccacccgccaggttttagagctagaaatagcaagttaaaataaggctagtccgttatcaacttgaaaaagtggcaccgagtcggtgctttt

SEQ ID NO: 817 - RNA, 100 nt, synthetic construct
  source 1..100: mol_type = other RNA
  ncRNA 1..100: ncRNA_class = sgRNA
gtcgtgctggtcaccgctgcgttttagagctagaaatagcaagttaaaataaggctagtccgttatcaacttgaaaaagtggcaccgagtcggtgctttt

SEQ ID NO: 818 - RNA, 100 nt, synthetic construct
  source 1..100: mol_type = other RNA
  ncRNA 1..100: ncRNA_class = sgRNA
tcaccgctgccggcaacttcgttttagagctagaaatagcaagttaaaataaggctagtccgttatcaacttgaaaaagtggcaccgagtcggtgctttt

SEQ ID NO: 819 - RNA, 100 nt, synthetic construct
  source 1..100: mol_type = other RNA
  ncRNA 1..100: ncRNA_class = sgRNA
caccgctgccggcaacttccgttttagagctagaaatagcaagttaaaataaggctagtccgttatcaacttgaaaaagtggcaccgagtcggtgctttt

SEQ ID NO: 820 - RNA, 100 nt, synthetic construct
  source 1..100: mol_type = other RNA
  ncRNA 1..100: ncRNA_class = sgRNA
gtcccggaagttgccggcaggttttagagctagaaatagcaagttaaaataaggctagtccgttatcaacttgaaaaagtggcaccgagtcggtgctttt

SEQ ID NO: 821 - RNA, 100 nt, synthetic construct
  source 1..100: mol_type = other RNA
  ncRNA 1..100: ncRNA_class = sgRNA
ggcatcgtcccggaagttgcgttttagagctagaaatagcaagttaaaataaggctagtccgttatcaacttgaaaaagtggcaccgagtcggtgctttt

SEQ ID NO: 822 - RNA, 100 nt, synthetic construct
  source 1..100: mol_type = other RNA
  ncRNA 1..100: ncRNA_class = sgRNA
agtagaggcaggcatcgtccgttttagagctagaaatagcaagttaaaataaggctagtccgttatcaacttgaaaaagtggcaccgagtcggtgctttt

SEQ ID NO: 823 - RNA, 100 nt, synthetic construct
  source 1..100: mol_type = other RNA
  ncRNA 1..100: ncRNA_class = sgRNA
ccagcctcagctcccgaggtgttttagagctagaaatagcaagttaaaataaggctagtccgttatcaacttgaaaaagtggcaccgagtcggtgctttt

SEQ ID NO: 824 - RNA, 100 nt, synthetic construct
  source 1..100: mol_type = other RNA
  ncRNA 1..100: ncRNA_class = sgRNA
agcacctacctcgggagctggttttagagctagaaatagcaagttaaaataaggctagtccgttatcaacttgaaaaagtggcaccgagtcggtgctttt

SEQ ID NO: 825 - RNA, 100 nt, synthetic construct
  source 1..100: mol_type = other RNA
  ncRNA 1..100: ncRNA_class = sgRNA
ctttccaggtcatcacagttgttttagagctagaaatagcaagttaaaataaggctagtccgttatcaacttgaaaaagtggcaccgagtcggtgctttt

SEQ ID NO: 826 - RNA, 100 nt, synthetic construct
  source 1..100: mol_type = other RNA
  ncRNA 1..100: ncRNA_class = sgRNA
tttccaggtcatcacagttggttttagagctagaaatagcaagttaaaataaggctagtccgttatcaacttgaaaaagtggcaccgagtcggtgctttt

SEQ ID NO: 827 - RNA, 100 nt, synthetic construct
  source 1..100: mol_type = other RNA
  ncRNA 1..100: ncRNA_class = sgRNA
tggccccaactgtgatgaccgttttagagctagaaatagcaagttaaaataaggctagtccgttatcaacttgaaaaagtggcaccgagtcggtgctttt

SEQ ID NO: 828 - RNA, 100 nt, synthetic construct
  source 1..100: mol_type = other RNA
  ncRNA 1..100: ncRNA_class = sgRNA
cggctggtcttgggcattgggttttagagctagaaatagcaagttaaaataaggctagtccgttatcaacttgaaaaagtggcaccgagtcggtgctttt

SEQ ID NO: 829 - RNA, 100 nt, synthetic construct
  source 1..100: mol_type = other RNA
  ncRNA 1..100: ncRNA_class = sgRNA
caccggctggtcttgggcatgttttagagctagaaatagcaagttaaaataaggctagtccgttatcaacttgaaaaagtggcaccgagtcggtgctttt

SEQ ID NO: 830 - RNA, 100 nt, synthetic construct
  source 1..100: mol_type = other RNA
  ncRNA 1..100: ncRNA_class = sgRNA
caagaccagccggtgaccctgttttagagctagaaatagcaagttaaaataaggctagtccgttatcaacttgaaaaagtggcaccgagtcggtgctttt

SEQ ID NO: 831 - RNA, 100 nt, synthetic construct
  source 1..100: mol_type = other RNA
  ncRNA 1..100: ncRNA_class = sgRNA
aagaccagccggtgaccctggttttagagctagaaatagcaagttaaaataaggctagtccgttatcaacttgaaaaagtggcaccgagtcggtgctttt

SEQ ID NO: 832 - RNA, 100 nt, synthetic construct
  source 1..100: mol_type = other RNA
  ncRNA 1..100: ncRNA_class = sgRNA
cagggtcaccggctggtcttgttttagagctagaaatagcaagttaaaataaggctagtccgttatcaacttgaaaaagtggcaccgagtcggtgctttt

SEQ ID NO: 833 - RNA, 100 nt, synthetic construct
  source 1..100: mol_type = other RNA
  ncRNA 1..100: ncRNA_class = sgRNA
gaccaactttggccgctgtggttttagagctagaaatagcaagttaaaataaggctagtccgttatcaacttgaaaaagtggcaccgagtcggtgctttt

SEQ ID NO: 834 - RNA, 100 nt, synthetic construct
  source 1..100: mol_type = other RNA
  ncRNA 1..100: ncRNA_class = sgRNA
gtccacacagcggccaaagtgttttagagctagaaatagcaagttaaaataaggctagtccgttatcaacttgaaaaagtggcaccgagtcggtgctttt

SEQ ID NO: 835 - RNA, 100 nt, synthetic construct
  source 1..100: mol_type = other RNA
  ncRNA 1..100: ncRNA_class = sgRNA
gggcaaagaggtccacacaggttttagagctagaaatagcaagttaaaataaggctagtccgttatcaacttgaaaaagtggcaccgagtcggtgctttt

SEQ ID NO: 836 - RNA, 100 nt, synthetic construct
  source 1..100: mol_type = other RNA
  ncRNA 1..100: ncRNA_class = sgRNA
gtgtggacctctttgccccagttttagagctagaaatagcaagttaaaataaggctagtccgttatcaacttgaaaaagtggcaccgagtcggtgctttt

SEQ ID NO: 837 - RNA, 100 nt, synthetic construct
  source 1..100: mol_type = other RNA
  ncRNA 1..100: ncRNA_class = sgRNA
tgtcctcccctggggcaaaggttttagagctagaaatagcaagttaaaataaggctagtccgttatcaacttgaaaaagtggcaccgagtcggtgctttt

SEQ ID NO: 838 - RNA, 100 nt, synthetic construct
  source 1..100: mol_type = other RNA
  ncRNA 1..100: ncRNA_class = sgRNA
acctgctttgtgtcacagaggttttagagctagaaatagcaagttaaaataaggctagtccgttatcaacttgaaaaagtggcaccgagtcggtgctttt

SEQ ID NO: 839 - RNA, 100 nt, synthetic construct
  source 1..100: mol_type = other RNA
  ncRNA 1..100: ncRNA_class = sgRNA
cctgctttgtgtcacagagtgttttagagctagaaatagcaagttaaaataaggctagtccgttatcaacttgaaaaagtggcaccgagtcggtgctttt

SEQ ID NO: 840 - RNA, 100 nt, synthetic construct
  source 1..100: mol_type = other RNA
  ncRNA 1..100: ncRNA_class = sgRNA
cccactctgtgacacaaagcgttttagagctagaaatagcaagttaaaataaggctagtccgttatcaacttgaaaaagtggcaccgagtcggtgctttt

SEQ ID NO: 841 - RNA, 100 nt, synthetic construct
  source 1..100: mol_type = other RNA
  ncRNA 1..100: ncRNA_class = sgRNA
gtcacagagtgggacatcacgttttagagctagaaatagcaagttaaaataaggctagtccgttatcaacttgaaaaagtggcaccgagtcggtgctttt

SEQ ID NO: 842 - RNA, 100 nt, synthetic construct
  source 1..100: mol_type = other RNA
  ncRNA 1..100: ncRNA_class = sgRNA
tggtgacttaccagccacgtgttttagagctagaaatagcaagttaaaataaggctagtccgttatcaacttgaaaaagtggcaccgagtcggtgctttt

SEQ ID NO: 843 - RNA, 100 nt, synthetic construct
  source 1..100: mol_type = other RNA
  ncRNA 1..100: ncRNA_class = sgRNA
gtggtgacttaccagccacggttttagagctagaaatagcaagttaaaataaggctagtccgttatcaacttgaaaaagtggcaccgagtcggtgctttt

SEQ ID NO: 844 - RNA, 100 nt, synthetic construct
  source 1..100: mol_type = other RNA
  ncRNA 1..100: ncRNA_class = sgRNA
tgccgagccggagctcacccgttttagagctagaaatagcaagttaaaataaggctagtccgttatcaacttgaaaaagtggcaccgagtcggtgctttt

SEQ ID NO: 845 - RNA, 100 nt, synthetic construct
  source 1..100: mol_type = other RNA
  ncRNA 1..100: ncRNA_class = sgRNA
ggccagggtgagctccggctgttttagagctagaaatagcaagttaaaataaggctagtccgttatcaacttgaaaaagtggcaccgagtcggtgctttt

SEQ ID NO: 846 - RNA, 100 nt, synthetic construct
  source 1..100: mol_type = other RNA
  ncRNA 1..100: ncRNA_class = sgRNA
aactcggccagggtgagctcgttttagagctagaaatagcaagttaaaataaggctagtccgttatcaacttgaaaaagtggcaccgagtcggtgctttt

SEQ ID NO: 847 - RNA, 100 nt, synthetic construct
  source 1..100: mol_type = other RNA
  ncRNA 1..100: ncRNA_class = sgRNA
gtctctgcctcaactcggccgttttagagctagaaatagcaagttaaaataaggctagtccgttatcaacttgaaaaagtggcaccgagtcggtgctttt

SEQ ID NO: 848 - RNA, 100 nt, synthetic construct
  source 1..100: mol_type = other RNA
  ncRNA 1..100: ncRNA_class = sgRNA
gatcagtctctgcctcaactgttttagagctagaaatagcaagttaaaataaggctagtccgttatcaacttgaaaaagtggcaccgagtcggtgctttt

SEQ ID NO: 849 - RNA, 100 nt, synthetic construct
  source 1..100: mol_type = other RNA
  ncRNA 1..100: ncRNA_class = sgRNA
tgacatctttggcagagaaggttttagagctagaaatagcaagttaaaataaggctagtccgttatcaacttgaaaaagtggcaccgagtcggtgctttt

SEQ ID NO: 850 - RNA, 100 nt, synthetic construct
  source 1..100: mol_type = other RNA
  ncRNA 1..100: ncRNA_class = sgRNA
aagatgtcatcaatgaggccgttttagagctagaaatagcaagttaaaataaggctagtccgttatcaacttgaaaaagtggcaccgagtcggtgctttt

SEQ ID NO: 851 - RNA, 100 nt, synthetic construct
  source 1..100: mol_type = other RNA
  ncRNA 1..100: ncRNA_class = sgRNA
caatgaggcctggttccctggttttagagctagaaatagcaagttaaaataaggctagtccgttatcaacttgaaaaagtggcaccgagtcggtgctttt

SEQ ID NO: 852 - RNA, 100 nt, synthetic construct
  source 1..100: mol_type = other RNA
  ncRNA 1..100: ncRNA_class = sgRNA
tcagtacccgctggtcctcagttttagagctagaaatagcaagttaaaataaggctagtccgttatcaacttgaaaaagtggcaccgagtcggtgctttt

SEQ ID NO: 853 - RNA, 100 nt, synthetic construct
  source 1..100: mol_type = other RNA
  ncRNA 1..100: ncRNA_class = sgRNA
gcgggtactgacccccaaccgttttagagctagaaatagcaagttaaaataaggctagtccgttatcaacttgaaaaagtggcaccgagtcggtgctttt

SEQ ID NO: 854 - RNA, 100 nt, synthetic construct
  source 1..100: mol_type = other RNA
  ncRNA 1..100: ncRNA_class = sgRNA
ggttgggggtcagtacccgcgttttagagctagaaatagcaagttaaaataaggctagtccgttatcaacttgaaaaagtggcaccgagtcggtgctttt

SEQ ID NO: 855 - RNA, 100 nt, synthetic construct
  source 1..100: mol_type = other RNA
  ncRNA 1..100: ncRNA_class = sgRNA
ggtactgacccccaacctgggttttagagctagaaatagcaagttaaaataaggctagtccgttatcaacttgaaaaagtggcaccgagtcggtgctttt

SEQ ID NO: 856 - RNA, 100 nt, synthetic construct
  source 1..100: mol_type = other RNA
  ncRNA 1..100: ncRNA_class = sgRNA
cctgccccccagcacccatggttttagagctagaaatagcaagttaaaataaggctagtccgttatcaacttgaaaaagtggcaccgagtcggtgctttt

SEQ ID NO: 857 - RNA, 100 nt, synthetic construct
  source 1..100: mol_type = other RNA
  ncRNA 1..100: ncRNA_class = sgRNA
ttacctgccccatgggtgctgttttagagctagaaatagcaagttaaaataaggctagtccgttatcaacttgaaaaagtggcaccgagtcggtgctttt

SEQ ID NO: 858 - RNA, 100 nt, synthetic construct
  source 1..100: mol_type = other RNA
  ncRNA 1..100: ncRNA_class = sgRNA
gccaacctgcaaaaagggccgttttagagctagaaatagcaagttaaaataaggctagtccgttatcaacttgaaaaagtggcaccgagtcggtgctttt

SEQ ID NO: 859 - RNA, 100 nt, synthetic construct
  source 1..100: mol_type = other RNA
  ncRNA 1..100: ncRNA_class = sgRNA
gactgtatggtcagcacactgttttagagctagaaatagcaagttaaaataaggctagtccgttatcaacttgaaaaagtggcaccgagtcggtgctttt

SEQ ID NO: 860 - RNA, 100 nt, synthetic construct
  source 1..100: mol_type = other RNA
  ncRNA 1..100: ncRNA_class = sgRNA
actgtatggtcagcacactcgttttagagctagaaatagcaagttaaaataaggctagtccgttatcaacttgaaaaagtggcaccgagtcggtgctttt

SEQ ID NO: 861 - RNA, 100 nt, synthetic construct
  source 1..100: mol_type = other RNA
  ncRNA 1..100: ncRNA_class = sgRNA
ctgtatggtcagcacactcggttttagagctagaaatagcaagttaaaataaggctagtccgttatcaacttgaaaaagtggcaccgagtcggtgctttt

SEQ ID NO: 862 - RNA, 100 nt, synthetic construct
  source 1..100: mol_type = other RNA
  ncRNA 1..100: ncRNA_class = sgRNA
cagcacactcggggcctacagttttagagctagaaatagcaagttaaaataaggctagtccgttatcaacttgaaaaagtggcaccgagtcggtgctttt

SEQ ID NO: 863 - RNA, 100 nt, synthetic construct
  source 1..100: mol_type = other RNA
  ncRNA 1..100: ncRNA_class = sgRNA
acactcggggcctacacggagttttagagctagaaatagcaagttaaaataaggctagtccgttatcaacttgaaaaagtggcaccgagtcggtgctttt

SEQ ID NO: 864 - RNA, 100 nt, synthetic construct
  source 1..100: mol_type = other RNA
  ncRNA 1..100: ncRNA_class = sgRNA
acggctgtggccatccgtgtgttttagagctagaaatagcaagttaaaataaggctagtccgttatcaacttgaaaaagtggcaccgagtcggtgctttt

SEQ ID NO: 865 - RNA, 100 nt, synthetic construct
  source 1..100: mol_type = other RNA
  ncRNA 1..100: ncRNA_class = sgRNA
gctcctcatctggggcgcaggttttagagctagaaatagcaagttaaaataaggctagtccgttatcaacttgaaaaagtggcaccgagtcggtgctttt

SEQ ID NO: 866 - RNA, 100 nt, synthetic construct
  source 1..100: mol_type = other RNA
  ncRNA 1..100: ncRNA_class = sgRNA
gcagaccagcttgcccccttgttttagagctagaaatagcaagttaaaataaggctagtccgttatcaacttgaaaaagtggcaccgagtcggtgctttt

SEQ ID NO: 867 - RNA, 100 nt, synthetic construct
  source 1..100: mol_type = other RNA
  ncRNA 1..100: ncRNA_class = sgRNA
tgccgggcccacaacgctttgttttagagctagaaatagcaagttaaaataaggctagtccgttatcaacttgaaaaagtggcaccgagtcggtgctttt

SEQ ID NO: 868 - RNA, 100 nt, synthetic construct
  source 1..100: mol_type = other RNA
  ncRNA 1..100: ncRNA_class = sgRNA
gccgggcccacaacgcttttgttttagagctagaaatagcaagttaaaataaggctagtccgttatcaacttgaaaaagtggcaccgagtcggtgctttt

SEQ ID NO: 869 - RNA, 100 nt, synthetic construct
  source 1..100: mol_type = other RNA
  ncRNA 1..100: ncRNA_class = sgRNA
ccgggcccacaacgcttttggttttagagctagaaatagcaagttaaaataaggctagtccgttatcaacttgaaaaagtggcaccgagtcggtgctttt

SEQ ID NO: 870 - RNA, 100 nt, synthetic construct
  source 1..100: mol_type = other RNA
  ncRNA 1..100: ncRNA_class = sgRNA
cgggcccacaacgcttttgggttttagagctagaaatagcaagttaaaataaggctagtccgttatcaacttgaaaaagtggcaccgagtcggtgctttt

SEQ ID NO: 871 - RNA, 100 nt, synthetic construct
  source 1..100: mol_type = other RNA
  ncRNA 1..100: ncRNA_class = sgRNA
ccacaacgcttttgggggtggttttagagctagaaatagcaagttaaaataaggctagtccgttatcaacttgaaaaagtggcaccgagtcggtgctttt

SEQ ID NO: 872 - RNA, 100 nt, synthetic construct
  source 1..100: mol_type = other RNA
  ncRNA 1..100: ncRNA_class = sgRNA
ctcacccccaaaagcgttgtgttttagagctagaaatagcaagttaaaataaggctagtccgttatcaacttgaaaaagtggcaccgagtcggtgctttt

SEQ ID NO: 873 - RNA, 100 nt, synthetic construct
  source 1..100: mol_type = other RNA
  ncRNA 1..100: ncRNA_class = sgRNA
agggtgtctacgccattgccgttttagagctagaaatagcaagttaaaataaggctagtccgttatcaacttgaaaaagtggcaccgagtcggtgctttt

SEQ ID NO: 874 - RNA, 100 nt, synthetic construct
  source 1..100: mol_type = other RNA
  ncRNA 1..100: ncRNA_class = sgRNA
gtggacacgggtccccatgcgttttagagctagaaatagcaagttaaaataaggctagtccgttatcaacttgaaaaagtggcaccgagtcggtgctttt

SEQ ID NO: 875 - RNA, 100 nt, synthetic construct
  source 1..100: mol_type = other RNA
  ncRNA 1..100: ncRNA_class = sgRNA
aaggtcctccacctcccagtgttttagagctagaaatagcaagttaaaataaggctagtccgttatcaacttgaaaaagtggcaccgagtcggtgctttt

SEQ ID NO: 876 - RNA, 100 nt, synthetic construct
  source 1..100: mol_type = other RNA
  ncRNA 1..100: ncRNA_class = sgRNA
cctcagcacaggcggcttgtgttttagagctagaaatagcaagttaaaataaggctagtccgttatcaacttgaaaaagtggcaccgagtcggtgctttt

SEQ ID NO: 877 - RNA, 100 nt, synthetic construct
  source 1..100: mol_type = other RNA
  ncRNA 1..100: ncRNA_class = sgRNA
cactggttgggctgacctcggttttagagctagaaatagcaagttaaaataaggctagtccgttatcaacttgaaaaagtggcaccgagtcggtgctttt

SEQ ID NO: 878 - RNA, 100 nt, synthetic construct
  source 1..100: mol_type = other RNA
  ncRNA 1..100: ncRNA_class = sgRNA
aggtcagcccaaccagtgcggttttagagctagaaatagcaagttaaaataaggctagtccgttatcaacttgaaaaagtggcaccgagtcggtgctttt

SEQ ID NO: 879 - RNA, 100 nt, synthetic construct
  source 1..100: mol_type = other RNA
  ncRNA 1..100: ncRNA_class = sgRNA
ggtcagcccaaccagtgcgtgttttagagctagaaatagcaagttaaaataaggctagtccgttatcaacttgaaaaagtggcaccgagtcggtgctttt

SEQ ID NO: 880 - RNA, 100 nt, synthetic construct
  source 1..100: mol_type = other RNA
  ncRNA 1..100: ncRNA_class = sgRNA
caaccagtgcgtgggccacagttttagagctagaaatagcaagttaaaataaggctagtccgttatcaacttgaaaaagtggcaccgagtcggtgctttt

SEQ ID NO: 881 - RNA, 100 nt, synthetic construct
  source 1..100: mol_type = other RNA
  ncRNA 1..100: ncRNA_class = sgRNA
tagacaacacgtgtgtagtcgttttagagctagaaatagcaagttaaaataaggctagtccgttatcaacttgaaaaagtggcaccgagtcggtgctttt

SEQ ID NO: 882 - RNA, 100 nt, synthetic construct
  source 1..100: mol_type = other RNA
  ncRNA 1..100: ncRNA_class = sgRNA
gactacacacgtgttgtctagttttagagctagaaatagcaagttaaaataaggctagtccgttatcaacttgaaaaagtggcaccgagtcggtgctttt

SEQ ID NO: 883 - RNA, 100 nt, synthetic construct
  source 1..100: mol_type = other RNA
  ncRNA 1..100: ncRNA_class = sgRNA
cacgtgtgtagtcaggagccgttttagagctagaaatagcaagttaaaataaggctagtccgttatcaacttgaaaaagtggcaccgagtcggtgctttt

SEQ ID NO: 884 - RNA, 100 nt, synthetic construct
  source 1..100: mol_type = other RNA
  ncRNA 1..100: ncRNA_class = sgRNA
agccgggacgtcagcactacgttttagagctagaaatagcaagttaaaataaggctagtccgttatcaacttgaaaaagtggcaccgagtcggtgctttt

SEQ ID NO: 885 - RNA, 100 nt, synthetic construct
  source 1..100: mol_type = other RNA
  ncRNA 1..100: ncRNA_class = sgRNA
tgcctgtagtgctgacgtccgttttagagctagaaatagcaagttaaaataaggctagtccgttatcaacttgaaaaagtggcaccgagtcggtgctttt

SEQ ID NO: 886 - RNA, 100 nt, synthetic construct
  source 1..100: mol_type = other RNA
  ncRNA 1..100: ncRNA_class = sgRNA
actacaggcagcaccagcgagttttagagctagaaatagcaagttaaaataaggctagtccgttatcaacttgaaaaagtggcaccgagtcggtgctttt

SEQ ID NO: 887 - RNA, 100 nt, synthetic construct
  source 1..100: mol_type = other RNA
  ncRNA 1..100: ncRNA_class = sgRNA
tacaggcagcaccagcgaaggttttagagctagaaatagcaagttaaaataaggctagtccgttatcaacttgaaaaagtggcaccgagtcggtgctttt

SEQ ID NO: 888 - RNA, 100 nt, synthetic construct
  source 1..100: mol_type = other RNA
  ncRNA 1..100: ncRNA_class = sgRNA
gcagatggcaacggctgtcagttttagagctagaaatagcaagttaaaataaggctagtccgttatcaacttgaaaaagtggcaccgagtcggtgctttt

SEQ ID NO: 889 - RNA, 22 nt, synthetic construct
  source 1..22: mol_type = other RNA
gttttagagctatgctgttttg

SEQ ID NO: 890 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 891-900 - skipped (10 entries)

SEQ ID NO: 901 - protein, 1379 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 902 - RNA, 4140 nt, synthetic construct
  source 1..4140: mol_type = other RNA
  mRNA 1..4140: note = mRNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 903 - RNA, 4134 nt, synthetic construct
  source 1..4134: mol_type = other RNA
  mRNA 1..4134: note = mRNA
gacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtc

SEQ ID NO: 904 - RNA, 74 nt, synthetic construct
  source 1..74: mol_type = other RNA
aacagcatagcaagttaaaataaggctagtccgttatcaacttgaaaaagtggcaccgagtcggtgcttttttt

SEQ ID NO: 905 - RNA, 12 nt, synthetic construct
  source 1..12: mol_type = other RNA
gttttagagcta

SEQ ID NO: 906 - RNA, 65 nt, synthetic construct
  source 1..65: mol_type = other RNA
atagcaagttaaaataaggctagtccgttatcaacttgaaaaagtggcaccgagtcggtgctttt

SEQ ID NO: 907 - RNA, 100 nt, synthetic construct
  source 1..100: mol_type = other RNA
  modified_base 1..3: mod_base = OTHER; note = 2-Prime-O-Me nucleotides
  modified_base 29..40: mod_base = OTHER; note = 2-Prime-O-Me nucleotides
  modified_base 69..100: mod_base = OTHER; note = 2-Prime-O-Me nucleotides
  modified_base 1..4: mod_base = OTHER; note = Phosphorothioate linkage
  modified_base 97..100: mod_base = OTHER; note = Phosphorothioate linkage
nnnnnnnnnnnnnnnnnnnngttttagagctagaaatagcaagttaaaataaggctagtccgttatcaacttgaaaaagtggcaccgagtcggtgctttt

SEQ ID NO: 908 - DNA, 20 bp, synthetic construct
  source 1..20: mol_type = other DNA
  misc_feature 1: note = DNA
  misc_feature 2..20: note = RNA
cgtgcgcaggaggacgagga

SEQ ID NO: 909 - DNA, 20 bp, synthetic construct
  source 1..20: mol_type = other DNA
  misc_feature 1..2: note = DNA
  misc_feature 3..20: note = RNA
cgtgcgcaggaggacgagga

SEQ ID NO: 910 - DNA, 20 bp, synthetic construct
  source 1..20: mol_type = other DNA
  misc_feature 19: note = DNA
  misc_feature order(1..18,20): note = RNA
cgtgcgcaggaggacgagga

SEQ ID NO: 911 - DNA, 20 bp, synthetic construct
  source 1..20: mol_type = other DNA
  misc_feature 19..20: note = DNA
  misc_feature 1..18: note = RNA
cgtgcgcaggaggacgagga

SEQ ID NO: 912 - DNA, 20 bp, synthetic construct
  source 1..20: mol_type = other DNA
  misc_feature order(2,20): note = DNA
  misc_feature order(1,3..19): note = RNA
cgtgcgcaggaggacgagga

SEQ ID NO: 913 - DNA, 20 bp, synthetic construct
  source 1..20: mol_type = other DNA
  misc_feature order(1..2,19..20): note = DNA
  misc_feature 3..18: note = RNA
cgtgcgcaggaggacgagga

SEQ ID NO: 914 - DNA, 20 bp, synthetic construct
  source 1..20: mol_type = other DNA
  misc_feature 3: note = DNA
  misc_feature order(1..2,4..20): note = RNA
cgtgcgcaggaggacgagga

SEQ ID NO: 915 - DNA, 20 bp, synthetic construct
  source 1..20: mol_type = other DNA
  misc_feature 3..4: note = DNA
  misc_feature order(1..2,5..20): note = RNA
cgtgcgcaggaggacgagga

SEQ ID NO: 916 - DNA, 20 bp, synthetic construct
  source 1..20: mol_type = other DNA
  misc_feature 3..5: note = DNA
  misc_feature order(1..2,6..20): note = RNA
cgtgcgcaggaggacgagga

SEQ ID NO: 917 - DNA, 20 bp, synthetic construct
  source 1..20: mol_type = other DNA
  misc_feature 3..6: note = DNA
  misc_feature order(1..2,7..20): note = RNA
cgtgcgcaggaggacgagga

SEQ ID NO: 918 - DNA, 20 bp, synthetic construct
  source 1..20: mol_type = other DNA
  misc_feature 3..7: note = DNA
  misc_feature order(1..2,8..20): note = RNA
cgtgcgcaggaggacgagga

SEQ ID NO: 919 - DNA, 20 bp, synthetic construct
  source 1..20: mol_type = other DNA
  misc_feature 1: note = DNA
  misc_feature 2..20: note = RNA
ggtgctagccttgcgttccg

SEQ ID NO: 920 - DNA, 20 bp, synthetic construct
  source 1..20: mol_type = other DNA
  misc_feature 20: note = DNA
  misc_feature 1..19: note = RNA
ggtgctagccttgcgttccg

SEQ ID NO: 921 - DNA, 20 bp, synthetic construct
  source 1..20: mol_type = other DNA
  misc_feature order(1,20): note = DNA
  misc_feature 2..19: note = RNA
ggtgctagccttgcgttccg

SEQ ID NO: 922 - DNA, 20 bp, synthetic construct
  source 1..20: mol_type = other DNA
  misc_feature 3: note = DNA
  misc_feature order(1..2,4..20): note = RNA
ggtgctagccttgcgttccg

SEQ ID NO: 923 - DNA, 20 bp, synthetic construct
  source 1..20: mol_type = other DNA
  misc_feature order(3,6): note = DNA
  misc_feature order(1..2,4..5,7..20): note = RNA
ggtgctagccttgcgttccg

SEQ ID NO: 924 - DNA, 20 bp, synthetic construct
  source 1..20: mol_type = other DNA
  misc_feature 4: note = DNA
  misc_feature order(1..3,5..20): note = RNA
ggcttcctggtgaagatgag

SEQ ID NO: 925 - DNA, 20 bp, synthetic construct
  source 1..20: mol_type = other DNA
  misc_feature 4..5: note = DNA
  misc_feature order(1..3,6..20): note = RNA
ggcttcctggtgaagatgag

SEQ ID NO: 926 - DNA, 20 bp, synthetic construct
  source 1..20: mol_type = other DNA
  misc_feature order(4..5,8): note = DNA
  misc_feature order(1..3,6..7,9..20): note = RNA
ggcttcctggtgaagatgag

SEQ ID NO: 927 - DNA, 20 bp, synthetic construct
  source 1..20: mol_type = other DNA
  misc_feature order(4..5,11): note = DNA
  misc_feature order(1..3,6..10,12..20): note = RNA
ggcttcctggtgaagatgag

SEQ ID NO: 928 - DNA, 20 bp, synthetic construct
  source 1..20: mol_type = other DNA
  misc_feature order(4..5,17): note = DNA
  misc_feature order(1..3,6..16,18..20): note = RNA
ggcttcctggtgaagatgag

SEQ ID NO: 929 - DNA, 20 bp, synthetic construct
  source 1..20: mol_type = other DNA
  misc_feature order(4..5,8,11): note = DNA
  misc_feature order(1..3,6..7,9..10,12..20): note = RNA
ggcttcctggtgaagatgag

SEQ ID NO: 930 - DNA, 20 bp, synthetic construct
  source 1..20: mol_type = other DNA
  misc_feature 1: note = DNA
  misc_feature 2..20: note = RNA
gtgctcaactgccaagggaa

SEQ ID NO: 931 - DNA, 20 bp, synthetic construct
  source 1..20: mol_type = other DNA
  misc_feature 19: note = DNA
  misc_feature order(1..18,20): note = RNA
gtgctcaactgccaagggaa

SEQ ID NO: 932 - DNA, 20 bp, synthetic construct
  source 1..20: mol_type = other DNA
  misc_feature order(1,19): note = DNA
  misc_feature order(2..18,20): note = RNA
gtgctcaactgccaagggaa

SEQ ID NO: 933 - DNA, 20 bp, synthetic construct
  source 1..20: mol_type = other DNA
  misc_feature 2: note = DNA
  misc_feature order(1,3..20): note = RNA
gtgctcaactgccaagggaa

SEQ ID NO: 934 - DNA, 20 bp, synthetic construct
  source 1..20: mol_type = other DNA
  misc_feature order(2,5): note = DNA
  misc_feature order(1,3..4,6..20): note = RNA
gtgctcaactgccaagggaa

SEQ ID NO: 935 - DNA, 20 bp, synthetic construct
  source 1..20: mol_type = other DNA
  misc_feature order(2,5,10): note = DNA
  misc_feature order(1,3..4,6..9,11..20): note = RNA
gtgctcaactgccaagggaa

SEQ ID NO: 936 - DNA, 20 bp, synthetic construct
  source 1..20: mol_type = other DNA
  misc_feature 2: note = DNA
  misc_feature order(1,3..20): note = RNA
gtgtggacctctttgcccca

SEQ ID NO: 937 - DNA, 20 bp, synthetic construct
  source 1..20: mol_type = other DNA
  misc_feature order(2,4): note = DNA
  misc_feature order(1,3,5..20): note = RNA
gtgtggacctctttgcccca

SEQ ID NO: 938 - DNA, 20 bp, synthetic construct
  source 1..20: mol_type = other DNA
  misc_feature order(2,10): note = DNA
  misc_feature order(1,3..9,11..20): note = RNA
gtgtggacctctttgcccca

SEQ ID NO: 939 - DNA, 20 bp, synthetic construct
  source 1..20: mol_type = other DNA
  misc_feature order(2,12): note = DNA
  misc_feature order(1,3..11,13..20): note = RNA
gtgtggacctctttgcccca

SEQ ID NO: 940 - DNA, 20 bp, synthetic construct
  source 1..20: mol_type = other DNA
  misc_feature order(2,4,10): note = DNA
  misc_feature order(1,3,5..9,11..20): note = RNA
gtgtggacctctttgcccca

SEQ ID NO: 941 - RNA, 37 nt, synthetic construct
  source 1..37: mol_type = other RNA
  mRNA 1..37: note = mRNA
agggacccgcagctcagctacagcacagatcagcacc

SEQ ID NO: 942 - RNA, 58 nt, synthetic construct
  source 1..58: mol_type = other RNA
  mRNA 1..58: note = mRNA
agcaatcctttctttcagctggagtgctcctcaggagccagccccacccttagaaaag

SEQ ID NO: 943 - RNA, 73 nt, synthetic construct
  source 1..73: mol_type = other RNA
  mRNA 1..73: note = mRNA
agtatattagtgctaatttccctccgtttgtcctagcttttctcttctgtcaaccccacacgcctttggcaca

SEQ ID NO: 944 - RNA, 36 nt, synthetic construct
  source 1..36: mol_type = other RNA
  mRNA 1..36: note = mRNA
agcactgcctggctccacgtgcctcctggtctcagt

SEQ ID NO: 945 - RNA, 70 nt, synthetic construct
  source 1..70: mol_type = other RNA
  mRNA 1..70: note = mRNA
actataaatagcagccacctctccctggcagacagggacccgcagctcagctacagcacagatcagcacc

SEQ ID NO: 946 - RNA, 58 nt, synthetic construct
  source 1..58: mol_type = other RNA
  mRNA 1..58: note = mRNA
aggcacagacaccaaggacagagacgctggctaggccgccctccccactgttaccaac

SEQ ID NO: 947 - RNA, 252 nt, synthetic construct
  source 1..252: mol_type = other RNA
  mRNA 1..252: note = mRNA
tcggggagctcggctcttgagacaggaatcttgcccattccccgaacgaataaaccccttccttaactcagcgtctgaggaattttgtctgcggctcctcctgctacattctgagtggggaaagggactaaggtggtctgaggaccccacagagtcaggaagattgagagcctgataaaggtcctgcgggcaggacaggacctcccaaccaagccctccagcaaggattcagagtgcccctccggcctcgcc

SEQ ID NO: 948 - RNA, 112 nt, synthetic construct
  source 1..112: mol_type = other RNA
  mRNA 1..112: note = mRNA
gcttcctcttcactctgctctcaggagatctggctgtgaggccctcagggcagggatacaaagcggggagagggtacacaatgggtatctaataaatacttaagaggtggaa

SEQ ID NO: 949 - RNA, 217 nt, synthetic construct
  source 1..217: mol_type = other RNA
  mRNA 1..217: note = mRNA
actaagttaaatatttctgcacagtgttcccatggccccttgcatttccttcttaactctctgttacacgtcattgaaactacacttttttggtctgtttttgtgctagactgtaagttccttgggggcagggcctttgtctgtctcatctctgtattcccaaatgcctaacagtacagagccatgactcaataaatacatgttaaatggatgaatg

SEQ ID NO: 950 - RNA, 120 nt, synthetic construct
  source 1..120: mol_type = other RNA
  mRNA 1..120: note = mRNA
caggacacagccttggatcaggacagagacttgggggccatcctgcccctccaacccgacatgtgtacctcagctttttccctcacttgcatcaataaagcttctgtgtttggaacagct

SEQ ID NO: 951 - RNA, 135 nt, synthetic construct
  source 1..135: mol_type = other RNA
  mRNA 1..135: note = mRNA
gcttcctcttcactctgctctcaggagacctggctatgaggccctcggggcagggatacaaagttagtgaggtctatgtccagagaagctgagatatggcatataataggcatctaataaatgcttaagaggtgg

SEQ ID NO: 952 - RNA, 112 nt, synthetic construct
  source 1..112: mol_type = other RNA
  mRNA 1..112: note = mRNA
agtgtccagaccattgtcttccaaccccagctggcctctagaacacccactggccagtcctagagctcctgtccctacccactctttgctacaataaatgctgaatgaatcc

SEQ ID NO: 953 - RNA, 112 nt, synthetic construct
  source 1..112: mol_type = other RNA
  mRNA 1..112: note = mRNA
ggacctgaagggtgacatcccaggaggggcctctgaaatttcccacaccccagcgcctgtgctgaggactccctccatgtggccccaggtgccaccaataaaaatcctacag

SEQ ID NO: 954 - RNA, 4137 nt, synthetic construct
  source 1..4137: mol_type = other RNA
atggacaagaaatatagcatcgggctggatatcggcaccaactctgtcggctgggcggtaatcaccgacgagtacaaggttcctagcaagaagttcaaggtgctgggaaacaccgaccggcacagcatcaagaagaacctgatcggcgctttgctgttcgatagcggcgaaaccgccgaagccaccaggctgaagagaaccgccaggcggcgttacacaagaagaaaaaaccggatctgttacctgcaggagattttcagcaacgagatggccaaggtggatgacagcttcttccacagactggaagagagcttcctggtggaggaagataaaaagcacgaaagacaccccatctttggcaatatcgtggacgaggttgcataccacgagaagtaccctactatctaccacctgagaaagaagttggtggactctacagacaaggctgatctgcggctgatctacctggcgctggcccacatgatcaagttccgggggcatttcctgatcgaaggcgatctgaaccccgacaacagcgacgtggacaagctgttcatccaactggttcagacatataatcaactgttcgaggagaaccctatcaacgcctcaggcgtggacgccaaggccatcttaagcgccagactgtcaaaatctagacggctggaaaacctgatcgcccagctgcccggcgaaaagaagaacgggctgttcggcaacctgatcgccctgagccttggtctgacacctaacttcaagagcaatttcgatctggctgaggatgccaagctccagctcagcaaagacacctacgacgacgatctggacaatctgctggctcaaatcggcgaccagtacgctgacctgttcctggctgccaagaacctgtctgacgccatcctgctcagcgacatcctgagagttaacacagagattacaaaggcccctctgtctgccagcatgatcaagcggtacgacgagcaccaccaggacctgaccctgctgaaggccctggtgagacagcagctgcctgagaagtacaaggaaatctttttcgaccagagcaagaacggatatgccggctatatcgatggcggcgccagtcaggaagagttctacaagttcatcaaacctatcctcgaaaagatggacggcacagaggaactgctagtgaagctgaacagagaagatctgctgagaaagcagcggacattcgataacggatccattcctcaccagatccacctgggcgagctgcatgccatccttcgaaggcaagaagatttctaccctttcctgaaagataacagagagaagatcgagaaaatcctgacattcagaatcccctactacgtgggacctctggccagaggcaacagccggttcgcctggatgacccggaaaagcgaggagacaatcacaccttggaacttcgaggaggtcgtggacaaaggcgccagcgctcagagcttcatcgagagaatgaccaatttcgacaaaaacctgcccaacgagaaggtgctccctaagcacagcctgctgtacgagtactttaccgtctacaacgagctgaccaaggtcaagtatgtgaccgagggtatgcggaagcctgctttcctgagcggcgagcagaagaaggccatcgtggacctgctgttcaagaccaacagaaaagtgaccgtgaagcagctgaaagaggactactttaagaagatcgagtgcttcgattcggtggaaatcagcggcgtggaggacaggttcaacgcctccctgggcacataccacgacctgctgaagatcatcaaggataaggattttctcgacaatgaagagaacgaggacatcctggaggacatcgtgctcacactgacactgtttgaagacagagaaatgatcgaggaaagactgaagacctacgctcacctgtttgacgacaaagtcatgaaacagctgaagcggagacgctacaccggctggggccggctgagcagaaagctgatcaacggcatcagagacaagcaaagcggcaagaccattctggatttcctgaagagcgacggcttcgccaaccggaacttcatgcagctgatccacgacgactctctgaccttcaaagaagacatccagaaggcccaagtgagtggccagggcgactccctccatgaacacatagccaacctggccggctctccagccatcaagaagggaatcttgcagaccgtgaaggtggtggatgaactggtgaaagtgatgggcagacataagcccgagaacatcgtgatcgaaatggccagagagaatcagaccacccagaagggccagaagaactctcgggaaagaatgaagcggatcgaggagggcatcaaggagctgggcagccagatcctcaaggaacatcccgtggaaaacacacagctgcagaatgagaagctgtacttgtactacctgcagaacggtcgagatatgtacgtggaccaggagctggacatcaacagactgtccgactacgatgtggatcacatcgtgcctcagagcttcctgaaggacgattccatcgataacaaggtgctgaccagaagcgataagaacagagggaagagcgacaacgtgccatctgaagaagtggtgaagaagatgaagaattactggagacagctgctgaacgccaaactgatcacacaaagaaaattcgacaacctgacgaaggccgaacggggaggattgagcgaacttgacaaagccggctttatcaagagacagctggtcgaaaccagacagatcaccaaacacgtagcccagatcctggacagccggatgaacaccaagtacgacgagaatgacaagctgatacgggaggtgaaggttatcaccctgaaatctaagctggtgagcgacttcagaaaggacttccagttctacaaagtgcgggagatcaacaactaccaccacgcccacgatgcctacctcaacgcggtggtgggcaccgccctgatcaagaagtaccctaagctggaatctgagttcgtgtacggcgattacaaggtctacgacgtgcggaagatgattgccaagtccgagcaggagatcggcaaggccaccgccaagtactttttctactcgaacatcatgaacttctttaaaaccgagatcacactggccaatggagagatcagaaagcggcctctgattgaaacaaacggcgaaacaggcgagattgtctgggacaaaggcagggatttcgctacagtgaggaaggtgctgagcatgcctcaggtcaatatcgtgaagaagaccgaggtgcagaccggcggattcagcaaggagtccatcctgcctaagagaaactctgacaaactgatcgcccggaaaaaggactgggatcctaagaagtatggcggcttcgacagccccaccgtggcctacagcgtgctggtggtggccaaggtggaaaagggcaagagcaaaaaactgaagtctgtgaaagagctgctgggcatcacaatcatggaaagaagcagctttgagaagaaccccatagacttcctggaagccaagggctacaaggaggtgaaaaaggatctgatcatcaagctgccaaaatacagcctgtttgaactggagaacggcagaaagcgcatgttggccagcgccggcgagctgcaaaaaggaaacgagctggctctgcccagcaagtacgtgaacttcctgtacctggccagccactacgagaagctgaagggctcacccgaagataatgagcagaagcagttattcgtggagcagcacaagcactacttggacgagatcattgaacagatcagcgagttcagcaagagagtgatcctggccgatgccaacctggacaaggtgctgagcgcctacaacaagcaccgggacaagcctatcagggaacaggccgaaaatatcatccacctgtttaccctcaccaatctgggagcccctgccgccttcaagtatttcgacacaaccatcgacagaaagagatatacctccaccaaggaggtactcgacgccaccctgatccaccagagcattaccggcctgtacgaaaccagaatcgacctgtctcagctgggaggcgacggtggtggcagcccgaagaagaaaagaaaggtg

SEQ ID NO: 955 - RNA, 4137 nt, synthetic construct
  source 1..4137: mol_type = other RNA
atggataagaaatattccattggcctggacatcgggaccaacagcgtgggctgggccgtgatcaccgacgagtacaaggtccccagcaagaagttcaaggtgctgggaaacaccgaccggcactccatcaagaagaacttgatcggggccttgctgttcgacagcggcgagaccgccgaggccacgcggttgaagcggaccgcccgcaggcggtacactcgccggaagaacaggatctgctacctgcaggagatcttctcaaacgagatggccaaggtggacgactccttcttccaccggctggaggagtcgttcctggtcgaggaggacaagaagcacgagcggcaccccatcttcggcaacatcgtggacgaggtggcctaccacgagaagtaccccaccatctaccacctgcggaagaagctggtggactccactgacaaggccgacctgcggctgatctacctggccctggcccacatgatcaagttcaggggccacttcctgatcgagggcgatctgaaccccgataacagcgatgtggacaagctgttcatccagctggtgcagacctacaaccagctgttcgaggagaatcccatcaacgccagcggcgtggacgccaaggccatcctgagcgccaggctgagcaagagccggcgcttagagaacctgatcgcccagctgcccggggagaagaagaacgggctgttcgggaacctgatcgccctcagcctgggcctgacccccaacttcaagtcaaacttcgacttggccgaggacgccaagttgcagttgtccaaggacacctacgacgacgacttggacaacctcctggcacagatcggggatcagtacgcagacctgtttctggccgccaagaacttgagcgacgccatcctgctgtcagacatcctgcgggtgaacaccgagatcaccaaggcccccctgtccgcgtccatgatcaagcggtacgacgagcaccaccaggacctgaccctgctgaaggccctggtgaggcagcagttgcccgagaagtacaaggagatcttcttcgatcagagcaagaacggctacgctggctacatcgacggcggggccagccaggaggagttctacaagttcatcaagcccatcctggagaagatggacggcaccgaggagctgctcgtgaagctgaaccgggaggacctgctgcggaagcagcggaccttcgacaacggttcaatcccccaccagatccacctgggggagctgcacgccatcttgcggcggcaggaggatttctatccttttctgaaggataaccgggagaagatcgagaagatcctcaccttccggatcccctactacgtggggcccctcgctcgcggcaactcccggttcgcctggatgaccaggaagtccgaggagaccatcaccccctggaacttcgaggaggtggtggacaagggcgcctccgctcagagcttcatcgagcggatgaccaacttcgacaagaacctgcccaacgagaaggtgctgccaaagcacagcttgttgtacgagtacttcaccgtctataacgagctgaccaaggtgaagtacgtgaccgaggggatgcggaagcccgcattcctctcgggcgagcagaagaaggctatcgtggacctgctgttcaagaccaaccggaaggtcaccgtgaagcagctgaaggaggactacttcaagaagatcgagtgcttcgattccgtcgagattagcggagtcgaggaccgatttaacgcttcactgggcacctaccacgacctgctgaagatcatcaaggacaaggacttcctggacaacgaggagaacgaggacatcttggaggacatcgtgctgaccctgaccttgttcgaggaccgggagatgatcgaggagcgcttgaagacctacgctcacctgttcgacgacaaggtgatgaagcagctgaagcggcgccggtacactggctggggccgcttgagcaggaagctgatcaacggcatccgcgacaagcagagcggcaagaccatcctggacttcctgaagtccgatggttttgccaaccgcaacttcatgcagctgatccacgacgacagcctgaccttcaaggaggacatccagaaggctcaggtgtcagggcagggagacagcctgcatgagcacatcgctaacctggctgggagccccgccatcaagaaggggatcctgcagaccgtgaaggtcgtggatgagctggtgaaggtgatgggccggcacaagcctgagaacatcgtgatcgaaatggctcgggagaaccagaccacccagaagggtcagaagaacagccgggagcggatgaagcggatcgaggagggcatcaaggagctcggctcacagatcctgaaggagcaccccgtggagaacacgcagctccagaacgagaagctgtatctgtactacctgcagaatggccgcgacatgtatgtcgaccaggagctggacatcaaccggctgtccgactacgatgtggaccacatcgtgccccagagcttcctgaaggacgatagcatcgacaacaaggtgctgacgcggtccgacaagaatcggggcaagagcgacaacgttcccagcgaggaggtggtgaagaagatgaagaactactggcggcagctgctgaacgccaagctgatcacccagcggaagttcgacaacttgaccaaggccgagcggggcggcctgagcgagctggacaaggccggcttcatcaagcggcagctggtggagaccaggcagatcaccaagcacgtcgctcagatcttggacagcaggatgaacaccaagtacgacgaaaacgataagctgatcagggaggtgaaggtgatcacgctgaagtcaaagctggtgagcgacttcaggaaggacttccagttctacaaggtgagggagatcaacaactaccaccacgcccacgacgcttacctgaacgccgtggtgggcaccgccttgatcaagaagtaccccaagctggagtccgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccttggccaacggtgagatcaggaagcggcctctgatcgagaccaacggcgagaccggggagatcgtgtgggacaagggccgggacttcgccaccgtgaggaaggtgctgagcatgccccaggtcaacatcgtgaagaagaccgaggtgcagaccgggggcttctcaaaggagtcaatcctgcctaagcggaactccgacaagctgatcgccaggaagaaggactgggatcccaagaagtacggcggcttcgactcccccaccgtggcgtacagcgtgctggtcgtggccaaggtggagaagggcaagtcgaagaagctgaagagcgtgaaggagctcttgggcatcaccatcatggagcggagtagcttcgagaagaatcccatcgattttctcgaggctaagggctacaaggaggtcaagaaggacctcatcatcaagctccccaagtacagcttgttcgagctggagaacgggaggaagcgcatgctggcctccgccggcgagctgcagaagggcaacgagctcgccctgccctccaagtacgtgaacttcctgtacttggccagccactacgagaagctgaagggcagccctgaggacaacgagcagaagcagttgttcgtggagcagcacaagcactacctggacgagatcatcgagcagatctcagagttcagcaagcgggtcatcctggccgacgcgaacctggacaaggtgctgtccgcctacaacaagcaccgggacaagcccatccgggagcaggccgagaacatcatccacctgttcaccctgaccaacctgggcgctcccgccgctttcaagtacttcgacaccaccatcgacaggaagcggtacacctccaccaaggaggtgttggacgccaccttgatccaccagagcatcaccgggctgtacgagacccggatcgacctgagccagctgggcggcgacggcgggggcagccccaagaagaagcggaaggtg

SEQ ID NO: 956 - RNA, 4137 nt, synthetic construct
  source 1..4137: mol_type = other RNA
atggataagaagtacagcatcgggctggacatcggcaccaacagcgtgggctgggccgtgatcaccgacgagtacaaggtgcccagcaagaagttcaaggtgctgggcaacaccgaccggcactccatcaagaagaacttgatcggagccctgctgttcgacagcggggagaccgccgaggccacccggctgaagcggaccgcccggcggcggtacacccggcggaagaaccggatctgctacctgcaggagatcttcagcaacgagatggccaaggtggacgactccttcttccaccggctggaggagtcattcctggtcgaggaggacaagaagcacgagcggcaccccatcttcggcaacatcgtggatgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgataacagcgacgtggacaagctgttcatccagctggtgcagacctacaaccagctgttcgaggagaaccccatcaacgctagcggggtggacgccaaggccatcctgagcgccaggctgagcaagagccgcaggttggagaacctgatcgcccagctgcccggggagaagaagaacggcctgttcggcaatctgatcgccctctccctgggcctgacccccaacttcaagtccaacttcgacctggccgaggacgccaagctgcagctgtccaaggacacctacgacgacgacctggacaacctgctggcccagatcggcgaccagtacgccgatctgttcctggccgccaagaacctgtccgacgccatcctgctgtcagacatcctgcgggtgaacaccgagatcaccaaggcccccctgagcgctagcatgatcaagcggtacgacgagcaccaccaggacctgaccctgctgaaggccctggtgcggcagcagctgcccgagaagtacaaggagatcttcttcgaccagtccaagaacggatacgccggctacatcgacgggggcgccagccaggaggagttctacaagttcatcaagcccatcctggagaagatggacggcaccgaggagctgctcgtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcttgaccttccggatcccctactacgtggggcccctggcccggggcaactcccggtttgcctggatgaccaggaagagcgaggagaccatcaccccctggaacttcgaggaggtggtggacaagggggcctccgcccagagcttcatcgagcggatgaccaacttcgacaagaacctgcccaacgagaaggtgctgccaaagcactccctgctgtacgagtacttcaccgtgtacaacgagctgaccaaggtgaagtacgtgacagagggaatgcggaagcccgccttcctgagcggcgagcagaagaaggccatcgtggacctgctgttcaagaccaaccggaaggtcaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggataacgaggagaacgaggacatcctggaggacatcgtgttgaccctgaccctgttcgaggacagggagatgatcgaggagcggttgaagacctacgcccacctgttcgatgacaaggtgatgaagcagctgaagcggaggcggtacaccggctgggggcggctgagccggaagctgatcaacgggatccgggacaagcagtccggcaagaccatcctggacttcctgaagagcgacggcttcgccaaccggaacttcatgcagctgatccacgacgacagcctgaccttcaaggaggacatccagaaggcccaggtgtcaggccagggcgacagcctgcacgagcacatcgccaacctggccggcagccccgccatcaagaagggcatcttgcagaccgtgaaggtcgtggacgagctggtgaaggtgatgggccggcacaagcccgagaacatcgtgatcgagatggcccgggagaaccagaccacccagaagggccagaagaacagcagggagcggatgaagcggatcgaggaggggatcaaggagctgggctcccagatcctgaaggagcaccccgtcgagaacacccagctgcagaacgagaagctgtacctgtactacctgcagaacgggagggacatgtacgtggaccaggagctggacatcaaccggctcagcgactacgacgtggaccacatcgtgccccagtccttcctgaaggacgacagcatcgataacaaggtgctgacccggagcgataagaaccggggcaagagcgataacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggactccaggatgaacaccaagtacgacgagaacgacaagctgatccgggaggtgaaggtgatcaccctgaagagcaagctggtgtcagacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggggagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcgggttcagcaaggagtccatcctgcccaagcggaacagcgacaagctgatcgctcggaagaaggattgggaccccaagaagtacggcggcttcgacagccccaccgtggcctacagcgtgctggtggtggccaaggtggagaagggcaagtcaaagaagctgaagtcagtgaaggagctgctgggcatcaccatcatggagcggagcagcttcgagaagaaccccatcgacttcttggaggccaagggctacaaggaggtgaagaaggatctgatcatcaagctgcccaagtactcactgttcgagctggagaacggccggaagcggatgctggcctccgccggcgagctgcagaagggaaacgagctggccctgcccagcaagtacgtgaacttcttgtacctggccagccactacgagaagttgaagggatcacccgaggacaacgagcagaagcagctgttcgtggagcagcacaagcactacctggacgagatcatcgagcagatctcagagttcagcaagcgggtgatcctggccgacgccaacctggacaaggtgctcagcgcctacaacaagcaccgggacaagcccatccgggagcaggccgagaacatcatccacctgttcaccctgaccaacctgggcgcccccgccgctttcaagtacttcgacaccaccatcgaccggaagcggtacaccagcaccaaggaggtgctggacgccactctgatccaccagagcatcaccgggctgtacgagacccggatcgacctgagccagctgggcggcgacggcgggggcagccccaagaagaagcggaaggtg

SEQ ID NO: 957 - RNA, 4137 nt, synthetic construct
  source 1..4137: mol_type = other RNA
atggataagaaatatagcatcggcctggatatcggcacaaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgataactccgacgtggacaagctgttcatccagctggtgcagacctacaaccagctgttcgaggagaaccccatcaacgcctcaggcgtggacgccaaggccatcttgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgatctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggggccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggatcgggagatgatcgaggagcggctgaagacctacgcccacctgttcgacgacaaggtgatgaagcagctgaagcggcggcggtacaccggctggggccggctgtcccggaagctgatcaacggcatccgggacaagcagagcggcaagaccatcctggacttcctgaagtccgacggattcgccaaccggaacttcatgcagctgatccacgacgacagcctgaccttcaaggaggacatccagaaggcccaggtgtcaggccagggcgacagcctgcacgagcacatcgccaacctggccggcagccccgccatcaagaagggcatcctgcagaccgtgaaggtggtggacgagctggtgaaggtgatggggcggcacaagcccgagaacatcgtgatcgagatggccagggagaaccagaccacccagaagggccagaagaacagccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggtccgacaagaaccggggcaagagcgacaacgtgccctccgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggggagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcgggttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagtcaaagaagctgaagagcgtgaaggagctgctgggcatcaccatcatggagcggagcagcttcgagaagaaccccatcgacttcttggaggccaagggctacaaggaggtgaagaaggacctgatcatcaagctgcccaagtactcactgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatctcagagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacggcgggggcagccccaagaagaagcggaaggtg

SEQ ID NO: 958 - RNA, 4137 nt, synthetic construct
  source 1..4137: mol_type = other RNA
atggataagaagtactccatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgataacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgatctgttcctggccgccaagaacctgtccgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagtccaagaatggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagtccttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgtcaggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggatatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagtcaggcaagaccatcctggacttcctgaagagcgacggcttcgccaaccggaacttcatgcagctgatccacgatgacagcctgaccttcaaggaggacatccagaaggcccaggtgagcggccagggcgacagcctgcacgagcacatcgccaacctggccggcagccccgccatcaagaagggcatcctgcagaccgtgaaggtggtggatgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagtcattcctgaaggacgacagcatcgacaacaaggtgctgacccggagcgacaagaaccggggcaagagcgacaacgtgccctccgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggataacgagcagaagcagctgttcgtggagcagcacaagcactacctggacgagatcatcgagcagatctcagagttcagcaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacggcggcggcagccccaagaagaagcggaaggtg

SEQ ID NO: 959 - RNA, 4137 nt, synthetic construct
  source 1..4137: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtg

SEQ ID NO: 960 - RNA, 4137 nt, synthetic construct
  source 1..4137: mol_type = other RNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggcggcggcagccccaagaagaagcggaaggtg

SEQ ID NO: 961 - RNA, 61 nt, synthetic construct
  source 1..61: mol_type = other RNA
tcccgcagtcggcgtccagcggctctgcttgttcgtgtgtgtgtcgttgcaggccttattc

SEQ ID NO: 962 - RNA, 183 nt, synthetic construct
  source 1..183: mol_type = other RNA
catcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaa

SEQ ID NO: 963 - RNA, 40 nt, synthetic construct
  source 1..40: mol_type = other RNA
aaaaaaaaaaaaaaaaaaaaaaaaaaaaaaacccccccca

SEQ ID NO: 964 - RNA, 60 nt, synthetic construct
  source 1..60: mol_type = other RNA
aaaaaaaaaaaaaaaaaaaaaaaaaaaaaaaaaaaaaaaaaaaaaaaaaaacccccccca

SEQ ID NO: 965 - RNA, 69 nt, synthetic construct
  source 1..69: mol_type = other RNA
aaaaaaaaaaaaaaaaaaaaaaaaaaaaaaaaaaaaaaaaaaaaaaaaaaaaaaaaaaaacccccccca

SEQ ID NO: 966 - RNA, 81 nt, synthetic construct
  source 1..81: mol_type = other RNA
aaaaaaaaaaaaaaaaaaaaaaaaaaaaaaaaaaaaaaaaaaaaaaaaaaaaaaaaaaaacccccccccccccccccccca

SEQ ID NO: 967 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaacccccccccccccccccccca

SEQ ID NO: 968 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 969 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 970 - RNA, 108 nt, synthetic construct
  source 1..108: mol_type = other RNA
aaaaaaaaaaaaaaaaaaaaaaaaaaaaaagcgaaaaaaaaaaaaaaaaaaaaaaaaaaaaaaccgaaaaaaaaaaaaaaaaaaaaaaaaaaaaaaaaaaaaaaaaaa

SEQ ID NO: 971 - RNA, 109 nt, synthetic construct
  source 1..109: mol_type = other RNA
aaaaaaaaaaaaaaaaaaaaaaaaaaaaaagcgaaaaaaaaaaaaaaaaaaaaaaaaaaaaaaccgaaaaaaaaaaaaaaaaaaaaaaaaaaaaaaaaaaaaaaaaaag

SEQ ID NO: 972 - RNA, 110 nt, synthetic construct
  source 1..110: mol_type = other RNA
aaaaaaaaaaaaaaaaaaaaaaaaaaaaaagcgaaaaaaaaaaaaaaaaaaaaaaaaaaaaaaccgaaaaaaaaaaaaaaaaaaaaaaaaaaaaaaaaaaaaaaaaaagg

SEQ ID NO: 973 - RNA, 20 nt, synthetic construct
  source 1..20: mol_type = other RNA
aaaggctgctgatgacacct

SEQ ID NO: 974 - protein, 4 aa, synthetic construct
RFHR